(19) United States
(12) Patent Application Publication
(10) Pub. No.: US 2026/0256924 A1
(43) Pub. Date: Sep. 3, 2026

(54) INDOCYANINE GREEN (ICG) MODIFICATION FOR TREATMENT OF LIVER CANCER

(71) Applicant: The Children's Medical Center Corporation, Boston, MA (US)

(72) Inventor: Khashayar Vakili, Watertown, MA (US)

(73) Assignee: The Children's Medical Center Corporation, Boston, MA (US)

(21) Appl. No.: 18/877,812

(22) PCT Filed: Jun. 23, 2023
(86) PCT No.: PCT/US2023/026034

§ 371 Date: Dec. 20, 2024

Related U.S. Application Data
(60) Provisional application No. 63/355,074, filed on Jun. 23, 2022.
(60) Provisional application No. 63/414,386, filed on Oct. 7, 2022.

(51) Int. Cl. A61K 47/55; A61K 47/54; A61K 51/04; A61P 35/00; C07D 209/60

(52) U.S. Cl. CPC A61K 47/55; A61K 47/545; A61K 51/0446; A61K 51/0497; A61P 35/00; C07D 209/60

(57) ABSTRACT

The present disclosure provides compounds of Formula (I) and (I′), which are derivatives of indocyanine green (ICG), and pharmaceutically acceptable salts, solvates, hydrates, polymorphs, co-crystals, tautomers, stereoisomers, and prodrugs thereof. The present disclosure also provides pharmaceutical compositions and kits comprising the compounds, or pharmaceutically acceptable salts, solvates, hydrates, polymorphs, co-crystals, tautomers, stereoisomers, or prodrugs thereof, and methods of diagnosing, treating, or preventing diseases by administering to a subject in need thereof the compounds, or pharmaceutically acceptable salts, solvates, hydrates, polymorphs, co-crystals, tautomers, stereoisomers, or prodrugs thereof, or pharmaceutical compositions thereof. The present disclosure also provides methods of preparation of compounds of Formula (I) and (I′).

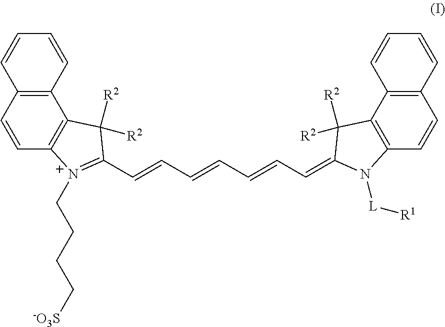

### RELATED APPLICATIONS

This application claims priority under 35 U.S.C. § 119(e) to U.S. Provisional Application, U.S. Ser. No. 63/355,074, filed Jun. 23, 2022, and U.S. Provisional Application, U.S. Ser. No. 63/414,386, filed Oct. 7, 2022, which are incorporated herein by reference in their entireties.

### BACKGROUND

Compounds containing radioisotopes allow for the collection of information regarding the functioning of specific organs, biological processes, and the state or diagnosis of various disease states in a patient. Radioisotope-containing compounds can also be used for treating diseases, such as cancer. However, many radioisotope-containing compounds distribute broadly throughout the patient's body, limiting the ability to collect localized information regarding disease states. Additionally, radioisotope-containing compounds that distribute broadly are limited in ability to treat localized disease processes, such as cancerous tumors. Accordingly, there is a need for radioisotope-containing compounds that are able to localize selectively to various tissue types, enhancing the ability to diagnose and treat diseases.

Liver cancer is highly lethal due to the lack of effective therapeutic agents. The most common types of liver cancer are epithelial in origin and include hepatocellular carcinoma (HCC), hepatoblastoma (HB), and fibrolamellar carcinoma (FLC). Even though the oncogenic driver and cellular programming is different between these cancer types, they all express liver-specific organic acid solute transporters (OATP). These transporters are uniquely expressed by hepatocytes and are responsible for the uptake of bile salts and bilirubin from the blood. These transporters are also responsible for the uptake of indocyanine green (ICG), an FDA-approved near-infrared fluorescent dye that is used clinically to assess liver function and tissue perfusion. Hepatocytes import ICG via the OATP transporters and excrete it into the biliary system via the MDR3 efflux transporter. Since tumor cells express the OATP transporters but do not have a biliary system, ICG accumulates in tumor tissue but is rapidly cleared from normal hepatocytes. This difference in the local pharmacokinetics of ICG is used intraoperatively to localize liver tumors. In clinical practice, ICG is administered intravenously 48-72 h prior to surgery; and tumor tissue, which accumulates ICG, is detected intraoperatively using near-infrared videography.

### SUMMARY

Hepatocyte-specific positron-emission tomography (PET) tracers have been developed by labeling indocyanine green (ICG) with radioisotopes (e.g., ¹⁸F, ⁶⁴Cu, ⁶⁷Cu). These tracers demonstrate liver-specific uptake and suitable in vivo kinetics in wild-type mice, indicating that labeling ICG with radioisotopes does not significantly alter its ability to accumulate in hepatocytes. Given that ICG uptake by liver cancer has been validated preclinically and clinically, ICG may serve as a vector for the delivery of diagnostic and therapeutic radionuclides to liver cancer cells.

Accordingly, in one aspect, the present disclosure provides compounds of Formula I:

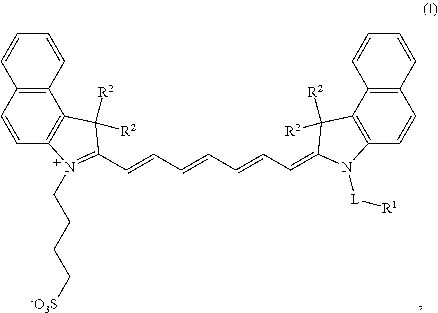

and pharmaceutically acceptable salts, solvates, hydrates, polymorphs, co-crystals, tautomers, stereoisomers, and prodrugs thereof, wherein the moieties and variables included in Formula I are as defined herein. The provided compounds may be able to localize to liver cancer cells.

In another aspect, the present disclosure provides compounds of Formula I′:

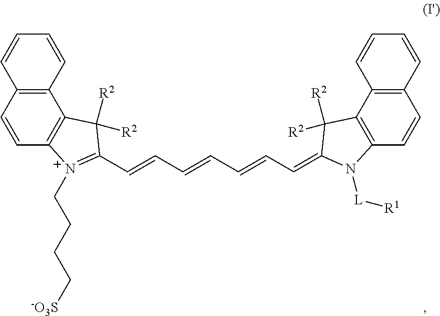

and pharmaceutically acceptable salts, solvates, hydrates, polymorphs, co-crystals, tautomers, stereoisomers, and prodrugs thereof, wherein the moieties and variables included in Formula I′ are as defined herein.

In another aspect, the present disclosure provides pharmaceutical compositions comprising a provided compound and a pharmaceutically acceptable excipient.

In another aspect, the present disclosure provides kits comprising a provided compound or pharmaceutical composition and instructions for using the provided compound or pharmaceutical composition.

In another aspect, the present disclosure provides methods for diagnosing diseases in a subject in need thereof, the methods comprising administering to the subject an effective amount of a provided compound or pharmaceutical composition.

In another aspect, the present disclosure provides methods for treating diseases in a subject in need thereof, the methods comprising administering to the subject an effective amount of a provided compound or pharmaceutical composition.

In another aspect, the present disclosure provides methods for preventing diseases in a subject in need thereof, the methods comprising administering to the subject an effective amount of a provided compound or pharmaceutical composition.

In certain embodiments, the disease is cancer.

In another aspect, the present disclosure provides methods for inhibiting cell proliferation or promoting apoptosis in a subject in need thereof or in a cell, tissue, or biological sample, comprising administering to the subject or contacting the cell, tissue, or biological sample with an effective amount of a provided compound or pharmaceutical composition, wherein the cell, tissue, or biological sample is in vivo.

In another aspect, the present disclosure provides methods for inhibiting cell proliferation or promoting apoptosis in a subject in need thereof or in a cell, tissue, or biological sample, comprising administering to the subject or contacting the cell, tissue, or biological sample with an effective amount of a provided compound or pharmaceutical composition, wherein the cell, tissue, or biological sample is in vitro.

The details of one or more embodiments of the disclosure are set forth herein. Other features, objects, and advantages of the disclosure will be apparent from the Detailed Description, Examples, Figures, and Claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1B show synthetic schemes for [¹⁸F]SFB (FIG. 1A) and [¹⁸F]FPyTFP (FIG. 1B) and their reactions with ICG-amine.

FIGS. 2A-2D show HPLC traces of the reaction to form [¹⁸F]SFB-ICG (C18-2). FIG. 2A shows after 15 minutes. FIG. 2B shows after 30 minutes. FIG. 2C shows after 45 min. FIG. 2D shows after 60 minutes.

FIGS. 3A-3D show HPLC traces of the unpurified [¹⁸F]SFB-ICG Reaction (Polar-RP). FIG. 3A shows [¹⁸F]SFB prior to the reaction. FIG. 3B shows [¹⁸F]SFB and desired product after 30 minutes (hydrolysis). FIG. 3C shows the desired product after 15 minutes. FIG. 3D shows the desired product after 45 minutes. The column utilized was Polar RP. The mobile phase utilized Acetonitrile: Water (0.1% TFA) (20:80), Flow Rate: 1 mL/minute.

FIG. 4 shows HPLC traces of the reaction to form [¹⁸F]SFB-ICG: Polar-RP, using the Semi-Prep Column: Polar RP-C18. The mobile phase utilized Ethanol: PBS (1×) (10:90), with a flow rate of 5 mL/minute.

FIGS. 5A-5B show HPLC traces of collected fractions of the reaction to form [¹⁸F]SFB-ICG. FIG. 5A shows the fractions collected between 7 and 8 minutes. FIG. 5B shows the fractions collected between 9 and 10 minutes. The analytical column used was Hydro RP-C18. The mobile phase utilized Acetonitrile: Water (TFA 0.1%) (20:80) at a flow rate of 1 mL/minute.

FIGS. 6A-6B show reference standards of FBA and SFB upon using a Hydro RP-C18 column, Acetonitrile: Water (TFA 0.1%) (35:65) for the mobile phase with a flow through rate of 1 mL/minute. FIG. 6A shows the reference standard for FBA; FIG. 6B shows the reference standard for SFB.

FIGS. 7A-7D show HPLC traces of the reaction to form [¹⁸F]SFB-ICG, unpurified and cold. FIG. 7A shows after 5 minutes, with both FBA and SFB present, as well as possible product. FIG. 7B shows after 30 minutes. FIG. 7C shows after 15 minutes. FIG. 7D shows after 45 minutes.

FIGS. 8A-8B show HPLC traces of the reaction to form [¹⁸F]SFB-ICG. FIG. 8A shows [¹⁸F]SFB-ICG crude. FIG. 8B shows [¹⁸F]SFB-ICG semi-prep. The total activity (EOS) was 17 mCi, 2 mCi/mL with a RCP of 38%.

FIGS. 9A-9B show HPLC traces of [¹⁸F]SFB-ICG purifications with varying mobile phases. FIG. 9A shows [¹⁸F]SFB-ICG as a purified product from semi-prep column; Polar-RP-C18, with a mobile phase utilizing Ethanol: PBS (1×) (10:90). The flow through rate was 5 mL/minute. FIG. 9B shows ¹⁸F]SFB-ICG as a purified product from semi-prep column; Polar-RP-C18, with a mobile phase utilizing Ethanol: PBS (1×) (8:92), with a flow through rate of 5 mL/minute.

FIGS. 10A-10B show HPLC traces of [¹⁸F]SFB-ICG (FIG. 10A) and [¹⁸F]FBA (FIG. 10B). Both were purification products from the Semi-prep Column Hydro RP-C18, with a mobile phase utilizing Ethanol: Sodium Dihydrogen phosphate (25 mM) (10:90), with a flow through rate of 5 mL/minute.

FIGS. 11A-11B show HPLC traces of [¹⁸F]SFB-ICG stability at 1 hour, and 2 hours. FIG. 11A shows [¹⁸F]SFB-ICG at 1 hour, with a 95% RCP. FIG. 11B shows [¹⁸F]SFB-ICG at 2 hours, with a 96% RCP.

FIG. 12 shows the cold reaction using the HPLC method of XBD-C18 4.6×150 mm; Solv.A: 30% H2O; solv.B: 70% MeCN; flow: 1.5 ml/minute; λ=254 nm.

FIG. 13 shows the cold reaction spiked using the HPLC method: XBD-C18 4.6×150 mm; Solv.A: 30% H2O; solv.B: 70% MeCN; flow: 1.5 ml/minute; λ=254 nm.

FIGS. 14A-14D show [¹⁸F]ICG-A/B reaction-chromatograms. FIG. 14A shows a table delineating the temperatures and their respective percent conversions. FIG. 14B shows the radioactive traces at RT (15) and RT (30)+50 (15). FIG. 14C shows the radioactive trace at RT 30+90 (15). FIG. 14D shows the UV trace at RT (30)+50 (30)+90 (15).

FIG. 15 shows the ICG-DBCO reference upon the use of the HPLC method: XBD-C18 4.6×150 mm; Solv.A: 30% H2O; solv.B: 70% MeCN; flow: 1.5 ml/minute; λ=254 nm.

FIG. 16 shows the reference 1-azido-2-fluoroethane upon use of the HPLC method: XBD-C18 4.6×150 mm; Solv.A: 30% H2O; solv.B: 70% MeCN; flow: 1.5 ml/minute; 2=254 nm.

FIGS. 17A-17D show PET/CT images of a representative wild type mouse.

FIGS. 18A-18B show Time Activity Curves for ¹⁸F-SFB-ICG-All Organs of Interest. FIG. 18A shows the activity concentration in Standardized Uptake Value (SUV). FIG. 18B shows the activity uptake in percentage Injected Dose (% ID) for fully segmented organs. For large intestines and cecum, PET signal was not observed until the 4 hour time point, and thus these organs were not segmented (nor plotted) prior to that timepoint.

FIGS. 19A-19B show Time Activity Curves for ¹⁸F-SFB-ICG-Liver. FIG. 19A shows the activity concentration in Standardized Uptake Value (SUV). FIG. 19B shows the activity uptake in percentage Injected Dose (% ID) for fully segmented organ.

FIGS. 20A-20B show Time Activity Curves for ¹⁸F-SFB-ICG-Gallbladder. FIG. 20A shows the activity concentration in Standardized Uptake Value (SUV). FIG. 20B shows the activity uptake in percentage Injected Dose (% ID) for fully segmented organ.

FIGS. 21A-21B show Time Activity Curves for ¹⁸F-SFB-ICG-Small Intestine. FIG. 21A shows the activity concentration in Standardized Uptake Value (SUV). FIG. 21B shows the activity uptake in percentage Injected Dose (% ID) for fully segmented organ.

FIGS. 22A-22B show Time Activity Curves for ¹⁸F-SFB-ICG-Heart. FIG. 22A shows the activity concentration in Standardized Uptake Value (SUV). FIG. 22B shows the activity uptake in percentage Injected Dose (% ID) for fully segmented organ.

FIGS. 23A-23B show Time Activity Curves for ¹⁸F-SFB-ICG-Spleen and Lungs. FIG. 23A shows the activity concentration in Standardized Uptake Value (SUV) in the spleen. FIG. 23B shows the activity concentration in Standardized Uptake Value (SUV) in the lungs.

FIGS. 24A-24B show Gamma Counting for ¹⁸F-SFB-ICG-Whole Blood. FIG. 24A shows the activity concentration in Standardized Uptake Value (SUV). FIG. 24B shows the activity concentration (activity uptake) in percentage Injected Dose per gram (% ID/g) for whole blood.

FIGS. 25A-25D show ¹⁸F-SFB-ICG distribution in group 1a mice at 0 (FIG. 25A), 15 (FIG. 25B), 30 (FIG. 25C), and 45 minutes (FIG. 25D) post-tracer injection (IV), as determined through whole body PET/CT at the specified time point.

FIGS. 26A-26D show ¹⁸F-SFB-ICG distribution in group 1b mice at 0 (FIG. 26A), 15 (FIG. 26B), 30 (FIG. 26C), and 45 minutes (FIG. 26D) post-tracer injection (IV), as determined through whole body PET/CT at the specified time point. By 5 minutes, the majority of the tracer is localized to the liver and by 45 minutes there is evidence of excretion of [¹⁸F]ICG into the small intestine (arrow).

FIGS. 27A-27D show ¹⁸F-SFB-ICG distribution in group 1a mice, 15 minute frame at 45 minutes post-tracer injection, as determined by whole body PET/CT at 45-60 minutes post tracer-injection (IV).

FIGS. 28A-28D show ¹⁸F-SFB-ICG distribution in group 1b mice, 15 minute frame at 45 minutes post-tracer injection, as determined by whole body PET/CT at 45-60 minutes post tracer-injection (IV).

FIGS. 29A-29D show ¹⁸F-SFB-ICG distribution in group 1a mice, 15 minute frame at 4 hours post-tracer injection, as determined by whole body PET/CT at 4 hours post tracer-injection (IV). The most intense signal is present within the lumen of the intestines.

FIGS. 30A-30D show ¹⁸F-SFB-ICG distribution in group 1b mice, 15 minute frame at 4 hours post-tracer injection, as determined by whole body PET/CT at 4 hours post tracer-injection (IV). The most intense signal is present within the lumen of the intestines.

FIGS. 31A-31D show gross and microscopic ICG localization in FLC. The patient was administered intravenous ICG 48 hours prior to surgery and the white fluorescent areas correspond to ICG accumulation. FIG. 31A shows that T denotes tumor in the liver and NL denotes normal liver. There is clear demarcation between the tumor and normal liver. FIG. 31B shows that a black arrow points to an exophytic portion of the tumor extending from the liver. White arrow points to a lymph node outside of the liver and in the porta hepatis, demonstrating accumulation of ICG in metastatic tumor bed. FIG. 31C shows a fluorescent microscopic image (@789 nm excitation wavelength) of unstained metastatic FLC section demonstrating fluorescence (white) within tumor cells. FIG. 31D shows a control sample of processed tumor section in the absence of ICG demonstrating lack of fluorescence.

FIG. 32 shows in vivo fluorescence imaging following ICG administration. Implanted Huh7 cells demonstrate uptake at 0.5 mg/kg dose. Other areas of high signal represents concentration within the intestinal lumen. Control mouse without tumor is shown on the left side.

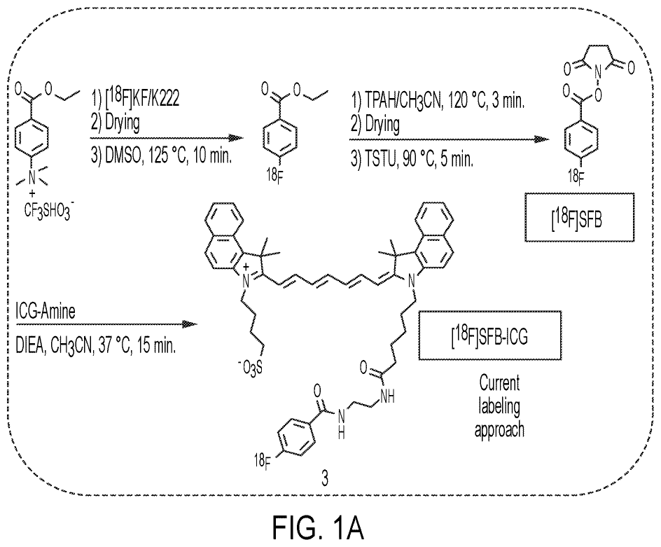

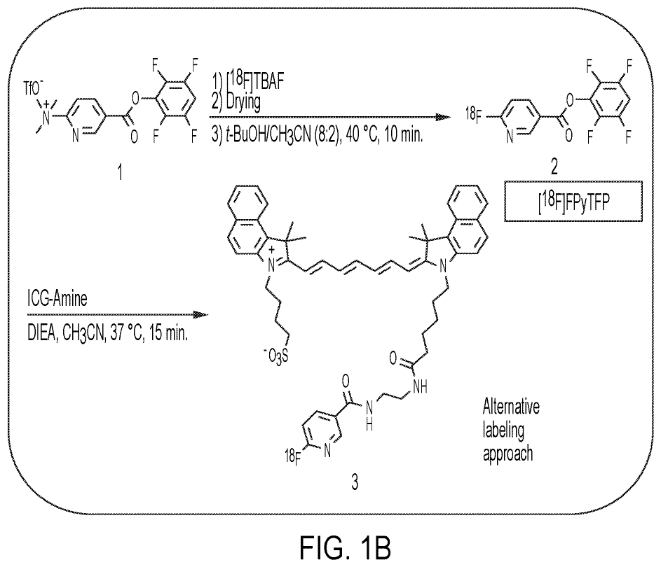

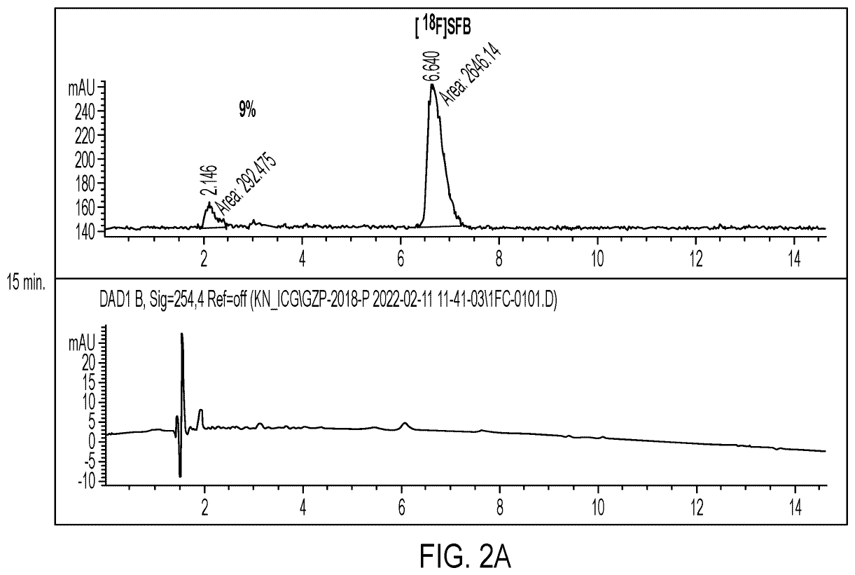

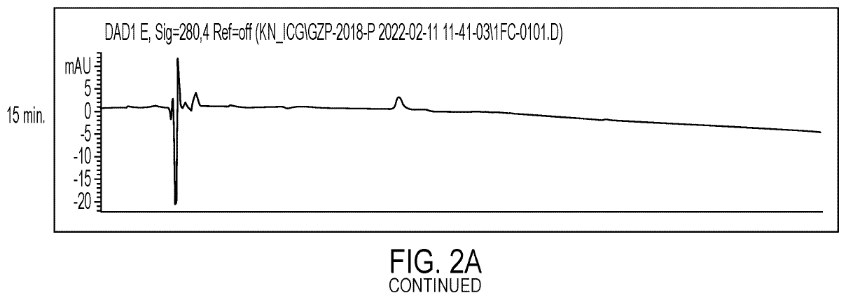

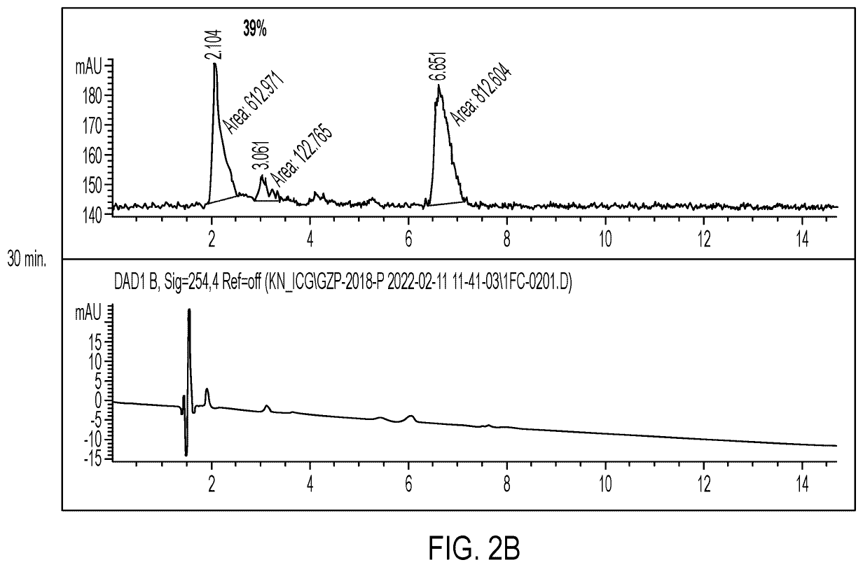

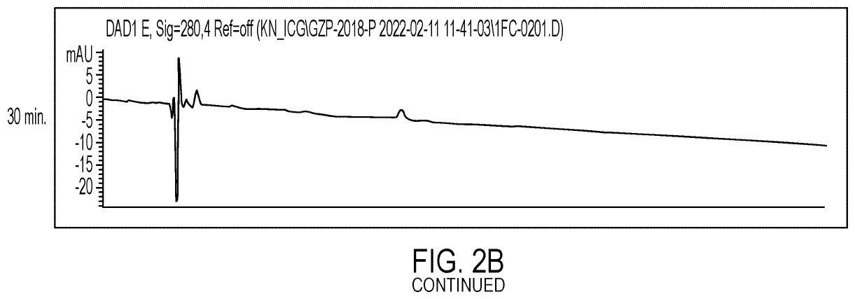

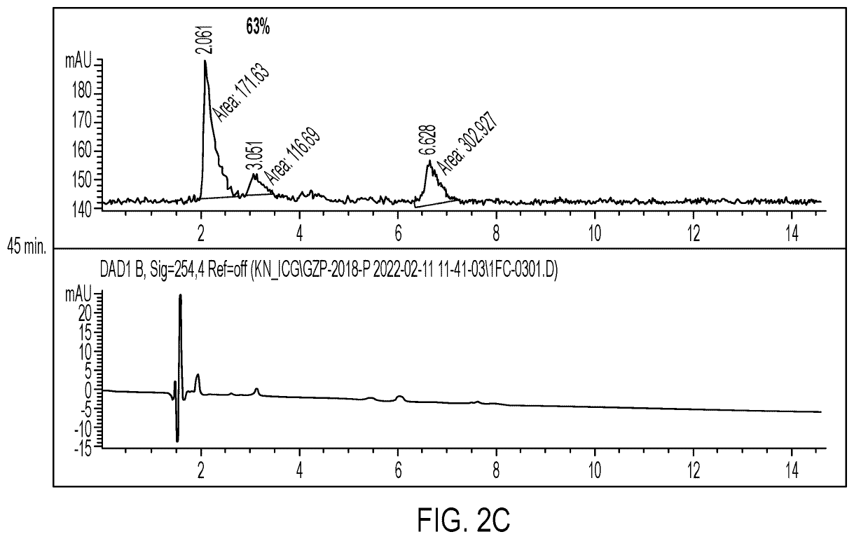

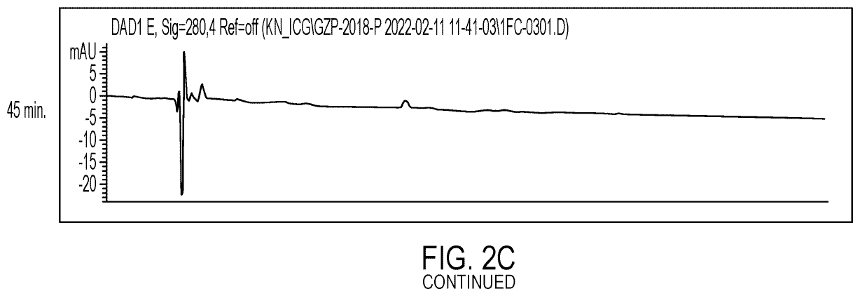

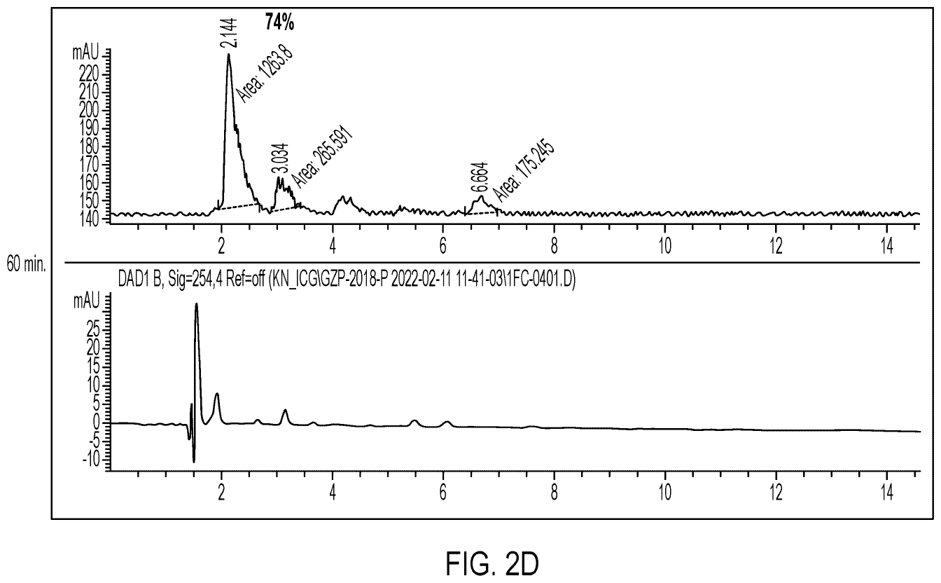

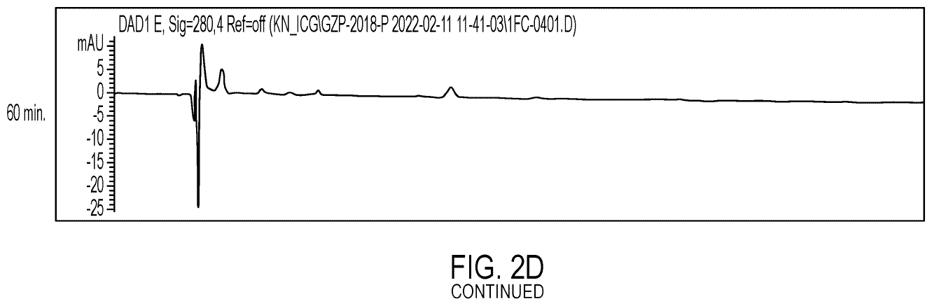

### Definitions

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The disclosure additionally encompasses compounds as individual isomers substantially free of other isomers, and alternatively, as mixtures of various isomers.

In a formula, [symbol] is a single bond where the stereochemistry of the moieties immediately attached thereto is not specified, - - - is absent or a single bond, and [symbol] or [symbol] is a single or double bond.

Unless otherwise provided, a formula depicted herein includes compounds that do not include isotopically enriched atoms, and also includes compounds that include isotopically enriched atoms. Compounds that include isotopically enriched atoms may be useful as, for example, analytical tools, and/or probes in biological assays.

When a range of values (“range”) is listed, it is intended to encompass each value and subrange within the range. A range is inclusive of the values at the two ends of the range unless otherwise provided. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C2-6 alkyl”). Examples of C1-6 alkyl groups include methyl (C1), ethyl (C2), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃(Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 12 carbon atoms (“C₁₋₁₂ haloalkyl”). In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are replaced with fluoro to provide a perfluoroalkyl group. In some embodiments, all of the haloalkyl hydrogen atoms are replaced with chloro to provide a “perchloroalkyl” group. Examples of haloalkyl groups include —CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl” or “C₁₋₂₀ heteroalkyl”). In certain embodiments, a heteroalkyl group refers to a saturated group having from 1 to 12 carbon atoms and 1 or more heteroatoms within the parent chain (“heteroC₁₋₁₂ alkyl”). In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds) (“C₁₋₂₀ alkenyl”). In some embodiments, an alkenyl group is ═CH₂. In some embodiments, an alkenyl group has 2 to 20 carbon atoms (“C₂₋₂₀ alkenyl”). In some embodiments, an alkenyl group is ═CH₂. In some embodiments, an alkenyl group has 2 to 12 carbon atoms (“C₂₋₁₂ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or) may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 20 carbon atoms, at least one double bond, and 1 or more heteroatoms within the parent chain (“heteroC₂₋₂₀ alkenyl” or “C₂₋₂₀ heteroalkenyl”). In certain embodiments, a heteroalkenyl group refers to a group having from 2 to 12 carbon atoms, at least one double bond, and 1 or more heteroatoms within the parent chain (“heteroC₂₋₁₂ alkenyl”). In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group is ≡CH. In some embodiments, an alkynyl group has 2 to 20 carbon atoms (“C₂₋₂₀ alkynyl”). In some embodiments, an alkynyl group has 2 to 12 carbon atoms (“C₂₋₁₂ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 20 carbon atoms, at least one triple bond, and 1 or more heteroatoms within the parent chain (“heteroC₂₋₂₀ alkynyl” or “C₂₋₂₀ heteralkynyl”). In certain embodiments, a heteroalkynyl group refers to a group having from 2 to 12 carbon atoms, at least one triple bond, and 1 or more heteroatoms within the parent chain (“heteroC₂₋₁₂ alkynyl”). In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂-alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (Cs), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl. In certain embodiments, the carbocyclyl is substituted or unsubstituted, 3- to 8-membered, and monocyclic. In certain embodiments, the carbocyclyl is substituted or unsubstituted, 5- to 14-membered, and bicyclic. In certain embodiments, the carbocyclyl is substituted or unsubstituted, 6- to 14-membered, and tricyclic.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continues to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 8-membered, monocyclic heterocyclyl, wherein 1, 2, or 3 atoms in the heterocyclic ring system are independently oxygen, nitrogen, or sulfur, as valency permits. In certain embodiments, the heterocyclyl is substituted or unsubstituted, 5- to 14-membered, bicyclic heterocyclyl, wherein 1, 2, 3, or 4 atoms in the heterocyclic ring system are independently oxygen, nitrogen, or sulfur, as valency permits. In certain embodiments, the heterocyclyl is substituted or unsubstituted, 6- to 14-membered, tricyclic heterocyclyl, wherein 1, 2, 3, or 4 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include aziridinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl,” e.g., unsubstituted phenyl (“Ph”)) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continues to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, i.e., the moiety only contains single bonds.

Affixing the suffix “ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not intended to be limited in any manner by the exemplary substituents described herein.

In certain embodiments, each carbon atom of a moiety described herein is independently unsubstituted or substituted as valency permits (e.g., at valency 4). A substituent on a carbon atom is a “carbon atom substituent.”

In certain embodiments, each nitrogen atom of a moiety described herein is independently unsubstituted or substituted as valency permits (e.g., at valency 3 or 5). A substituent on a nitrogen atom is a “nitrogen atom substituent.”

In certain embodiments, each oxygen atom of a moiety described herein is independently unsubstituted or substituted as valency permits (e.g., at valency 2). A substituent on an oxygen atom is a “oxygen atom substituent.”

In certain embodiments, each sulfur atom of a moiety described herein is independently unsubstituted or substituted as valency permits (e.g., at valency 2, 4, or 6). A substituent on a sulfur atom is a “sulfur atom substituent.”

In certain embodiments, each carbon atom substituent is independently selected from the group consisting of halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR)R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR)₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(RC)₂, —P(RC)₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, 5-14 membered heteroaryl, ═O, ═S, ═NN(R^{bb})₂, ═NNR^{bb}C(═O)R^{aa}, ═NNR^{bb}C(═O)OR^{aa}, =NNRbbs (═O)₂R^{aa}, ═NR^{bb}, and ═NOR^{cc}, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd}.

Each instance of R^{aa} is independently selected from the group consisting of C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂-10alkynyl, C₃₋₁₀ carbocyclyl, 3-14 membered heterocyclyl, C₆₋₁₄ aryl, and 5-14 membered heteroaryl, or two R^{aa} are joined to form 3-14 membered heterocyclyl or 5-14 membered heteroaryl, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd}.

each instance of R^{bb} is independently selected from the group consisting of hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(RC)₂, —SO₂RC, —SO₂OR^{cc}, —SOR^{aa}, C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} are joined to form 3-14 membered heterocyclyl (e.g., monocyclic heterocyclyl, bicyclic heterocyclyl, tricyclic heterocyclyl) or 5-14 membered heteroaryl, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd};
each instance of R^{cc} is independently selected from the group consisting of hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} are joined to form 3-14 membered heterocyclyl or 5-14 membered heteroaryl, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd};

each instance of R^{dd} is independently selected from the group consisting of halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO2R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NRffSO2R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, ═O, and ═S, or two occurrences of R^{dd} are joined together with their intervening atoms to form an optionally substituted carbocyclyl, optionally substituted heterocyclyl, optionally substituted aryl, or optionally substituted heteroaryl, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg};
each instance of R^{ee} is independently selected from the group consisting of C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg};
each instance of R^{ff} is independently selected from the group consisting of hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, and 5-10 membered heteroaryl, or two R^{ff} are joined to form 3-10 membered heterocyclyl or 5-10 membered heteroaryl, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg};
each instance of R^{gg} is independently selected from the group consisting of halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂+X⁻, —NH₂ (C₁₋₆ alkyl)+X⁻, —NH₃⁺X⁻, —N(OC₁₋₆ alkyl) (C₁₋₆ alkyl), —N(OH)(C₁₋₆ alkyl), —NH (OH), —SH, —SC₁₋₆ alkyl, —SS(C₁₋₆ alkyl), C(═O)(C₁₋₆ alkyl), CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl) C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂₀C₁₋₆ alkyl, —OSO₂C₁₋₆ alkyl, —SOC₁₋₆ alkyl, —Si(C₁₋₆ alkyl)₃, —OSi(C₁₋₆ alkyl) 3-C(═S)N(C₁₋₆ alkyl)₂, C(═S) NH(C₁₋₆ alkyl), C(═S) NH₂, —C(═O) S (C₁₋₆ alkyl), —C(═S) SC₁₋₆ alkyl, —SC(═S) SC₁₋₆ alkyl, —P(═O) 2 (C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl, ═O, and ═S; and
X⁻ is a counterion.

In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₆ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, the carbon atom substituents are independently halogen, substituted (e.g., substituted with one or more halogen moieties) or unsubstituted C₁₋₆ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₆ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₆ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(RC)₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═O)SR^{aa}, —SC(═O)OR^{aa}, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(NR^{bb})₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(NR^{bb})₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “phosphino” refers to the group —P(R^{cc})₃, wherein R^{cc} is as defined herein. An exemplary phosphino group is triphenylphosphine.

The term “oxo” refers to the group ═O, and the term “thiooxo” refers to the group ═S.

In certain embodiments, a nitrogen atom of a moiety described herein is a primary, secondary, tertiary, or quaternary nitrogen atom. In certain embodiments, each nitrogen atom substituent is independently selected from the group consisting of nitrogen protecting groups, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(RC)₂, —SO₂R, —SO₂ORE, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, 5-14 membered heteroaryl, and ═O, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd}.

In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the nitrogen atom substituents are independently substituted (e.g., substituted with one or more halogen) or unsubstituted C₁₋₆ alkyl or a nitrogen protecting group.

Nitrogen protecting groups may also be referred to as “amino protecting groups”. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, each nitrogen protecting group is independently selected from the group consisting of —OH, —OR^{aa}, —N(RC)₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(RC)₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups.

For example, nitrogen protecting groups such as amide groups (e.g., —C(═O)R^{aa}) include formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamino) acetamide, 3-(p-hydroxyphenyl) propanamide, 3-(o-nitrophenyl) propanamide, 2-methyl-2-(0-nitrophenoxy) propanamide, 2-methyl-2-(o-phenylazophenoxy) propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include methyl carbamate, ethyl carbamate, 9-fluorenylmethyl carbamate (Fmoc), 9-(2-sulfo) fluorenylmethyl carbamate, 9-(2,7-dibromo) fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate, 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl (o-nitrophenyl) methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido) benzyl carbamate, 1,1-dimethyl-3-(N,N-dimethylcarboxamido) propyl carbamate, 1,1-dimethylpropynyl carbamate, di (2-pyridyl) methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium) benzyl carbamate, and 2,4,6-trimethylbenzyl carbamate.

Nitrogen protecting groups such as sulfonamide groups (e.g., —S(═O)₂R^{aa}) include p-toluenesulfonamide (Ts), benzenesulfonamide, 2,3,6, —trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl) ethoxy]methylamine (SEM), N-3-acetoxypropylamine, N-(1-isopropyl-4-nitro-2-oxo-3-pyrrolin-3-yl) amine, quaternary ammonium salts, N-benzylamine, N-di (4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl) mesityl]methyleneamine, N-(N′,N′-dimethylaminomethylene) amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl) amine, N-borane derivative, N-diphenylborinic acid derivative, N-[phenyl (pentaacylchromium- or tungsten) acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, a nitrogen protecting group is Bn, Boc, Cbz, Fmoc, trifluoroacetyl, triphenylmethyl, acetyl, or Ts.

In certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group. In certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the oxygen atom substituents are independently substituted (e.g., substituted with one or more halogen) or unsubstituted C₁₋₆ alkyl or an oxygen protecting group.

Oxygen protecting groups may also be referred to as “hydroxyl protecting groups”. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, each oxygen protecting group is independently selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, —P(═O)(NR^{bb})₂, methyl, methoxymethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy) methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl) xanthenyl, 9-(9-phenyl-10-oxo) anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl) ethyl carbonate (TMSEC), 2-(phenylsulfonyl)ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, an oxygen protecting group is silyl, TBDPS, TBDMS, TIPS, TES, TMS, MOM, THP, t-Bu, Bn, allyl, acetyl, pivaloyl, or benzoyl.

In certain embodiments, each of the sulfur atom substituents is independently substituted (e.g., substituted with one or more halogen) or unsubstituted C₁₋₆ alkyl, —C(═O)R^{aa}, —CO₂R^{aa}, —C(═O)N(R^{bb})₂, a sulfur protecting group, or ═O. In certain embodiments, each of the sulfur atom substituents is independently substituted (e.g., substituted with one or more halogen) or unsubstituted C₁₋₆ alkyl, —C(═O)R^{aa}, —CO₂R^{aa}, —C(═O)N(R^{bb})₂, a sulfur protecting group, or ═O, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, each of the sulfur atom substituents is independently substituted (e.g., substituted with one or more halogen) or unsubstituted C₁₋₆ alkyl or a sulfur protecting group.

Sulfur protecting groups may also be referred to as “thiol protecting groups”. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, each sulfur protecting group is independently selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, C(═NR^{bb})N(R^{bb})₂, —S(═O)R^{aa}, —SO₂R^{aa}, —Si(R^{aa})₃, —P(RC)₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂. In certain embodiments, a sulfur protecting group is acetamidomethyl, t-Bu, 3-nitro-2-pyridine sulfenyl, 2-pyridine-sulfenyl, or triphenylmethyl.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methanesulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(RC)₂, —OP(RC)₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties. In some embodiments, the leaving group is optionally substituted amino. In some embodiments, the leaving group is trisubstituted amino (e.g., —N(R^{bb})₃⁺X⁻, wherein R^{bb} is as defined herein and X is a counterion).

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

Compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers,” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “co-crystal” refers to a crystalline structure comprising at least two different components (e.g., a compound and an acid), wherein each of the components is independently an atom, ion, or molecule. In certain embodiments, none of the components is a solvent. In certain embodiments, at least one of the components is a solvent. A co-crystal of a compound and an acid is different from a salt formed from a compound and the acid. In the salt, a compound is complexed with the acid in a way that proton transfer (e.g., a complete proton transfer) from the acid to a compound easily occurs at room temperature. In the co-crystal, however, a compound is complexed with the acid in a way that proton transfer from the acid to a herein does not easily occur at room temperature. In certain embodiments, in the co-crystal, there is substantially no proton transfer from the acid to a compound. In certain embodiments, in the co-crystal, there is partial proton transfer from the acid to a compound. Co-crystals may be useful to improve the properties (e.g., solubility, stability, and ease of formulation) of a compound.

The term “prodrugs” refers to compounds that have cleavable groups and become by solvolysis or under physiological conditions the compounds described herein, which are pharmaceutically active in vivo. Such examples include choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgaard, H., Design of Prodrugs, pp. 7-9, 21-24, Elsevier, Amsterdam 1985). Prodrugs include, for example, esters prepared by reaction of the parent acid with a suitable alcohol, or amides prepared by reaction of the parent acid compound with a substituted or unsubstituted amine, or acid anhydrides, or mixed anhydrides.

The terms “pharmaceutical composition,” “composition,” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. A “patient” refers to a human subject in need of diagnosis or treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a pharmaceutical composition thereof, in or on a subject.

The terms “diagnosis, “diagnose,” and “diagnosing” refer to recognizing or identifying a disease described herein in a subject. In some embodiments, the subject has not previously been diagnosed with the disease. In other embodiments, the subject has previously been diagnosed with the disease. In certain embodiments, the subject is at a higher risk of developing the disease or at a higher risk of regression of the disease than an average healthy member of a population of subjects. In certain embodiments, diagnosis further comprises treatment or prevention.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay and/or prevent recurrence. The treatment may be therapeutic treatment (not including prevention or prophylactic treatment).

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population of subjects.

The terms “condition,” “disease,” and “disorder” are used interchangeably.

An “effective amount” of a compound refers to an amount sufficient to elicit the desired biological response. An effective amount of a compound may vary depending on such factors as the desired biological endpoint, the pharmacokinetics of the compound, the condition being treated, the mode of administration, and the age and health of the subject. In certain embodiments, the effective amount is a therapeutically effective amount. In certain embodiments, the effective amount is a prophylactically effective amount. In certain embodiments, the effective amount is the amount of the compound in a single dose. In certain embodiments, the effective amount is the combined amounts of the compound in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound is an amount sufficient to provide a therapeutic benefit in the treatment of a condition or to delay or minimize one or more signs and/or symptoms associated with the condition, or to diagnose a condition. In certain embodiments, the therapeutically effective amount is an amount that improves overall therapy, reduces or avoids symptoms, signs, or causes of the condition, and/or enhances the therapeutic efficacy of another therapeutic agent. In certain embodiments, a therapeutically effective amount is an amount sufficient for detection in the subject. In certain embodiments, a therapeutically effective amount is an amount sufficient for reducing the size of a tumor (e.g., reduction in size of a tumor by at least 5%, at least 10%, at least 20%, at least 30%, at least 40%, at least 50%, at least 60%, at least 70%, at least 80%, at least 90%, at least 95%, or at least 99%). In certain embodiments, a therapeutically effective amount is an amount sufficient for treating a disease. In certain embodiments, a therapeutically effective amount is an amount sufficient for reducing the size of a tumor and treating a disease.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”) and benign neoplasms.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. The cancer may be a solid tumor. The cancer may be a hematological malignancy. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ophthalmic cancer (e.g., intraocular melanoma, retinoblastoma); familiar hypereosinophilia; gall bladder cancer (e.g., gall bladder carcinoma); gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematological cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma, hepatic carcinoma); lung cancer (e.g., bronchogenic carcinoma, small cell lung cancer (SCLC), non-small cell lung cancer (NSCLC), adenocarcinoma of the lung, squamous carcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); cancer of the peritoneum; pinealoma; pituitary cancer; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer (e.g., salivary gland carcinoma); skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “immunotherapy” refers to a therapeutic agent that promotes the treatment of disease by inducing, enhancing, or suppressing an immune response. Immunotherapies designed to elicit or amplify an immune response are classified as activation immunotherapies, while immunotherapies that reduce or suppress are classified as suppression immunotherapies. Immunotherapies are typically, but not always, biotherapeutic agents. Numerous immunotherapies are used to treat cancer. These include, but are not limited to, monoclonal antibodies, adoptive cell transfer, cytokines, chemokines, vaccines, and small molecule inhibitors.

The term “liver disease” or “hepatic disease” refers to damage to or a disease of the liver. Non-limiting examples of liver disease include intrahepatic cholestasis (e.g., alagille syndrome, biliary liver cirrhosis), fatty liver (e.g., alcoholic fatty liver, Reye's syndrome), hepatic vein thrombosis, hepatolenticular degeneration (i.e., Wilson's disease), hepatomegaly, liver abscess (e.g., amebic liver abscess), liver cirrhosis (e.g., alcoholic, biliary, and experimental liver cirrhosis), alcoholic liver diseases (e.g., fatty liver, hepatitis, cirrhosis), parasitic liver disease (e.g., hepatic echinococcosis, fascioliasis, amebic liver abscess), jaundice (e.g., hemolytic, hepatocellular, cholestatic jaundice), cholestasis, portal hypertension, liver enlargement, ascites, hepatitis (e.g., alcoholic hepatitis, animal hepatitis, chronic hepatitis (e.g., autoimmune, hepatitis B, hepatitis C, hepatitis D, drug induced chronic hepatitis), toxic hepatitis, viral human hepatitis (e.g., hepatitis A, hepatitis B, hepatitis C, hepatitis D, hepatitis E), granulomatous hepatitis, secondary biliary cirrhosis, hepatic encephalopathy, varices, primary biliary cirrhosis, primary sclerosing cholangitis, hepatocellular adenoma, hemangiomas, bile stones, liver failure (e.g., hepatic encephalopathy, acute liver failure), angiomyolipoma, calcified liver metastases, cystic liver metastases, fibrolamellar hepatocarcinoma, hepatic adenoma, hepatoma, hepatic cysts (e.g., Simple cysts, Polycystic liver disease, hepatobiliary cystadenoma, choledochal cyst), mesenchymal tumors (mesenchymal hamartoma, infantile hemangioendothelioma, hemangioma, peliosis hepatis, lipomas, inflammatory pseudotumor), epithelial tumors (e.g., bile duct hamartoma, bile duct adenoma), focal nodular hyperplasia, nodular regenerative hyperplasia, hepatoblastoma, hepatocellular carcinoma, cholangiocarcinoma, cystadenocarcinoma, tumors of blood vessels, angiosarcoma, Karposi's sarcoma, hemangioendothelioma, embryonal sarcoma, fibrosarcoma, leiomyosarcoma, rhabdomyosarcoma, carcinosarcoma, teratoma, carcinoid, squamous carcinoma, primary lymphoma, peliosis hepatis, erythrohepatic porphyria, hepatic porphyria (e.g., acute intermittent porphyria, porphyria cutanea tarda), and Zellweger syndrome.

The term “small molecule” or “small molecule therapeutic” refers to molecules, whether naturally occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present disclosure.

The term “therapeutic agent” refers to any substance having therapeutic properties that produce a desired, usually beneficial, effect. For example, therapeutic agents may treat and/or ameliorate a disease. Therapeutic agents, as disclosed herein, may be biologics or small molecule therapeutics, or combinations thereof.

The term “isotopes” refers to variants of a particular chemical element such that, while all isotopes of a given element share the same number of protons in each atom of the element, those isotopes differ in the number of neutrons. The term “radioisotope” refers to an unstable isotope, the nucleus of which loses energy by emitting particles or rays (e.g., alpha particles, beta particles, and gamma rays) of ionizing radiation. Exemplary radioisotopes include, but are not limited to, ¹¹C, ¹⁸F, ¹³N, ¹⁵O, ⁶⁴Cu, ⁶⁷Ga, ⁶⁸Ga, ⁷⁵Se, ^{81m}Kr, ⁸²Rb, ^{99m}Tc, ¹¹¹In, ¹²³I, ¹²⁵I, ¹³¹I, ¹³³Xe. The term “radioactivity” or “radioactive decay” refers to the process by which a nucleus of an unstable isotope (e.g., ¹⁸F) loses energy by emitting particles or rays (e.g., alpha particles, beta particles, and gamma rays) of ionizing radiation. Such an unstable isotope or a material including the unstable isotope is referred to as “radioactive.” The Curie (Ci) is a non-SI (non-International System of Units) unit of radioactivity and is defined as 1 Ci=3.7×10¹⁰ decays per second. The term “specific activity” refers to the unit radioactivity of a material (e.g., a compound of Formula I, or a pharmaceutically acceptable salt, solvate, hydrate, polymorph, co-crystal, tautomer, stereoisomer, or prodrug thereof). In certain embodiments, the term “specific activity” refers to the radioactivity of a material per micromole (μmol) of the material. The term “standardized uptake value (SUV)” refers to the ratio of activity per unit volume of a region of interest (e.g., a tissue) to the activity per unit whole body volume. In certain embodiments, the term “standardized uptake value” is determined using positron emission tomography (PET) imaging. The term “time-activity curve” refers to a curve of radioactivity plotted against time. In certain embodiments, the radioactivity is measured in a region of interest (e.g., a tissue).

The term “linker” refers to a bond or a divalent chemical moiety that is bonded to (i.e., that connects) two separate monovalent chemical moieties (e.g., N and R¹ in Formula I).

### DETAILED DESCRIPTION OF CERTAIN EMBODIMENTS

Provided herein are compounds (e.g. compounds of Formula I, Formula I′), and pharmaceutically acceptable salts, solvates, hydrates, polymorphs, co-crystals, tautomers, stereoisomers, and prodrugs thereof, and pharmaceutical compositions and kits thereof. The compounds provided herein are indocyanine green (ICG) derivatives and can be used to diagnose, treat, and/or prevent diseases in a subject. Also provided herein are methods of diagnosing, treating, and/or preventing a disease (e.g. a proliferative disease) in a subject comprising administering an effective amount of a compound or pharmaceutical composition provided herein to the subject. The compound or pharmaceutical composition may be administered as a monotherapy or in combination with another therapy, as described herein. The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

### Compounds

The present disclosure provides compounds of Formula I:

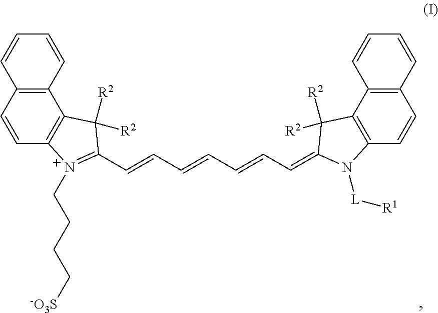

and pharmaceutically acceptable salts, solvates, hydrates, polymorphs, co-crystals, tautomers, stereoisomers, and prodrugs thereof, wherein:
L is a linker;
R¹ is halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(—NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂, and contains at least one radioisotope;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring; and
each occurrence of R² is independently hydrogen, halogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl, or two occurrences of R² are joined together with their intervening atom to form an optionally substituted carbocyclic ring or optionally substituted heterocyclic ring.

The present disclosure provides compounds of Formula I′:

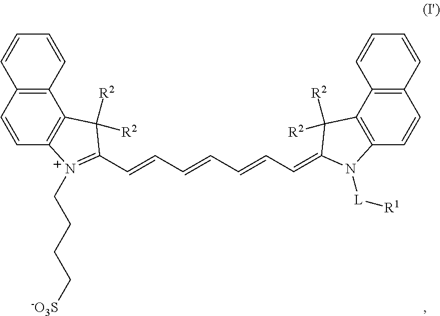

and pharmaceutically acceptable salts, solvates, hydrates, polymorphs, co-crystals, tautomers, stereoisomers, and prodrugs thereof, wherein:
L is a linker;
R¹ is halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring; and
each occurrence of R² is independently hydrogen, halogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl, or two occurrences of R² are joined together with their intervening atom to form an optionally substituted carbocyclic ring or optionally substituted heterocyclic ring.

### L

As described herein, L is a linker. In certain embodiments, L is a bond; substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted heteroalkynylene; substituted or unsubstituted heterocyclylene; substituted or unsubstituted carbocyclylene; substituted or unsubstituted arylene; substituted or unsubstituted heteroarylene; peptidyl groups; dipeptidyl groups; polypeptidyl groups; or combination thereof. In certain embodiments, L is substituted or unsubstituted alkylene. In certain embodiments, L is substituted alkylene. In certain embodiments, L is unsubstituted alkylene. In certain embodiments, L is alkylene substituted with an acyl group.

In certain embodiments, L is of formula

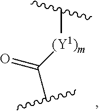

wherein:
each occurrence of Y¹ is independently C(R^{E1})₂, NR^{E2}, or —O—;
each occurrence of R^{E1} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂, or two occurrences of R^{E1} are optionally taken together with the intervening atoms to form an optionally substituted cyclic moiety;
each occurrence of R^{E2} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, or a nitrogen protecting group, or two occurrences of R^{E2} are optionally taken together with the intervening atoms to form an optionally substituted cyclic moiety; and
m is 0, 1, 2, 3, 4, 5, 6, 7, 8, 9, or 10.

In certain embodiments, L is of formula

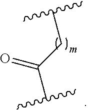

In certain embodiments, L is of formula

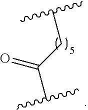

### Y¹ and m

As described herein, each occurrence of Y¹ is independently C(R^{E1})₂, NR^{E2}, or —O—; and m is 0, 1, 2, 3, 4, 5, 6, 7, 8, 9, or 10. In certain embodiments, at least one occurrence of Y¹ is C(R^{E1})₂. In certain embodiments, at least one occurrence of Y¹ is CH₂. In certain embodiments, at least one occurrence of Y¹ is NR^{E2}. In certain embodiments, at least one occurrence of Y¹ is NH. In certain embodiments, at least one occurrence of Y¹ is oxygen.

In certain embodiments, at least one occurrence of Y¹ is CH₂, R₁ is —N(R^{A})₂ and contains at least one radioisotope, —OR^{A} and contains at least one radioisotope, or —C(R^{A})₃ and contains at least one radioisotope. In certain embodiments, at least one occurrence of Y¹ is CH₂ and R¹ is —N(R^{A})² and contains at least one radioisotope. In certain embodiments, at least one occurrence of Y¹ is CH₂ and R¹ is —NHR^{A} and contains at least one radioisotope. In certain embodiments, at least one occurrence of Y¹ is CH₂ and R¹ is —OR^{A} and contains at least one radioisotope. In certain embodiments, at least one occurrence of Y¹ is CH₂ and R¹ is —C(R^{A})³ and contains at least one radioisotope.

In certain embodiments, m is 0. In certain embodiments, m is 1. In certain embodiments, m is 2. In certain embodiments, m is 3. In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10. In certain embodiments, m is 5 and at least one occurrence of Y¹ is CH₂. In certain embodiments, (Y¹)ₘ is (CH₂)₅. In certain embodiments, (Y¹)ₘ is (CH₂)₅, R¹ is —N(R^{A})₂ and contains at least one radioisotope, —OR^{A} and contains at least one radioisotope, or —C(R^{A})₃ and contains at least one radioisotope. In certain embodiments, (Y¹)ₘ is (CH₂)₅ and R¹ is —N(R^{A})₂ and contains at least one radioisotope. In certain embodiments, (Y¹)ₘ is (CH₂)₅ and R¹ is —NHR^{A} and contains at least one radioisotope. In certain embodiments, (Y¹)ₘ is (CH₂)₅ and R¹ is —OR^{A} and contains at least one radioisotope. In certain embodiments, (Y¹)ₘ is (CH₂)₅ and R¹ is —C(R^{A})₃ and contains at least one radioisotope.

### R^{E1}

As described herein, each occurrence of R^{E1} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂, or two occurrences of R^{E1} are optionally taken together with the intervening atoms to form an optionally substituted cyclic moiety. In certain embodiments, at least one occurrence of R^{E1} is hydrogen. In certain embodiments, at least one occurrence of R^{E1} is halogen. In certain embodiments, at least one occurrence of R^{E1} is fluorine, chlorine, or bromine. In certain embodiments, at least one occurrence of R^{E1} is fluorine. In certain embodiments, at least one occurrence of R^{E1} is optionally substituted C₁₋₁₂ alkyl. In certain embodiments, at least one occurrence of R^{E1} is optionally substituted C₁₋₆ alkyl. In certain embodiments, at least one occurrence of R^{E1} is unsubstituted C₁₋₆ alkyl. In certain embodiments, at least one occurrence of R^{E1} is substituted C₁₋₆ alkyl. In certain embodiments, at least one occurrence of R^{E1} is substituted or unsubstituted methyl, substituted or unsubstituted ethyl, substituted or unsubstituted n-propyl, substituted or unsubstituted isopropyl, substituted or unsubstituted n-butyl, substituted or unsubstituted tert-butyl, substituted or unsubstituted sec-butyl, substituted or unsubstituted isobutyl, substituted or unsubstituted n-pentyl, substituted or unsubstituted 3-pentanyl, substituted or unsubstituted amyl, substituted or unsubstituted neopentyl, substituted or unsubstituted 3-methyl-2-butanyl, substituted or unsubstituted tert-amyl, or substituted or unsubstituted n-hexyl. In certain embodiments, at least one occurrence of R^{E1} is optionally substituted C₂₋₆ alkenyl. In certain embodiments, at least one occurrence of R^{E1} is substituted or unsubstituted ethenyl, substituted or unsubstituted 1-propenyl, substituted or unsubstituted 2-propenyl, substituted or unsubstituted 1-butenyl, substituted or unsubstituted 2-butenyl, substituted or unsubstituted butadienyl, substituted or unsubstituted pentenyl, substituted or unsubstituted pentadienyl, or substituted or unsubstituted hexenyl. In certain embodiments, at least one occurrence of R^{E1} is optionally substituted C₂₋₆ alkynyl. In certain embodiments, at least one occurrence of R^{E1} is substituted or unsubstituted ethynyl, substituted or unsubstituted 1-propynyl, substituted or unsubstituted 2-propynyl, substituted or unsubstituted 1-butynyl, substituted or unsubstituted 2-butynyl, substituted or unsubstituted pentynyl, or substituted or unsubstituted hexynyl. In certain embodiments, at least one occurrence of R^{E1} is optionally substituted heteroC₁₋₆ alkyl. In certain embodiments, at least one occurrence of R^{E1} is optionally substituted heteroC₁₋₆ alkenyl. In certain embodiments, at least one occurrence of R^{E1} is optionally substituted heteroC₁₋₁₂ alkynyl. In certain embodiments, at least one occurrence of R^{E1} is optionally substituted heteroC₁₋₆ alkynyl. In certain embodiments, at least one occurrence of R^{E1} is —CN, —OR^{A}, —SR^{A}, or —N(R^{A})₂.

### R^{E2}

As described herein, each occurrence of R^{E2} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, or a nitrogen protecting group, or two occurrences of R^{E2} are optionally taken together with the intervening atoms to form an optionally substituted cyclic moiety.

In certain embodiments, at least one occurrence of R^{E2} is hydrogen. In certain embodiments, at least one occurrence of R^{E2} is optionally substituted C₁₋₁₂ alkyl. In certain embodiments, at least one occurrence of R^{E2} is optionally substituted C₁₋₆ alkyl. In certain embodiments, at least one occurrence of R^{E2} is unsubstituted C₁₋₆ alkyl. In certain embodiments, at least one occurrence of R^{E2} is substituted C₁₋₆ alkyl. In certain embodiments, at least one occurrence of R^{E2} is substituted or unsubstituted methyl, substituted or unsubstituted ethyl, substituted or unsubstituted n-propyl, substituted or unsubstituted isopropyl, substituted or unsubstituted n-butyl, substituted or unsubstituted tert-butyl, substituted or unsubstituted sec-butyl, substituted or unsubstituted isobutyl, substituted or unsubstituted n-pentyl, substituted or unsubstituted 3-pentanyl, substituted or unsubstituted amyl, substituted or unsubstituted neopentyl, substituted or unsubstituted 3-methyl-2-butanyl, substituted or unsubstituted tert-amyl, or substituted or unsubstituted n-hexyl. In certain embodiments, at least one occurrence of R^{E2} is optionally substituted C₂₋₆ alkenyl. In certain embodiments, at least one occurrence of R^{E2} is substituted or unsubstituted ethenyl, substituted or unsubstituted 1-propenyl, substituted or unsubstituted 2-propenyl, substituted or unsubstituted 1-butenyl, substituted or unsubstituted 2-butenyl, substituted or unsubstituted butadienyl, substituted or unsubstituted pentenyl, substituted or unsubstituted pentadienyl, or substituted or unsubstituted hexenyl. In certain embodiments, at least one occurrence of R^{E2} is optionally substituted C₂₋₆ alkynyl. In certain embodiments, at least one occurrence of R^{E2} is substituted or unsubstituted ethynyl, substituted or unsubstituted 1-propynyl, substituted or unsubstituted 2-propynyl, substituted or unsubstituted 1-butynyl, substituted or unsubstituted 2-butynyl, substituted or unsubstituted pentynyl, or substituted or unsubstituted hexynyl. In certain embodiments, at least one occurrence of R^{E2} is optionally substituted heteroC₁₋₆ alkyl. In certain embodiments, at least one occurrence of R^{E2} is optionally substituted heteroC₁₋₆ alkenyl. In certain embodiments, at least one occurrence of R^{E2} is optionally substituted heteroC₁₋₁₂ alkynyl. In certain embodiments, at least one occurrence of R^{E2} is optionally substituted heteroC₁₋₆ alkynyl. In certain embodiments, at least one occurrence of R^{E2} is a nitrogen protecting group.

### R¹

As described herein for compounds of Formula (I), R¹ is halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂, and contains at least one radioisotope.

In certain embodiments, R¹ is a halogen radioisotope. In certain embodiments, R¹ is a fluorine radioisotope (e.g., ¹⁸F), a chlorine radioisotope, or a bromine radioisotope. In certain embodiments, R¹ is a copper radioisotope (e.g., ⁶⁴Cu, ⁶⁷Cu). In certain embodiments, R¹ is optionally substituted C₁₋₁₂ alkyl and contains at least one radioisotope. In certain embodiments, R¹ is optionally substituted C₁₋₆ alkyl and contains at least one radioisotope. In certain embodiments, R¹ is unsubstituted C₁₋₆ alkyl and contains at least one radioisotope. In certain embodiments, R¹ is substituted C₁₋₆ alkyl and contains at least one radioisotope. In certain embodiments, R¹ is substituted or unsubstituted methyl and contains at least one radioisotope, substituted or unsubstituted ethyl and contains at least one radioisotope, substituted or unsubstituted n-propyl and contains at least one radioisotope, substituted or unsubstituted isopropyl and contains at least one radioisotope, substituted or unsubstituted n-butyl and contains at least one radioisotope, substituted or unsubstituted tert-butyl and contains at least one radioisotope, substituted or unsubstituted sec-butyl and contains at least one radioisotope, substituted or unsubstituted isobutyl and contains at least one radioisotope, substituted or unsubstituted n-pentyl and contains at least one radioisotope, substituted or unsubstituted 3-pentanyl and contains at least one radioisotope, substituted or unsubstituted amyl and contains at least one radioisotope, substituted or unsubstituted neopentyl and contains at least one radioisotope, substituted or unsubstituted 3-methyl-2-butanyl and contains at least one radioisotope, substituted or unsubstituted tert-amyl and contains at least one radioisotope, or substituted or unsubstituted n-hexyl and contains at least one radioisotope. In certain embodiments, R¹ is optionally substituted C₂₋₁₂ alkenyl and contains at least one radioisotope. In certain embodiments, R¹ is optionally substituted C₂₋₆ alkenyl and contains at least one radioisotope. In certain embodiments, R¹ is substituted or unsubstituted ethenyl and contains at least one radioisotope, substituted or unsubstituted 1-propenyl and contains at least one radioisotope, substituted or unsubstituted 2-propenyl and contains at least one radioisotope, substituted or unsubstituted 1-butenyl and contains at least one radioisotope, substituted or unsubstituted 2-butenyl and contains at least one radioisotope, substituted or unsubstituted butadienyl and contains at least one radioisotope, substituted or unsubstituted pentenyl and contains at least one radioisotope, substituted or unsubstituted pentadienyl and contains at least one radioisotope, or substituted or unsubstituted hexenyl and contains at least one radioisotope. In certain embodiments, R¹ is optionally substituted C₂₋₁₂ alkynyl and contains at least one radioisotope. In certain embodiments, R¹ is optionally substituted C₂₋₆ alkynyl and contains at least one radioisotope. In certain embodiments, R¹ is substituted or unsubstituted ethynyl and contains at least one radioisotope, substituted or unsubstituted 1-propynyl and contains at least one radioisotope, substituted or unsubstituted 2-propynyl and contains at least one radioisotope, substituted or unsubstituted 1-butynyl and contains at least one radioisotope, substituted or unsubstituted 2-butynyl and contains at least one radioisotope, substituted or unsubstituted pentynyl and contains at least one radioisotope, or substituted or unsubstituted hexynyl and contains at least one radioisotope. In certain embodiments, R¹ is optionally substituted heteroC₁₋₁₂ alkyl and contains at least one radioisotope. In certain embodiments, R¹ is optionally substituted heteroC₁₋₆ alkyl and contains at least one radioisotope. In certain embodiments, R¹ is optionally substituted heteroC₁₋₁₂ alkenyl and contains at least one radioisotope. In certain embodiments, R¹ is optionally substituted heteroC₁₋₆ alkenyl and contains at least one radioisotope. In certain embodiments, R¹ is optionally substituted heteroC₁₋₁₂ alkynyl and contains at least one radioisotope. In certain embodiments, R¹ is optionally substituted heteroC₁₋₆ alkynyl and contains at least one radioisotope. In certain embodiments, R¹ is optionally substituted C₃₋₁₄ cycloalkyl and contains at least one radioisotope. In certain embodiments, R¹ is optionally substituted 5-10 membered heterocyclyl and contains at least one radioisotope. In certain embodiments, R¹ is optionally substituted 6-14 membered aryl and contains at least one radioisotope. In certain embodiments, R¹ is optionally substituted 5-14 membered heteroaryl and contains at least one radioisotope.

In certain embodiments, R¹ is —N(R^{A})₂ and contains at least one radioisotope, —OR^{A} and contains at least one radioisotope, or —C(R^{A})₃ and contains at least one radioisotope. In certain embodiments, R¹ is —N(R^{A})₂ and contains at least one radioisotope. In certain embodiments, R¹ is —NHR^{A} and contains at least one radioisotope. In certain embodiments, R¹ is —OR^{A} and contains at least one radioisotope. In certain embodiments, R¹ is —C(R^{A})₃ and contains at least one radioisotope. In certain embodiments, R¹ is —C(═O)N(R^{A})₂ and contains at least one radioisotope, —C(═O)OR^{A} and contains at least one radioisotope, or —C(═O) C(R^{A})₃ and contains at least one radioisotope. In certain embodiments, R¹ is —C(═O)N(R^{A})₂ and contains at least one radioisotope. In certain embodiments, R¹ is —C(═O)NHR^{A} and contains at least one radioisotope. In certain embodiments, R¹ is —C(═O)OR^{A} and contains at least one radioisotope. In certain embodiments, R¹ is —C(═O) C(R^{A})₃ and contains at least one radioisotope.

As described herein for compounds of Formula (I′), R¹ is halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂.

In certain embodiments, R¹ is a halogen. In certain embodiments, R¹ is a fluorine, a chlorine, or a bromine. In certain embodiments, R¹ is optionally substituted C₁₋₁₂ alkyl. In certain embodiments, R¹ is optionally substituted C₁₋₆ alkyl. In certain embodiments, R¹ is unsubstituted C₁₋₆ alkyl. In certain embodiments, R¹ is substituted C₁₋₆ alkyl. In certain embodiments, R¹ is substituted or unsubstituted methyl, substituted or unsubstituted ethyl, substituted or unsubstituted n-propyl, substituted or unsubstituted isopropyl, substituted or unsubstituted n-butyl, substituted or unsubstituted tert-butyl, substituted or unsubstituted sec-butyl, substituted or unsubstituted isobutyl, substituted or unsubstituted n-pentyl, substituted or unsubstituted 3-pentanyl, substituted or unsubstituted amyl, substituted or unsubstituted neopentyl, substituted or unsubstituted 3-methyl-2-butanyl, substituted or unsubstituted tert-amyl, or substituted or unsubstituted n-hexyl. In certain embodiments, R¹ is optionally substituted C₂₋₁₂ alkenyl. In certain embodiments, R¹ is optionally substituted C₂₋₆ alkenyl. In certain embodiments, R¹ is substituted or unsubstituted ethenyl, substituted or unsubstituted 1-propenyl, substituted or unsubstituted 2-propenyl, substituted or unsubstituted 1-butenyl, substituted or unsubstituted 2-butenyl, substituted or unsubstituted butadienyl, substituted or unsubstituted pentenyl, substituted or unsubstituted pentadienyl, or substituted or unsubstituted hexenyl. In certain embodiments, R¹ is optionally substituted C₂₋₁₂ alkynyl. In certain embodiments, R¹ is optionally substituted C₂₋₆ alkynyl. In certain embodiments, R¹ is substituted or unsubstituted ethynyl, substituted or unsubstituted 1-propynyl, substituted or unsubstituted 2-propynyl, substituted or unsubstituted 1-butynyl, substituted or unsubstituted 2-butynyl, substituted or unsubstituted pentynyl, or substituted or unsubstituted hexynyl. In certain embodiments, R¹ is optionally substituted heteroC₁₋₁₂ alkyl. In certain embodiments, R¹ is optionally substituted heteroC₁₋₆ alkyl. In certain embodiments, R¹ is optionally substituted heteroC₁₋₁₂ alkenyl. In certain embodiments, R¹ is optionally substituted heteroC₁₋₆ alkenyl. In certain embodiments, R¹ is optionally substituted heteroC₁₋₁₂ alkynyl. In certain embodiments, R¹ is optionally substituted heteroC₁₋₆ alkynyl. In certain embodiments, R¹ is optionally substituted C₃₋₁₄ cycloalkyl. In certain embodiments, R¹ is optionally substituted 5-10 membered heterocyclyl. In certain embodiments, R¹ is optionally substituted 6-14 membered aryl. In certain embodiments, R¹ is optionally substituted 5-14 membered heteroaryl.

In certain embodiments, R¹ is —N(R^{A})₂, —OR^{A}, or —C(R^{A})₃. In certain embodiments, R¹ is —N(R^{A})₂. In certain embodiments, R¹ is —NHRA. In certain embodiments, R¹ is-ORA. In certain embodiments, R¹ is —C(R^{A})₃. In certain embodiments, R¹ is —C(═O)N(R^{A})₂, —C(═O)OR^{A}, or —C(═O) C(R^{A})₃. In certain embodiments, R¹ is —C(═O)N(R^{A})₂. In certain embodiments, R¹ is —C(═O)NHRA. In certain embodiments, R¹ is —C(═O)ORA. In certain embodiments, R¹ is-C(═O) C(R^{A})₃.

### R^{A}

As described herein, each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring.

In certain embodiments, at least one occurrence of R^{A} contains at least one radioisotope.

In certain embodiments, at least one occurrence of R^{A} is hydrogen. In certain embodiments, at least one occurrence of R^{A} is optionally substituted acyl. In certain embodiments, at least one occurrence of R^{A} is optionally substituted C₁₋₁₂ alkyl. In certain embodiments, at least one occurrence of R^{A} is optionally substituted C₁₋₆ alkyl. In certain embodiments, at least one occurrence of R^{A} is unsubstituted C₁₋₆ alkyl. In certain embodiments, at least one occurrence of R^{A} is substituted C₁₋₆ alkyl. In certain embodiments, at least one occurrence of R^{A} is substituted or unsubstituted methyl, substituted or unsubstituted ethyl, substituted or unsubstituted n-propyl, substituted or unsubstituted isopropyl, substituted or unsubstituted n-butyl, substituted or unsubstituted tert-butyl, substituted or unsubstituted sec-butyl, substituted or unsubstituted isobutyl, substituted or unsubstituted n-pentyl, substituted or unsubstituted 3-pentanyl, substituted or unsubstituted amyl, substituted or unsubstituted neopentyl, substituted or unsubstituted 3-methyl-2-butanyl, substituted or unsubstituted tert-amyl, or substituted or unsubstituted n-hexyl.

In certain embodiments, at least one occurrence of R^{A} is alkyl substituted with an optionally substituted amino group, alkyl substituted with an acyl group, or alkyl substituted with halogen. In certain embodiments, at least one occurrence of R^{A} is alkyl substituted with an optionally substituted amino group. In certain embodiments, at least one occurrence of R^{A} is C₁₋₁₂ alkyl substituted with an optionally substituted amino group. In certain embodiments, at least one occurrence of R^{A} is C₁₋₆ alkyl substituted with an optionally substituted amino group. In certain embodiments, at least one occurrence of R^{A} is methyl substituted with an optionally substituted amino group, ethyl substituted with an optionally substituted amino group, n-propyl substituted with an optionally substituted amino group, isopropyl substituted with an optionally substituted amino group, n-butyl substituted with an optionally substituted amino group, tert-butyl substituted with an optionally substituted amino group, sec-butyl substituted with an optionally substituted amino group, isobutyl substituted with an optionally substituted amino group, n-pentyl substituted with an optionally substituted amino group, 3-pentanyl substituted with an optionally substituted amino group, amyl substituted with an optionally substituted amino group, neopentyl substituted with an optionally substituted amino group, 3-methyl-2-butanyl substituted with an optionally substituted amino group, tert-amyl substituted with an optionally substituted amino group, or n-hexyl substituted with an optionally substituted amino group. In certain embodiments, at least one occurrence of R^{A} is alkyl substituted with an amino group substituted with an acyl group. In certain embodiments, at least one occurrence of R^{A} is C₁₋₁₂ alkyl substituted with an amino group substituted with an acyl group. In certain embodiments, at least one occurrence of R^{A} is C₁₋₆ alkyl substituted with an amino group substituted with an acyl group. In certain embodiments, at least one occurrence of R^{A} is methyl substituted with an amino group substituted with an acyl group, ethyl substituted with an amino group substituted with an acyl group, n-propyl substituted with an amino group substituted with an acyl group, isopropyl substituted with an amino group substituted with an acyl group, n-butyl substituted with an amino group substituted with an acyl group, tert-butyl substituted with an amino group substituted with an acyl group, sec-butyl substituted with an amino group substituted with an acyl group, isobutyl substituted with an amino group substituted with an acyl group, n-pentyl substituted with an amino group substituted with an acyl group, 3-pentanyl substituted with an amino group substituted with an acyl group, amyl substituted with an amino group substituted with an acyl group, neopentyl substituted with an amino group substituted with an acyl group, 3-methyl-2-butanyl substituted with an amino group substituted with an acyl group, tert-amyl substituted with an amino group substituted with an acyl group, or n-hexyl substituted with an amino group substituted with an acyl group.

In certain embodiments, at least one occurrence of R^{A} is alkyl substituted with an acyl group. In certain embodiments, at least one occurrence of R^{A} is C₁₋₁₂ alkyl substituted with an acyl group. In certain embodiments, at least one occurrence of R^{A} is C₁₋₆ alkyl substituted with an acyl group. In certain embodiments, at least one occurrence of R^{A} is methyl substituted with an acyl group, ethyl substituted with an acyl group, n-propyl substituted with an acyl group, isopropyl substituted with an acyl group, n-butyl substituted with an acyl group, tert-butyl substituted with an acyl group, sec-butyl substituted with an acyl group, isobutyl substituted with an acyl group, n-pentyl substituted with an acyl group, 3-pentanyl substituted with an acyl group, amyl substituted with an acyl group, neopentyl substituted with an acyl group, 3-methyl-2-butanyl substituted with an acyl group, tert-amyl substituted with an acyl group, or n-hexyl substituted with an acyl group. In certain embodiments, at least one occurrence of R^{A} is alkyl substituted with an amide group (e.g., —C(═O)N(R^{bb})₂, —C(═O)NR^{bb}SO₂R^{aa}, —C(═S)N(R^{bb})₂), wherein R^{aa} and R^{bb} are as defined herein. In certain embodiments, at least one occurrence of R^{A} is C₁₋₁₂ alkyl substituted with an amide group. In certain embodiments, at least one occurrence of R^{A} is C₁₋₆ alkyl substituted with an amide group. In certain embodiments, at least one occurrence of R^{A} is methyl substituted with an amide group, ethyl substituted with an amide group, n-propyl substituted with an amide group, isopropyl substituted with an amide group, n-butyl substituted with an amide group, tert-butyl substituted with an amide group, sec-butyl substituted with an amide group, isobutyl substituted with an amide group, n-pentyl substituted with an amide group, 3-pentanyl substituted with an amide group, amyl substituted with an amide group, neopentyl substituted with an amide group, 3-methyl-2-butanyl substituted with an amide group, tert-amyl substituted with an amide group, or n-hexyl substituted with an amide group.

In certain embodiments, at least one occurrence of R^{A} is alkyl substituted with halogen. In certain embodiments, at least one occurrence of R^{A} is C₁₋₁₂ alkyl substituted with halogen. In certain embodiments, at least one occurrence of R^{A} is C₁₋₆ alkyl substituted with halogen. In certain embodiments, at least one occurrence of R^{A} is methyl substituted with halogen, ethyl substituted with halogen, n-propyl substituted with halogen, isopropyl substituted with halogen, n-butyl substituted with halogen, tert-butyl substituted with halogen, sec-butyl substituted with halogen, isobutyl substituted with halogen, n-pentyl substituted with halogen, 3-pentanyl substituted with halogen, amyl substituted with halogen, neopentyl substituted with halogen, 3-methyl-2-butanyl substituted with halogen, tert-amyl substituted with halogen, or n-hexyl substituted with halogen. In certain embodiments, at least one occurrence of R^{A} is alkyl substituted with fluorine, chlorine, or bromine. In certain embodiments, at least one occurrence of R^{A} is alkyl substituted with fluorine. In certain embodiments, at least one occurrence of R^{A} is C₁₋₁₂ alkyl substituted with fluorine. In certain embodiments, at least one occurrence of R^{A} is C₁₋₆ alkyl substituted with fluorine. In certain embodiments, at least one occurrence of R^{A} is methyl substituted with fluorine, ethyl substituted with fluorine, n-propyl substituted with fluorine, isopropyl substituted with fluorine, n-butyl substituted with fluorine, tert-butyl substituted with fluorine, sec-butyl substituted with fluorine, isobutyl substituted with fluorine, n-pentyl substituted with fluorine, 3-pentanyl substituted with fluorine, amyl substituted with fluorine, neopentyl substituted with fluorine, 3-methyl-2-butanyl substituted with fluorine, tert-amyl substituted with fluorine, or n-hexyl substituted with fluorine. In certain embodiments, at least one occurrence of R^{A} is ethyl substituted with fluorine. In certain embodiments, at least one occurrence of R^{A} is alkyl substituted with ¹⁸F. In certain embodiments, at least one occurrence of R^{A} is C₁₋₁₂ alkyl substituted with ¹⁸F. In certain embodiments, at least one occurrence of R^{A} is C₁₋₆ alkyl substituted with ¹⁸F. In certain embodiments, at least one occurrence of R^{A} is methyl substituted with ¹⁸F, ethyl substituted with ¹⁸F, n-propyl substituted with ¹⁸F, isopropyl substituted with ¹⁸F, n-butyl substituted with ¹⁸F, tert-butyl substituted with ¹⁸F, sec-butyl substituted with ¹⁸F, isobutyl substituted with ¹⁸F, n-pentyl substituted with ¹⁸F, 3-pentanyl substituted with ¹⁸F, amyl substituted with ¹⁸F, neopentyl substituted with ¹⁸F, 3-methyl-2-butanyl substituted with ¹⁸F, tert-amyl substituted with ¹⁸F, or n-hexyl substituted with ¹⁸F. In certain embodiments, at least one occurrence of R^{A} is ethyl substituted with ¹⁸F.

In certain embodiments, at least one occurrence of R^{A} is optionally substituted C₂₋₁₂ alkenyl. In certain embodiments, at least one occurrence of R^{A} is optionally substituted C₂₋₆ alkenyl. In certain embodiments, at least one occurrence of R^{A} is substituted or unsubstituted ethenyl, substituted or unsubstituted 1-propenyl, substituted or unsubstituted 2-propenyl, substituted or unsubstituted 1-butenyl, substituted or unsubstituted 2-butenyl, substituted or unsubstituted butadienyl, substituted or unsubstituted pentenyl, substituted or unsubstituted pentadienyl, or substituted or unsubstituted hexenyl. In certain embodiments, at least one occurrence of R^{A} is optionally substituted C₂₋₁₂ alkynyl. In certain embodiments, at least one occurrence of R^{A} is optionally substituted C₂₋₆ alkynyl. In certain embodiments, at least one occurrence of R^{A} is substituted or unsubstituted ethynyl, substituted or unsubstituted 1-propynyl, substituted or unsubstituted 2-propynyl, substituted or unsubstituted 1-butynyl, substituted or unsubstituted 2-butynyl, substituted or unsubstituted pentynyl, or substituted or unsubstituted hexynyl. In certain embodiments, at least one occurrence of R^{A} is optionally substituted heteroC₁₋₁₂ alkyl. In certain embodiments, at least one occurrence of R^{A} is optionally substituted heteroC₁₋₆ alkyl. In certain embodiments, at least one occurrence of R^{A} is optionally substituted heteroC₁₋₁₂ alkenyl. In certain embodiments, at least one occurrence of R^{A} is optionally substituted heteroC₁₋₆ alkenyl. In certain embodiments, at least one occurrence of R^{A} is optionally substituted heteroC₁₋₁₂ alkynyl. In certain embodiments, at least one occurrence of R^{A} is optionally substituted heteroC₁₋₆ alkynyl. In certain embodiments, at least one occurrence of R^{A} is optionally substituted C₃₋₁₄ cycloalkyl. In certain embodiments, at least one occurrence of R^{A} is optionally substituted 5-10 membered heterocyclyl. In certain embodiments, at least one occurrence of R^{A} is optionally substituted 6-14 membered aryl. In certain embodiments, at least one occurrence of R^{A} is optionally substituted 5-14 membered heteroaryl. In certain embodiments, at least one occurrence of R^{A} is a nitrogen protecting group when attached to a nitrogen atom. In certain embodiments, at least one occurrence of R^{A} is an oxygen protecting group when attached to an oxygen atom. In certain embodiments, at least one occurrence of R^{A} is a sulfur protecting group when attached to a sulfur atom. In certain embodiments, at least two occurrences of R^{A} are joined together with their intervening atom to form an optionally substituted 5-10 membered heterocyclic ring. In certain embodiments, at least two occurrences of R^{A} are joined together with their intervening atom to form an optionally substituted 5-14 membered heteroaryl ring. In certain embodiments, each occurrence of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl.

In certain embodiments, at least one occurrence of R^{A} is

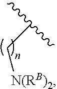

wherein:
each occurrence of R^{B} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or two occurrences of R^{B} are joined together with their intervening atom to form an optionally substituted heterocyclic ring or optionally substituted heteroaryl ring; and
n is 1, 2, 3, 4, 5, 6, 7, 8, 9, or 10.

In certain embodiments, at least one occurrence of R^{A} is

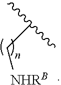

In certain embodiments, at least one occurrence of R^{A} is

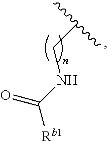

wherein:
R^{b1} is optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl.

In certain embodiments, at least one occurrence of R^{A} is

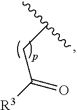

wherein:
R³ is halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{C}, —SCN, —SR^{C}, —SSR^{C}, —N₃, —NO, —N(R^{C})₂, —NO₂, —C(═O)R^{C}, —C(═O)OR^{C}, —C(═O)SR^{C}, —C(═O)N(R^{C})₂, —C(═NR^{C})R^{C}, —C(═NR^{C})OR^{C}, —C(═NR^{C})SR^{C}, —C(═NR^{C})N(R^{C})₂, —S(═O)R^{C}, —S(═O)OR^{C}, —S(═O)SR^{C}, —S(═O)N(R^{C})₂, —S(═O)₂R^{C}, —S(═O)₂OR^{C}, —S(═O)₂SR^{C}, —S(═O)₂N(R^{C})₂, —OC(═O)R^{C}, —OC(═O)OR^{C}, —OC(═O)SR^{C}, —OC(═O)N(R^{C})₂, —OC(═NR^{C})R^{C}, —OC(═NR^{C})OR^{C}, —OC(═NR^{C})SR^{C}, —OC(═NR^{C})N(R^{C})₂, —OS(═O)R^{C}, —OS(═O)OR^{C}, —OS(═O)SR^{C}, —OS(═O)N(R^{C})₂, —OS(═O)₂R^{C}, —OS(═O)₂OR^{C}, —OS(═O)₂SR^{C}, —OS(═O)₂N(R^{C})₂, —ON(R^{C})₂, —SC(═O)R^{C}, —SC(═O)OR^{C}, —SC(═O)SR^{C}, —SC(═O)N(R^{C})₂, —SC(═NR^{C})R^{C}, —SC(═NR^{C})OR^{C}, —SC(═NR^{C})SR^{C}, —SC(═NR^{C})N(R^{C})₂, —NR^{C}C(═O)R^{C}, —NR^{C}C(═O)OR^{C}, —NR^{C}C(═O)SR^{C}, —NR^{C}C(═O)N(R^{C})₂, —NR^{C}C(═NR^{C})R^{C}, —NR^{C}C(═NR^{C})OR^{C}, —NR^{C}C(═NR^{C})SR^{C}, —NR^{C}C(═NR^{C})N(R^{C})₂, —NR^{C}S(═O)R^{C}, —NR^{C}S(═O)OR^{C}, —NR^{C}S(═O)SR^{C}, —NR^{C}S(═O)N(R^{C})₂, —NR^{C}S(═O)₂R^{C}, —NR^{C}S(═O)₂OR^{C}, —NR^{C}S(═O)₂SR^{C}, —NR^{C}S(═O)₂N(R^{C})₂, —Si(R^{C})₃, —Si(R^{C})₂OR^{C}, —Si(R^{C}) (OR)₂, —Si(OR^{C})₃, —OSi(R^{C})₃, —OSi(R^{C})₂OR^{C}, —OSi(R^{C}) (OR^{C})₂, —OSi(OR^{C})₃, or —B(OR^{C})₂;
each occurrence of R^{C} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{C} are joined together with their intervening atom to form an optionally substituted heterocyclic ring or optionally substituted heteroaryl ring; and
p is 0, 1, 2, 3, 4, 5, 6, 7, 8, 9, or 10.

In certain embodiments, at least one occurrence of R^{A} is

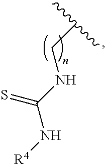

wherein:
R⁴ is optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl.

### R^{B}

As described herein, each occurrence of R^{B} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or two occurrences of R^{B} are joined together with their intervening atom to form an optionally substituted heterocyclic ring or optionally substituted heteroaryl ring.

In certain embodiments, at least one occurrence of R^{B} contains at least one radioisotope.

In certain embodiments, at least one occurrence of R^{B} is hydrogen. In certain embodiments, at least one occurrence of R^{B} is optionally substituted acyl. In certain embodiments, at least one occurrence of R^{B} is

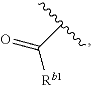

wherein R^{b1} is as defined herein. In certain embodiments, at least one occurrence of R^{B} is optionally substituted C₁₋₁₂ alkyl. In certain embodiments, at least one occurrence of R^{B} is optionally substituted C₁₋₆ alkyl. In certain embodiments, at least one occurrence of R^{B} is unsubstituted C₁₋₆ alkyl. In certain embodiments, at least one occurrence of R^{B} is substituted C₁₋₆ alkyl. In certain embodiments, at least one occurrence of R^{B} is substituted or unsubstituted methyl, substituted or unsubstituted ethyl, substituted or unsubstituted n-propyl, substituted or unsubstituted isopropyl, substituted or unsubstituted n-butyl, substituted or unsubstituted tert-butyl, substituted or unsubstituted sec-butyl, substituted or unsubstituted isobutyl, substituted or unsubstituted n-pentyl, substituted or unsubstituted 3-pentanyl, substituted or unsubstituted amyl, substituted or unsubstituted neopentyl, substituted or unsubstituted 3-methyl-2-butanyl, substituted or unsubstituted tert-amyl, or substituted or unsubstituted n-hexyl. In certain embodiments, at least one occurrence of R^{B} is optionally substituted C₂₋₁₂ alkenyl. In certain embodiments, at least one occurrence of RB is optionally substituted C₂₋₆ alkenyl. In certain embodiments, at least one occurrence of R^{B} is substituted or unsubstituted ethenyl, substituted or unsubstituted 1-propenyl, substituted or unsubstituted 2-propenyl, substituted or unsubstituted 1-butenyl, substituted or unsubstituted 2-butenyl, substituted or unsubstituted butadienyl, substituted or unsubstituted pentenyl, substituted or unsubstituted pentadienyl, or substituted or unsubstituted hexenyl. In certain embodiments, at least one occurrence of R^{B} is optionally substituted C₂₋₁₂ alkynyl. In certain embodiments, at least one occurrence of R^{B} is optionally substituted C₂₋₆ alkynyl. In certain embodiments, at least one occurrence of R^{B} is substituted or unsubstituted ethynyl, substituted or unsubstituted 1-propynyl, substituted or unsubstituted 2-propynyl, substituted or unsubstituted 1-butynyl, substituted or unsubstituted 2-butynyl, substituted or unsubstituted pentynyl, or substituted or unsubstituted hexynyl. In certain embodiments, at least one occurrence of R^{B} is optionally substituted heteroC₁₋₁₂ alkyl. In certain embodiments, at least one occurrence of R^{B} is optionally substituted heteroC₁₋₆ alkyl. In certain embodiments, at least one occurrence of R^{B} is optionally substituted heteroC₁₋₁₂ alkenyl. In certain embodiments, at least one occurrence of R^{B} is optionally substituted heteroC₁₋₆ alkenyl. In certain embodiments, at least one occurrence of R^{B} is optionally substituted heteroC₁₋₁₂ alkynyl. In certain embodiments, at least one occurrence of RB is optionally substituted heteroC₁₋₆ alkynyl. In certain embodiments, at least one occurrence of R^{B} is optionally substituted C₃₋₁₄ cycloalkyl. In certain embodiments, at least one occurrence of R^{B} is optionally substituted 5-10 membered heterocyclyl. In certain embodiments, at least one occurrence of R^{B} is optionally substituted 6-14 membered aryl. In certain embodiments, at least one occurrence of R^{B} is optionally substituted 5-14 membered heteroaryl. In certain embodiments, at least one occurrence of R^{B} is a nitrogen protecting group when attached to a nitrogen atom. In certain embodiments, at least two occurrences of R^{B} are joined together with their intervening atom to form an optionally substituted 5-10 membered carbocyclic ring. In certain embodiments, at least two occurrences of R^{B} are joined together with their intervening atom to form an optionally substituted 5-10 membered heterocyclic ring.

### R^{b1}

As described herein, R^{b1} is optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl.

In certain embodiments, R^{b1} contains at least one radioisotope.

In certain embodiments, R^{b1} is optionally substituted C₁₋₁₂ alkyl. In certain embodiments, at least one occurrence of R^{b1} is optionally substituted C₁₋₆ alkyl. In certain embodiments, R^{b1} is unsubstituted C₁₋₆ alkyl. In certain embodiments, R^{b1} is substituted C₁₋₆ alkyl. In certain embodiments, R^{b1} is substituted or unsubstituted methyl, substituted or unsubstituted ethyl, substituted or unsubstituted n-propyl, substituted or unsubstituted isopropyl, substituted or unsubstituted n-butyl, substituted or unsubstituted tert-butyl, substituted or unsubstituted sec-butyl, substituted or unsubstituted isobutyl, substituted or unsubstituted n-pentyl, substituted or unsubstituted 3-pentanyl, substituted or unsubstituted amyl, substituted or unsubstituted neopentyl, substituted or unsubstituted 3-methyl-2-butanyl, substituted or unsubstituted tert-amyl, or substituted or unsubstituted n-hexyl. In certain embodiments, R^{b1} is substituted or unsubstituted methyl. In certain embodiments, R^{b1} is unsubstituted methyl. In certain embodiments, R^{b1} is optionally substituted C₂₋₁₂ alkenyl. In certain embodiments, R^{b1} is optionally substituted C₂₋₆ alkenyl. In certain embodiments, R^{b1} is substituted or unsubstituted ethenyl, substituted or unsubstituted 1-propenyl, substituted or unsubstituted 2-propenyl, substituted or unsubstituted 1-butenyl, substituted or unsubstituted 2-butenyl, substituted or unsubstituted butadienyl, substituted or unsubstituted pentenyl, substituted or unsubstituted pentadienyl, or substituted or unsubstituted hexenyl. In certain embodiments, R^{b1} is optionally substituted C₂₋₁₂ alkynyl. In certain embodiments, R^{b1} is optionally substituted C₂₋₆ alkynyl. In certain embodiments, R^{b1} is substituted or unsubstituted ethynyl, substituted or unsubstituted 1-propynyl, substituted or unsubstituted 2-propynyl, substituted or unsubstituted 1-butynyl, substituted or unsubstituted 2-butynyl, substituted or unsubstituted pentynyl, or substituted or unsubstituted hexynyl. In certain embodiments, R^{b1} is optionally substituted heteroC₁₋₁₂ alkyl. In certain embodiments, R^{b1} is optionally substituted heteroC₁₋₆ alkyl. In certain embodiments, R^{b1} is optionally substituted heteroC₁₋₁₂ alkenyl. In certain embodiments, R^{b1} is optionally substituted heteroC₁₋₆ alkenyl. In certain embodiments, R^{b1} is optionally substituted heteroC₁₋₁₂ alkynyl. In certain embodiments, R^{b1} is optionally substituted heteroC₁₋₆ alkynyl. In certain embodiments, R^{b1} is optionally substituted C₃₋₁₄ cycloalkyl. In certain embodiments, R^{b1} is optionally substituted 5-10 membered heterocyclyl.

In certain embodiments, R^{b1} is optionally substituted aryl or optionally substituted heteroaryl. In certain embodiments, R^{b1} is optionally substituted aryl. In certain embodiments, R^{b1} is aryl substituted with halogen. In certain embodiments, R^{b1} is aryl substituted with fluorine. In certain embodiments, R^{b1} is aryl substituted with ¹⁸F. In certain embodiments, R^{b1} is optionally substituted 6-14 membered aryl. In certain embodiments, R^{b1} is 6-14 membered aryl substituted with halogen. In certain embodiments, R^{b1} is 6-14 membered aryl substituted with fluorine. In certain embodiments, R^{b1} is 6-14 membered aryl substituted with ¹⁸F. In certain embodiments, R^{b1} is phenyl substituted with fluorine. In certain embodiments, R^{b1} is phenyl substituted with ¹⁸F. In certain embodiments, R^{b1} is

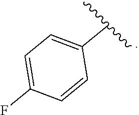 [SMILES: *c1ccc(F)cc1]

In certain embodiments, R^{b1} is

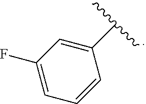 [SMILES: *c1cccc(F)c1]

In certain embodiments, R^{b1} is

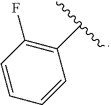 [SMILES: *c1ccccc1F]

In certain embodiments, R^{b1} is phenyl substituted with ¹⁸F. In certain embodiments, R^{b1} is

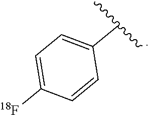 [SMILES: *c1ccc([18F])cc1]

In certain embodiments, R^{b1} is

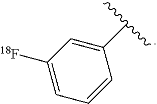 [SMILES: *c1cccc([18F])c1]

In certain embodiments, R^{b1} is

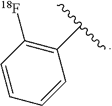 [SMILES: *c1ccccc1[18F]]

In certain embodiments, R^{b1} is optionally substituted heteroaryl. In certain embodiments, R^{b1} is heteroaryl substituted with halogen. In certain embodiments, R^{b1} is heteroaryl substituted with fluorine. In certain embodiments, R^{b1} is heteroaryl substituted with ¹⁸F. In certain embodiments, R^{b1} is optionally substituted 5-14 membered heteroaryl. In certain embodiments, R^{b1} is 5-14 membered heteroaryl substituted with halogen. In certain embodiments, R^{b1} is 5-14 membered heteroaryl substituted with fluorine. In certain embodiments, R^{b1} is 5-14 membered heteroaryl substituted with ¹⁸F. In certain embodiments, R^{b1} is pyridyl substituted with fluorine. In certain embodiments, R^{b1} is pyridyl substituted with ¹⁸F. In certain embodiments, R^{b1} is

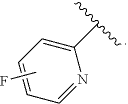

In certain embodiments, R^{b1} is

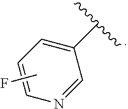

In certain embodiments, R^{b1} is

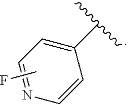

In certain embodiments, R^{b1} is

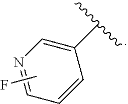

In certain embodiments, R^{b1} is

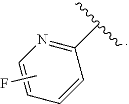

In certain embodiments, R^{b1} is

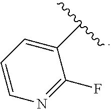 [SMILES: *c1cccnc1F]

In certain embodiments, R^{b1} is

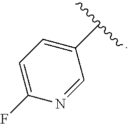 [SMILES: *c1ccc(F)nc1]

In certain embodiments, R^{b1} is

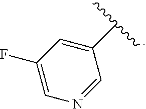 [SMILES: *c1cncc(F)c1]

In certain embodiments, R^{b1} is

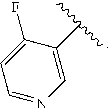 [SMILES: *c1cnccc1F]

In certain embodiments, R^{b1} is

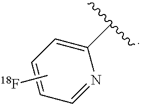

In certain embodiments, R^{b1} is

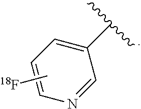

In certain embodiments, R^{b1} is

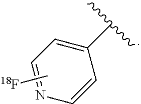

In certain embodiments, R^{b1} is

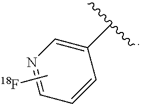

In certain embodiments, R^{b1} is

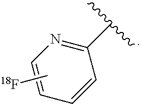

In certain embodiments, R^{b1} is

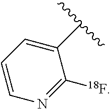

In certain embodiments, R^{b1} is

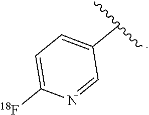 [SMILES: *c1ccc([18F])nc1]

In certain embodiments, R^{b1} is

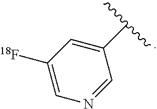 [SMILES: *c1cncc([18F])c1]

In certain embodiments, R^{b1} is

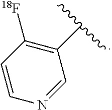 [SMILES: *c1cnccc1[18F]]

As described herein, n is 1, 2, 3, 4, 5, 6, 7, 8, 9, or 10. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10.

In certain embodiments, n is 2 and R^{b1} is optionally substituted aryl or optionally substituted heteroaryl. In certain embodiments, n is 2 and R^{b1} is optionally substituted aryl. In certain embodiments, n is 2 and R^{b1} is aryl substituted with halogen. In certain embodiments, n is 2 and R^{b1} is aryl substituted with fluorine. In certain embodiments, n is 2 and R^{b1} is aryl substituted with ¹⁸F. In certain embodiments, n is 2 and R^{b1} is optionally substituted 6-14 membered aryl. In certain embodiments, n is 2 and R^{b1} is 6-14 membered aryl substituted with halogen. In certain embodiments, n is 2 and R^{b1} is 6-14 membered aryl substituted with fluorine. In certain embodiments, n is 2 and R^{b1} is 6-14 membered aryl substituted with ¹⁸F. In certain embodiments, n is 2 and R^{b1} is optionally substituted heteroaryl. In certain embodiments, n is 2 and R^{b1} is heteroaryl substituted with halogen. In certain embodiments, n is 2 and R^{b1} is heteroaryl substituted with fluorine. In certain embodiments, n is 2 and R^{b1} is heteroaryl substituted with ¹⁸F. In certain embodiments, n is 2 and R^{b1} is optionally substituted 5-14 membered heteroaryl. In certain embodiments, n is 2 and R^{b1} is 5-14 membered heteroaryl substituted with halogen. In certain embodiments, n is 2 and R^{b1} is 5-14 membered heteroaryl substituted with fluorine. In certain embodiments, n is 2 and R^{b1} is 5-14 membered heteroaryl substituted with ¹⁸F.

In certain embodiments, n is 2 and m is 5. In certain embodiments, n is 2, m is 5, and at least one occurrence of Y¹ is CH₂. In certain embodiments, n is 2 and (Y¹)ₘ is (CH₂)₅. In certain embodiments, n is 2, (Y¹)ₘ is (CH₂)₅, and R¹ is —N(R^{A})₂ and contains at least one radioisotope. In certain embodiments, n is 2, (Y¹)ₘ is (CH₂)₅, and R¹ is —NHR^{A} and contains at least one radioisotope. In certain embodiments, n is 2, (Y¹)ₘ is (CH₂)₅, R¹ is —NHR^{A} and contains at least one radioisotope, and R^{A} is

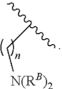

In certain embodiments, n is 2, (Y¹)ₘ is (CH₂)₅, R¹ is —NHR^{A} and contains at least one radioisotope, and R^{A} is

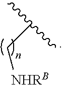

In certain embodiments, n is 2, (Y¹)ₘ is (CH₂)₅, R¹ is —NHR^{A} and contains at least one radioisotope, and R^{A} is

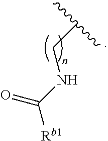

In certain embodiments, n is 2, (Y¹)ₘ is (CH₂)₅, R¹ is —NHR^{A} and contains at least one radioisotope, R^{A} is

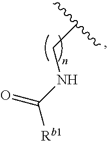

and R^{b1} is optionally substituted aryl or optionally substituted heteroaryl. In certain embodiments, n is 2, (Y¹)ₘ is (CH₂)₅, R¹ is —NHR^{A} and contains at least one radioisotope, R^{A} is

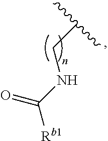

and R^{b1} is optionally substituted aryl. In certain embodiments, n is 2, (Y¹)ₘ is (CH₂)₅, R¹ is —NHR^{A} and contains at least one radioisotope, R^{A} is

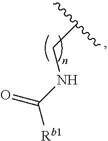

and R^{b1} is aryl substituted with halogen. In certain embodiments, n is 2, (Y¹)ₘ is (CH₂)₅, R¹ is —NHR^{A} and contains at least one radioisotope, R^{A} is

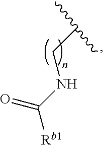

and R^{b1} is aryl substituted with fluorine. In certain embodiments, n is 2, (Y¹)ₘ is (CH₂)₅, R¹ is —NHR^{A} and contains at least one radioisotope, R^{A} is

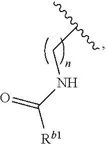

and R^{b1} is aryl substituted with ¹⁸F. In certain embodiments, n is 2, (Y¹)ₘ is (CH₂)₅, R¹ is —NHR^{A} and contains at least one radioisotope, R^{A} is

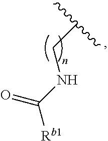

and R^{b1} is optionally substituted 6-14 membered aryl. In certain embodiments, n is 2, (Y¹)ₘ is (CH₂)₅, R¹ is —NHR^{A} and contains at least one radioisotope, R^{A} is

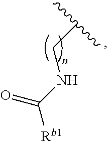

and R^{b1} is 6-14 membered aryl substituted with halogen. In certain embodiments, n is 2, (Y¹)ₘ is (CH₂)₅, R¹ is —NHR^{A} and contains at least one radioisotope, R^{A} is

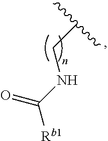

and R^{b1} is 6-14 membered aryl substituted with fluorine. In certain embodiments, n is 2, (Y¹)ₘ is (CH₂)₅, R¹ is —NHR^{A} and contains at least one radioisotope, R^{A} is

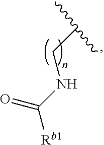

and R^{b1} is 6-14 membered aryl substituted with ¹⁸F. In certain embodiments, n is 2, (Y¹)ₘ is (CH₂)₅, R¹ is —NHR^{A} and contains at least one radioisotope, R^{A} is

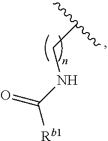

and R^{b1} is phenyl substituted with fluorine. In certain embodiments, n is 2, (Y¹)ₘ is (CH₂)₅, R¹ is —NHR^{A} and contains at least one radioisotope, R^{A} is

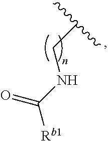

and R^{b1} is phenyl substituted with ¹⁸F. In certain embodiments, n is 2, (Y¹)ₘ is (CH₂)₅, R¹ is —NHR^{A} and contains at least one radioisotope, R^{A} is

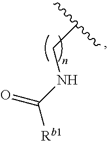

### and R^{b1} is

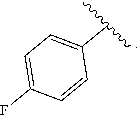 [SMILES: *c1ccc(F)cc1]

In certain embodiments, n is 2, (Y¹)ₘ is (CH₂)₅, R¹ is —NHR^{A} and contains at least one radioisotope, R^{A} is

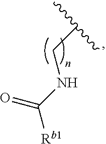

### and R^{b1} is

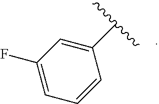 [SMILES: *c1cccc(F)c1]

In certain embodiments, n is 2, (Y¹)ₘ is (CH₂)₅, R¹ is —NHR^{A} and contains at least one radioisotope, R^{A} is

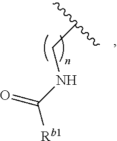

### and R^{b1} is

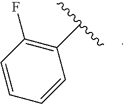 [SMILES: *c1ccccc1F]

In certain embodiments, n is 2, (Y¹)ₘ is (CH₂)₅, R¹ is —NHR^{A} and contains at least one radioisotope, R^{A} is

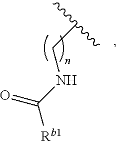

and R^{b1} is phenyl substituted with ¹⁸F. In certain embodiments, n is 2, (Y¹)ₘ is (CH₂)₅, R¹ is —NHR^{A} and contains at least one radioisotope, R^{A} is

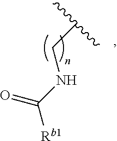

### and R^{b1} is

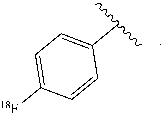 [SMILES: *c1ccc([18F])cc1]

In certain embodiments, n is 2, (Y¹)ₘ is (CH₂)₅, R¹ is —NHR^{A} and contains at least one radioisotope, R^{A} is

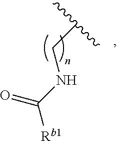

### and R^{b1} is

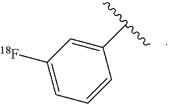 [SMILES: *c1cccc([18F])c1]

In certain embodiments, n is 2, (Y¹)ₘ is (CH₂)₅, R¹ is —NHR^{A} and contains at least one radioisotope, R^{A} is

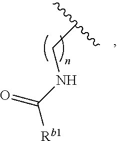

### and R^{b1} is

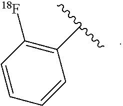 [SMILES: *c1ccccc1[18F]]

In certain embodiments, n is 2, (Y¹)ₘ is (CH₂)₅, R¹ is —NHR^{A} and contains at least one radioisotope, R^{A} is

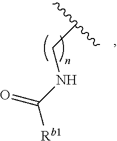

and R^{b1} is optionally substituted heteroaryl. In certain embodiments, n is 2, (Y¹)ₘ is (CH₂)₅, R¹ is —NHR^{A} and contains at least one radioisotope, R^{A} is

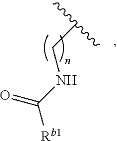

and R^{b1} is heteroaryl substituted with halogen. In certain embodiments, n is 2, (Y¹)ₘ is (CH₂)₅, R¹ is —NHR^{A} and contains at least one radioisotope, R^{A} is

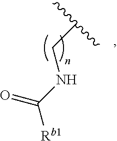

and R^{b1} is heteroaryl substituted with fluorine. In certain embodiments, n is 2, (Y¹)ₘ is (CH₂)₅, R¹ is —NHR^{A} and contains at least one radioisotope, R^{A} is

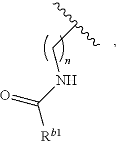

and R^{b1} is heteroaryl substituted with ¹⁸F. In certain embodiments, n is 2, (Y¹)ₘ is (CH₂)₅, R¹ is —NHR^{A} and contains at least one radioisotope, R^{A} is

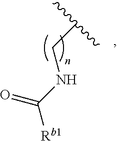

and R^{b1} is optionally substituted 5-14 membered heteroaryl. In certain embodiments, n is 2, (Y¹)ₘ is (CH₂)₅, R¹ is —NHR^{A} and contains at least one radioisotope, R^{A} is

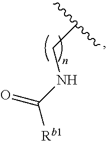

and R^{b1} is 5-14 membered heteroaryl substituted with halogen. In certain embodiments, n is 2, (Y¹)ₘ is (CH₂)₅, R¹ is —NHR^{A} and contains at least one radioisotope, R^{A} is

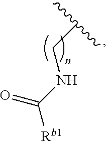

and R^{b1} is 5-14 membered heteroaryl substituted with fluorine. In certain embodiments, n is 2, (Y¹)ₘ is (CH₂)₅, R¹ is —NHR^{A} and contains at least one radioisotope, R^{A} is

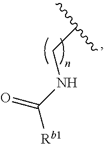

and R^{b1} is 5-14 membered heteroaryl substituted with ¹⁸F. In certain embodiments, n is 2, (Y¹)ₘ is (CH₂)₅, R¹ is —NHR^{A} and contains at least one radioisotope, R^{A} is

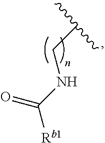

and R^{b1} is pyridyl substituted with fluorine. In certain embodiments, n is 2, (Y¹)ₘ is (CH₂)₅, R¹ is —NHR^{A} and contains at least one radioisotope, R^{A} is

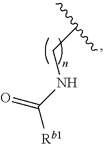

and R^{b1} is pyridyl substituted with ¹⁸F. In certain embodiments, n is 2, (Y¹)ₘ is (CH₂)₅, R¹ is —NHR^{A} and contains at least one radioisotope, R^{A} is

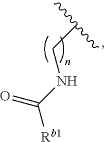

### and R^{b1} is

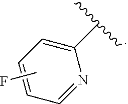

In certain embodiments, n is 2, (Y¹)ₘ is (CH₂)₅, R¹ is —NHR^{A} and contains at least one radioisotope, R^{A} is

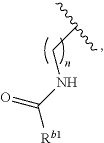

### and R^{b1} is

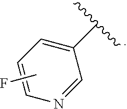

In certain embodiments, n is 2, (Y¹)ₘ is (CH₂)₅, R¹ is —NHR^{A} and contains at least one radioisotope, R^{A} is

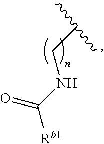

### and R^{b1} is

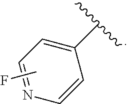

In certain embodiments, n is 2, (Y¹)ₘ is (CH₂)₅, R¹ is —NHR^{A} and contains at least one radioisotope, R^{A} is

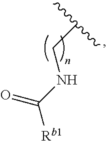

### and R^{b1} is

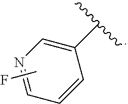

In certain embodiments, n is 2, (Y¹)ₘ is (CH₂)₅, R¹ is —NHR^{A} and contains at least one radioisotope, R^{A} is

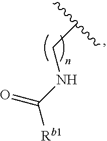

### and R^{b1} is

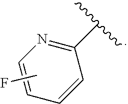

In certain embodiments, n is 2, (Y¹)ₘ is (CH₂)₅, R¹ is —NHR^{A} and contains at least one radioisotope, R^{A} is

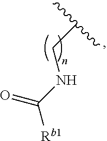

### and R^{b1} is

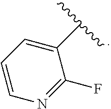 [SMILES: *c1cccnc1F]

In certain embodiments, n is 2, (Y¹)ₘ is (CH₂)₅, R¹ is —NHR^{A} and contains at least one radioisotope, R^{A} is

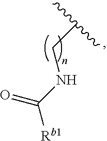

### and R^{b1} is

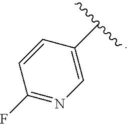 [SMILES: *c1ccc(F)nc1]

In certain embodiments, n is 2, (Y¹)ₘ is (CH₂)₅, R¹ is —NHR^{A} and contains at least one radioisotope, R^{A} is

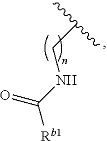

### and R^{b1} is

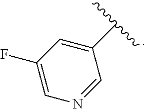 [SMILES: *c1cncc(F)c1]

In certain embodiments, n is 2, (Y¹)ₘ is (CH₂)₅, R¹ is —NHR^{A} and contains at least one radioisotope, R^{A} is

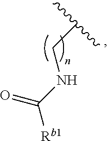

### and R^{b1} is

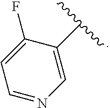 [SMILES: *c1cnccc1F]

In certain embodiments, n is 2, (Y¹)ₘ is (CH₂)₅, R¹ is —NHR^{A} and contains at least one radioisotope, R^{A} is

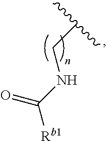

### and R^{b1} is

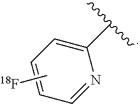

In certain embodiments, n is 2, (Y¹)ₘ is (CH₂)₅, R¹ is —NHR^{A} and contains at least one radioisotope, R^{A} is

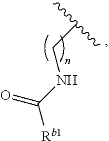

### and R^{b1} is

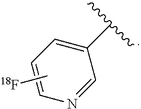

In certain embodiments, n is 2, (Y¹)ₘ is (CH₂)₅, R¹ is —NHR^{A} and contains at least one radioisotope, R^{A} is

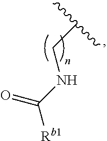

### and R^{b1} is

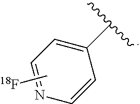

In certain embodiments, n is 2, (Y¹)ₘ is (CH₂)₅, R¹ is —NHR^{A} and contains at least one radioisotope, R^{A} is

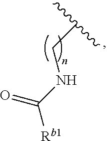

### and R^{b1} is

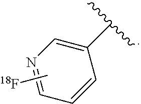

In certain embodiments, n is 2, (Y¹)ₘ is (CH₂)₅, R¹ is —NHR^{A} and contains at least one radioisotope, R^{A} is

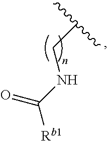

### and R^{b1} is

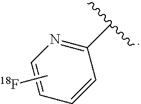

In certain embodiments, n is 2, (Y¹)ₘ is (CH₂)₅, R¹ is —NHR^{A} and contains at least one radioisotope, R^{A} is

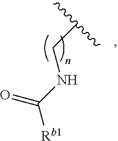

### and R^{b1} is

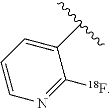

In certain embodiments, n is 2, (Y¹)ₘ is (CH₂)₅, R¹ is —NHR^{A} and contains at least one radioisotope, R^{A} is

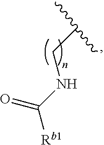

### and R^{b1} is

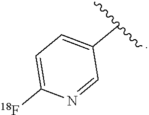 [SMILES: *c1ccc([18F])nc1]

In certain embodiments, n is 2, (Y¹)ₘ is (CH₂)₅, R¹ is —NHR^{A} and contains at least one radioisotope, R^{A} is

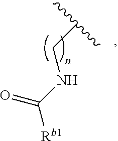

### and R^{b1} is

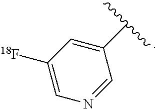 [SMILES: *c1cncc([18F])c1]

In certain embodiments, n is 2, (Y¹)ₘ is (CH₂)₅, R¹ is-NHR^{A} and contains at least one radioisotope, R^{A} is

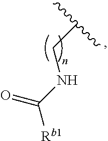

### and R^{b1} is

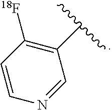 [SMILES: *c1cnccc1[18F]]

### R³

As described herein, R³ is halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{C}, —SCN, —SR^{C}, —SSR^{C}, —N₃, —NO, —N(R^{C})₂, —NO₂, —C(═O)R^{C}, —C(═O)OR^{C}, —C(═O)SR^{C}, —C(═O)N(R^{C})₂, —C(═NR^{C})R^{C}, —C(═NR^{C})OR^{C}, C(═NR^{C})SR^{C}, —C(═NR^{C})N(R^{C})₂, —S(═O)R^{C}, —S(═O)OR^{C}, —S(═O)SR^{C}, —S(═O)N(R^{C})₂, —S(═O)₂R^{C}, —S(═O)₂OR^{C}, —S(═O)₂SR^{C}, —S(═O)₂N(R^{C})₂, —OC(═O)R^{C}, —OC(═O)OR^{C}, —OC(═O)SR^{C}, —OC(═O)N(R^{C})₂, —OC(═NR^{C})R^{C}, —OC(═NR^{C})OR^{C}, —OC(═NR^{C})SR^{C}, —OC(═NR^{C})N(R^{C})₂, —OS(═O)R^{C}, —OS(═O)OR^{C}, —OS(═O)SR^{C}, —OS(═O)N(R^{C})₂, —OS(═O)₂R^{C}, —OS(═O)₂OR^{C}, —OS(═O)₂SR^{C}, —OS(═O)₂N(R^{C})₂, —ON(R^{C})₂, —SC(═O)R^{C}, —SC(═O)OR^{C}, —SC(═O)SR^{C}, —SC(═O)N(R^{C})₂, —SC(═NR^{C})R^{C}, —SC(═NR^{C})OR^{C}, —SC(═NR^{C})SR^{C}, —SC(═NR^{C})N(R^{C})₂, —NR^{C}C(═O)R^{C}, —NR^{C}C(═O)OR^{C}, —NR^{C}C(═O)SR^{C}, —NR^{C}C(═O)N(R^{C})₂, —NR^{C}C(═NR^{C})R^{C}, —NR^{C}C(═NR^{C})OR^{C}, —NR^{C}C(═NR^{C})SR^{C}, —NR^{C}C(═NR^{C})N(R^{C})₂, —NRCS(═O)R^{C}, —NRCS(═O)OR^{C}, —NRCS(═O)SR^{C}, —NRCS(═O)N(R^{C})₂, —NRCS(═O)₂R^{C}, —NRCS(═O)₂OR^{C}, —NRCS(═O)₂SR^{C}, —NRCS(═O)₂N(R^{C})₂, —Si(R^{C})₃, —Si(R^{C})₂OR^{C}, —Si(R^{C}) (OR^{C})₂, —Si(OR)₃, —OSi(R^{C})₃, —OSi(R^{C})₂OR^{C}, —OSi(R^{C}) (OR^{C})₂, —OSi(OR^{C})₃, or —B(OR^{C}) 2.

In certain embodiments, R³ contains at least one radioisotope.

In certain embodiments, R³ is optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl. In certain embodiments, R³ is optionally substituted C₁₋₁₂ alkyl. In certain embodiments, R³ is optionally substituted C₁₋₆ alkyl. In certain embodiments, R³ is unsubstituted C₁₋₆ alkyl. In certain embodiments, R³ is substituted C₁₋₆ alkyl. In certain embodiments, R³ is substituted or unsubstituted methyl, substituted or unsubstituted ethyl, substituted or unsubstituted n-propyl, substituted or unsubstituted isopropyl, substituted or unsubstituted n-butyl, substituted or unsubstituted tert-butyl, substituted or unsubstituted sec-butyl, substituted or unsubstituted isobutyl, substituted or unsubstituted n-pentyl, substituted or unsubstituted 3-pentanyl, substituted or unsubstituted amyl, substituted or unsubstituted neopentyl, substituted or unsubstituted 3-methyl-2-butanyl, substituted or unsubstituted tert-amyl, or substituted or unsubstituted n-hexyl. In certain embodiments, R³ is substituted or unsubstituted methyl. In certain embodiments, R³ is unsubstituted methyl. In certain embodiments, R³ is optionally substituted C₂₋₁₂ alkenyl. In certain embodiments, R³ is optionally substituted C₂₋₆ alkenyl. In certain embodiments, R³ is substituted or unsubstituted ethenyl, substituted or unsubstituted 1-propenyl, substituted or unsubstituted 2-propenyl, substituted or unsubstituted 1-butenyl, substituted or unsubstituted 2-butenyl, substituted or unsubstituted butadienyl, substituted or unsubstituted pentenyl, substituted or unsubstituted pentadienyl, or substituted or unsubstituted hexenyl. In certain embodiments, R³ is optionally substituted C₂₋₁₂ alkynyl. In certain embodiments, R³ is optionally substituted C₂₋₆ alkynyl. In certain embodiments, R³ is substituted or unsubstituted ethynyl, substituted or unsubstituted 1-propynyl, substituted or unsubstituted 2-propynyl, substituted or unsubstituted 1-butynyl, substituted or unsubstituted 2-butynyl, substituted or unsubstituted pentynyl, or substituted or unsubstituted hexynyl. In certain embodiments, R³ is optionally substituted heteroC₁₋₁₂ alkyl. In certain embodiments, R³ is optionally substituted heteroC₁₋₆ alkyl. In certain embodiments, R³ is optionally substituted heteroC₁₋₁₂ alkenyl. In certain embodiments, R³ is optionally substituted heteroC₁₋₆ alkenyl. In certain embodiments, R³ is optionally substituted heteroC₁₋₁₂ alkynyl. In certain embodiments, R³ is optionally substituted heteroC₁₋₆ alkynyl. In certain embodiments, R³ is optionally substituted C₃₋₁₄ cycloalkyl. In certain embodiments, R³ is optionally substituted 6-14 membered aryl. In certain embodiments, R³ is optionally substituted 5-14 membered heteroaryl. In certain embodiments, R³ is optionally substituted 5-10 membered heterocyclyl. In certain embodiments, R³ is optionally substituted azacyclooctyne. In certain embodiments, R³ is an optionally substituted aza-dibenzocyclooctyne.

In certain embodiments, R³ is wherein:

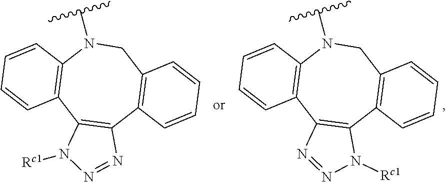

R^{c1} is optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl.

### R^{C}

As described herein, each occurrence of R^{C} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{C} are joined together with their intervening atom to form an optionally substituted heterocyclic ring or optionally substituted heteroaryl ring.

In certain embodiments, at least one occurrence of R^{C} contains at least one radioisotope.

In certain embodiments, at least one occurrence of R^{C} is hydrogen. In certain embodiments, at least one occurrence of R^{C} is optionally substituted acyl. In certain embodiments, at least one occurrence of R^{C} is optionally substituted C₁₋₁₂ alkyl. In certain embodiments, at least one occurrence of R^{C} is optionally substituted C₁₋₆ alkyl. In certain embodiments, at least one occurrence of R^{C} is unsubstituted C₁₋₆ alkyl. In certain embodiments, at least one occurrence of R^{C} is substituted C₁₋₆ alkyl. In certain embodiments, at least one occurrence of R^{C} is substituted or unsubstituted methyl, substituted or unsubstituted ethyl, substituted or unsubstituted n-propyl, substituted or unsubstituted isopropyl, substituted or unsubstituted n-butyl, substituted or unsubstituted tert-butyl, substituted or unsubstituted sec-butyl, substituted or unsubstituted isobutyl, substituted or unsubstituted n-pentyl, substituted or unsubstituted 3-pentanyl, substituted or unsubstituted amyl, substituted or unsubstituted neopentyl, substituted or unsubstituted 3-methyl-2-butanyl, substituted or unsubstituted tert-amyl, or substituted or unsubstituted n-hexyl. In certain embodiments, at least one occurrence of R^{C} is optionally substituted C₂₋₆ alkenyl. In certain embodiments, at least one occurrence of R^{C} is substituted or unsubstituted ethenyl, substituted or unsubstituted 1-propenyl, substituted or unsubstituted 2-propenyl, substituted or unsubstituted 1-butenyl, substituted or unsubstituted 2-butenyl, substituted or unsubstituted butadienyl, substituted or unsubstituted pentenyl, substituted or unsubstituted pentadienyl, or substituted or unsubstituted hexenyl. In certain embodiments, at least one occurrence of R^{C} is optionally substituted C₂₋₆ alkynyl. In certain embodiments, at least one occurrence of R^{C} is substituted or unsubstituted ethynyl, substituted or unsubstituted 1-propynyl, substituted or unsubstituted 2-propynyl, substituted or unsubstituted 1-butynyl, substituted or unsubstituted 2-butynyl, substituted or unsubstituted pentynyl, or substituted or unsubstituted hexynyl. In certain embodiments, at least one occurrence of R^{C} is optionally substituted heteroC₁₋₆ alkyl. In certain embodiments, at least one occurrence of R^{C} is optionally substituted heteroC₁₋₆ alkenyl. In certain embodiments, at least one occurrence of R^{C} is optionally substituted heteroC₁₋₁₂ alkynyl. In certain embodiments, at least one occurrence of RC is optionally substituted heteroC₁₋₆ alkynyl. In certain embodiments, at least one occurrence of R^{C} is optionally substituted C₃₋₁₄ cycloalkyl. In certain embodiments, at least one occurrence of R^{C} is optionally substituted 5-10 membered heterocyclyl. In certain embodiments, at least one occurrence of R^{C} is optionally substituted 6-14 membered aryl. In certain embodiments, at least one occurrence of R^{C} is optionally substituted 5-14 membered heteroaryl. In certain embodiments, at least one occurrence of R^{C} is a nitrogen protecting group. In certain embodiments, at least one occurrence of R^{C} is an oxygen protecting group. In certain embodiments, at least one occurrence of R^{C} is a sulfur protecting group. In certain embodiments, at least two occurrences of R^{C} are joined together with their intervening atom to form an optionally substituted 5-10 membered heterocyclic ring. In certain embodiments, at least two occurrences of R^{C} are joined together with their intervening atom to form an optionally substituted 5-14 membered heteroaryl ring.

In certain embodiments, R³ is —N(R^{C})₂ and two occurrences of R^{C} are joined together with their intervening atom to form an optionally substituted 5-10 membered heterocyclyl. In certain embodiments, R³ is —N(R^{C})₂ and two occurrences of R^{C} are joined together with their intervening atom to form an optionally substituted 8 membered heterocyclyl. In certain embodiments, R³ is —N(R^{C})₂ and two occurrences of R^{C} are joined together with their intervening atom to form an optionally substituted azacyclooctyne. In certain embodiments, R³ is —N(R^{C})₂ and two occurrences of R^{C} are joined together with their intervening atom to form an optionally substituted aza-dibenzocyclooctyne. In certain embodiments, R³ is —N(R^{C})₂ and two occurrences of R^{C} are joined together with their intervening atom to form

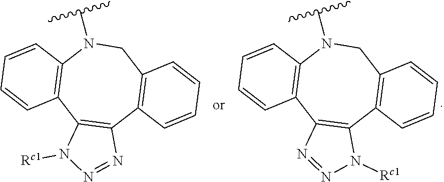

### R^{c1}

As described herein, each occurrence of R^{c1} is optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl.

In certain embodiments, at least one occurrence of R^{c1} contains at least one radioisotope.

In certain embodiments, R^{c1} is optionally substituted alkyl. In certain embodiments, R^{c1} is alkyl substituted with halogen. In certain embodiments, R^{c1} is alkyl substituted with fluorine, chlorine, or bromine. In certain embodiments, R^{c1} is alkyl substituted with fluorine. In certain embodiments, R^{c1} is optionally substituted C₁₋₁₂ alkyl. In certain embodiments, R^{c1} is C₁₋₁₂ alkyl substituted with halogen. In certain embodiments, R^{c1} is optionally substituted C₁₋₆ alkyl. In certain embodiments, R^{c1} is C₁₋₆ alkyl substituted with halogen. In certain embodiments, R^{c1} is unsubstituted C₁₋₆ alkyl. In certain embodiments, R^{c1} is substituted C₁₋₆ alkyl. In certain embodiments, R^{c1} is optionally substituted C₁₋₆ alkyl optionally substituted with one or more halogen or optionally substituted alkyl groups. In certain embodiments, R^{c1} is substituted or unsubstituted methyl, substituted or unsubstituted ethyl, substituted or unsubstituted n-propyl, substituted or unsubstituted isopropyl, substituted or unsubstituted n-butyl, substituted or unsubstituted tert-butyl, substituted or unsubstituted sec-butyl, substituted or unsubstituted isobutyl, substituted or unsubstituted n-pentyl, substituted or unsubstituted 3-pentanyl, substituted or unsubstituted amyl, substituted or unsubstituted neopentyl, substituted or unsubstituted 3-methyl-2-butanyl, substituted or unsubstituted tert-amyl, or substituted or unsubstituted n-hexyl. In certain embodiments, R^{c1} is methyl substituted with halogen, ethyl substituted with halogen, n-propyl substituted with halogen, isopropyl substituted with halogen, n-butyl substituted with halogen, tert-butyl substituted with halogen, sec-butyl substituted with halogen, isobutyl substituted with halogen, n-pentyl substituted with halogen, 3-pentanyl substituted with halogen, amyl substituted with halogen, neopentyl substituted with halogen, 3-methyl-2-butanyl substituted with halogen, tert-amyl substituted with halogen, or n-hexyl substituted with halogen. In certain embodiments, R^{c1} is C₁₋₁₂ alkyl substituted with fluorine. In certain embodiments, R^{c1} is C₁₋₆ alkyl substituted with fluorine. In certain embodiments, R^{c1} is methyl substituted with fluorine, ethyl substituted with fluorine, n-propyl substituted with fluorine, isopropyl substituted with fluorine, n-butyl substituted with fluorine, tert-butyl substituted with fluorine, sec-butyl substituted with fluorine, isobutyl substituted with fluorine, n-pentyl substituted with fluorine, 3-pentanyl substituted with fluorine, amyl substituted with fluorine, neopentyl substituted with fluorine, 3-methyl-2-butanyl substituted with fluorine, tert-amyl substituted with fluorine, or n-hexyl substituted with fluorine. In certain embodiments, R^{c1} is ethyl substituted with fluorine. In certain embodiments, R^{c1} is alkyl substituted with ¹⁸F. In certain embodiments, R^{c1} is C₁₋₁₂ alkyl substituted with ¹⁸F. In certain embodiments, R^{c1} is C₁₋₆ alkyl substituted with ¹⁸F. In certain embodiments, R^{c1} is methyl substituted with ¹⁸F, ethyl substituted with ¹⁸F, n-propyl substituted with ¹⁸F, isopropyl substituted with ¹⁸F, n-butyl substituted with ¹⁸F, tert-butyl substituted with ¹⁸F, sec-butyl substituted with ¹⁸F, isobutyl substituted with ¹⁸F, n-pentyl substituted with ¹⁸F, 3-pentanyl substituted with ¹⁸F, amyl substituted with ¹⁸F, neopentyl substituted with ¹⁸F, 3-methyl-2-butanyl substituted with ¹⁸F, tert-amyl substituted with ¹⁸F, or n-hexyl substituted with ¹⁸F. In certain embodiments, R^{c1} is ethyl substituted with ¹⁸F.

In certain embodiments, R³ is

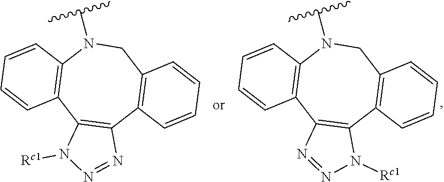

and R^{c1} is optionally substituted alkyl. In certain embodiments, R³ is

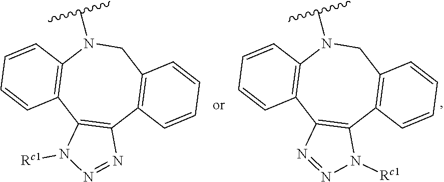

and R^{c1} is alkyl substituted with halogen. In certain embodiments, R³ is

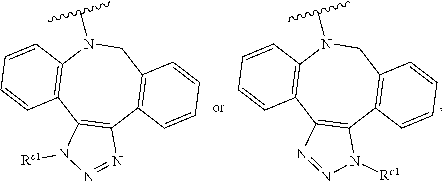

and R^{c1} is alkyl substituted with fluorine, chlorine, or bromine. In certain embodiments, R³ is

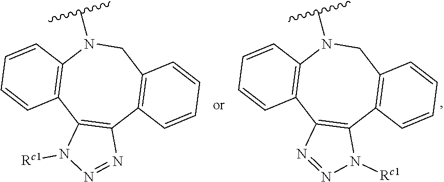

and R^{c1} is alkyl substituted with fluorine. In certain embodiments, R³ is

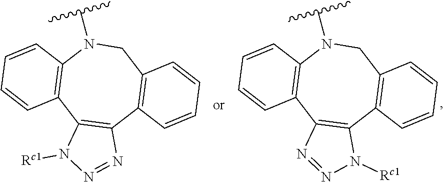

and R^{c1} is optionally substituted C₁₋₁₂ alkyl. In certain embodiments, R³ is

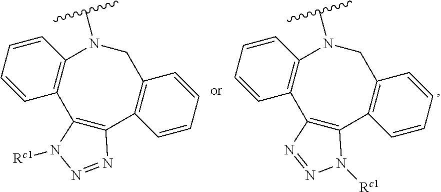

and R^{c1} is C₁₋₁₂ alkyl substituted with halogen. In certain embodiments, R³ is

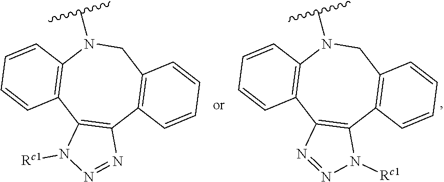

and R^{c1} is optionally substituted C₁₋₆ alkyl. In certain embodiments, R³ is

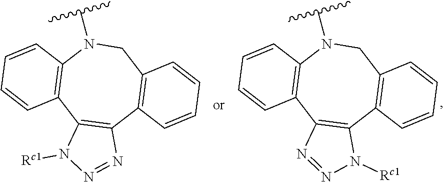

and R^{c1} is C₁₋₆ alkyl substituted with halogen. In certain embodiments, R³ is

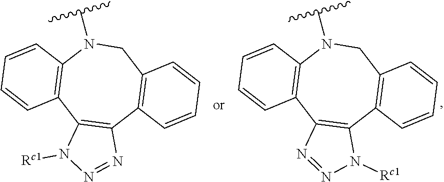

and R^{c1} is unsubstituted C₁₋₆ alkyl. In certain embodiments, R³ is

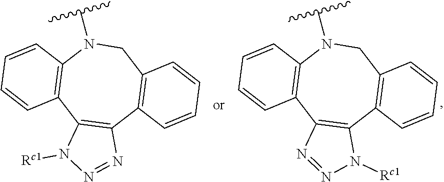

and R^{c1} is substituted C₁₋₆ alkyl. In certain embodiments, R³ is

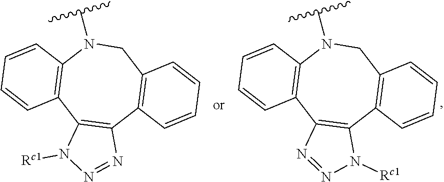

and R^{c1} is optionally substituted C₁₋₆ alkyl optionally substituted with one or more halogen or optionally substituted alkyl groups. In certain embodiments, R³ is

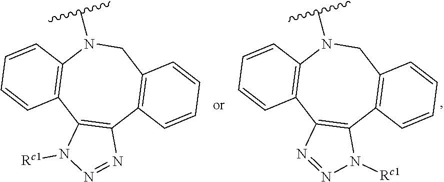

and R^{c1} is substituted or unsubstituted methyl, substituted or unsubstituted ethyl, substituted or unsubstituted n-propyl, substituted or unsubstituted isopropyl, substituted or unsubstituted n-butyl, substituted or unsubstituted tert-butyl, substituted or unsubstituted sec-butyl, substituted or unsubstituted isobutyl, substituted or unsubstituted n-pentyl, substituted or unsubstituted 3-pentanyl, substituted or unsubstituted amyl, substituted or unsubstituted neopentyl, substituted or unsubstituted 3-methyl-2-butanyl, substituted or unsubstituted tert-amyl, or substituted or unsubstituted n-hexyl. In certain embodiments, R³ is

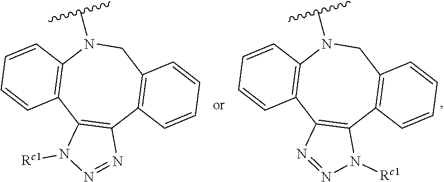

and R^{c1} is methyl substituted with halogen, ethyl substituted with halogen, n-propyl substituted with halogen, isopropyl substituted with halogen, n-butyl substituted with halogen, tert-butyl substituted with halogen, sec-butyl substituted with halogen, isobutyl substituted with halogen, n-pentyl substituted with halogen, 3-pentanyl substituted with halogen, amyl substituted with halogen, neopentyl substituted with halogen, 3-methyl-2-butanyl substituted with halogen, tert-amyl substituted with halogen, or n-hexyl substituted with halogen. In certain embodiments, R³ is

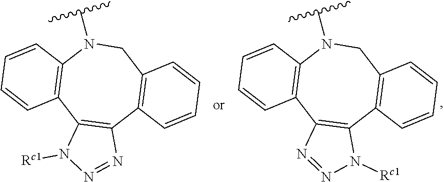

and R^{c1} is C₁₋₁₂ alkyl substituted with fluorine. In certain embodiments, R³ is

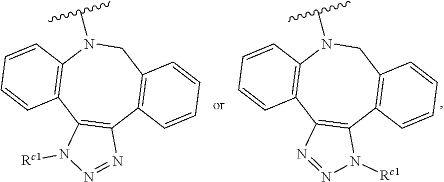

and R^{c1} is C₁₋₆ alkyl substituted with fluorine. In certain embodiments, R³ is

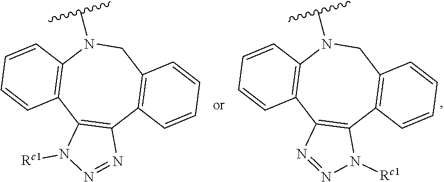

and R^{c1} is methyl substituted with fluorine, ethyl substituted with fluorine, n-propyl substituted with fluorine, isopropyl substituted with fluorine, n-butyl substituted with fluorine, tert-butyl substituted with fluorine, sec-butyl substituted with fluorine, isobutyl substituted with fluorine, n-pentyl substituted with fluorine, 3-pentanyl substituted with fluorine, amyl substituted with fluorine, neopentyl substituted with fluorine, 3-methyl-2-butanyl substituted with fluorine, tert-amyl substituted with fluorine, or n-hexyl substituted with fluorine. In certain embodiments, R³ is

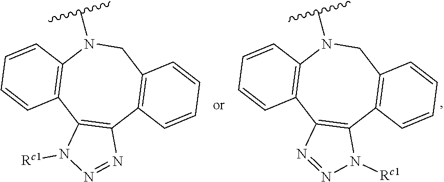

and R^{c1} is ethyl substituted with fluorine. In certain embodiments, R³ is

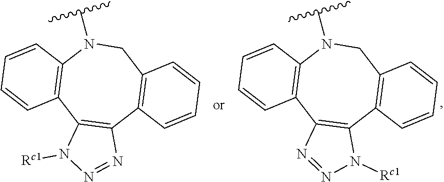

and R^{c1} is alkyl substituted with ¹⁸F. In certain embodiments, R³ is

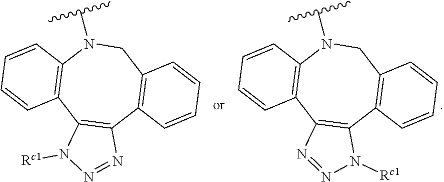

and R^{c1} is C₁₋₁₂ alkyl substituted with ¹⁸F. In certain embodiments, R³ is

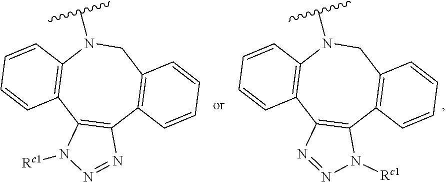

and R^{c1} is C₁₋₆ alkyl substituted with ¹⁸F. In certain embodiments, R³ is

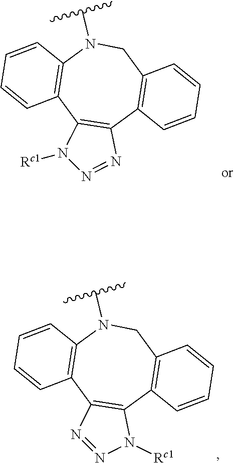

and R^{c1} is methyl substituted with ¹⁸F, ethyl substituted with ¹⁸F, n-propyl substituted with ¹⁸F, isopropyl substituted with ¹⁸F, n-butyl substituted with ¹⁸F, tert-butyl substituted with ¹⁸F, sec-butyl substituted with ¹⁸F, isobutyl substituted with ¹⁸F, n-pentyl substituted with ¹⁸F, 3-pentanyl substituted with ¹⁸F, amyl substituted with ¹⁸F, neopentyl substituted with ¹⁸F, 3-methyl-2-butanyl substituted with ¹⁸F, tert-amyl substituted with ¹⁸F, or n-hexyl substituted with ¹⁸F. In certain embodiments, R³ is

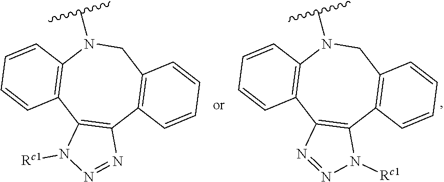

and R^{c1} is ethyl substituted with ¹⁸F.

In certain embodiments, R^{c1} is optionally substituted C₂₋₆ alkenyl. In certain embodiments, R^{c1} is substituted or unsubstituted ethenyl, substituted or unsubstituted 1-propenyl, substituted or unsubstituted 2-propenyl, substituted or unsubstituted 1-butenyl, substituted or unsubstituted 2-butenyl, substituted or unsubstituted butadienyl, substituted or unsubstituted pentenyl, substituted or unsubstituted pentadienyl, or substituted or unsubstituted hexenyl. In certain embodiments, R^{c1} is optionally substituted C₂₋₆ alkynyl. In certain embodiments, R^{c1} is substituted or unsubstituted ethynyl, substituted or unsubstituted 1-propynyl, substituted or unsubstituted 2-propynyl, substituted or unsubstituted 1-butynyl, substituted or unsubstituted 2-butynyl, substituted or unsubstituted pentynyl, or substituted or unsubstituted hexynyl. In certain embodiments, R^{c1} is optionally substituted heteroC₁₋₆ alkyl. In certain embodiments, R^{c1} is optionally substituted heteroC₁₋₆ alkenyl. In certain embodiments, R^{c1} is optionally substituted heteroC₁₋₁₂ alkynyl. In certain embodiments, R^{c1} is optionally substituted heteroC₁₋₆ alkynyl. In certain embodiments, R^{c1} is optionally substituted C₃₋₁₄ cycloalkyl. In certain embodiments, R^{c1} is optionally substituted 5-10 membered heterocyclyl. In certain embodiments, R^{c1} is optionally substituted 6-14 membered aryl. In certain embodiments, R^{c1} is optionally substituted 5-14 membered heteroaryl.

### R⁴

As described herein, R⁴ is optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl.

In certain embodiments, R⁴ contains at least one radioisotope.

In certain embodiments, R⁴ is optionally substituted aryl. In certain embodiments, R⁴ is aryl substituted with optionally substituted alkyl. In certain embodiments, R⁴ is 6-14 membered aryl substituted with optionally substituted alkyl. In certain embodiments, R⁴ is aryl substituted with optionally substituted alkyl substituted with optionally substituted heterocyclyl. In certain embodiments, R⁴ is 6-14 membered aryl substituted with optionally substituted alkyl substituted with optionally substituted heterocyclyl. In certain embodiments, R⁴ is phenyl substituted with optionally substituted alkyl substituted with optionally substituted heterocyclyl. In certain embodiments, R⁴ comprises a chelating moiety. In certain embodiments, R⁴ comprises dodecane tetraacetic acid (DOTA). In certain embodiments, R⁴ comprises 1,4,7-triazacyclononane-1,4,7-triacetic acid (NOTA). In certain embodiments, R⁴ is

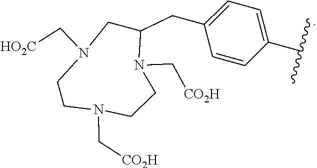 [SMILES: *c1ccc(CC2CN(CC(=O)O)CCN(CC(=O)O)CCN2CC(=O)O)cc1]

In certain embodiments, R⁴ is

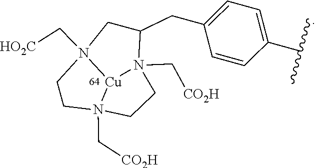

In certain embodiments, R⁴ is

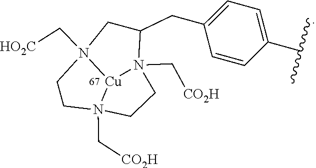

In certain embodiments, R⁴ comprises

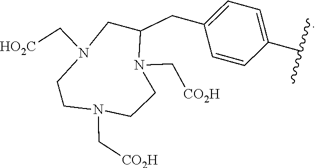 [SMILES: *c1ccc(CC2CN(CC(=O)O)CCN(CC(=O)O)CCN2CC(=O)O)cc1]

In certain embodiments, R⁴ comprises a copper radioisotope (e.g., ⁶⁴Cu, ⁶⁷Cu). In certain embodiments, R⁴ comprises ⁶⁴Cu. In certain embodiments, R⁴ comprises ⁶⁷Cu. In certain embodiments, R⁴ comprises

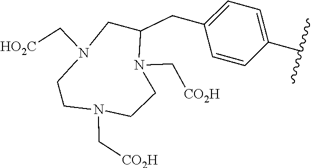 [SMILES: *c1ccc(CC2CN(CC(=O)O)CCN(CC(=O)O)CCN2CC(=O)O)cc1]

and ⁶⁴Cu. In certain embodiments, R⁴ comprises

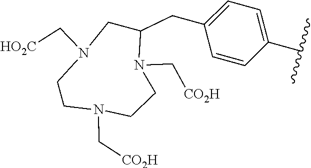 [SMILES: *c1ccc(CC2CN(CC(=O)O)CCN(CC(=O)O)CCN2CC(=O)O)cc1]

and ⁶⁷Cu. In certain embodiments, R⁴ comprises a chelate of

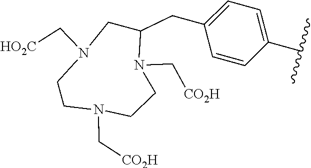 [SMILES: *c1ccc(CC2CN(CC(=O)O)CCN(CC(=O)O)CCN2CC(=O)O)cc1]

and ⁶⁴Cu. In certain embodiments, R⁴ comprises a chelate of

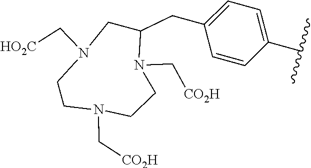 [SMILES: *c1ccc(CC2CN(CC(=O)O)CCN(CC(=O)O)CCN2CC(=O)O)cc1]

### and ⁶⁷Cu.

In certain embodiments, n is 2, (Y¹)ₘ is (CH₂)₅, R¹ is —NHR^{A} and contains at least one radioisotope, and R^{A} is

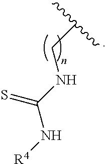

In certain embodiments, n is 2, (Y¹)ₘ is (CH₂)₅, R¹ is —NHR^{A} and contains at least one radioisotope, R^{A} is

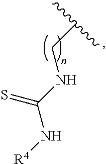

and R⁴ is optionally substituted aryl or optionally substituted heteroaryl. In certain embodiments, n is 2, (Y¹)ₘ is (CH₂)₅, R¹ is —NHR^{A} and contains at least one radioisotope, R^{A} is

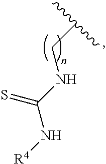

and R⁴ is optionally substituted aryl. In certain embodiments, n is 2, (Y¹)ₘ is (CH₂)₅, R¹ is —NHR^{A} and contains at least one radioisotope, R^{A} is

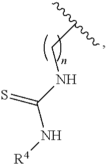

and R⁴ is aryl substituted with optionally substituted alkyl. In certain embodiments, n is 2, (Y¹)ₘ is (CH₂)₅, R¹ is —NHR^{A} and contains at least one radioisotope, R^{A} is

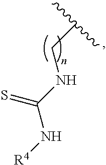

and R⁴ is 6-14 membered aryl substituted with optionally substituted alkyl. In certain embodiments, n is 2, (Y¹)ₘ is (CH₂)₅, R¹ is —NHR^{A} and contains at least one radioisotope, R^{A} is

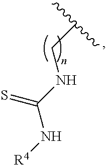

and R⁴ is aryl substituted with optionally substituted alkyl substituted with optionally substituted heterocyclyl. In certain embodiments, n is 2, (Y¹)ₘ is (CH₂)₅, R¹ is —NHR^{A} and contains at least one radioisotope, R^{A} is

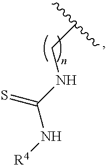

and R⁴ is 6-14 membered aryl substituted with optionally substituted alkyl substituted with optionally substituted heterocyclyl. In certain embodiments, n is 2, (Y¹)ₘ is (CH₂)₅, R¹ is —NHR^{A} and contains at least one radioisotope, R^{A} is

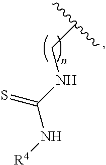

and R⁴ is phenyl substituted with optionally substituted alkyl substituted with optionally substituted heterocyclyl. In certain embodiments, n is 2, (Y¹)ₘ is (CH₂)₅, R¹ is —NHR^{A} and contains at least one radioisotope, R^{A} is

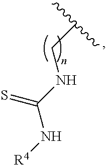

### and R⁴ is

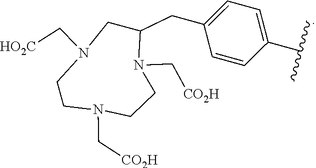 [SMILES: *c1ccc(CC2CN(CC(=O)O)CCN(CC(=O)O)CCN2CC(=O)O)cc1]

In certain embodiments, n is 2, (Y¹)ₘ is (CH₂)₅, R¹ is —NHR^{A} and contains at least one radioisotope, R^{A} is

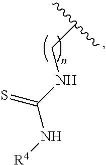

and R⁴ comprises

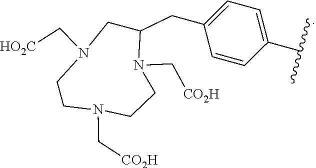 [SMILES: *c1ccc(CC2CN(CC(=O)O)CCN(CC(=O)O)CCN2CC(=O)O)cc1]

In certain embodiments, n is 2, (Y¹)ₘ is (CH₂)₅, R¹ is —NHR^{A} and contains at least one radioisotope, R^{A} is

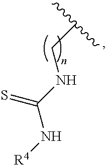

and R⁴ comprises a copper radioisotope (e.g., ⁶⁴Cu, ⁶⁷Cu). In certain embodiments, n is 2, (Y¹)ₘ is (CH₂)₅, R¹ is —NHR^{A} and contains at least one radioisotope, R^{A} is

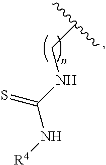

and R⁴ comprises ⁶⁴Cu. In certain embodiments, n is 2, (Y¹)ₘ is (CH₂)₅, R¹ is —NHR^{A} and contains at least one radioisotope, R^{A} is

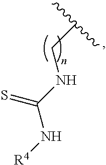

and R⁴ comprises ⁶⁷Cu. In certain embodiments, n is 2, (Y¹)ₘ is (CH₂)₅, R¹ is —NHR^{A} and contains at least one radioisotope, R^{A} is

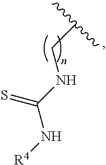

and R⁴ comprises

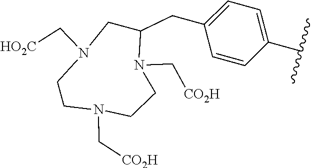 [SMILES: *c1ccc(CC2CN(CC(=O)O)CCN(CC(=O)O)CCN2CC(=O)O)cc1]

and ⁶⁴Cu. In certain embodiments, n is 2, (Y¹)ₘ is (CH₂)₅, R¹ is —NHR^{A} and contains at least one radioisotope, R^{A} is

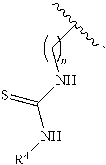

and R⁴ comprises

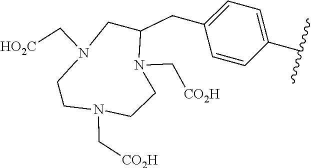 [SMILES: *c1ccc(CC2CN(CC(=O)O)CCN(CC(=O)O)CCN2CC(=O)O)cc1]

and ⁶⁷Cu. In certain embodiments, n is 2, (Y¹)ₘ is (CH₂)₅, R¹ is —NHR^{A} and contains at least one radioisotope, R^{A} is

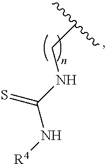

and R⁴ comprises a chelate of

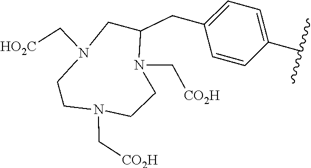 [SMILES: *c1ccc(CC2CN(CC(=O)O)CCN(CC(=O)O)CCN2CC(=O)O)cc1]

and ⁶⁴Cu. In certain embodiments, n is 2, (Y¹)ₘ is (CH₂)₅, R¹ is —NHR^{A} and contains at least one radioisotope, R^{A} is

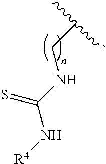

and R⁴ comprises a chelate of

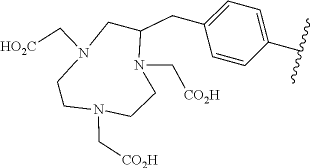 [SMILES: *c1ccc(CC2CN(CC(=O)O)CCN(CC(=O)O)CCN2CC(=O)O)cc1]

### and ⁶⁷Cu.

p

As described herein, p is 0, 1, 2, 3, 4, 5, 6, 7, 8, 9, or 10. In certain embodiments, p is 0. In certain embodiments, p is 1. In certain embodiments, p is 2. In certain embodiments, p is 3. In certain embodiments, p is 4. In certain embodiments, p is 5. In certain embodiments, p is 6. In certain embodiments, p is 7. In certain embodiments, p is 8. In certain embodiments, p is 9. In certain embodiments, p is 10.

In certain embodiments, p is 2 and m is 5. In certain embodiments, p is 2, m is 5, and at least one occurrence of Y¹ is CH₂. In certain embodiments, p is 2 and (Y¹)ₘ is (CH₂)₅. In certain embodiments, p is 2, (Y¹)ₘ is (CH₂)₅, and R¹ is —N(R^{A})₂ and contains at least one radioisotope. In certain embodiments, p is 2, (Y¹)ₘ is (CH₂)₅, and R¹ is —NHR^{A} and contains at least one radioisotope. In certain embodiments, p is 2, (Y¹)ₘ is (CH₂)₅, R¹ is —NHR^{A} and contains at least one radioisotope, and R^{A} is

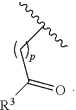

In certain embodiments, p is 2, (Y¹)ₘ is (CH₂)₅, R¹ is —NHR^{A} and contains at least one radioisotope, R^{A} is

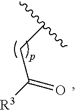

and R³ is optionally substituted 5-14 membered heteroaryl. In certain embodiments, p is 2, (Y¹)ₘ is (CH₂)₅, R¹ is —NHR^{A} and contains at least one radioisotope, R^{A} is

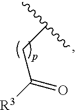

and R³ is optionally substituted 5-10 membered heterocyclyl. In certain embodiments, p is 2, (Y¹)ₘ is (CH₂)₅, R¹ is —NHR^{A} and contains at least one radioisotope, R^{A} is

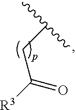

and R³ is optionally substituted azacyclooctyne. In certain embodiments, p is 2, (Y¹)ₘ is (CH₂)₅, R¹ is —NHR^{A} and contains at least one radioisotope, R^{A} is

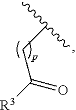

and R³ is an optionally substituted aza-dibenzocyclooctyne. In certain embodiments, p is 2, (Y¹)ₘ is (CH₂)₅, R¹ is —NHR^{A} and contains at least one radioisotope, R^{A} is

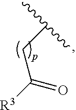

### and R³ is

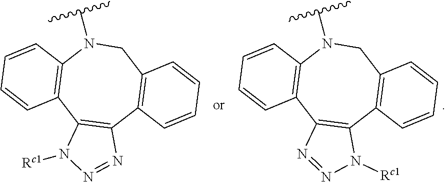

In certain embodiments, p is 2, (Y¹)ₘ is (CH₂)₅, R¹ is —NHR^{A} and contains at least one radioisotope, R^{A} is

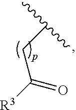

### R³ is

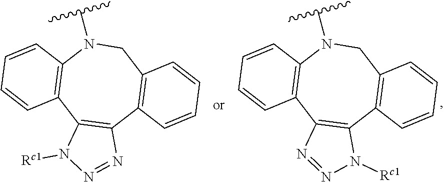

and R^{c1} is ethyl substituted with fluorine. In certain embodiments, p is 2, (Y¹)ₘ is (CH₂)₅, R¹ is —NHR^{A} and contains at least one radioisotope, R^{A} is

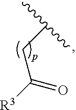

### R³ is

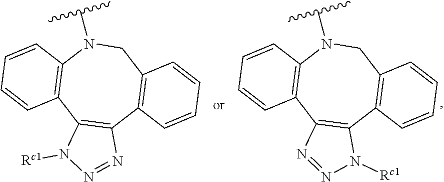

and R^{c1} is alkyl substituted with ¹⁸F. In certain embodiments, p is 2, (Y¹)ₘ is (CH₂)₅, R¹ is —NHR^{A} and contains at least one radioisotope, R^{A} is

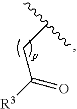

### R³ is

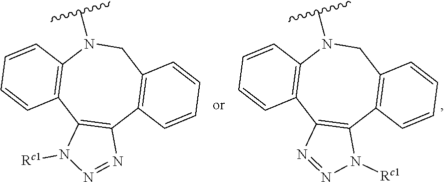

and R^{c1} is C₁₋₁₂ alkyl substituted with ¹⁸F. In certain embodiments, p is 2, (Y¹)ₘ is (CH₂)₅, R¹ is —NHR^{A} and contains at least one radioisotope, R^{A} is

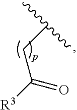

### R³ is

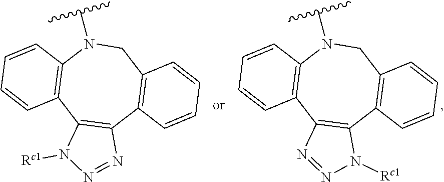

and R^{c1} is C₁₋₆ alkyl substituted with ¹⁸F. In certain embodiments, p is 2, (Y¹)ₘ is (CH₂)₅, R¹ is —NHR^{A} and contains at least one radioisotope, R^{A} is

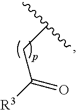

### R³ is

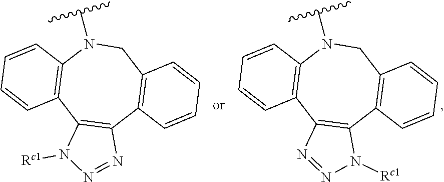

and R^{c1} is methyl substituted with ¹⁸F, ethyl substituted with ¹⁸F, n-propyl substituted with ¹⁸F, isopropyl substituted with ¹⁸F, n-butyl substituted with ¹⁸F, tert-butyl substituted with ¹⁸F, sec-butyl substituted with ¹⁸F, isobutyl substituted with ¹⁸F, n-pentyl substituted with ¹⁸F, 3-pentanyl substituted with ¹⁸F, amyl substituted with ¹⁸F, neopentyl substituted with ¹⁸F, 3-methyl-2-butanyl substituted with ¹⁸F, tert-amyl substituted with ¹⁸F, or n-hexyl substituted with ¹⁸F. In certain embodiments, p is 2, (Y¹)ₘ is (CH₂)₅, R¹ is —NHR^{A} and contains at least one radioisotope, R^{A} is

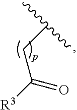

### R³ is

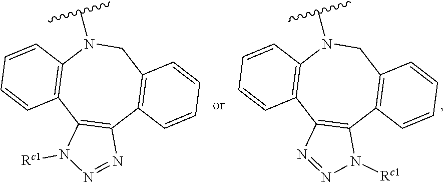

and R^{c1} is ethyl substituted with ¹⁸F. In certain embodiments, p is 2, (Y¹)ₘ is (CH₂)₅, R¹ is —NHR^{A} and contains at least one radioisotope, R^{A} is

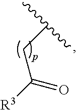

R³ is —N(RC)₂, and two occurrences of R^{C} are joined together with their intervening atom to form an optionally substituted 5-10 membered heterocyclyl. In certain embodiments, p is 2, (Y¹)ₘ is (CH₂)₅, R¹ is —NHR^{A} and contains at least one radioisotope, R^{A} is

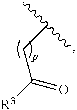

R³ is —N(RC)₂, and two occurrences of R^{C} are joined together with their intervening atom to form an optionally substituted 8 membered heterocyclyl. In certain embodiments, p is 2, (Y¹) m is (CH₂)₅, R¹ is —NHR^{A} and contains at least one radioisotope, R^{A} is

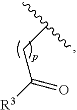

R³ is —N(RC)₂, and two occurrences of R^{C} are joined together with their intervening atom to form an optionally substituted azacyclooctyne. In certain embodiments, p is 2, (Y¹)ₘ is (CH₂)₅, R¹ is —NHR^{A} and contains at least one radioisotope, R^{A} is

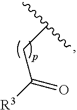

R³ is —N(RC)₂, and two occurrences of R^{C} are joined together with their intervening atom to form an optionally substituted aza-dibenzocyclooctyne. In certain embodiments, p is 2, (Y¹)ₘ is (CH₂)₅, R¹ is —NHR^{A} and contains at least one radioisotope, R^{A} is

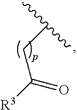

R³ is —N(RC)₂, and two occurrences of R^{C} are joined together with their intervening atom to form

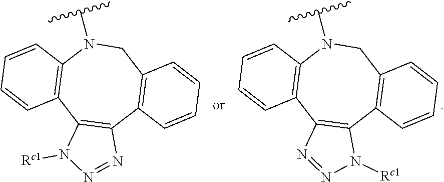

In certain embodiments, p is 2, (Y¹)ₘ is (CH₂)₅, R¹ is —NHR^{A} and contains at least one radioisotope, R^{A} is

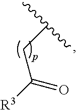

R³ is —N(RC)₂, two occurrences of R^{C} are joined together with their intervening atom to form

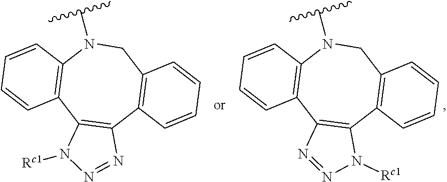

and R^{c1} is optionally substituted alkyl. In certain embodiments, p is 2, (Y¹)ₘ is (CH₂)₅, R¹ is —NHR^{A} and contains at least one radioisotope, R^{A} is

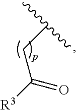

R³ is —N(RC)₂, two occurrences of R^{C} are joined together with their intervening atom to form

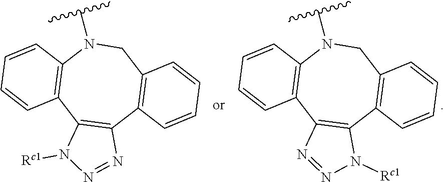

and R^{c1} is alkyl substituted with halogen. In certain embodiments, p is 2, (Y¹)ₘ is (CH₂)₅, R¹ is —NHR^{A} and contains at least one radioisotope, R^{A} is

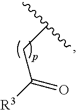

R³ is —N(RC)₂, two occurrences of R^{C} are joined together with their intervening atom to form

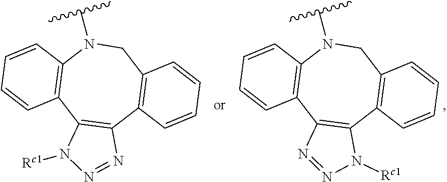

and R^{c1} is alkyl substituted with fluorine, chlorine, or bromine. In certain embodiments, R^{c1} is alkyl substituted with fluorine. In certain embodiments, p is 2, (Y¹)ₘ is (CH₂)₅, R¹ is —NHR^{A} and contains at least one radioisotope, R^{A} is

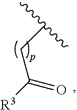

R³ is —N(RC)₂, two occurrences of R^{C} are joined together with their intervening atom to form

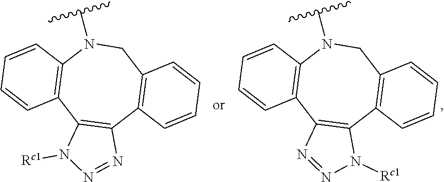

and R^{c1} is optionally substituted C₁₋₁₂ alkyl. In certain embodiments, p is 2, (Y¹)ₘ is (CH₂)₅, R¹ is —NHR^{A} and contains at least one radioisotope, R^{A} is

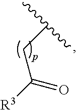

R³ is —N(RC)₂, two occurrences of R^{C} are joined together with their intervening atom to form

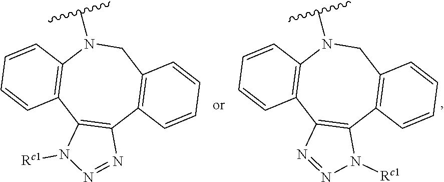

and R^{c1} is C₁₋₁₂ alkyl substituted with halogen. In certain embodiments, p is 2, (Y¹)ₘ is (CH₂)₅, R¹ is —NHR^{A} and contains at least one radioisotope, R^{A} is

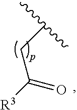

R³ is —N(RC)₂, two occurrences of R^{C} are joined together with their intervening atom to form

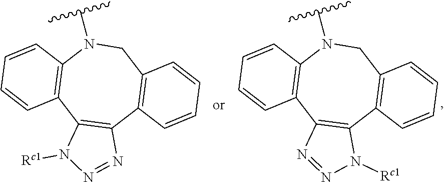

and R^{c1} is optionally substituted C₁₋₆ alkyl. In certain embodiments, p is 2, (Y¹)ₘ is (CH₂)₅, R¹ is —NHR^{A} and contains at least one radioisotope, R^{A} is

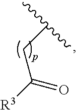

R³ is —N(RC)₂, two occurrences of R^{C} are joined together with their intervening atom to form

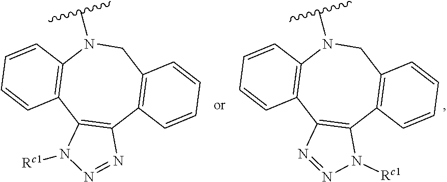

and R^{c1} is C₁₋₆ alkyl substituted with halogen. In certain embodiments, p is 2, (Y¹)ₘ is (CH₂)₅, R¹ is —NHR^{A} and contains at least one radioisotope, R^{A} is

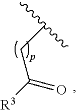

R³ is —N(RC)₂, two occurrences of R^{C} are joined together with their intervening atom to form

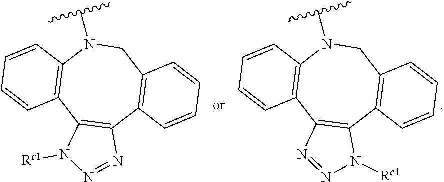

and R^{c1} is unsubstituted C₁₋₆ alkyl. In certain embodiments, p is 2, (Y¹)ₘ is (CH₂)₅, R¹ is —NHR^{A} and contains at least one radioisotope, R^{A} is

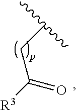

R³ is —N(RC)₂, two occurrences of R^{C} are joined together with their intervening atom to form

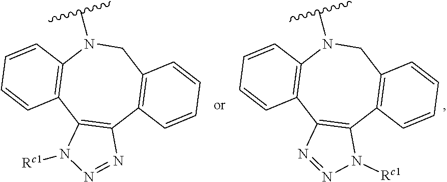

and R^{c1} is substituted C₁₋₆ alkyl. In certain embodiments, p is 2, (Y¹)ₘ is (CH₂)₅, R¹ is —NHR^{A} and contains at least one radioisotope, R^{A} is

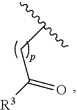

R³ is —N(RC)₂, two occurrences of R^{C} are joined together with their intervening atom to form

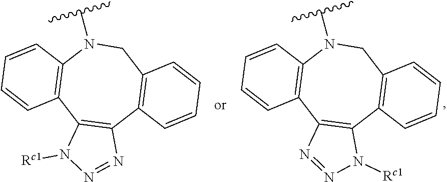

and R^{c1} is optionally substituted C₁₋₆ alkyl optionally substituted with one or more halogen or optionally substituted alkyl groups. In certain embodiments, p is 2, (Y¹)ₘ is (CH₂)₅, R¹ is —NHR^{A} and contains at least one radioisotope, R^{A} is

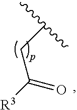

R³ is —N(RC)₂, two occurrences of R^{C} are joined together with their intervening atom to form

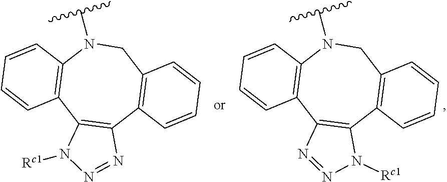

and R^{c1} is substituted or unsubstituted methyl, substituted or unsubstituted ethyl, substituted or unsubstituted n-propyl, substituted or unsubstituted isopropyl, substituted or unsubstituted n-butyl, substituted or unsubstituted tert-butyl, substituted or unsubstituted sec-butyl, substituted or unsubstituted isobutyl, substituted or unsubstituted n-pentyl, substituted or unsubstituted 3-pentanyl, substituted or unsubstituted amyl, substituted or unsubstituted neopentyl, substituted or unsubstituted 3-methyl-2-butanyl, substituted or unsubstituted tert-amyl, or substituted or unsubstituted n-hexyl. In certain embodiments, p is 2, (Y¹)ₘ is (CH₂)₅, R¹ is —NHR^{A} and contains at least one radioisotope, R^{A} is

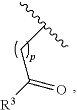

R³ is —N(RC)₂, two occurrences of R^{C} are joined together with their intervening atom to form

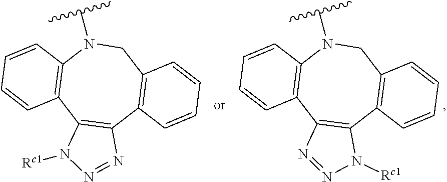

and R^{c1} is methyl substituted with halogen, ethyl substituted with halogen, n-propyl substituted with halogen, isopropyl substituted with halogen, n-butyl substituted with halogen, tert-butyl substituted with halogen, sec-butyl substituted with halogen, isobutyl substituted with halogen, n-pentyl substituted with halogen, 3-pentanyl substituted with halogen, amyl substituted with halogen, neopentyl substituted with halogen, 3-methyl-2-butanyl substituted with halogen, tert-amyl substituted with halogen, or n-hexyl substituted with halogen. In certain embodiments, p is 2, (Y¹)ₘ is (CH₂)₅, R¹ is —NHR^{A} and contains at least one radioisotope, R^{A} is

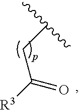

R³ is —N(RC)₂, two occurrences of R^{C} are joined together with their intervening atom to form

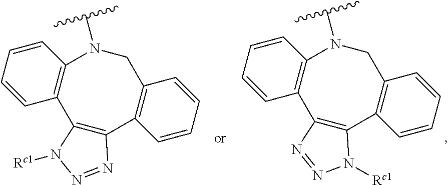

and R^{c1} is C₁₋₁₂ alkyl substituted with fluorine. In certain embodiments, p is 2, (Y¹)ₘ is (CH₂)₅, R¹ is —NHR^{A} and contains at least one radioisotope, R^{A} is

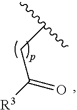

R³ is —N(RC)₂, two occurrences of R^{C} are joined together with their intervening atom to form

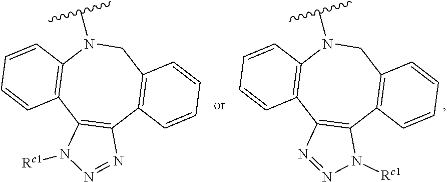

and R^{c1} is C₁₋₆ alkyl substituted with fluorine. In certain embodiments, p is 2, (Y¹)ₘ is (CH₂)₅, R¹ is —NHR^{A} and contains at least one radioisotope, R^{A} is

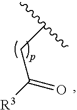

R³ is —N(RC)₂, two occurrences of R^{C} are joined together with their intervening atom to form

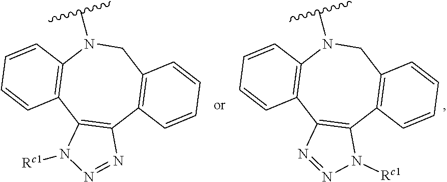

and R^{c1} is methyl substituted with fluorine, ethyl substituted with fluorine, n-propyl substituted with fluorine, isopropyl substituted with fluorine, n-butyl substituted with fluorine, tert-butyl substituted with fluorine, sec-butyl substituted with fluorine, isobutyl substituted with fluorine, n-pentyl substituted with fluorine, 3-pentanyl substituted with fluorine, amyl substituted with fluorine, neopentyl substituted with fluorine, 3-methyl-2-butanyl substituted with fluorine, tert-amyl substituted with fluorine, or n-hexyl substituted with fluorine. In certain embodiments, p is 2, (Y¹)ₘ is (CH₂)₅, R¹ is —NHR^{A} and contains at least one radioisotope, R^{A} is

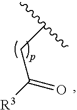

R³ is —N(RC)₂, two occurrences of R^{C} are joined together with their intervening atom to form

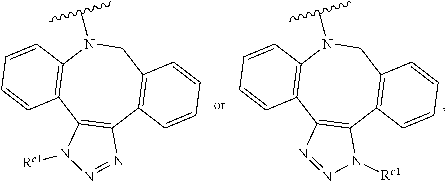

and R^{c1} is ethyl substituted with fluorine. In certain embodiments, p is 2, (Y¹)ₘ is (CH₂)₅, R¹ is —NHR^{A} and contains at least one radioisotope, R^{A} is

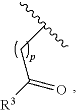

R³ is —N(RC)₂, two occurrences of R^{C} are joined together with their intervening atom to form

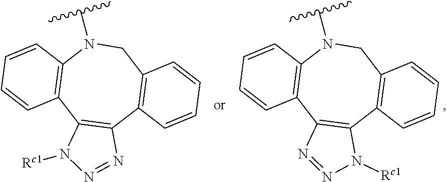

and R^{c1} is alkyl substituted with ¹⁸F. In certain embodiments, R^{c1} is C₁₋₁₂ alkyl substituted with ¹⁸F. In certain embodiments, p is 2, (Y¹)ₘ is (CH₂)₅, R¹ is —NHR^{A} and contains at least one radioisotope, R^{A} is

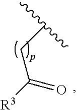

R³ is —N(RC)₂, two occurrences of R^{C} are joined together with their intervening atom to form

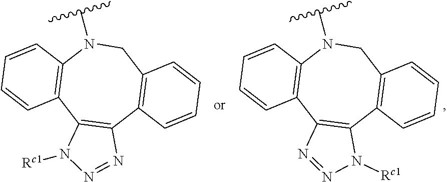

and R^{c1} is C₁₋₆ alkyl substituted with ¹⁸F. In certain embodiments, p is 2, (Y¹)ₘ is (CH₂)₅, R¹ is —NHR^{A} and contains at least one radioisotope, R^{A} is

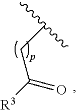

R³ is —N(RC)₂, two occurrences of R^{C} are joined together with their intervening atom to form

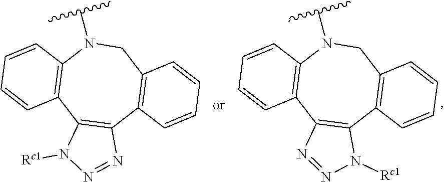

and R^{c1} is methyl substituted with ¹⁸F, ethyl substituted with ¹⁸F, n-propyl substituted with ¹⁸F, isopropyl substituted with ¹⁸F, n-butyl substituted with ¹⁸F, tert-butyl substituted with ¹⁸F, sec-butyl substituted with ¹⁸F, isobutyl substituted with ¹⁸F, n-pentyl substituted with ¹⁸F, 3-pentanyl substituted with ¹⁸F, amyl substituted with ¹⁸F, neopentyl substituted with ¹⁸F, 3-methyl-2-butanyl substituted with ¹⁸F, tert-amyl substituted with ¹⁸F, or n-hexyl substituted with ¹⁸F. In certain embodiments, p is 2, (Y)ₘ is (CH₂)₅, R¹ is —NHR^{A} and contains at least one radioisotope, R^{A} is

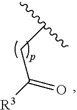

R³ is —N(RC)₂, two occurrences of R^{C} are joined together with their intervening atom to form

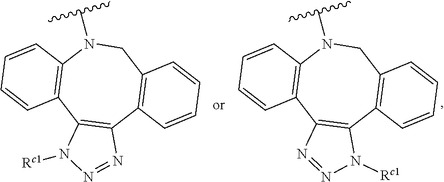

and R^{c1} is ethyl substituted with ¹⁸F.

### R²

As described herein, each occurrence of R² is independently hydrogen, halogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl, or two occurrences of R² are joined together with their intervening atom to form an optionally substituted carbocyclic ring or optionally substituted heterocyclic ring.

In certain embodiments, at least one occurrence of R² is hydrogen. In certain embodiments, R² is halogen. In certain embodiments, R² is fluorine, chlorine, or bromine. In certain embodiments, at least one occurrence of R² is optionally substituted acyl. In certain embodiments, at least one occurrence of R² is optionally substituted C₁₋₁₂ alkyl. In certain embodiments, at least one occurrence of R² is optionally substituted C₁₋₆ alkyl. In certain embodiments, at least one occurrence of R² is unsubstituted C₁₋₆ alkyl. In certain embodiments, at least one occurrence of R² is substituted C₁₋₆ alkyl. In certain embodiments, at least one occurrence of R² is substituted or unsubstituted methyl, substituted or unsubstituted ethyl, substituted or unsubstituted n-propyl, substituted or unsubstituted isopropyl, substituted or unsubstituted n-butyl, substituted or unsubstituted tert-butyl, substituted or unsubstituted sec-butyl, substituted or unsubstituted isobutyl, substituted or unsubstituted n-pentyl, substituted or unsubstituted 3-pentanyl, substituted or unsubstituted amyl, substituted or unsubstituted neopentyl, substituted or unsubstituted 3-methyl-2-butanyl, substituted or unsubstituted tert-amyl, or substituted or unsubstituted n-hexyl. In certain embodiments, at least one occurrence of R² is substituted or unsubstituted methyl. In certain embodiments, at least one occurrence of R² is unsubstituted methyl. In certain embodiments, at least one occurrence of R² is optionally substituted C₂₋₁₂ alkenyl. In certain embodiments, at least one occurrence of R² is optionally substituted C₂₋₆ alkenyl. In certain embodiments, at least one occurrence of R² is substituted or unsubstituted ethenyl, substituted or unsubstituted 1-propenyl, substituted or unsubstituted 2-propenyl, substituted or unsubstituted 1-butenyl, substituted or unsubstituted 2-butenyl, substituted or unsubstituted butadienyl, substituted or unsubstituted pentenyl, substituted or unsubstituted pentadienyl, or substituted or unsubstituted hexenyl. In certain embodiments, at least one occurrence of R² is optionally substituted C₂₋₁₂ alkynyl. In certain embodiments, at least one occurrence of R² is optionally substituted C₂₋₆ alkynyl. In certain embodiments, at least one occurrence of R² is substituted or unsubstituted ethynyl, substituted or unsubstituted 1-propynyl, substituted or unsubstituted 2-propynyl, substituted or unsubstituted 1-butynyl, substituted or unsubstituted 2-butynyl, substituted or unsubstituted pentynyl, or substituted or unsubstituted hexynyl. In certain embodiments, at least one occurrence of R² is optionally substituted heteroC₁₋₁₂ alkyl. In certain embodiments, at least one occurrence of R² is optionally substituted heteroC₁₋₆ alkyl. In certain embodiments, at least one occurrence of R² is optionally substituted heteroC₁₋₁₂ alkenyl. In certain embodiments, at least one occurrence of R² is optionally substituted heteroC₁₋₆ alkenyl. In certain embodiments, at least one occurrence of R² is optionally substituted heteroC₁₋₁₂ alkynyl. In certain embodiments, at least one occurrence of R² is optionally substituted heteroC₁₋₆ alkynyl. In certain embodiments, at least one occurrence of R² is optionally substituted C₃₋₁₄ cycloalkyl. In certain embodiments, at least one occurrence of R² is optionally substituted 5-10 membered heterocyclyl. In certain embodiments, at least one occurrence of R² is optionally substituted 6-14 membered aryl. In certain embodiments, at least one occurrence of R² is optionally substituted 5-14 membered heteroaryl. In certain embodiments, at least two occurrences of R² are joined together with their intervening atom to form an optionally substituted 5-10 membered carbocyclic ring. In certain embodiments, at least two occurrences of R² are joined together with their intervening atom to form an optionally substituted 5-10 membered heterocyclic ring.

In certain embodiments, at least two occurrences of R² are optionally substituted C₁₋₁₂ alkyl. In certain embodiments, at least two occurrences of R² are optionally substituted C₁₋₆ alkyl. In certain embodiments, at least two occurrences of R² are unsubstituted C₁₋₆ alkyl. In certain embodiments, at least two occurrences of R² are substituted C₁₋₆ alkyl. In certain embodiments, at least two occurrences of R² are substituted or unsubstituted methyl, substituted or unsubstituted ethyl, substituted or unsubstituted n-propyl, substituted or unsubstituted isopropyl, substituted or unsubstituted n-butyl, substituted or unsubstituted tert-butyl, substituted or unsubstituted sec-butyl, substituted or unsubstituted isobutyl, substituted or unsubstituted n-pentyl, substituted or unsubstituted 3-pentanyl, substituted or unsubstituted amyl, substituted or unsubstituted neopentyl, substituted or unsubstituted 3-methyl-2-butanyl, substituted or unsubstituted tert-amyl, or substituted or unsubstituted n-hexyl. In certain embodiments, at least two occurrences of R² are substituted or unsubstituted methyl. In certain embodiments, at least two occurrences of R² are unsubstituted methyl.

In certain embodiments, at least three occurrences of R² are optionally substituted C₁₋₁₂ alkyl. In certain embodiments, at least three occurrences of R² are optionally substituted C₁₋₆ alkyl. In certain embodiments, at least three occurrences of R² are unsubstituted C₁₋₆ alkyl. In certain embodiments, at least three occurrences of R² are substituted C₁₋₆ alkyl. In certain embodiments, at least three occurrences of R² are substituted or unsubstituted methyl, substituted or unsubstituted ethyl, substituted or unsubstituted n-propyl, substituted or unsubstituted isopropyl, substituted or unsubstituted n-butyl, substituted or unsubstituted tert-butyl, substituted or unsubstituted sec-butyl, substituted or unsubstituted isobutyl, substituted or unsubstituted n-pentyl, substituted or unsubstituted 3-pentanyl, substituted or unsubstituted amyl, substituted or unsubstituted neopentyl, substituted or unsubstituted 3-methyl-2-butanyl, substituted or unsubstituted tert-amyl, or substituted or unsubstituted n-hexyl. In certain embodiments, at least three occurrences of R² are substituted or unsubstituted methyl. In certain embodiments, at least three occurrences of R² are unsubstituted methyl.

In certain embodiments, at least four occurrences of R² are optionally substituted C₁₋₁₂ alkyl. In certain embodiments, at least four occurrences of R² are optionally substituted C₁₋₆ alkyl. In certain embodiments, at least four occurrences of R² are unsubstituted C₁₋₆ alkyl. In certain embodiments, at least four occurrences of R² are substituted C₁₋₆ alkyl. In certain embodiments, at least four occurrences of R² are substituted or unsubstituted methyl, substituted or unsubstituted ethyl, substituted or unsubstituted n-propyl, substituted or unsubstituted isopropyl, substituted or unsubstituted n-butyl, substituted or unsubstituted tert-butyl, substituted or unsubstituted sec-butyl, substituted or unsubstituted isobutyl, substituted or unsubstituted n-pentyl, substituted or unsubstituted 3-pentanyl, substituted or unsubstituted amyl, substituted or unsubstituted neopentyl, substituted or unsubstituted 3-methyl-2-butanyl, substituted or unsubstituted tert-amyl, or substituted or unsubstituted n-hexyl. In certain embodiments, at least four occurrences of R² are substituted or unsubstituted methyl. In certain embodiments, at least four occurrences of R² are unsubstituted methyl.

### Radioisotope

As described herein, a provided compound of Formula I contains at least one radioisotope (i.e., R¹ in Formula I contains at least one radioisotope). In certain embodiments, the at least one radioisotope is ¹¹C, ¹⁸F, ¹³N, ¹⁵O, ⁶⁴Cu, ⁶⁷Ga, ⁶⁸Ga, ⁷⁵Se, ^{81m}Kr, ⁸²Rb, ^{99m}Tc, ¹¹¹In, ¹²³I, ¹²⁵I, ¹³¹I, ¹³³Xe. In certain embodiments, the at least one radioisotope is ¹¹C, ¹⁸F, ¹³N, ¹⁵O, ⁶⁴Cu, ⁶⁷Cu, ⁶⁷Ga, ⁶⁸Ga, ⁷⁵Se, ^{81m}Kr, ⁸²Rb, ^{99m}Tc, ¹¹¹In, ¹²³I, ¹²⁵I, ¹³¹I, ¹³³Xe. In certain embodiments, the at least one radioisotope is ¹¹C, ¹⁸F, ¹³N, ¹⁵O, ⁷⁵Se, ¹²³I, ¹²⁵I, or ¹³¹I.

In certain embodiments, the at least one radioisotope is ¹¹C. In certain embodiments, the at least one radioisotope is ¹⁸F. In certain embodiments, the at least one radioisotope is ¹³N. In certain embodiments, the at least one radioisotope is ¹⁵⁰. In certain embodiments, the at least one radioisotope is ⁷⁵Se. In certain embodiments, the at least one radioisotope is ¹²³I. In certain embodiments, the at least one radioisotope is ¹²⁵I. In certain embodiments, the at least one radioisotope is ¹³¹I. In certain embodiments, the at least one radioisotope is ⁶⁴Cu. In certain embodiments, the at least one radioisotope is ⁶⁷Cu. In certain embodiments, the at least one radioisotope is ⁶⁷Ga. In certain embodiments, the at least one radioisotope is ⁶⁸Ga. In certain embodiments, the at least one radioisotope is ^{81m}Kr. In certain embodiments, the at least one radioisotope is ⁸²Rb. In certain embodiments, the at least one radioisotope is ^{99m}Tc. In certain embodiments, the at least one radioisotope is ¹¹¹In. In certain embodiments, the at least one radioisotope is ¹³³Xe.

### Additional Embodiments of Formula I

In certain embodiments, the provided compound is of Formula I-a-i:

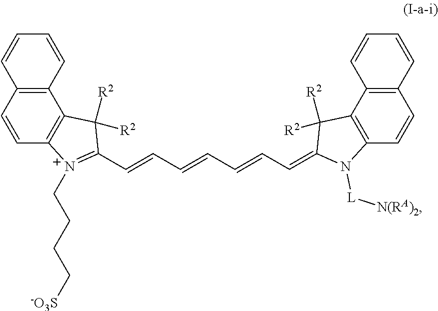

or a pharmaceutically acceptable salt, solvate, hydrate, polymorph, co-crystal, tautomer, stereoisomer, or prodrug thereof.

In certain embodiments, the provided compound is of Formula I-a-ii:

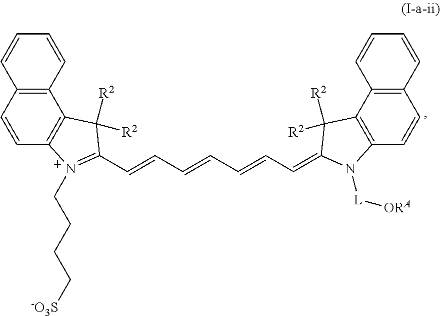

or a pharmaceutically acceptable salt, solvate, hydrate, polymorph, co-crystal, tautomer, stereoisomer, or prodrug thereof.

In certain embodiments, the provided compound is of Formula I-a-iii:

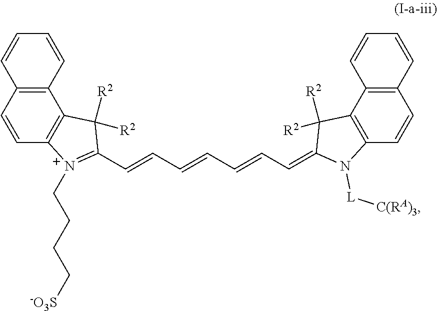

or a pharmaceutically acceptable salt, solvate, hydrate, polymorph, co-crystal, tautomer, stereoisomer, or prodrug thereof.

In certain embodiments, the provided compound is of Formula I-b:

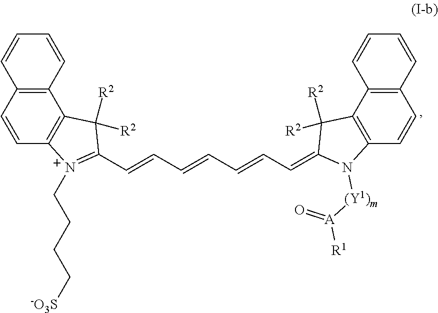

or a pharmaceutically acceptable salt, solvate, hydrate, polymorph, co-crystal, tautomer, stereoisomer, or prodrug thereof.

In certain embodiments, the provided compound is of Formula I-b-i:

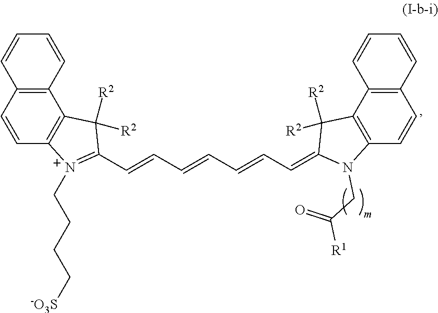

or a pharmaceutically acceptable salt, solvate, hydrate, polymorph, co-crystal, tautomer, stereoisomer, or prodrug thereof.

In certain embodiments, the provided compound is of Formula I-c:

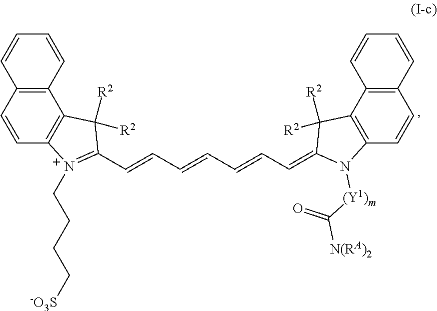

or a pharmaceutically acceptable salt, solvate, hydrate, polymorph, co-crystal, tautomer, stereoisomer, or prodrug thereof.

In certain embodiments, the provided compound is of Formula I-c-i:

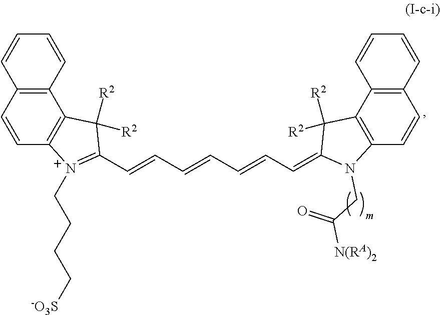

or a pharmaceutically acceptable salt, solvate, hydrate, polymorph, co-crystal, tautomer, stereoisomer, or prodrug thereof.

In certain embodiments, the provided compound is of Formula I-d:

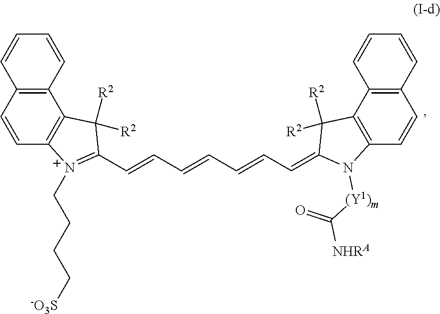

or a pharmaceutically acceptable salt, solvate, hydrate, polymorph, co-crystal, tautomer, stereoisomer, or prodrug thereof.

In certain embodiments, the provided compound is of Formula I-d-i:

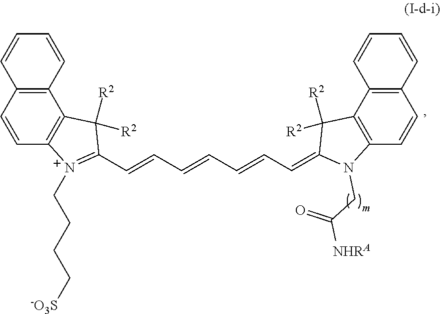

or a pharmaceutically acceptable salt, solvate, hydrate, polymorph, co-crystal, tautomer, stereoisomer, or prodrug thereof.

In certain embodiments, the provided compound is of Formula I-e:

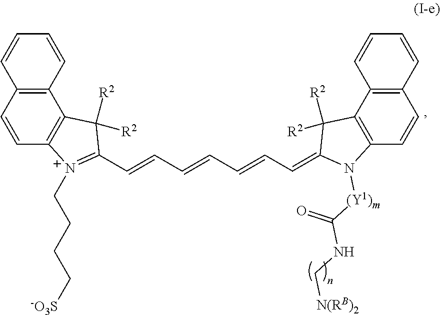

or a pharmaceutically acceptable salt, solvate, hydrate, polymorph, co-crystal, tautomer, stereoisomer, or prodrug thereof.

In certain embodiments, the provided compound is of Formula I-e-i:

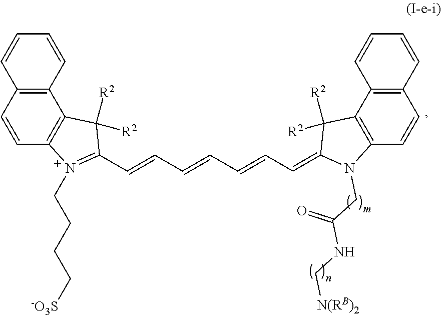

or a pharmaceutically acceptable salt, solvate, hydrate, polymorph, co-crystal, tautomer, stereoisomer, or prodrug thereof.

In certain embodiments, the provided compound is of Formula I-f:

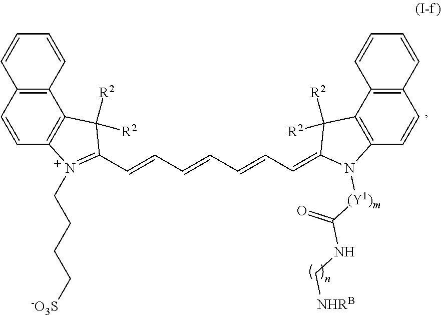

or a pharmaceutically acceptable salt, solvate, hydrate, polymorph, co-crystal, tautomer, stereoisomer, or prodrug thereof.

In certain embodiments, the provided compound is of Formula I-f-i:

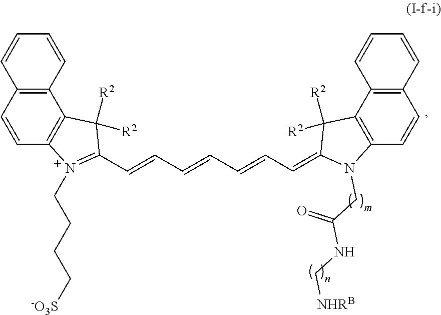

or a pharmaceutically acceptable salt, solvate, hydrate, polymorph, co-crystal, tautomer, stereoisomer, or prodrug thereof.

In certain embodiments, the provided compound is of Formula I-g:

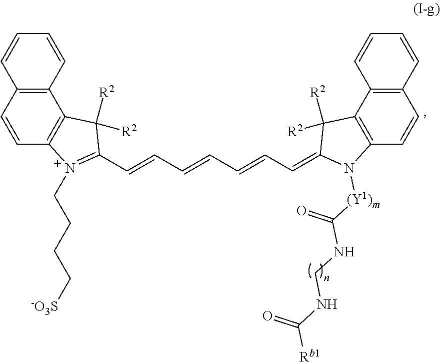

or a pharmaceutically acceptable salt, solvate, hydrate, polymorph, co-crystal, tautomer, stereoisomer, or prodrug thereof.

In certain embodiments, the provided compound is of Formula I-g-i:

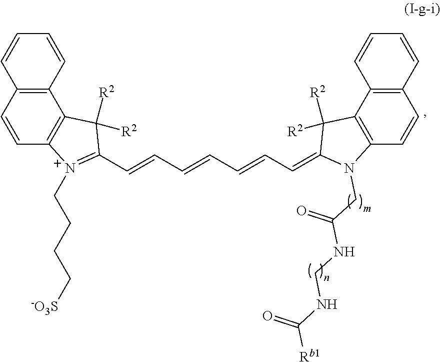

or a pharmaceutically acceptable salt, solvate, hydrate, polymorph, co-crystal, tautomer, stereoisomer, or prodrug thereof.

In certain embodiments, the provided compound is of Formula I-h:

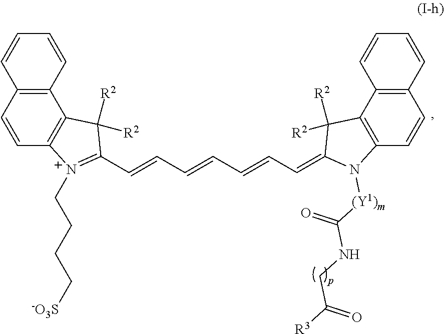

or a pharmaceutically acceptable salt, solvate, hydrate, polymorph, co-crystal, tautomer, stereoisomer, or prodrug thereof.

In certain embodiments, the provided compound is of Formula I-h-i:

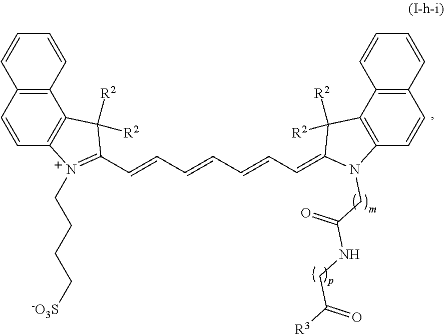

or a pharmaceutically acceptable salt, solvate, hydrate, polymorph, co-crystal, tautomer, stereoisomer, or prodrug thereof.

In certain embodiments, the provided compound is of Formula I-h-ii or Formula I-h-iii:

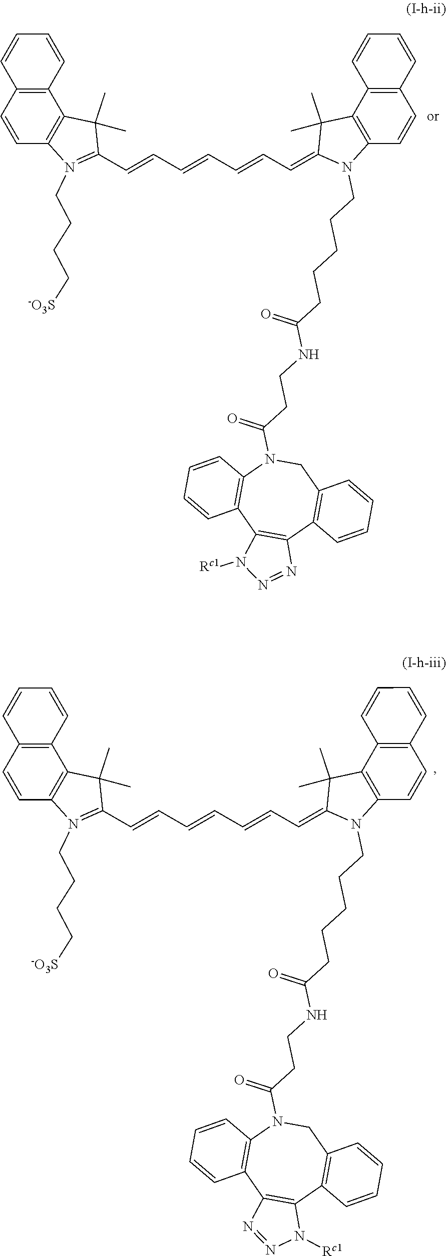

or a pharmaceutically acceptable salt, solvate, hydrate, polymorph, co-crystal, tautomer, stereoisomer, or prodrug thereof.

In certain embodiments, the provided compound is of Formula I-j:

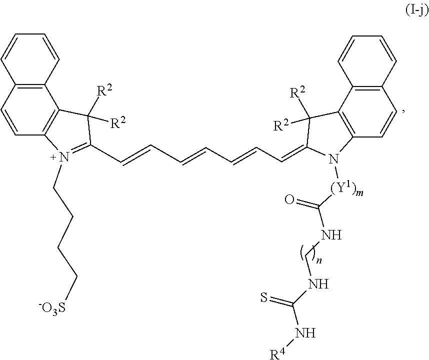

or a pharmaceutically acceptable salt, solvate, hydrate, polymorph, co-crystal, tautomer, stereoisomer, or prodrug thereof.

In certain embodiments, the provided compound is of Formula I-j-i:

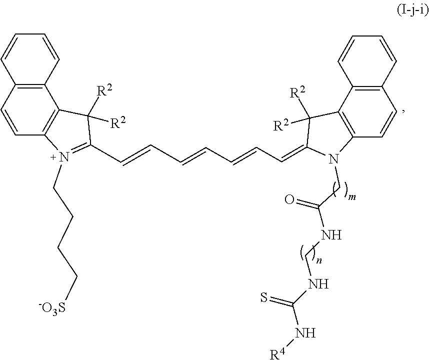

or a pharmaceutically acceptable salt, solvate, hydrate, polymorph, co-crystal, tautomer, stereoisomer, or prodrug thereof.

In certain embodiments, any of the formulae disclosed herein are subgenera of Formula (I′) and/or Formula (I), i.e., the formulae that are subgenera of Formula (I) contain at least one radioisotope in R¹ and the formulae that are subgenera of Formula (I′) are not required to contain at least one radioisotope in R¹.

In certain embodiments, the provided compound is of formula:

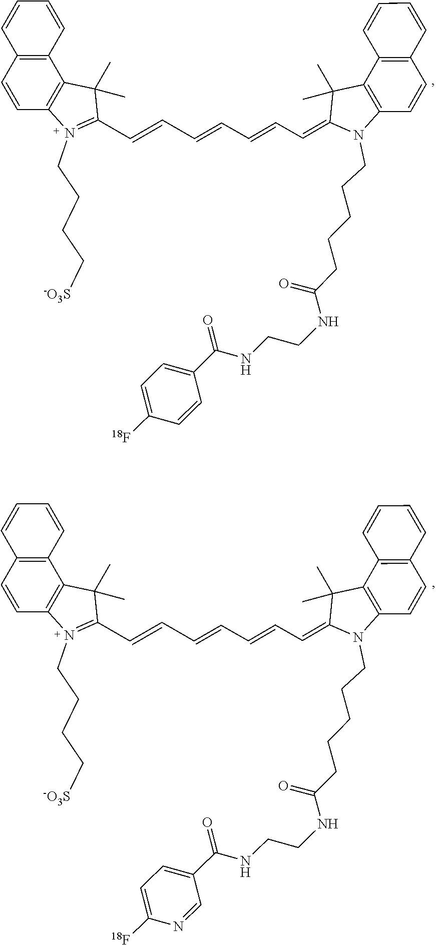

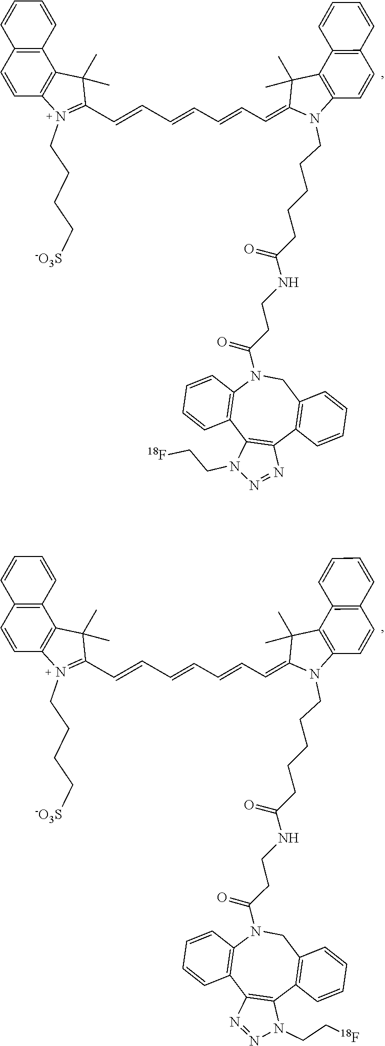

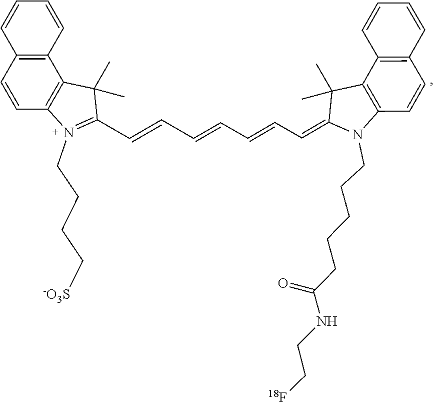

or a pharmaceutically acceptable salt, solvate, hydrate, polymorph, co-crystal, tautomer, stereoisomer, or prodrug thereof.

In certain embodiments, the provided compound is of formula:

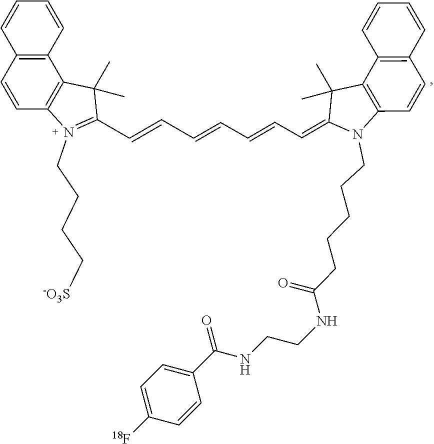

or a pharmaceutically acceptable salt, solvate, hydrate, polymorph, co-crystal, tautomer, stereoisomer, or prodrug thereof.

In certain embodiments, the provided compound is of formula:

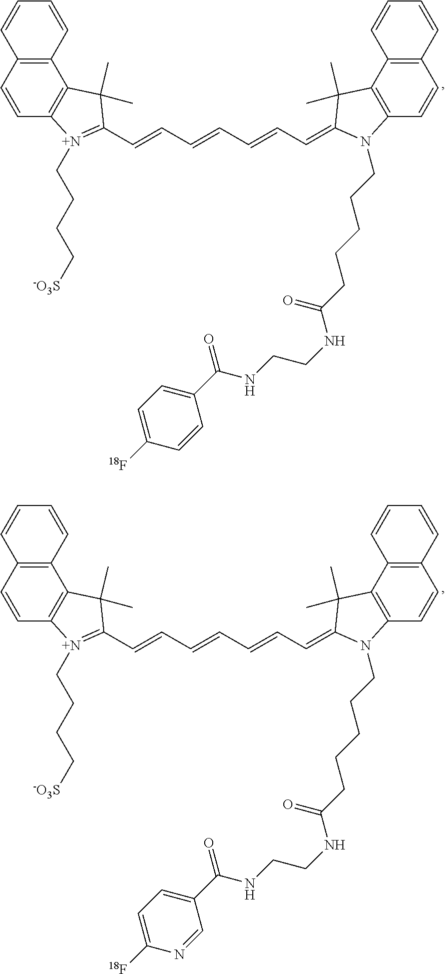

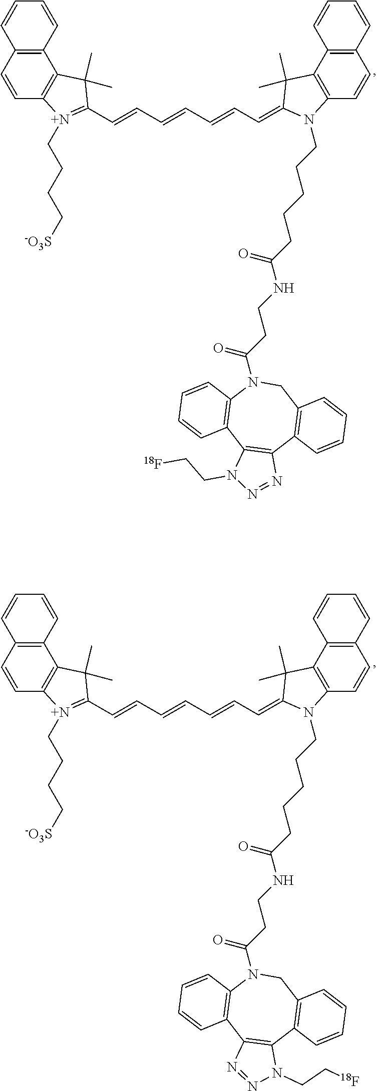

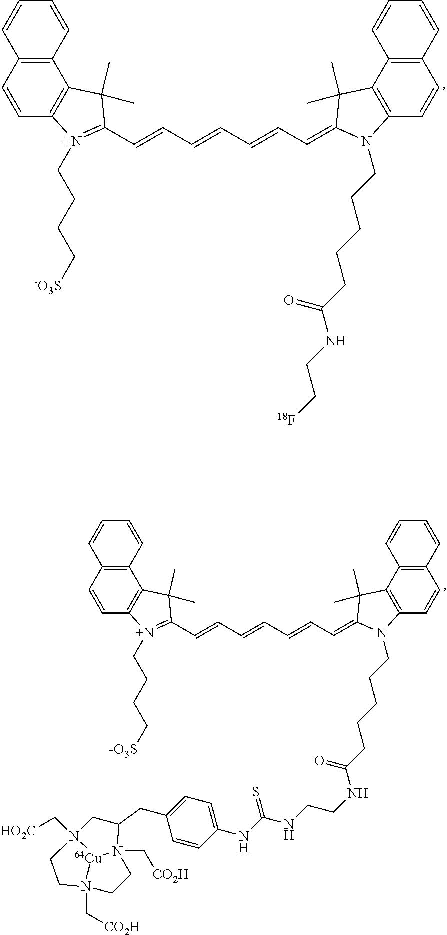

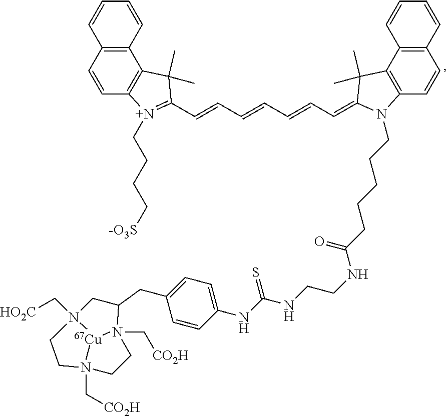

or a pharmaceutically acceptable salt, solvate, hydrate, polymorph, co-crystal, tautomer, stereoisomer, or prodrug thereof.

In certain embodiments, the provided compound is of formula:

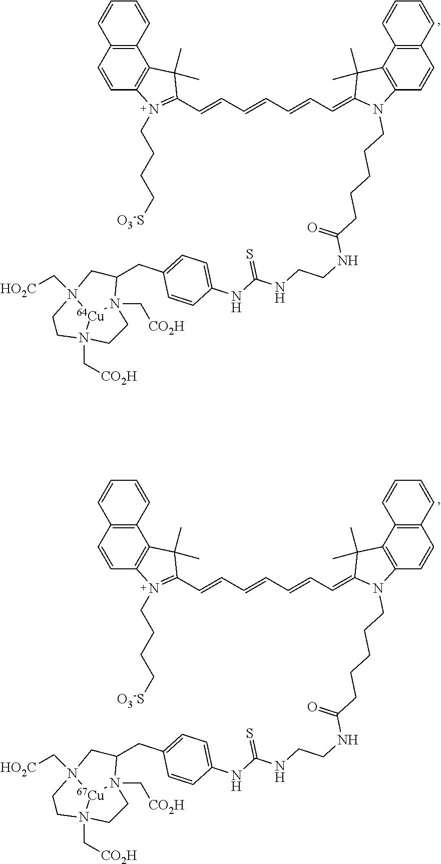

or a pharmaceutically acceptable salt, solvate, hydrate, polymorph, co-crystal, tautomer, stereoisomer, or prodrug thereof.

In certain embodiments, the provided compound is of formula:

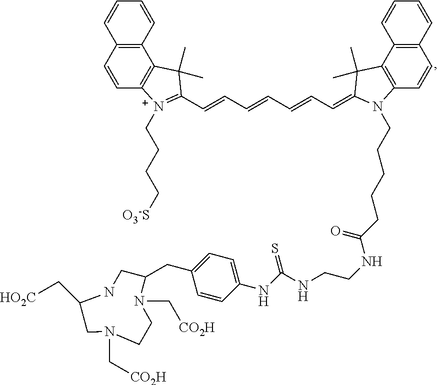

or a pharmaceutically acceptable salt, solvate, hydrate, polymorph, co-crystal, tautomer, stereoisomer, or prodrug thereof.

In certain embodiments, the provided compound degrades less than 10% 1 minute after preparing the compound. In certain embodiments, the provided compound degrades less than 10% 5 minutes after preparing the compound. In certain embodiments, the provided compound degrades less than 10% 10 minutes after preparing the compound. In certain embodiments, the provided compound degrades less than 10% 15 minutes after preparing the compound. In certain embodiments, the provided compound degrades less than 10% 20 minutes after preparing the compound. In certain embodiments, the provided compound degrades less than 10% 25 minutes after preparing the compound. In certain embodiments, the provided compound degrades less than 10% 30 minutes after preparing the compound. In certain embodiments, the provided compound degrades less than 10% 35 minutes after preparing the compound. In certain embodiments, the provided compound degrades less than 10% 40 minutes after preparing the compound. In certain embodiments, the provided compound degrades less than 10% 45 minutes after preparing the compound. In certain embodiments, the provided compound degrades less than 10% 50 minutes after preparing the compound. In certain embodiments, the provided compound degrades less than 10% 55 minutes after preparing the compound. In certain embodiments, the provided compound degrades less than 10% 1 hour after preparing the compound. In certain embodiments, the provided compound degrades less than 10% 2 hours after preparing the compound. In certain embodiments, the provided compound degrades less than 10% 3 hours after preparing the compound. In certain embodiments, the provided compound degrades less than 10% 4 hours after preparing the compound. In certain embodiments, the provided compound degrades less than 10% 5 hours after preparing the compound. In certain embodiments, the provided compound degrades less than 10% 6 hours after preparing the compound. In certain embodiments, the provided compound degrades less than 10% 7 hours after preparing the compound. In certain embodiments, the provided compound degrades less than 10% 8 hours after preparing the compound. In certain embodiments, the provided compound degrades less than 10% 9 hours after preparing the compound. In certain embodiments, the provided compound degrades less than 10% 10 hours after preparing the compound. In certain embodiments, the provided compound degrades less than 10% 11 hours after preparing the compound. In certain embodiments, the provided compound degrades less than 10% 12 hours after preparing the compound. In certain embodiments, the provided compound degrades less than 10% 16 hours after preparing the compound. In certain embodiments, the provided compound degrades less than 10% 20 hours after preparing the compound. In certain embodiments, the provided compound degrades less than 10% 1 day after preparing the compound. In certain embodiments, the provided compound degrades less than 10% 2 days after preparing the compound. In certain embodiments, the provided compound degrades less than 10% 3 days after preparing the compound. In certain embodiments, the provided compound degrades less than 10% 4 days after preparing the compound. In certain embodiments, the provided compound degrades less than 10% 5 days after preparing the compound. In certain embodiments, the provided compound degrades less than 10% 6 days after preparing the compound. In certain embodiments, the provided compound degrades less than 10% 1 week after preparing the compound. In certain embodiments, the provided compound degrades less than 10% 1 month after preparing the compound. In certain embodiments, the provided compound degrades less than 10% 6 months after preparing the compound. In certain embodiments, the provided compound degrades less than 10% 1 year after preparing the compound.

In certain embodiments, the composition is stable for at least 1 minute. In certain embodiments, the composition is stable for at least 5 minutes. In certain embodiments, the composition is stable for at least 10 minutes. In certain embodiments, the composition is stable for at least 15 minutes. In certain embodiments, the composition is stable for at least 20 minutes. In certain embodiments, the composition is stable for at least 25 minutes. In certain embodiments, the composition is stable for at least 30 minutes. In certain embodiments, the composition is stable for at least 35 minutes. In certain embodiments, the composition is stable for at least 40 minutes. In certain embodiments, the composition is stable for at least 45 minutes. In certain embodiments, the composition is stable for at least 50 minutes. In certain embodiments, the composition is stable for at least 55 minutes. In certain embodiments, the composition is stable for at least 1 hour. In certain embodiments, the composition is stable for at least 2 hours. In certain embodiments, the composition is stable for at least 3 hours. In certain embodiments, the composition is stable for at least 4 hours. In certain embodiments, the composition is stable for at least 5 hours. In certain embodiments, the composition is stable for at least 6 hours. In certain embodiments, the composition is stable for at least 7 hours. In certain embodiments, the composition is stable for at least 8 hours. In certain embodiments, the composition is stable for at least 9 hours. In certain embodiments, the composition is stable for at least 10 hours. In certain embodiments, the composition is stable for at least 11 hours. In certain embodiments, the composition is stable for at least 12 hours. In certain embodiments, the composition is stable for at least 16 hours. In certain embodiments, the composition is stable for at least 20 hours. In certain embodiments, the composition is stable for at least one day. In certain embodiments, the composition is stable for at least two days. In certain embodiments, the composition is stable for at least three days. In certain embodiments, the composition is stable for at least four days. In certain embodiments, the composition is stable for at least five days. In certain embodiments, the composition is stable for at least six days. In certain embodiments, the composition is stable for at least one week. In certain embodiments, the composition is stable for at least 1 month. In certain embodiments, the composition is stable for at least 6 months. In certain embodiments, the composition is stable for at least 1 year.

In certain embodiments, the composition is stable for at least 1 minute at room temperature (e.g., about 20-40° C.). In certain embodiments, the composition is stable for at least 5 minutes at room temperature (e.g., about 20-40° C.). In certain embodiments, the composition is stable for at least 10 minutes at room temperature (e.g., about 20-40° C.). In certain embodiments, the composition is stable for at least 15 minutes at room temperature (e.g., about 20-40° C.). In certain embodiments, the composition is stable for at least 20 minutes at room temperature (e.g., about 20-40° C.). In certain embodiments, the composition is stable for at least 25 minutes at room temperature (e.g., about 20-40° C.). In certain embodiments, the composition is stable for at least 30 minutes at room temperature (e.g., about 20-40° C.). In certain embodiments, the composition is stable for at least 35 minutes at room temperature (e.g., about 20-40° C.). In certain embodiments, the composition is stable for at least 40 minutes at room temperature (e.g., about 20-40° C.). In certain embodiments, the composition is stable for at least 45 minutes at room temperature (e.g., about 20-40° C.). In certain embodiments, the composition is stable for at least 50 minutes at room temperature (e.g., about 20-40° C.). In certain embodiments, the composition is stable for at least 55 minutes at room temperature (e.g., about 20-40° C.). In certain embodiments, the composition is stable for at least 1 hour at room temperature (e.g., about 20-40° C.). In certain embodiments, the composition is stable for at least 2 hours at room temperature (e.g., about 20-40° C.). In certain embodiments, the composition is stable for at least 3 hours at room temperature (e.g., about 20-40° C.). In certain embodiments, the composition is stable for at least 4 hours at room temperature (e.g., about 20-40° C.). In certain embodiments, the composition is stable for at least 5 hours at room temperature (e.g., about 20-40° C.). In certain embodiments, the composition is stable for at least 6 hours at room temperature (e.g., about 20-40° C.). In certain embodiments, the composition is stable for at least 7 hours at room temperature (e.g., about 20-40° C.). In certain embodiments, the composition is stable for at least 8 hours at room temperature (e.g., about 20-40° C.). In certain embodiments, the composition is stable for at least 9 hours at room temperature (e.g., about 20-40° C.). In certain embodiments, the composition is stable for at least 10 hours at room temperature (e.g., about 20-40° C.). In certain embodiments, the composition is stable for at least 11 hours at room temperature (e.g., about 20-40° C.). In certain embodiments, the composition is stable for at least 12 hours at room temperature (e.g., about 20-40° C.). In certain embodiments, the composition is stable for at least 16 hours at room temperature (e.g., about 20-40° C.). In certain embodiments, the composition is stable for at least 20 hours at room temperature (e.g., about 20-40° C.). In certain embodiments, the composition is stable for at least one day at room temperature (e.g., about 20-40° C.). In certain embodiments, the composition is stable for at least two days at room temperature (e.g., about 20-40° C.). In certain embodiments, the composition is stable for at least three days at room temperature (e.g., about 20-40° C.). In certain embodiments, the composition is stable for at least four days at room temperature (e.g., about 20-40° C.). In certain embodiments, the composition is stable for at least five days at room temperature (e.g., about 20-40° C.). In certain embodiments, the composition is stable for at least six days at room temperature (e.g., about 20-40° C.). In certain embodiments, the composition is stable for at least one week at room temperature (e.g., about 20-40° C.). In certain embodiments, the composition is stable for at least 1 month at room temperature (e.g., about 20-40° C.). In certain embodiments, the composition is stable for at least 6 months at room temperature (e.g., about 20-40° C.). In certain embodiments, the composition is stable for at least 1 year at room temperature (e.g., about 20-40° C.). In certain embodiments, the composition is stable at about 0-40° C. In certain embodiments, the composition is stable at about 0-20° C. In certain embodiments, the composition is stable at about 20-40° C. In certain embodiments, the composition is stable at about 0-10° C. In certain embodiments, the composition is stable at about 10-20° C. In certain embodiments, the composition is stable at about 20-30° C. In certain embodiments, the composition is stable at about 30-40° C. In certain embodiments, the composition is stable at about 0-5° C. In certain embodiments, the composition is stable at about 5-10° C. In certain embodiments, the composition is stable at about 10-15° C. In certain embodiments, the composition is stable at about 15-20° C. In certain embodiments, the composition is stable at about 20-25° C. In certain embodiments, the composition is stable at about 25-30° C. In certain embodiments, the composition is stable at about 30-35° C. In certain embodiments, the composition is stable at about 35-40° C. In certain embodiments, the composition is stable at about 4° C. In certain embodiments, the composition is stable at about 23.5° C. In certain embodiments, the composition is stable at about 37° C.

In certain embodiments, a compound or a composition comprising a compound of Formula (I) is stable when the compound degrades less than 1%, less than 2%, less than 3%, less than 4%, less than 5%, less than 6%, less than 7%, less than 8%, less than 9%, less than 10%, less than 15%, less than 20%, less than 25%, less than 30%, less than 35%, less than 40%, less than 45%, less than 50%, less than 55%, less than 60%, less than 70%, less than 80%, or less than 90% at any temperature and timepoint recited in the above paragraphs.

In certain embodiments, a provided compound (a compound described herein, a compound of the present disclosure) is a compound of Formula I or I′, or pharmaceutically acceptable salt, solvate, hydrate, polymorph, co-crystal, tautomer, stereoisomer, isotopically labeled compound, or prodrug thereof. In certain embodiments, a provided compound is a compound of Formula I or I′, or pharmaceutically acceptable salt, solvate, hydrate, polymorph, co-crystal, tautomer, stereoisomer, isotopically labeled compound, or prodrug thereof. In certain embodiments, a provided compound is a compound of Formula I or I′, or a pharmaceutically acceptable salt or tautomer thereof. In certain embodiments, a provided compound is a compound of Formula I or I′, or a pharmaceutically acceptable salt thereof.

In certain embodiments, a provided compound (a compound described herein, a compound of the present disclosure) is a compound of Formula I, or pharmaceutically acceptable salt, solvate, hydrate, polymorph, co-crystal, tautomer, stereoisomer, isotopically labeled compound, or prodrug thereof. In certain embodiments, a provided compound is a compound of Formula I, or pharmaceutically acceptable salt, solvate, hydrate, polymorph, co-crystal, tautomer, stereoisomer, isotopically labeled compound, or prodrug thereof. In certain embodiments, a provided compound is a compound of Formula I, or a pharmaceutically acceptable salt or tautomer thereof. In certain embodiments, a provided compound is a compound of Formula I, or a pharmaceutically acceptable salt thereof.

### Pharmaceutical Compositions, Kits, and Administration

The present disclosure provides pharmaceutical compositions comprising a provided compound, and optionally a pharmaceutically acceptable excipient. In certain embodiments, the pharmaceutical composition described herein comprises a provided compound, and a pharmaceutically acceptable excipient.

In certain embodiments, the pharmaceutical composition comprises an effective amount of the provided compound. In certain embodiments, the effective amount is a therapeutically effective amount. In certain embodiments, the effective amount is a prophylactically effective amount. In certain embodiments, the effective amount is an amount effective for diagnosing a disease in a subject in need thereof. In certain embodiments, the effective amount is an amount effective for treating a disease in a subject in need thereof. In certain embodiments, the effective amount is an amount effective for preventing a disease in a subject in need thereof. In certain embodiments, the effective amount is an amount effective for reducing the risk of developing a disease in a subject in need thereof. In certain embodiments, the effective amount is an amount effective for reducing the size of a tumor (e.g., in a subject, tissue, biological sample, or cell). In certain embodiments, the effective amount is an amount effective for promoting apoptosis in a subject in need thereof or in a cell, tissue, or biological sample, wherein the cell, tissue, or biological sample is in vivo. In certain embodiments, the effective amount is an amount effective for promoting apoptosis in a subject in need thereof or in a cell, tissue, or biological sample, wherein the cell, tissue, or biological sample is in vitro.

In certain embodiments, the subject is an animal. In certain embodiments, the subject is a human. In certain embodiments, the subject is a human aged 18 years or older. In certain embodiments, the subject is a human aged 12-18 years, exclusive. In certain embodiments, the subject is a human aged 2-12 years, inclusive. In certain embodiments, the subject is a human younger than 2 years. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the pharmaceutical composition is for use in diagnosing a disease. In certain embodiments, the pharmaceutical composition is for use in treating a disease. In certain embodiments, the pharmaceutical composition is for use in preventing a disease.

A provided compound or pharmaceutical composition, as described herein, can be administered in combination with one or more additional pharmaceutical agents (e.g., therapeutically and/or prophylactically active agents). The provided compounds or pharmaceutical compositions can be administered in combination with additional pharmaceutical agents that improve their activity (e.g., activity (e.g., potency and/or efficacy) in diagnosing a disease in a subject in need thereof, treating a disease in a subject in need thereof, in preventing a disease in a subject in need thereof, and/or in reducing the risk of developing a disease in a subject in need thereof), improve bioavailability, improve safety, reduce drug resistance, reduce and/or modify metabolism, inhibit excretion, and/or modify distribution in a subject or cell. It will also be appreciated that the additional pharmaceutical agents employed may achieve a desired effect for the same disorder, and/or it may achieve different effects. In certain embodiments, a pharmaceutical composition described herein including a provided compound described herein and an additional pharmaceutical agent exhibit a synergistic effect that is absent in a pharmaceutical composition including one of the provided compounds and the additional pharmaceutical agent, but not both. In some embodiments, the additional pharmaceutical agent achieves a desired effect for the same disorder. In some embodiments, the additional pharmaceutical agent achieves different effects.

The provided compound or pharmaceutical composition can be administered concurrently with, prior to, or subsequent to one or more additional pharmaceutical agents, which are different from the compound or pharmaceutical composition and may be useful as, e.g., combination therapies. Pharmaceutical agents include diagnostic agents. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides, synthetic proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for diagnosing, treating, and/or preventing a disease.

Each additional pharmaceutical agent may be administered at a dose and/or on a time schedule determined for that pharmaceutical agent. The additional pharmaceutical agents may also be administered together with each other and/or with the compound or pharmaceutical composition described herein in a single dose or administered separately in different doses. The particular combination to employ in a regimen will take into account compatibility of the compound described herein with the additional pharmaceutical agent(s) and/or the desired diagnostic, therapeutic, and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, cytotoxic agents, anti-angiogenesis agents, anti-inflammatory agents, and immunosuppressants. In certain embodiments, the additional pharmaceutical agent is an anti-inflammatory agent. In certain embodiments, the additional pharmaceutical agent is an immunotherapy. In certain embodiments, the additional pharmaceutical agent is an anti-proliferative agent. In certain embodiments, the additional pharmaceutical agent is an anti-cancer agent. In certain embodiments, the anti-cancer agents include, but are not limited to, epigenetic or transcriptional modulators (e.g., DNA methyltransferase inhibitors, HDAC inhibitors, lysine methyltransferase inhibitors), antimitotic drugs (e.g., taxanes and vinca alkaloids), cell signaling pathway inhibitors (e.g., tyrosine protein kinase inhibitors), modulators of protein stability (e.g., proteasome inhibitors), Hsp90 inhibitors, glucocorticoids, all-trans retinoic acids, anti-estrogens (e.g., tamoxifen, raloxifene, and megestrol), LHRH agonists (e.g., goscrclin and leuprolide), anti-androgens (e.g. flutamide and bicalutamide), photodynamic therapies (e.g., vertoporfin (BPD-MA), phthalocyanine, photosensitizer Pc4, and demethoxy-hypocrellin A (2BA-2-DMHA)), nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g., methotrexate, dichloromethotrexate, trimetrexate, edatrexate), IMP dehydrogenase inhibitors (e.g., mycophenolic acid, tiazofurin, ribavirin, and EICAR), ribonuclotide reductase inhibitors (e.g., hydroxyurea and deferoxamine), uracil analogs (e.g., 5-fluorouracil (5-FU), floxuridine, doxifluridine, ratitrexed, tegafur-uracil, capecitabine), cytosine analogs (e.g., cytarabine (ara C), cytosine arabinoside, and fludarabine), purine analogs (e.g., mercaptopurine and Thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g., verapamil), Ca2+ATPase inhibitors (e.g., thapsigargin), thalidomide, lenalidomide, pomalidomide, tyrosine kinase inhibitors (e.g., axitinib, bosutinib, cediranib (RECENTIN™), dasatinib (SPRYCEL®), erlotinib (TARCEVA®), gefitinib (IRESSA®), imatinib (Gleevec®), lapatinib (TYKERB®, TYVERB®), lestaurtinib, neratinib, nilotinib (TASIGNA®), semaxanib, sunitinib (SUTENT®), toceranib (PALLADIA®), vandetanib (ZACTIMA®, ZD6474), vatalanib (PTK787), nilotinib (TASIGNA®), sorafenib (NEXAVAR®), everolimus (AFINITOR®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, camptothecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

In certain embodiments, the additional pharmaceutical agent is an immunotherapy. In certain embodiments, the immunotherapy is useful in the treatment of a cancer. Exemplary immunotherapies include, but are not limited to, T-cell therapies, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies. In certain embodiments, the immunotherapy is a T-cell therapy. In certain embodiments, the T-cell therapy is chimeric antigen receptor T cells (CAR-T). In certain embodiments, the immunotherapy is an antibody. In certain embodiments, the antibody is an anti-PD-1 antibody, an anti-PD-L1 antibody, an anti-CTLA-4 antibody, an anti-TIM3 antibody, an anti-OX40 antibody, an anti-GITR antibody, an anti-LAG-3 antibody, an anti-CD137 antibody, an anti-CD27 antibody, an anti-CD28 antibody, an anti-CD28H antibody, an anti-CD30 antibody, an anti-CD39 antibody, an anti-CD40 antibody, an anti-CD47 antibody, an anti-CD48 antibody, an anti-CD70 antibody, an anti-CD73 antibody, an anti-CD96 antibody, an anti-CD160 antibody, an anti-CD200 antibody, an anti-CD244 antibody, an anti-ICOS antibody, an anti-TNFRSF25 antibody, an anti-TMIGD2 antibody, an anti-DNAM1 antibody, an anti-BTLA antibody, an anti-LIGHT antibody, an anti-TIGIT antibody, an anti-VISTA antibody, an anti-HVEM antibody, an anti-Siglec antibody, an anti-GAL1 antibody, an anti-GAL3 antibody, an anti-GAL9 antibody, an anti-BTNL2 (butrophylins) antibody, an anti-B7-H3 antibody, an anti-B7-H4 antibody, an anti-B7-H5 antibody, an anti-B7-H6 antibody, an anti-KIR antibody, an anti-LIR antibody, an anti-ILT antibody, an anti-MICA antibody, an anti-MICB antibody, an anti-NKG2D antibody, an anti-NKG2A antibody, an anti-TGFß antibody, an anti-TGFBR antibody, an anti-CXCR4 antibody, an anti-CXCL12 antibody, an anti-CCL2 antibody, an anti-IL-10 antibody, an anti-IL-13 antibody, an anti-IL-23 antibody, an anti-phosphatidylserine antibody, an anti-neuropilin antibody, an anti-GalCer antibody, an anti-HER2 antibody, an anti-VEGFA antibody, an anti-VEGFR antibody, an anti-EGFR antibody, or an anti-Tie2 antibody. In certain embodiments, the antibody is pembrolizumab, nivolumab, pidilizumab, ipilimumab, tremelimumab, durvalumab, atezolizumab, avelumab, PF-06801591, utomilumab, PDR001, PBF-509, MGB453, LAG525, AMP-224, INCSHR1210, INCAGN1876, INCAGN1949, samalizumab, PF-05082566, urelumab, lirilumab, lulizumab, BMS-936559, BMS-936561, BMS-986004, BMS-986012, BMS-986016, BMS-986178, IMP321, IPH2101, IPH2201, varilumab, ulocuplumab, monalizumab, MEDI0562, MEDI0680, MEDI1873, MEDI6383, MEDI6469, MEDI9447, AMG228, AMG820, CC-90002, CDX-1127, CGEN15001T, CGEN15022, CGEN15029, CGEN15049, CGEN15027, CGEN15052, CGEN15092, CX-072, CX-2009, CP-870893, lucatumumab, dacetuzumab, Chi Lob 7/4, RG6058, RG7686, RG7876, RG7888, TR^{X518}, MK-4166, MGA271, IMC-CS4, emactuzumab, pertuzumab, obinutuzumab, cabiralizumab, margetuximab, enoblituzumab, mogamulizumab, carlumab, bevacizumab, trastuzumab (HERCEPTIN®), bevacizumab (AVASTIN®), rituximab (RITUXAN®), cetuximab (ERBITUX®), panitumumab (VECTIBIX®), alemtuzumab (CAMPATH®), or ranibizumab (Lucentis®).

In certain embodiments, at least one of the additional pharmaceutical agents is a cytotoxic agent. In certain embodiments, at least one of the additional pharmaceutical agents is venetoclax, azacitidine, bortezomib, cladribine, cytarabine, doxorubicin, eribulin, etoposide, everolimus, ixabepilone, mitoxantrone, ixazomib, panobinostat, pemetrexed, SN-38, or topotecan, or a pharmaceutically acceptable salt, solvate, hydrate, polymorph, co-crystal, tautomer, stereoisomer, isotopically labeled compound, or prodrug thereof. In certain embodiments, at least one of the additional pharmaceutical agents is nivolumab, pidilizumab, pembrolizumab, MEDI-0680, REGN2810, AMP-224, atezolizumab, durvalumab, BMS-936559, avelumab, CA-170, ipilimumab, or tremelimumab, or a pharmaceutically acceptable salt, solvate, hydrate, polymorph, co-crystal, tautomer, stereoisomer, isotopically labeled compound, or prodrug thereof.

In certain embodiments, the provided compounds or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy.

In certain embodiments, the provided compound or pharmaceutical composition is a solid. In certain embodiments, the provided compound or pharmaceutical composition is a powder. In certain embodiments, the provided compound or pharmaceutical composition can be dissolved in a liquid to make a solution. In certain embodiments, the provided compound or pharmaceutical composition is dissolved in water to make an aqueous solution. In certain embodiments, the pharmaceutical composition is a liquid for parental injection. In certain embodiments, the pharmaceutical composition is a liquid for oral administration (e.g., ingestion). In certain embodiments, the pharmaceutical composition is a liquid (e.g., aqueous solution) for intravenous injection. In certain embodiments, the pharmaceutical composition is a liquid (e.g., aqueous solution) for subcutaneous injection.

Pharmaceutical compositions described herein can be prepared by any method known in the art of pharmacology. In general, such preparatory methods include the steps of bringing the composition comprising a provided compound (i.e., the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the provided compound, pharmaceutically acceptable excipient, agent, and/or any additional ingredients in a pharmaceutical composition described herein will vary, depending upon the identity, size, and/or condition of the subject diagnosed or treated and further depending upon the route by which the pharmaceutical composition is to be administered. The pharmaceutical composition may comprise between 0.1% and 100% (w/w) agent.

Pharmaceutically acceptable excipients used in manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients and accessory ingredients, such as cocoa butter and suppository waxes, coloring agents, coating agents, sweetening, flavoring, and perfuming agents, may also be present in the pharmaceutical composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan monostearate (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly (vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral pharmaceutical compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid pharmaceutical compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Pharmaceutical compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

Suitable devices for use in delivering injectable pharmaceutical compositions described herein include short needle devices. Injectable pharmaceutical compositions can be administered by devices which limit the effective penetration length of a needle into the skin. Alternatively or additionally, conventional syringes can be used in the classical mantoux method of administration. Jet injection devices which deliver liquid formulations via a liquid jet injector and/or via a needle. Ballistic powder/particle delivery devices which use compressed gas to accelerate the compound in powder form are suitable.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such pharmaceutical compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder pharmaceutical compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the pharmaceutical composition, and the active ingredient may constitute 0.1 to 20% (w/w) of the pharmaceutical composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such pharmaceutical compositions are generally suitable for administration to animals of all sorts. Modification of pharmaceutical compositions suitable for administration to humans in order to render the pharmaceutical compositions suitable for administration to various animals is well understood, and the ordinarily skilled veterinary pharmacologist can design and/or perform such modification with ordinary experimentation.

Provided compounds are typically formulated in dosage unit form for ease of administration and uniformity of dosage. It will be understood, however, that the total daily usage of the pharmaceutical compositions described herein will be decided by a physician within the scope of sound medical judgment. The specific therapeutically effective dose level for any particular subject or organism will depend upon a variety of factors including the disease being diagnosed or treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The provided compounds and pharmaceutical compositions provided herein can be administered by any route, including enteral (e.g., oral), parenteral, intravenous, intramuscular, intraarticular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically, contemplated routes are intraarticular administration, oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a provided compound required to achieve an effective amount will vary from subject to subject, depending, for example, on species, age, and general condition of a subject, severity of the side effects or disorder, identity of the particular compound of the disclosure, mode of administration, and the like. An effective amount may be included in a single dose (e.g., single oral dose) or multiple doses (e.g., multiple oral doses). In certain embodiments, when multiple doses are administered to a subject or applied to a biological sample, tissue, or cell, any two doses of the multiple doses include different or substantially the same amounts of an agent described herein.

In certain embodiments, a pharmaceutical composition comprising a provided compound is administered, orally or parenterally, at dosage levels of each pharmaceutical composition sufficient to deliver from about 0.001 mg/kg to about 200 mg/kg in one or more dose administrations for one or several days (depending on the mode of administration). In certain embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In certain embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the pharmaceutical composition described herein is administered at a dose that is below the dose at which the agent causes non-specific effects.

In certain embodiments, the pharmaceutical composition is administered at a dose of about 0.001 mg to about 1000 mg per unit dose. In certain embodiments, the pharmaceutical composition is administered at a dose of about 0.01 mg to about 200 mg per unit dose. In certain embodiments, the pharmaceutical composition is administered at a dose of about 0.01 mg to about 100 mg per unit dose. In certain embodiments, pharmaceutical composition is administered at a dose of about 0.01 mg to about 50 mg per unit dose. In certain embodiments, the pharmaceutical composition is administered at a dose of about 0.01 mg to about 10 mg per unit dose. In certain embodiments, the pharmaceutical composition is administered at a dose of about 0.1 mg to about 10 mg per unit dose.

Dose ranges as described herein provide guidance for the administration of provided compounds or pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

In certain embodiments, when multiple doses are administered to a subject or applied to a tissue or cell, the frequency of administering the multiple doses to the subject or applying the multiple doses to the tissue or cell may be, in non-limiting examples, three doses a day, two doses a day, one dose a day, one dose every other day, one dose every third day, one dose every week, one dose every two weeks, one dose every three weeks, or one dose every four weeks, or even slow dose controlled delivery over a selected period of time using a drug delivery device. In certain embodiments, when multiple doses are administered to a subject or applied to a biological sample, tissue, or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

Also encompassed by the present disclosure are kits (e.g., pharmaceutical packs). In certain embodiments, the kit comprises a provided compound or pharmaceutical composition described herein, and instructions for using the compound or pharmaceutical composition. In certain embodiments, the kit comprises a first container, wherein the first container includes the compound or pharmaceutical composition. In some embodiments, the kit further comprises a second container. In certain embodiments, the second container includes an excipient (e.g., an excipient for dilution or suspension of the compound or pharmaceutical composition). In certain embodiments, the second container includes an additional pharmaceutical agent. In some embodiments, the kit further comprises a third container. In certain embodiments, the third container includes an additional pharmaceutical agent. In some embodiments, the provided compound or pharmaceutical composition included in the first container and the excipient or additional pharmaceutical agent included in the second container are combined to form one unit dosage form. In some embodiments, the provided compound or pharmaceutical composition included in the first container, the excipient included in the second container, and the additional pharmaceutical agent included in the third container are combined to form one unit dosage form. In certain embodiments, each of the first, second, and third containers is independently a vial, ampule, bottle, syringe, dispenser package, tube, or inhaler.

In certain embodiments, the instructions are for administering the provided compound or pharmaceutical composition to a subject (e.g., a subject in need of diagnosis, treatment, or prevention of a disease described herein). In certain embodiments, the instructions are for contacting a biological sample or cell with the provided compound or pharmaceutical composition. In certain embodiments, the instructions comprise information required by a regulatory agency, such as the U.S. Food and Drug Administration (FDA) or the European Agency for the Evaluation of Medicinal Products (EMA). In certain embodiments, the instructions comprise prescribing information.

In certain embodiments, the kits and instructions provide for diagnosing a disease in a subject in need thereof. In certain embodiments, the kits and instructions provide for treating a disease in a subject in need thereof. In certain embodiments, the kits and instructions provide for preventing a disease in a subject in need thereof. In certain embodiments, the kits and instructions provide for reducing the risk of developing a disease in a subject in need thereof. A kit described herein may include one or more additional pharmaceutical agents described herein as a separate pharmaceutical composition.

### Methods of Diagnosis, Treatment, Prevention, and Use

The present disclosure also provides methods for diagnosing diseases in a subject in need thereof, the methods comprising administering to the subject an effective amount of a provided compound or pharmaceutical composition.

The present disclosure also provides methods for assessing liver function in a subject in need thereof, the methods comprising administering to the subject an effective amount of a provided compound or pharmaceutical composition.

The present disclosure also provides methods for assessing biliary obstruction (e.g., within a segment of the liver) in a subject in need thereof, the methods comprising administering to the subject an effective amount of a provided compound or pharmaceutical composition. In certain embodiments, the biliary obstruction is biliary atresia.

The present disclosure also provides methods for assessing bile leak in a subject in need thereof, the methods comprising administering to the subject an effective amount of a provided compound or pharmaceutical composition. In certain embodiments, the bile leak is a result of injury (e.g., trauma) or surgical procedure (e.g., liver resection, liver transplantation).

In certain embodiments, the method comprises imaging a subject, cell, tissue, or biological sample. In certain embodiments, the method comprises imaging a tissue. In certain embodiments, the method comprises imaging liver tissue. In certain embodiments, the method comprises imaging through positron emission tomography (PET). In certain embodiments, the method comprises imaging through computed tomography (CT). In certain embodiments, the method comprises imaging through PET/CT. In certain embodiments, the imaging comprises detecting a radioisotope. In certain embodiments, the imaging comprises scintillography. In certain embodiments, the imaging comprises detecting radiation from a radioisotope. In certain embodiments, the imaging comprises detecting radiation from a radioisotope-containing compound. In certain embodiments, the method comprises imaging a provided compound that degrades less than 10% one hour after preparing the provided compound. In certain embodiments, the method comprises imaging a provided compound that degrades less than 10% two hours after preparing the provided compound. In certain embodiments, the method comprises imaging a provided compound that degrades less than 10% four hours after preparing the provided compound. In certain embodiments, the method comprises imaging a provided compound that degrades less than 10% eight hours after preparing the provided compound. In certain embodiments, the method comprises imaging a provided compound that degrades less than 10% 12 hours after preparing the provided compound. In certain embodiments, the method comprises imaging a provided compound that degrades less than 10% 16 hours after preparing the provided compound. In certain embodiments, the method comprises imaging a provided compound that degrades less than 10% 24 hours after preparing the provided compound.

In certain embodiments, the method for assessing liver function in a subject in need thereof comprises administering to the subject an effective amount of a provided compound and imaging through positron emission tomography (PET). In certain embodiments, the provided compound is labeled with ¹⁸F.

In certain embodiments, the method for assessing biliary obstruction (e.g., within a segment of the liver) in a subject in need thereof comprises administering to the subject an effective amount of a provided compound and imaging through positron emission tomography (PET). In certain embodiments, the provided compound is labeled with ¹⁸F. In certain embodiments, the biliary obstruction is biliary atresia.

In certain embodiments, the method for assessing bile leak in a subject in need thereof comprises administering to the subject an effective amount of a provided compound and imaging through positron emission tomography (PET). In certain embodiments, the provided compound is labeled with ¹⁸F. In certain embodiments, the bile leak is a result of injury (e.g., trauma) or surgical procedure (e.g., liver resection, liver transplantation).

The present disclosure also provides methods for treating diseases in a subject in need thereof, the methods comprising administering to the subject an effective amount of a provided compound or pharmaceutical composition.

The present disclosure also provides methods for preventing diseases in a subject in need thereof, the methods comprising administering to the subject an effective amount of a provided compound or pharmaceutical composition.

Without being bound by any particular theory, the compounds and pharmaceutical compositions provided herein being useful as described herein may be at least in part due to the localization to liver cancer cells by the compounds and pharmaceutical compositions provided herein. Compared to known liver cancer cell-localizing compounds, the compounds and pharmaceutical compositions provided herein may increase the potency and/or efficacy in diagnosing, treating, and/or preventing liver cancer in a subject, cell, tissue, or biological sample. Compared to known liver cancer cell-localizing compounds, the compounds and pharmaceutical compositions provided herein may increase bioavailability, safety, and/or therapeutic window, reduce toxicity and/or resistance, and/or increase subject compliance, in a subject.

The present disclosure also provides methods for inhibiting cell proliferation or promoting apoptosis in a subject in need thereof or in a cell, tissue, or biological sample, comprising administering to the subject or contacting the cell, tissue, or biological sample with an effective amount of a provided compound or pharmaceutical composition, wherein the cell, tissue, or biological sample is in vivo.

The present disclosure also provides methods for inhibiting cell proliferation or promoting apoptosis in a cell, tissue, or biological sample, comprising contacting the cell, tissue, or biological sample with an effective amount of a provided compound or pharmaceutical composition, wherein the cell, tissue, or biological sample is in vitro.

The present disclosure also provides methods for imaging a subject, comprising administering to the subject an effective amount of a provided compound or pharmaceutical composition, and detecting a radioisotope. In certain embodiments, the method comprises imaging a tissue. In certain embodiments, the method comprises imaging liver tissue. In certain embodiments, the method comprises imaging through positron emission tomography (PET). In certain embodiments, the method comprises imaging through computed tomography (CT). In certain embodiments, the method comprises imaging through PET/CT. In certain embodiments, the imaging comprises detecting a radioisotope. In certain embodiments, the imaging comprises scintillography. In certain embodiments, the imaging comprises detecting radiation from a radioisotope. In certain embodiments, the imaging comprises detecting radiation from a radioisotope-containing compound. In certain embodiments, the method comprises imaging a provided compound that degrades less than 10% one hour after preparing the provided compound. In certain embodiments, the method comprises imaging a provided compound that degrades less than 10% two hours after preparing the provided compound.

In certain embodiments, the cell, tissue, or biological sample is present in vitro. In certain embodiments, the cell, tissue, or biological sample is present in vivo. In certain embodiments, the cell, tissue, or biological sample is present ex vivo. In certain embodiments, the cell is a cancer cell. In certain embodiments, the cell is a liver cancer cell. In certain embodiments, the cell is a hepatocellular carcinoma (HCC) cell. In certain embodiments, the tissue is liver tissue. In certain embodiments, the tissue is cancerous liver tissue. In certain embodiments, the tissue is liver tissue containing cancer cells. In certain embodiments, the tissue is liver tissue containing liver cancer cells. In certain embodiments, the tissue is liver tissue containing HCC cells. In certain embodiments, the method further comprises administering to the subject one or more additional pharmaceutical agents and/or radiation. In certain embodiments, the method further comprises contacting the cell, tissue, or biological sample with one or more additional pharmaceutical agents and/or radiation.

In certain embodiments, the subject has not been administered one or more additional pharmaceutical agents, surgery, radiation therapy, transplantation (e.g., stem cell transplantation, bone marrow transplantation), immunotherapy, and/or chemotherapy. In certain embodiments, the subject has been administered one or more additional pharmaceutical agents, surgery, radiation therapy, transplantation (e.g., stem cell transplantation, bone marrow transplantation), immunotherapy, and/or chemotherapy. In certain embodiments, the subject is resistant to one or more additional pharmaceutical agents, surgery, radiation therapy, transplantation (e.g., stem cell transplantation, bone marrow transplantation), immunotherapy, and/or chemotherapy. In certain embodiments, the subject has been administered one or more additional pharmaceutical agents, surgery, radiation therapy, transplantation (e.g., stem cell transplantation, bone marrow transplantation), immunotherapy, and/or chemotherapy, and has previously experienced a remission. In certain embodiments, the subject is immunocompromised. In certain embodiments, the subject has been diagnosed with liver cancer. In certain embodiments, the subject has been diagnosed with hepatocellular carcinoma (HCC). In certain embodiments, the subject suffers from underlying liver disease, chronic hepatitis, and/or cirrhosis. In certain embodiments, the subject has not been diagnosed with liver cancer. In certain embodiments, the subject has not been diagnosed with hepatocellular carcinoma (HCC). In certain embodiments, the subject does not suffer from underlying liver disease, chronic hepatitis, and/or cirrhosis. In certain embodiments, the subject is a human. In certain embodiments, the subject is a human aged 18 years or older. In certain embodiments, the subject is a human aged younger than 18 years.

In certain embodiments, the disease is a cancer. In certain embodiments, the cancer is a liver cancer. In certain embodiments, the cancer is a hepatocellular carcinoma (HCC).

In certain embodiments, the compound is detected in an organ or tissue within 15 minutes of administration, within 30 minutes of administration, within 45 minutes of administration, within 1 hour of administration, within 2 hours of administration, within 3 hours of administration, or within 4 hours of administration. In certain embodiments, the compound is detected in the cecum within 15 minutes of administration, within 30 minutes of administration, within 45 minutes of administration, within 1 hour of administration, within 2 hours of administration, within 3 hours of administration, or within 4 hours of administration. In certain embodiments, the compound is detected in the gallbladder within 15 minutes of administration, within 30 minutes of administration, within 45 minutes of administration, within 1 hour of administration, within 2 hours of administration, within 3 hours of administration, or within 4 hours of administration. In certain embodiments, the compound is detected in the large intestine within 15 minutes of administration, within 30 minutes of administration, within 45 minutes of administration, within 1 hour of administration, within 2 hours of administration, within 3 hours of administration, or within 4 hours of administration. In certain embodiments, the compound is detected in the liver within 15 minutes of administration, within 30 minutes of administration, within 45 minutes of administration, within 1 hour of administration, within 2 hours of administration, within 3 hours of administration, or within 4 hours of administration. In certain embodiments, the compound is detected in the liver within 15 minutes of administration. In certain embodiments, the compound is detected in the liver within 30 minutes of administration. In certain embodiments, the compound is detected in the liver within 45 minutes of administration. In certain embodiments, the compound is detected in the liver within 1 hour of administration. In certain embodiments, the compound is detected in the liver within 2 hours of administration. In certain embodiments, the compound is detected in the liver within 3 hours of administration. In certain embodiments, the compound is detected in the liver within 4 hours of administration. In certain embodiments, the compound is detected in the small intestine within 15 minutes of administration, within 30 minutes of administration, within 45 minutes of administration, within 1 hour of administration, within 2 hours of administration, within 3 hours of administration, or within 4 hours of administration. In certain embodiments, the compound is detected in the spleen within 15 minutes of administration, within 30 minutes of administration, within 45 minutes of administration, within 1 hour of administration, within 2 hours of administration, within 3 hours of administration, or within 4 hours of administration.

In certain embodiments, the compound is not detected in an organ or tissue 15 minutes after administration, 30 minutes after administration, 45 minutes after administration, 1 hour after administration, 2 hours after administration, 3 hours after administration, or 4 hours after administration. In certain embodiments, the compound is not detected in the heart 15 minutes after administration, 30 minutes after administration, 45 minutes after administration, 1 hour after administration, 2 hours after administration, 3 hours after administration, or 4 hours after administration. In certain embodiments, the compound is not detected in the lungs 15 minutes after administration, 30 minutes after administration, 45 minutes after administration, 1 hour after administration, 2 hours after administration, 3 hours after administration, or 4 hours after administration.

In certain embodiments, the activity concentration in an organ or tissue after administration is at least 5, at least 10, at least 15, at least 20, or at least 25. In certain embodiments, the activity concentration in the cecum after administration is at least 5, at least 10, at least 15, at least 20, or at least 25. In certain embodiments, the activity concentration in the gallbladder after administration is at least 5, at least 10, at least 15, at least 20, or at least 25. In certain embodiments, the activity concentration in the large intestine after administration is at least 5, at least 10, at least 15, at least 20, or at least 25. In certain embodiments, the activity concentration in the liver after administration is at least 5, at least 10, at least 15, at least 20, or at least 25. In certain embodiments, the activity concentration in the liver after administration is at least 5. In certain embodiments, the activity concentration in the liver after administration is at least 10. In certain embodiments, the activity concentration in the liver after administration is at least 15. In certain embodiments, the activity concentration in the liver after administration is at least 20. In certain embodiments, the activity concentration in the liver after administration is at least 25. In certain embodiments, the activity concentration in the small intestine after administration is at least 5, at least 10, at least 15, at least 20, or at least 25. In certain embodiments, the activity concentration in the spleen after administration is at least 5, at least 10, at least 15, at least 20, or at least 25. In certain embodiments, the activity concentration is measured by standardized uptake value (SUV).

In certain embodiments, the activity uptake in an organ or tissue after administration is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99%. In certain embodiments, the activity uptake in the cecum after administration is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99%. In certain embodiments, the activity uptake in the gallbladder after administration is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99%. In certain embodiments, the activity uptake in the large intestine after administration is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99%. In certain embodiments, the activity uptake in the liver after administration is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99%.

In certain embodiments, the activity uptake in the liver after administration is at least about 5%. In certain embodiments, the activity uptake in the liver after administration is at least about 10%. In certain embodiments, the activity uptake in the liver after administration is at least about 15%. In certain embodiments, the activity uptake in the liver after administration is at least about 20%. In certain embodiments, the activity uptake in the liver after administration is at least about 25%. In certain embodiments, the activity uptake in the liver after administration is at least about 30%. In certain embodiments, the activity uptake in the liver after administration is at least about 35%. In certain embodiments, the activity uptake in the liver after administration is at least about 40%. In certain embodiments, the activity uptake in the liver after administration is at least about 45%. In certain embodiments, the activity uptake in the liver after administration is at least about 50%. In certain embodiments, the activity uptake in the liver after administration is at least about 55%. In certain embodiments, the activity uptake in the liver after administration is at least about 60%. In certain embodiments, the activity uptake in the liver after administration is at least about 65%. In certain embodiments, the activity uptake in the liver after administration is at least about 70%. In certain embodiments, the activity uptake in the liver after administration is at least about 75%. In certain embodiments, the activity uptake in the liver after administration is at least about 80%. In certain embodiments, the activity uptake in the liver after administration is at least about 85%. In certain embodiments, the activity uptake in the liver after administration is at least about 90%. In certain embodiments, the activity uptake in the liver after administration is at least about 95%. In certain embodiments, the activity uptake in the liver after administration is at least about 98%. In certain embodiments, the activity uptake in the liver after administration is at least about 99%. In certain embodiments, the activity uptake in the small intestine after administration is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99%. In certain embodiments, the activity uptake in the spleen after administration is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99%. In certain embodiments, the activity uptake in an organ or tissue after administration is a range between a percentage described in this paragraph and another percentage described in this paragraph, inclusive. In certain embodiments, the activity uptake after administration is measured by percentage injected dose (% ID).

The present disclosure also provides a provided compound or pharmaceutical composition for use in diagnosing a disease in a subject in need thereof. The present disclosure also provides a provided compound or pharmaceutical composition for use in treating a disease in a subject in need thereof. The present disclosure also provides a provided compound or pharmaceutical composition for use in preventing a disease in a subject in need thereof.

The present disclosure also provides a provided compound or pharmaceutical composition for the manufacture of a medicament for diagnosis of a disease in a subject in need thereof. The present disclosure also provides a provided compound or pharmaceutical composition for the manufacture of a medicament for treatment of a disease in a subject in need thereof. The present disclosure also provides a provided compound or pharmaceutical composition for the manufacture of a medicament for prevention of a disease in a subject in need thereof.

In certain embodiments, in vitro methods provided herein can be carried out, e.g., in an assay, cell culture, or biological sample.

In some embodiments, the cell is obtained from an organism, such as a subject. In some embodiments, the cell is a liver cancer cell. In some embodiments, the cell is a hepatocellular carcinoma (HCC) cell.

In some embodiments, the methods further comprise measuring or assessing the level of one or more properties of the cell. In some embodiments, the level of one or more properties of the cell is assessed following contacting the cell with any of the compounds or pharmaceutical compositions described herein. In some embodiments, the level of one or more properties following contacting the cell with any of the compounds or pharmaceutical compositions described herein is compared to the level of one or more properties in a reference sample or prior to contacting the cell with the compounds or pharmaceutical composition. In some embodiments, the contacting the cell with any of the compounds or pharmaceutical compositions described herein increases one or more properties of the cell. In some aspects, the methods described herein may be used to determine whether a cell is susceptible to diagnosis or treatment with the compounds or pharmaceutical compositions described herein. In some embodiments, if the level of one or more properties is increased following contacting the cell with any of the compounds or pharmaceutical compositions described herein, the cell is determined to be susceptible to diagnosis or treatment with the compound or pharmaceutical composition. In some embodiments, if the level of one or more properties is increased following contacting the cell with any of the compounds or pharmaceutical compositions described herein, the compound or pharmaceutical composition is determined to be a candidate for a disease associated with the cell.

### Methods of Preparation

In some embodiments, the present disclosure provides methods of preparing compounds of Formula I-g-i:

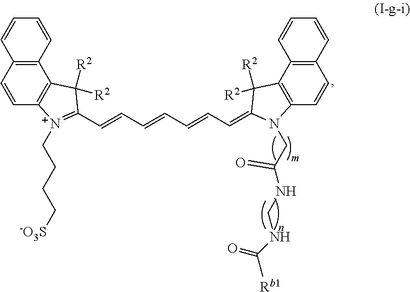

or pharmaceutically acceptable salt, solvate, hydrate, polymorph, co-crystal, tautomer, stereoisomer, or prodrug thereof, the method comprising reacting an amine of Formula I-f-ii:

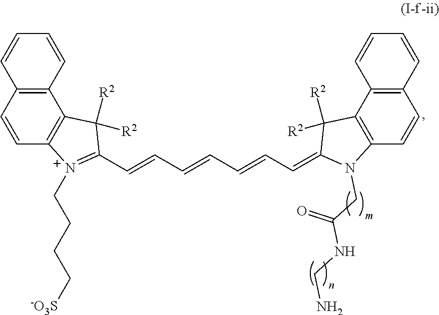

or pharmaceutically acceptable salt, solvate, hydrate, polymorph, co-crystal, tautomer, stereoisomer, or prodrug thereof, with a compound of Formula II:

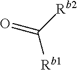

or salt thereof, wherein:
each occurrence of R² is independently hydrogen, halogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl;
R^{b1} is optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl, and contains at least one radioisotope;
R^{b2} is halogen or —ORA;
R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl;
m is 0, 1, 2, 3, 4, 5, 6, 7, 8, 9, or 10; and
n is 1, 2, 3, 4, 5, 6, 7, 8, 9, or 10.

In certain embodiments, the compound of Formula II is an activated ester of Formula II-a or Formula II-b:

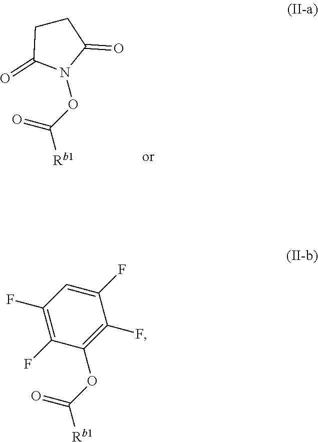

or salt thereof

In certain embodiments, the method further comprises converting an ester of Formula II-c:

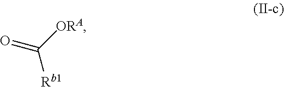

or salt thereof, to an activated ester of Formula II-a or Formula II-b:

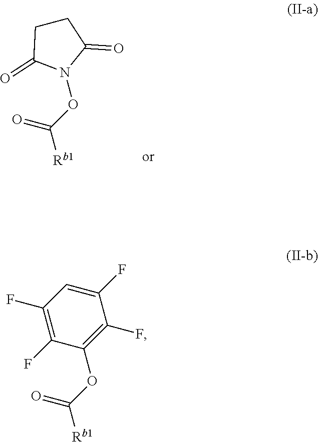

or salt thereof.

In certain embodiments, the method comprises reacting the ester of Formula II-c:

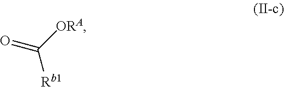

or salt thereof, with 2-(2,5-dioxopyrrolidin-1-yl)-1,1,3,3-tetramethylisouronium tetrafluoroborate (TSTU) to provide the activated ester of Formula II-a:

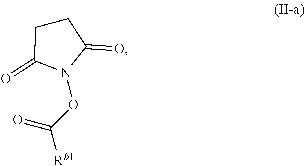

or salt thereof.

In certain embodiments, the method comprises reacting a compound of Formula II-d:

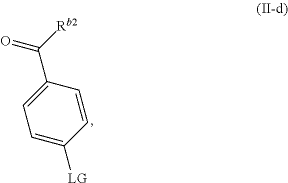

or salt thereof, with ¹⁸F to provide a compound of Formula II-e:

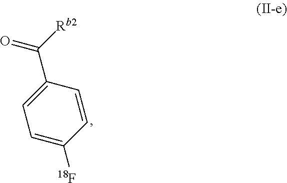

or salt thereof, wherein LG is a leaving group. In certain embodiments, the leaving group is NMe₃.

In certain embodiments, reacting the compound of Formula II-d with ¹⁸F comprises adding [¹⁸F]KF/K222. In certain embodiments, reacting the compound of Formula II-d with ¹⁸F-comprises adding [¹⁸F]TBAF.

In some embodiments, the present disclosure provides methods of preparing compounds of Formula I-j-i:

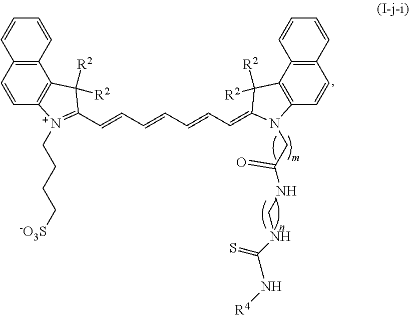

or pharmaceutically acceptable salt, solvate, hydrate, polymorph, co-crystal, tautomer, stereoisomer, or prodrug thereof, the method comprising reacting an amine of Formula I-f-ii:

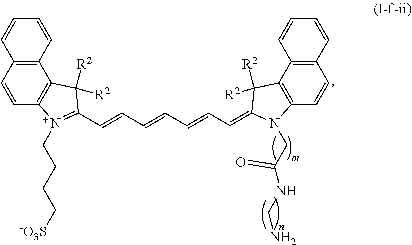

or pharmaceutically acceptable salt, solvate, hydrate, polymorph, co-crystal, tautomer, stereoisomer, or prodrug thereof, with a compound of formula:

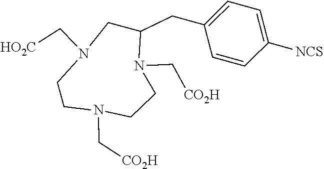

or salt thereof, wherein:
each occurrence of R² is independently hydrogen, halogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl;
R⁴ is

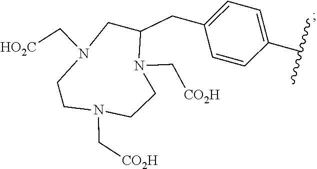 [SMILES: *c1ccc(CC2CN(CC(=O)O)CCN(CC(=O)O)CCN2CC(=O)O)cc1]

m is 0, 1, 2, 3, 4, 5, 6, 7, 8, 9, or 10; and
n is 1, 2, 3, 4, 5, 6, 7, 8, 9, or 10.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

Fibrolamellar carcinoma (FLC) is a primary liver cancer that most commonly arises in healthy adolescents and young adults in a background of normal liver tissue¹⁻³. Despite the historical categorization of FLC as a subset of hepatocellular carcinoma (HCC), it is now established that the two entities are different from a pathogenesis and molecular standpoint. Conventional HCC commonly arises in the background of cirrhosis due to viral hepatitis (B or C), alcohol, or non-alcoholic steatohepatitis and most often contains mutations in genes such as TERT, TP53, CTNNB1, and AXIN.⁴ In 2014, the DNAJB1-PRKACA (DP) gene fusion was initially described in a cohort of FLC patients⁵ with subsequent confirmatory studies demonstrating the presence of this fusion gene in the majority of FLC tumors⁶. Currently, the only chance of cure for primary liver tumors is surgical resection. However, unfortunately, a significant proportion of patients present with metastatic disease, rendering surgical resection a palliative intervention. The majority of ongoing or recent clinical trials for treatment of metastatic FLC or HCC focus on conventional chemotherapy (e.g. PLADO, Gemox), immunotherapeutics (+/−conventional chemotherapy), or targeted agents (e.g. EGFR inhibitor) in conjunction with surgical resection and interventional approaches when feasible. Despite the identification of the oncogenic driver in FLC and decades of research on HCC, these tumors continue to have a dismal prognosis and there is an urgent and unmet need for better therapeutic and diagnostic modalities.

Theranostic approaches to various cancers have made significant progress in recent years. This approach combines an agent to diagnose or localize disease and a related agent to treat the disease⁷ by taking advantage of radioisotopes of various elements with specific properties in regards to their particle and energy emission-determining their applicability for diagnostic imaging (positron emission tomography (PET), magnetic resonance imaging (MRI)) or therapeutic cytotoxicity. Current theranostic pairs used in clinical practice include agents for imaging and treatment of thyroid cancer, neuroblastoma, pheochromocytoma, hepatocellular carcinoma, prostate cancer, and neuroendocrine tumors. The approach for thyroid cancer is based on the sodium/iodide symporter, which is present on thyroid cancer cells and results in the uptake of ¹²³I for diagnostic imaging and ¹³¹I for therapy. In the case of neuroblastoma, ¹²³I/¹³¹I-labeled metaiodobenzylguanidine (mIBG), an analogue of norepinephrine, is transported through norepinephrine channels into tumor cells.⁸⁻¹⁰ In 2022, lutetium 177 conjugated to a human prostate-specific membrane antigen-targeting ligand (¹⁷⁷Lu-PSMA-617) was approved by the Food and Drug Administration (FDA) for treatment of prostate cancer. The currently approved theranostic pair for hepatocellular carcinoma uses ^{99m}Tc-macroaggregates of human serum albumin as a diagnostic agent and ⁹⁰Y microspheres as therapeutic agent. This radioembolization approach uses interventional techniques to deliver glass ⁹⁰Y microspheres directly into the feeding vessels of the tumor and due to the size of the microspheres they become lodged in the tumor. This is a mechanical approach as opposed to a molecular approach and therefore only applicable if the arterial supply of the tumor is suitable which may not be the case for many metastatic lesions.¹¹ Therefore development of molecular-based liver cancer-specific theranostic agents are desirable.

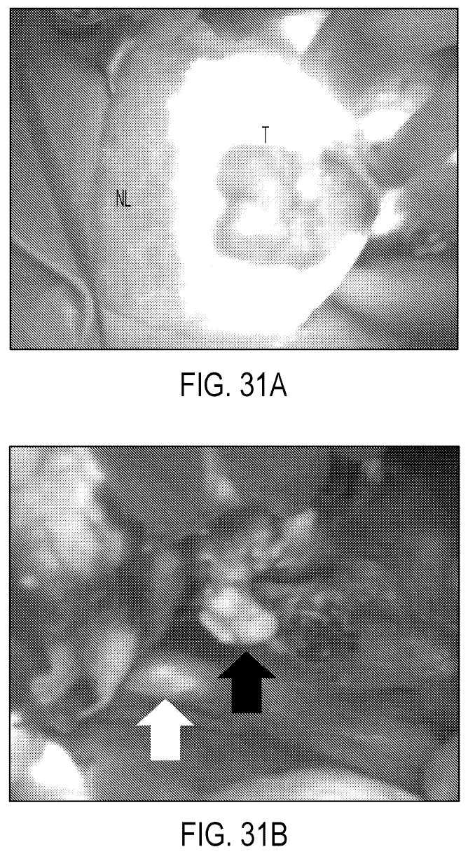

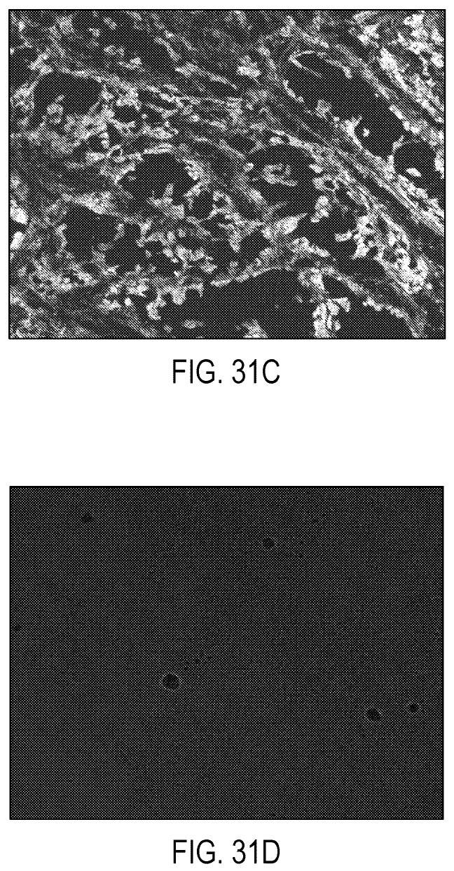

Despite the differences in the oncogenic drivers and molecular signatures of FLC and HCC, they share the ability to uptake indocyanine green (ICG). Thus, this property may provide a unique mechanism to target these cancers by using ICG as a vector to deliver radionuclides to the tumor cells. ICG is an FDA-approved drug, which has been used for various medical applications (cardiac function, hepatic function, angiography) since 1956. ICG is an amphiphilic, tricarbocyanine iodide dye with a near-infrared fluorescence property. The clearance of ICG following intravenous administration is through hepatic uptake and excretion into the bile¹² resulting in a serum half-life of approximately 4 minutes.^{13,14} As a result, ICG serum levels have been used as a surrogate for liver function.¹⁵ In addition, this property is used for identification of tumor nodules during surgery. To date, several groups have confirmed the utility of ICG for identification of primary liver and metastatic HCCs¹⁶⁻²¹. It is also demonstrated that ICG also accumulates in FLC (FIGS. 31A-31D).

ICG is administered several days prior to surgery, which subsequently is cleared from the blood and normal liver but is retained in tumor tissue. At the time of surgery, a near-infrared camera is used to identify ICG retaining tumor tissue since it fluoresces in response to near-infrared wave.^{22,23}

The uptake of ICG by hepatocytes has been shown to be mediated via several organic anion transporting polypeptides (OATP) such as OATP1B1 (SLCO1B1) and OATP2B1 (SLCO2B1)^{24,25} These solute transporters are normally responsible for uptake of bile salts by hepatocytes from plasma.²⁶ Excretion of ICG from hepatocytes into bile has been well demonstrated, however, the exact transport mechanisms are not yet fully elucidated. One study using knock-out animals demonstrated that the efflux of ICG from the hepatocytes into bile has been shown to be predominantly mediated by MDR3.27 MDR3 belongs to the ATP-binding cassette transporters, which are expressed on the canalicular membranes of hepatocytes and transport solutes from hepatocytes into the bile. Tumor cells also express MDR3, however, liver cancer cells do not form bile canaliculi, and therefore ICG is retained in the tumor tissue.²⁸

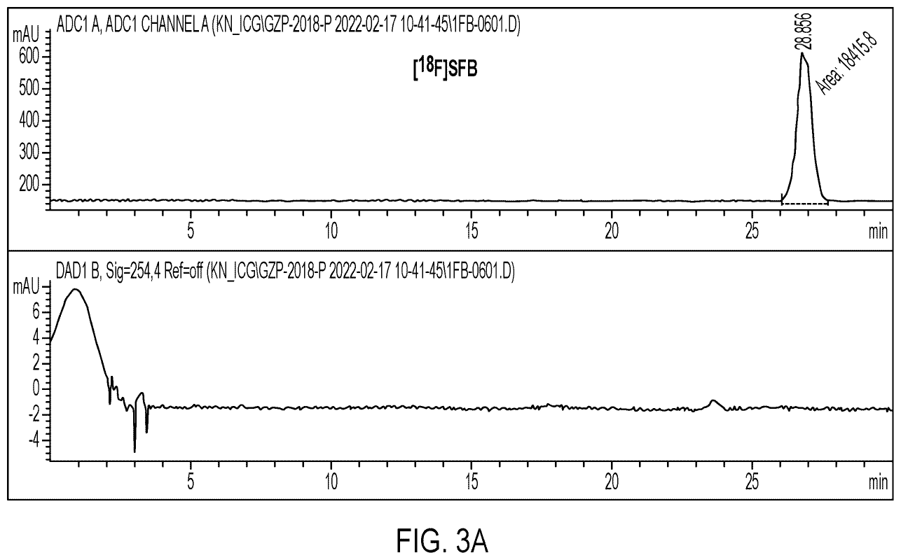

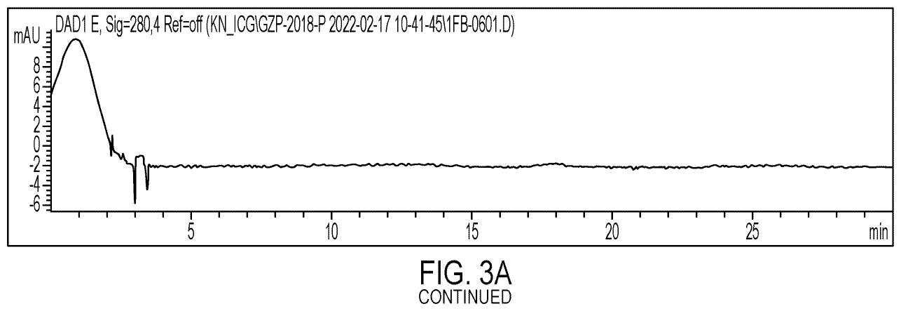

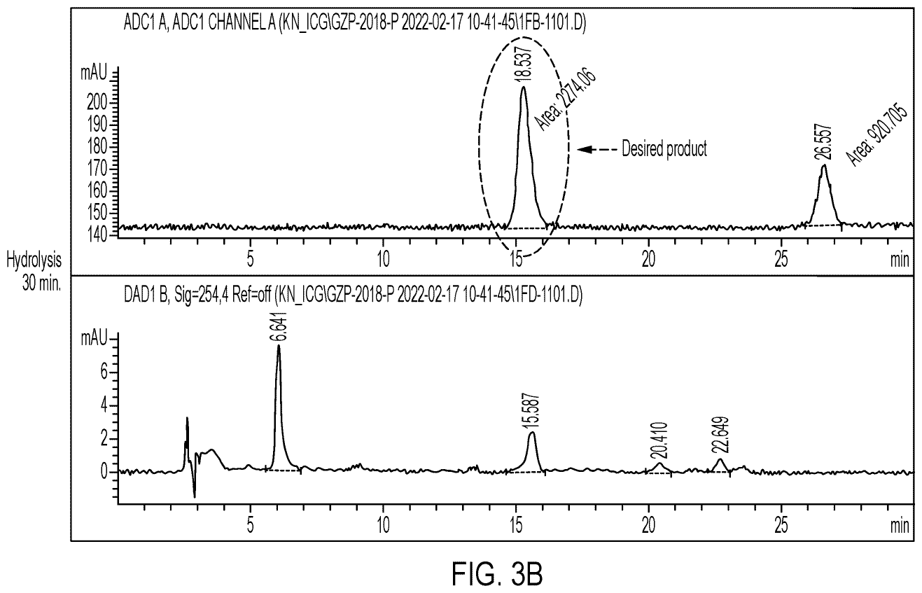

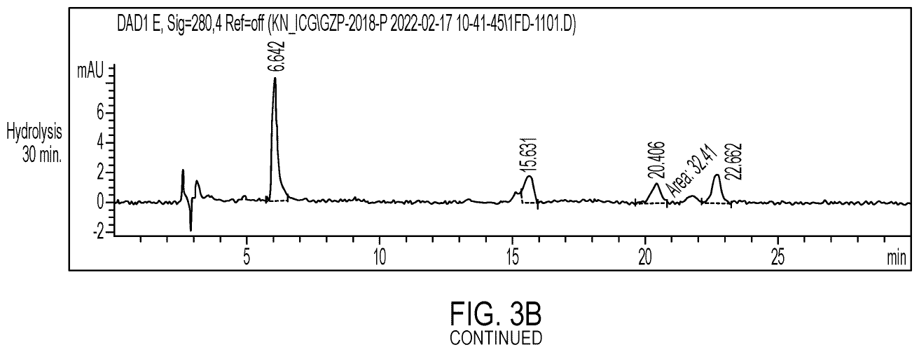

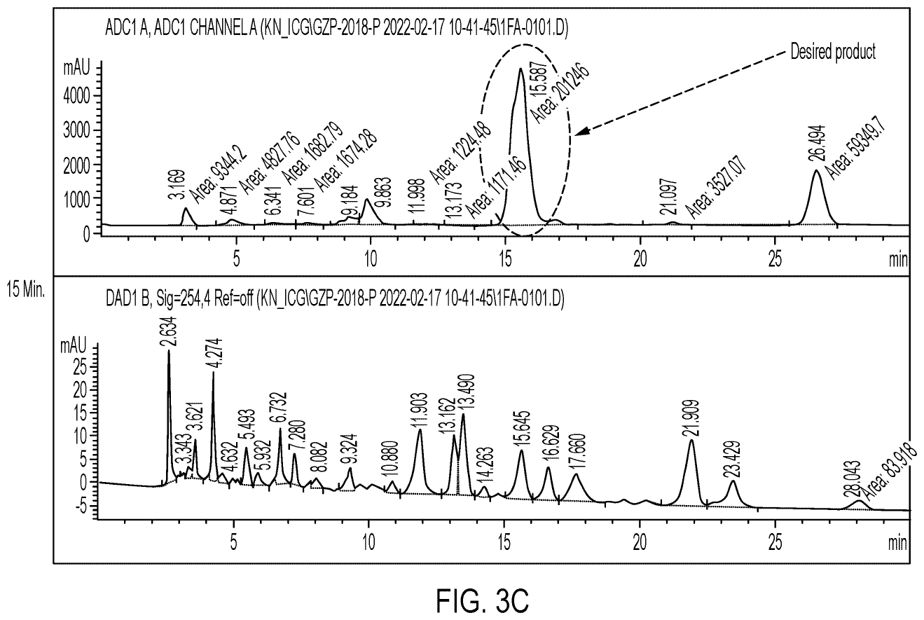

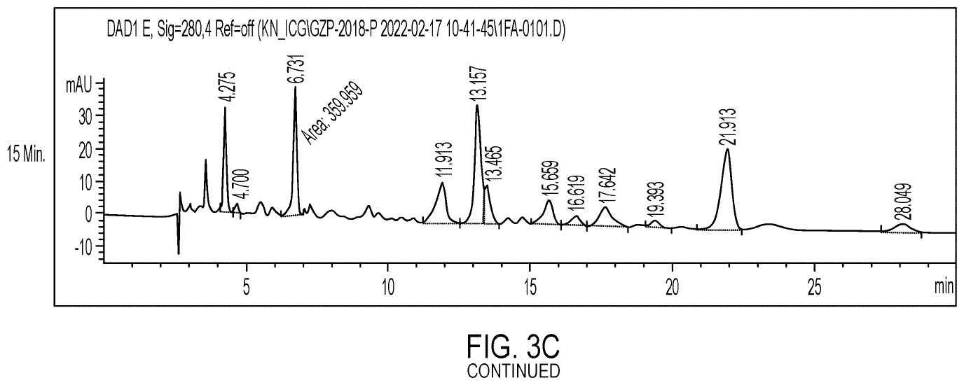

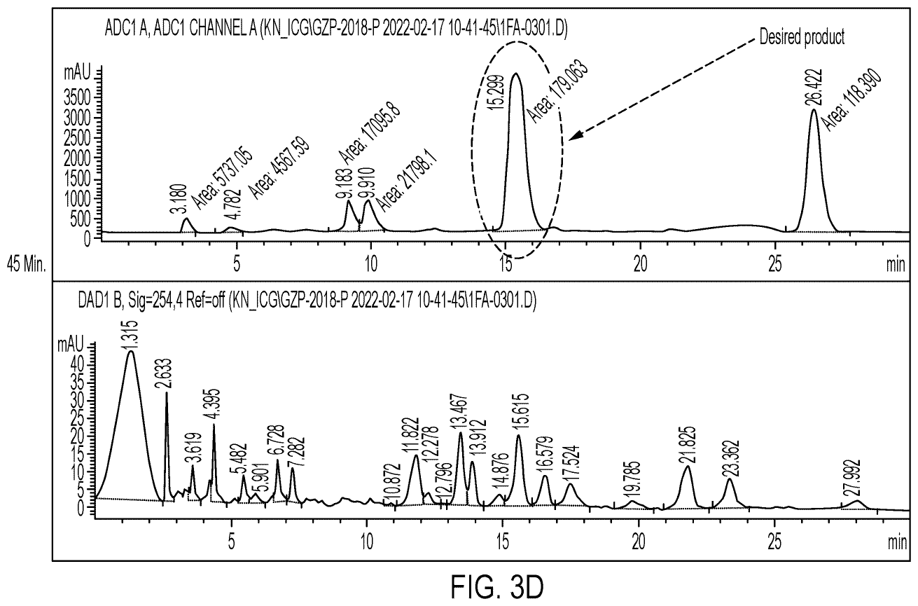

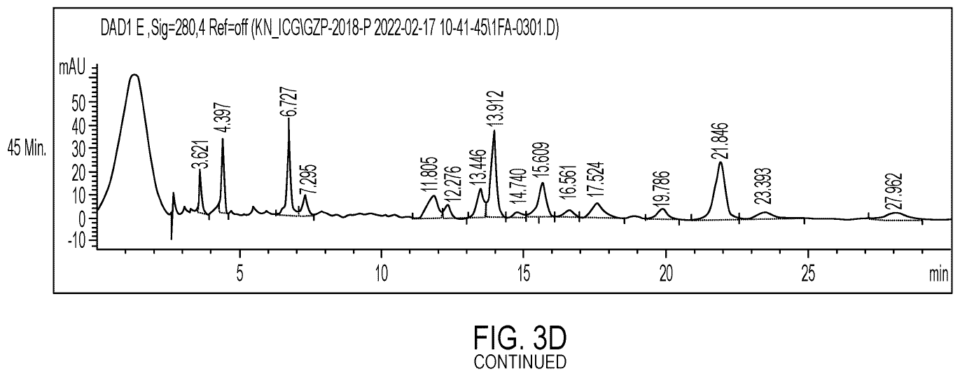

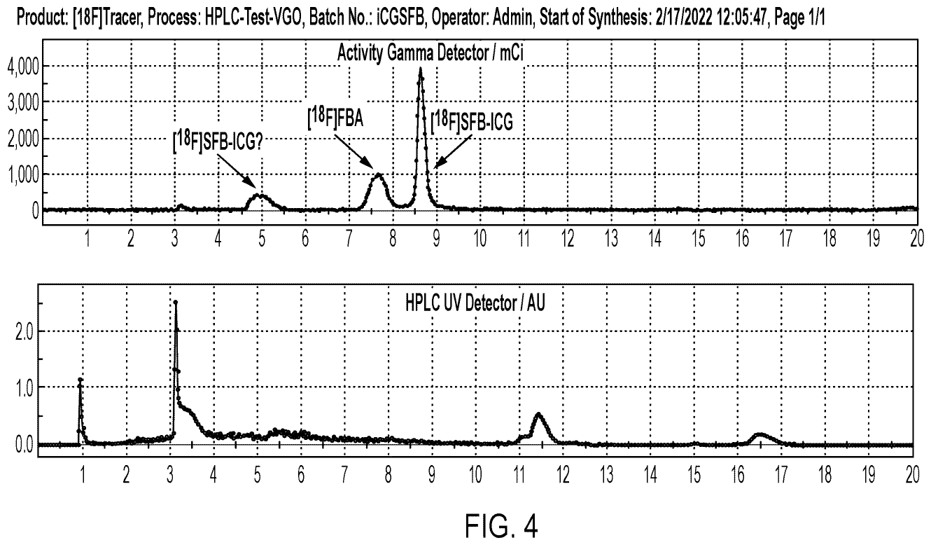

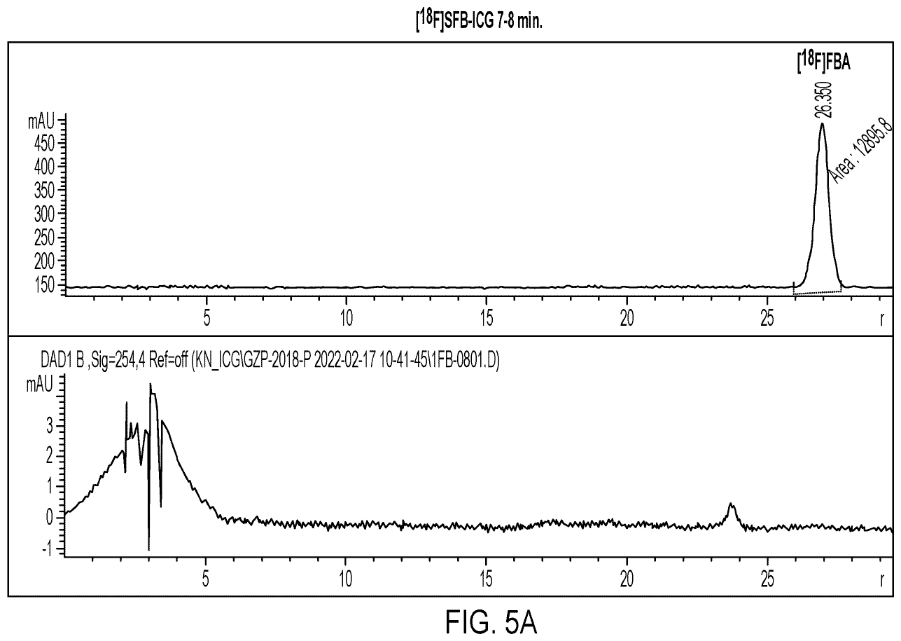

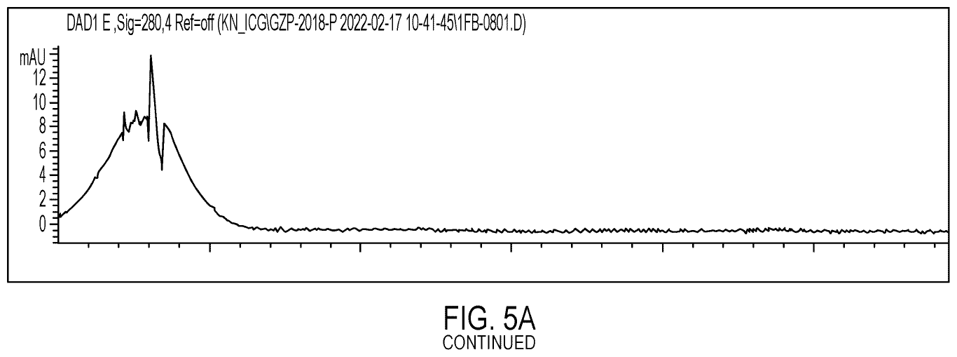

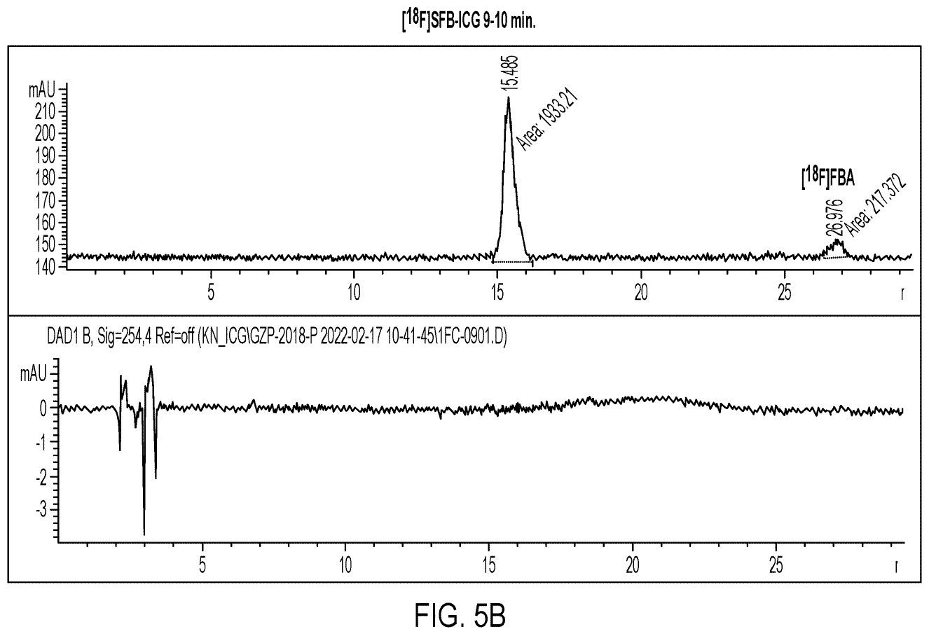

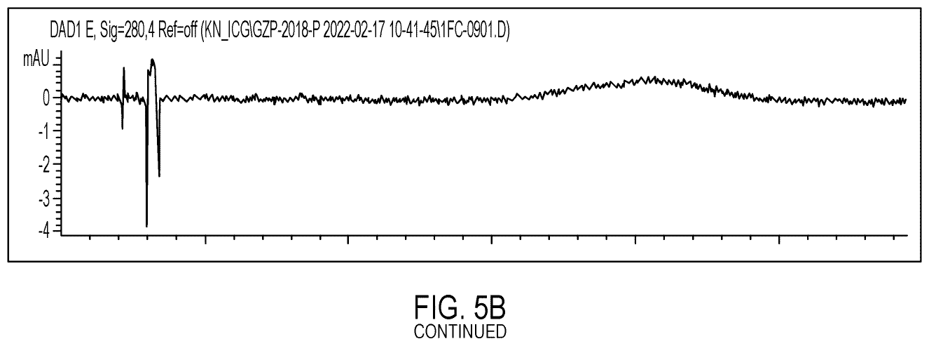

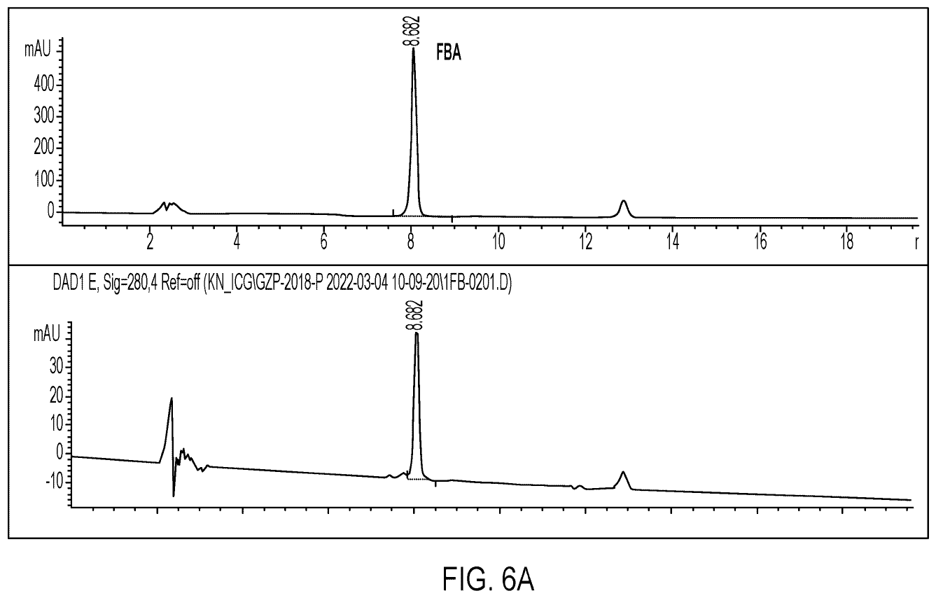

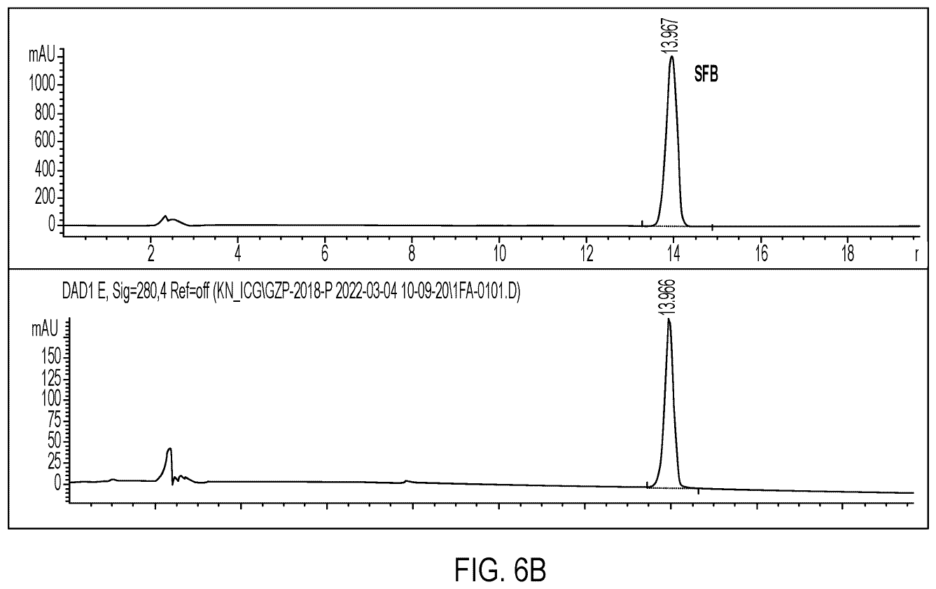

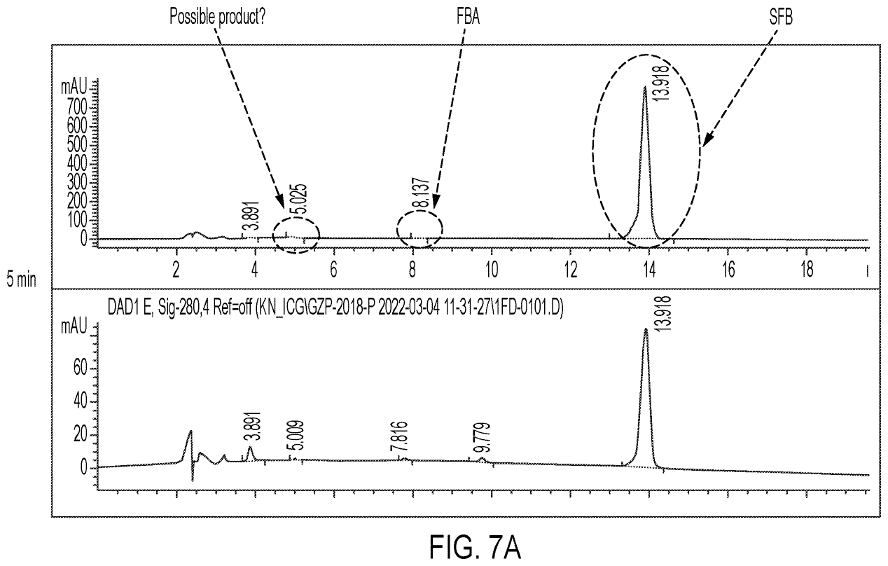

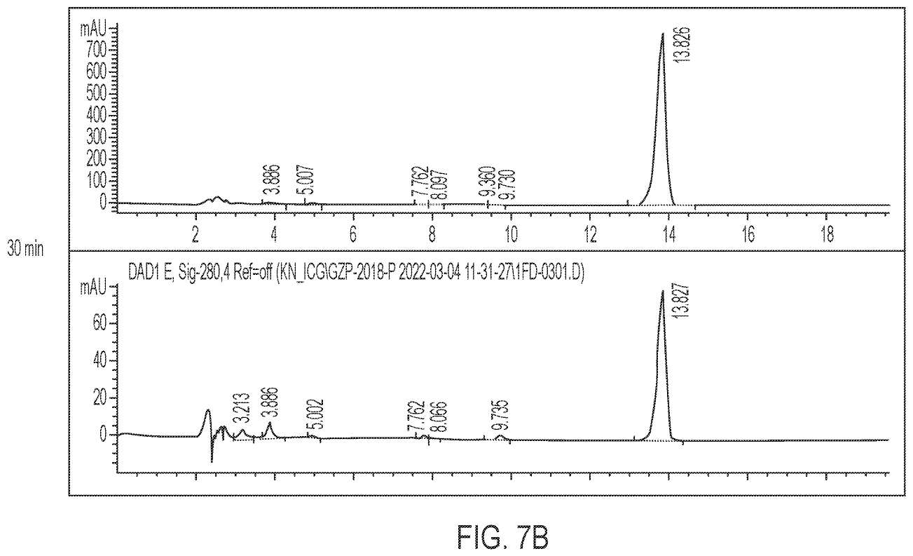

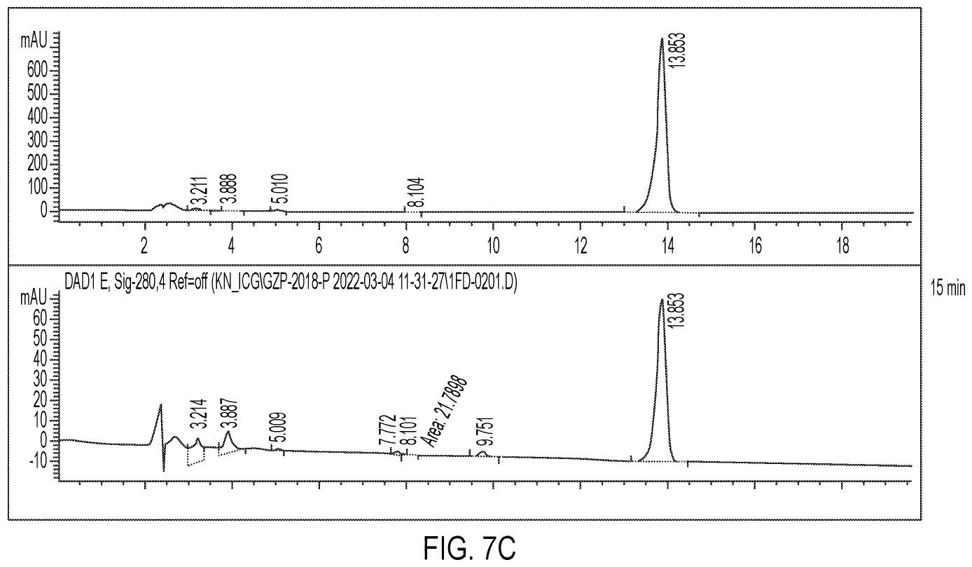

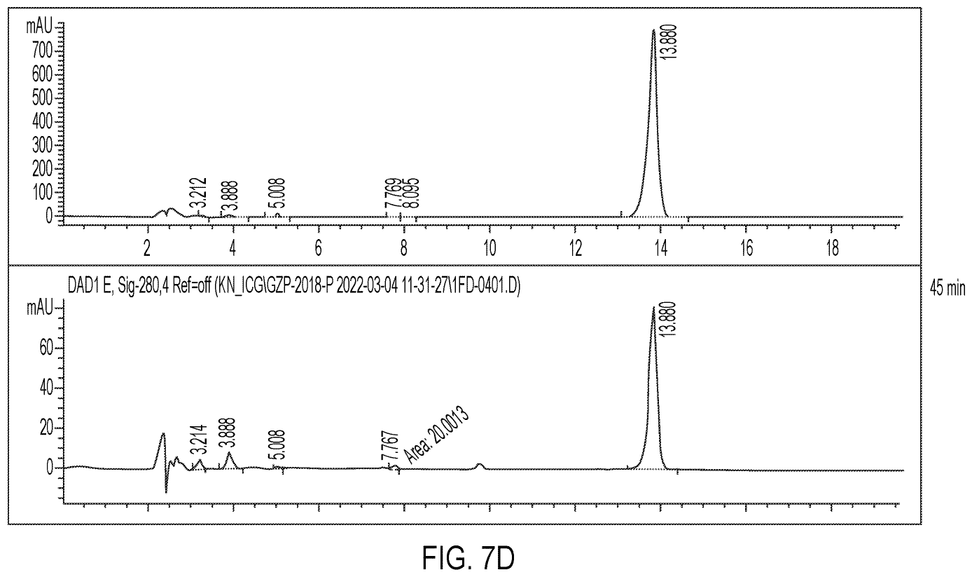

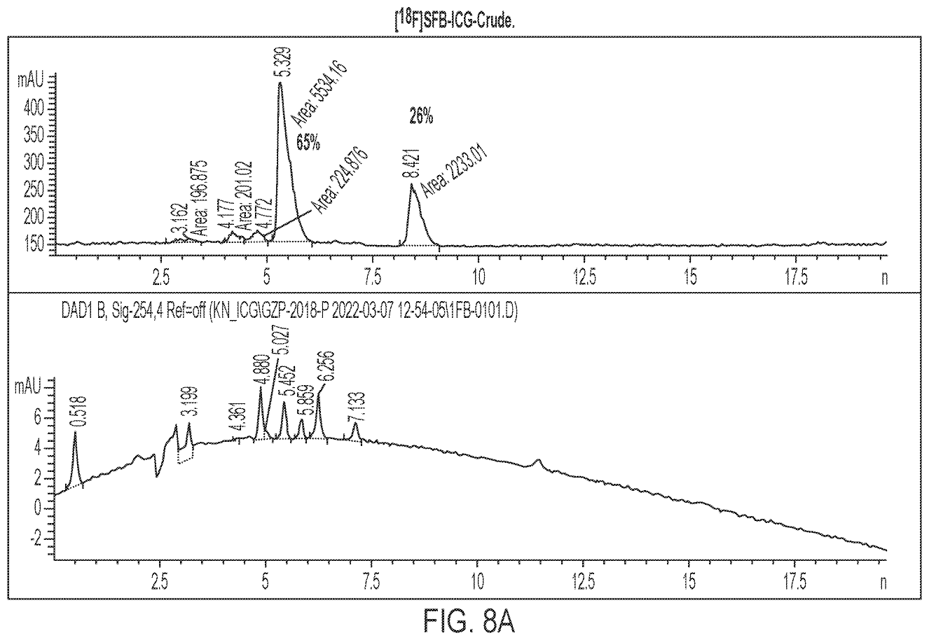

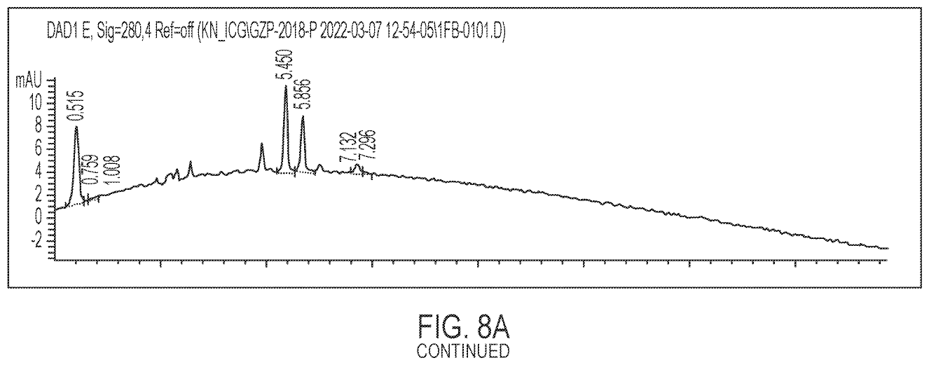

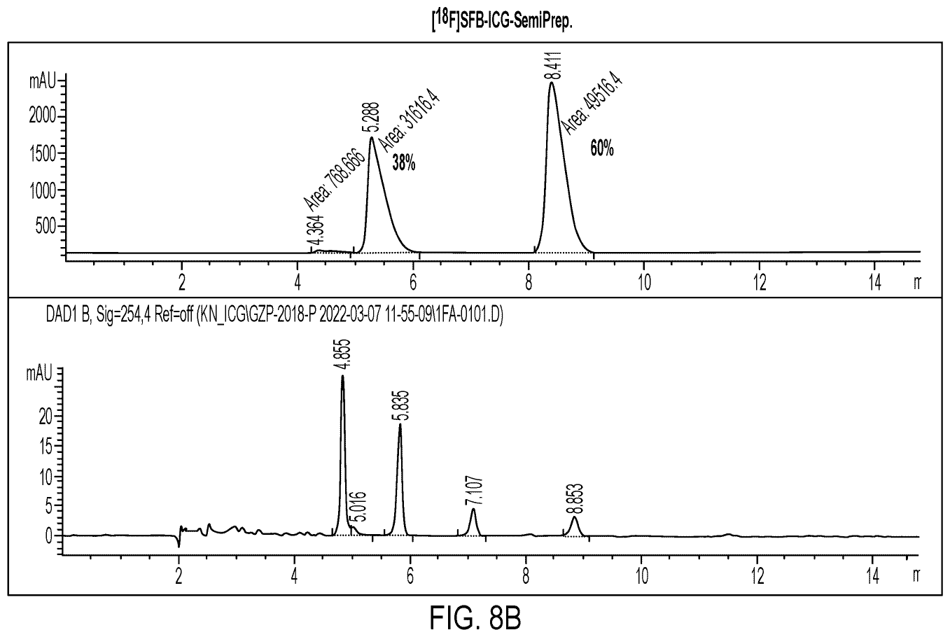

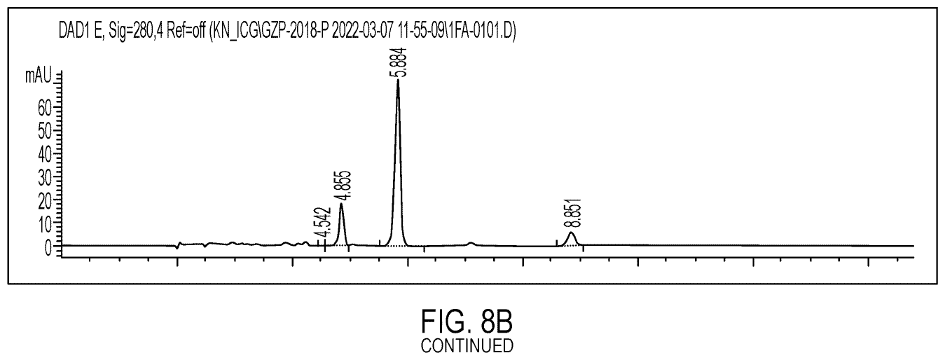

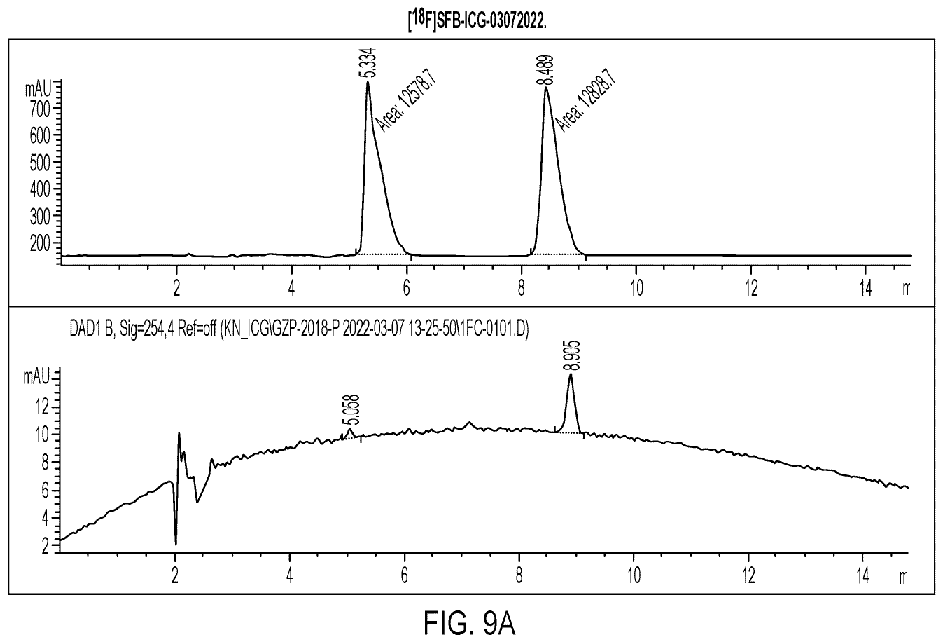

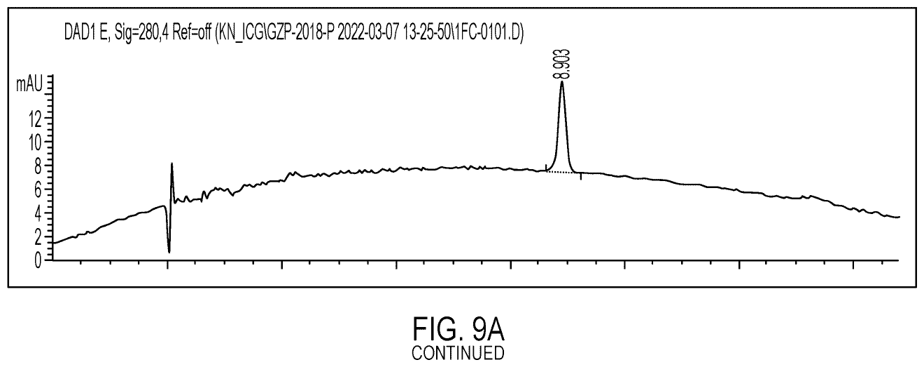

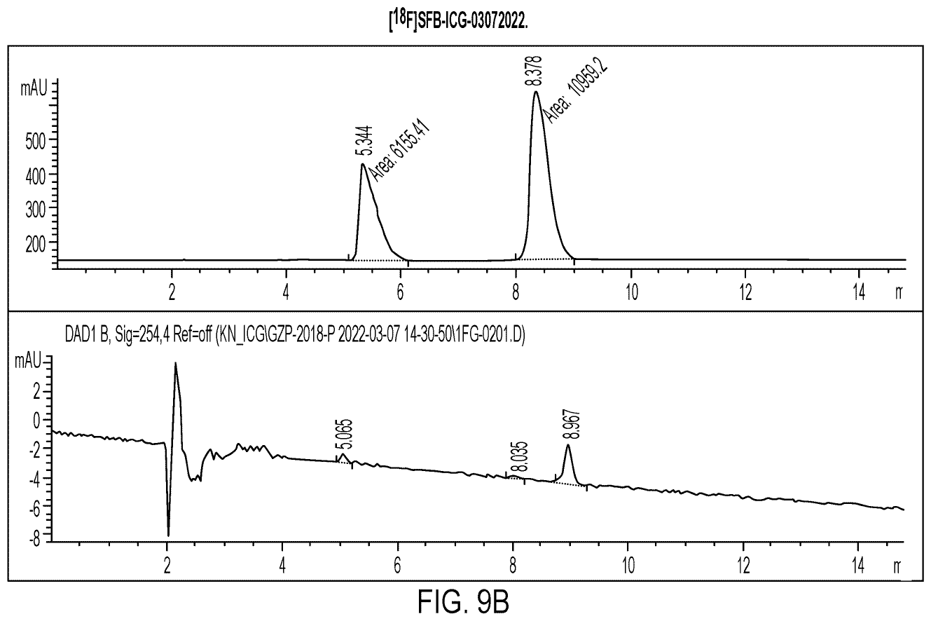

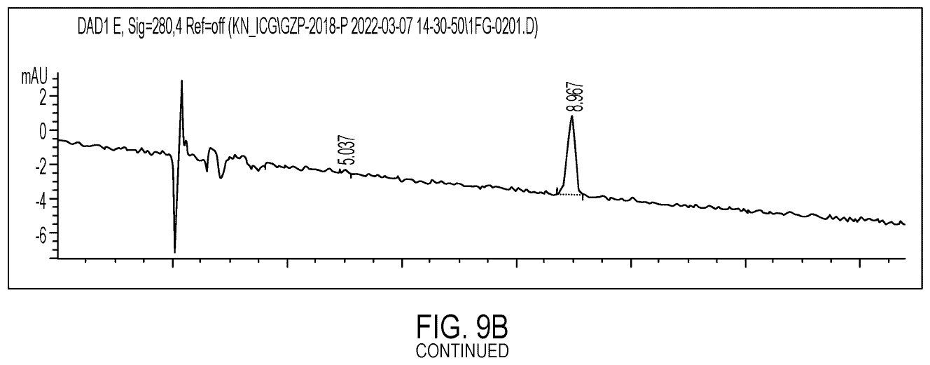

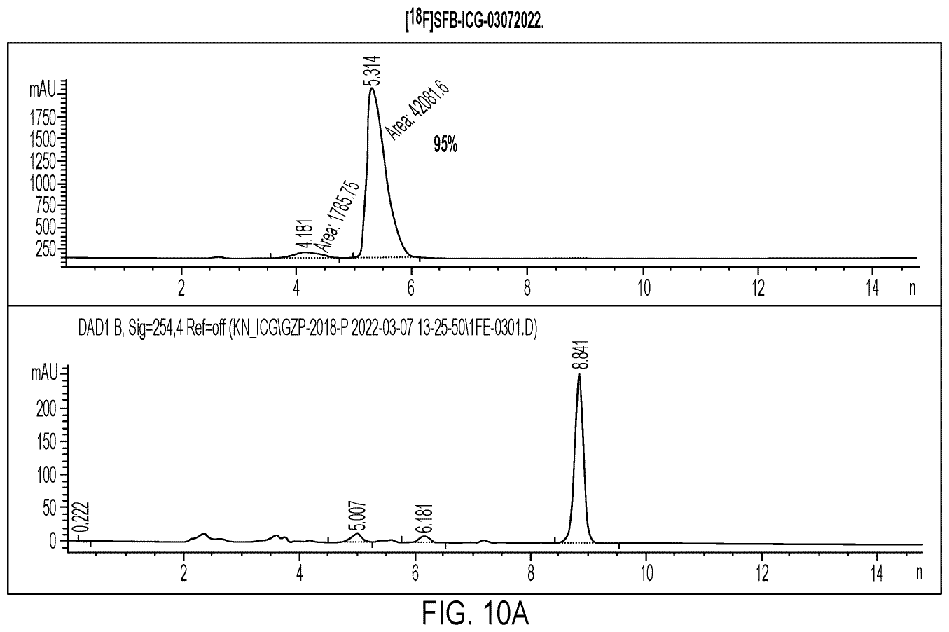

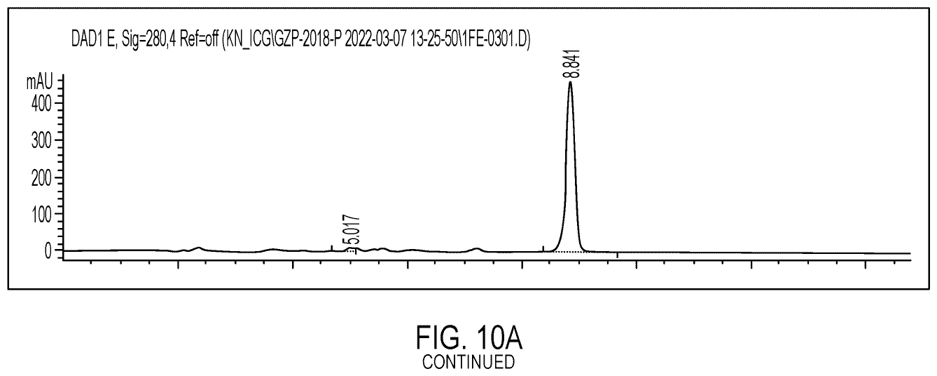

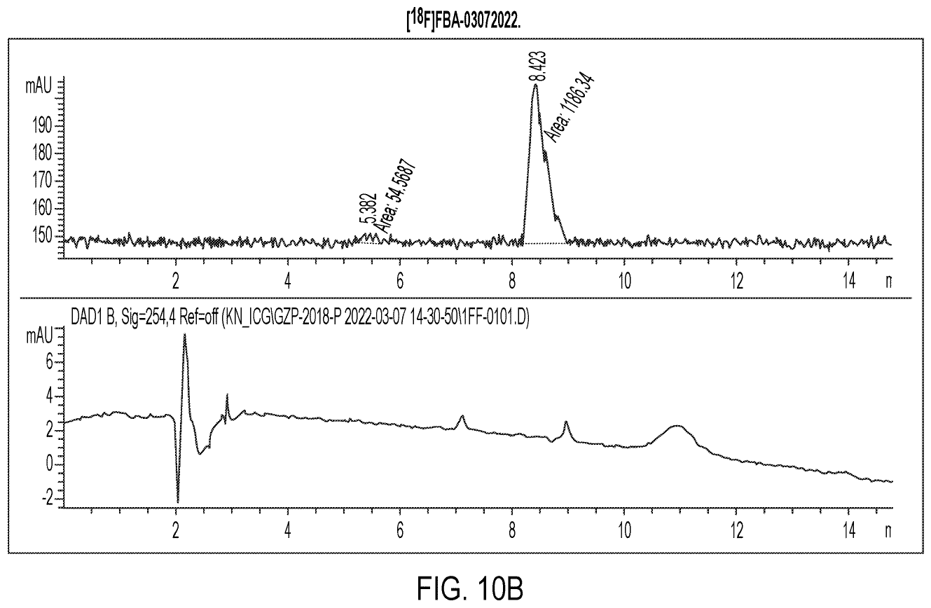

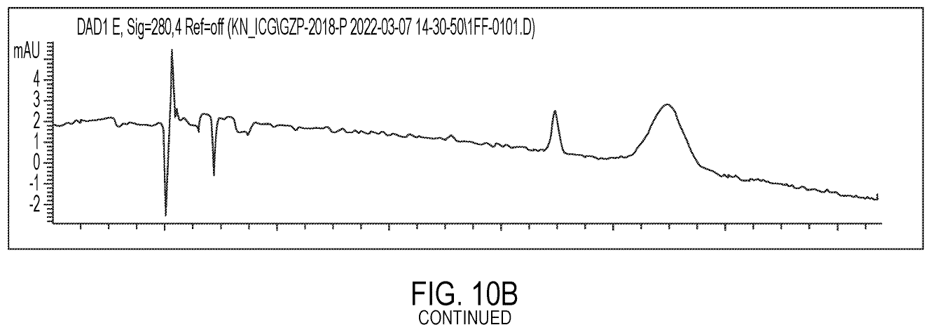

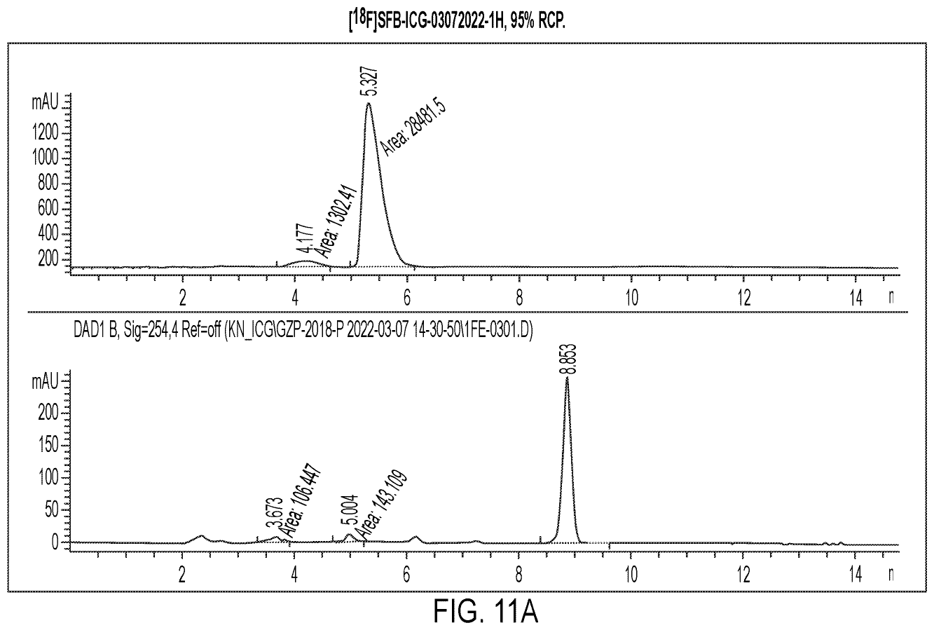

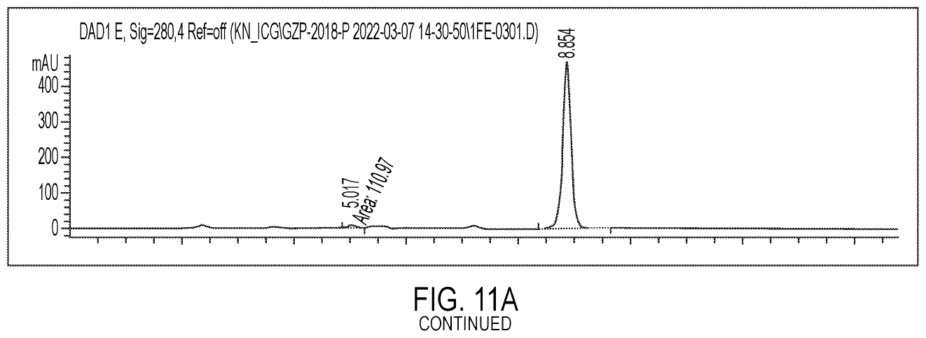

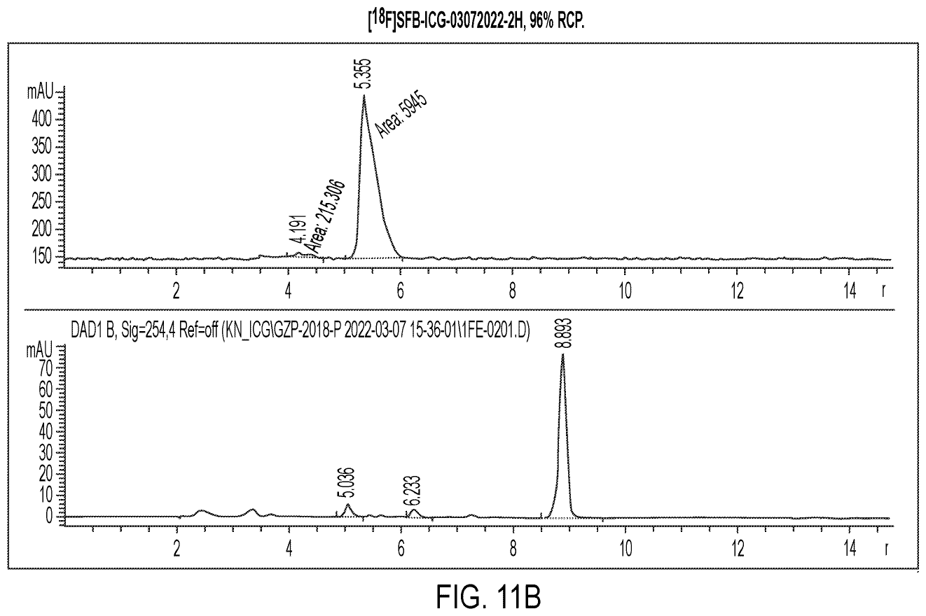

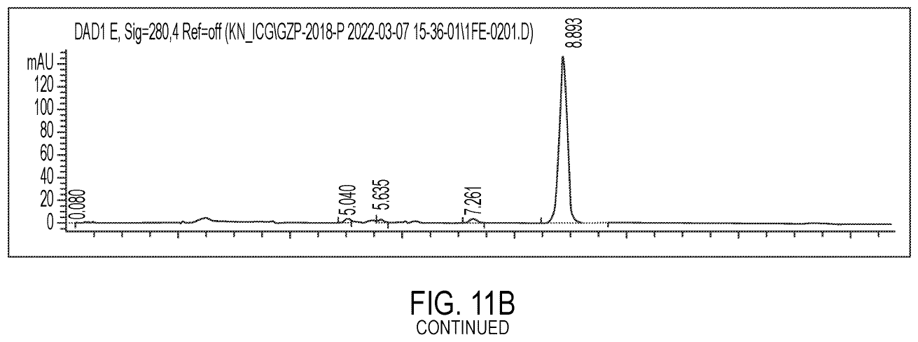

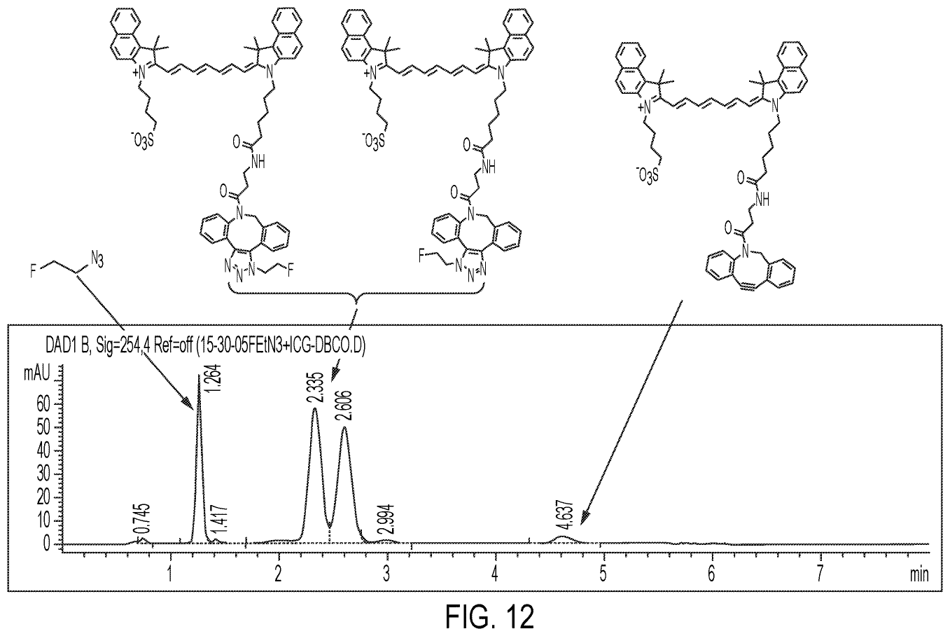

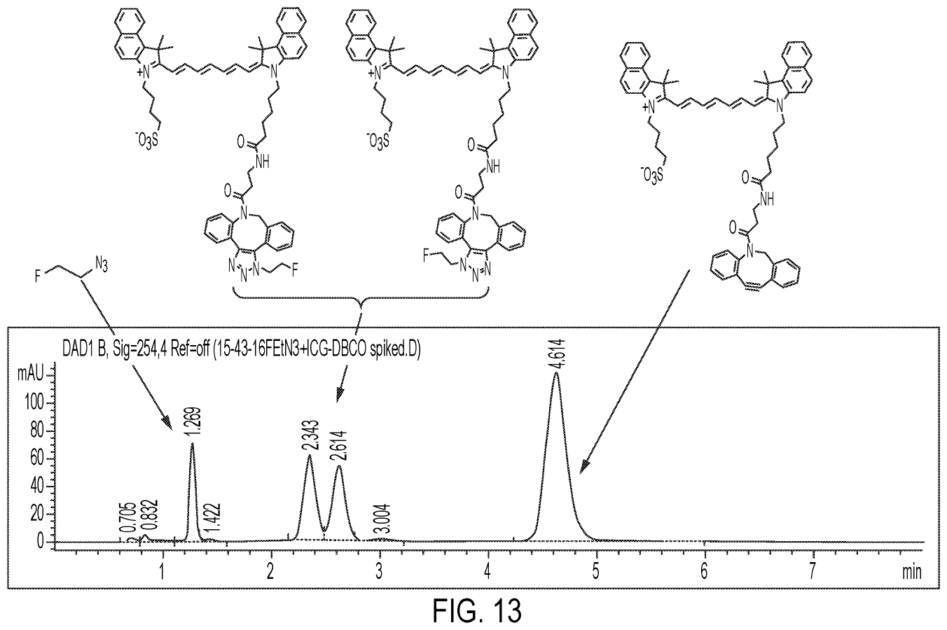

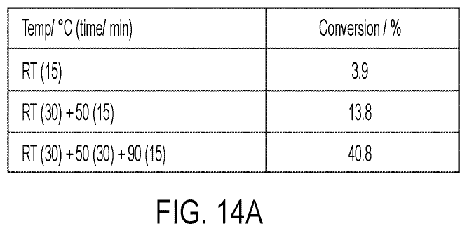

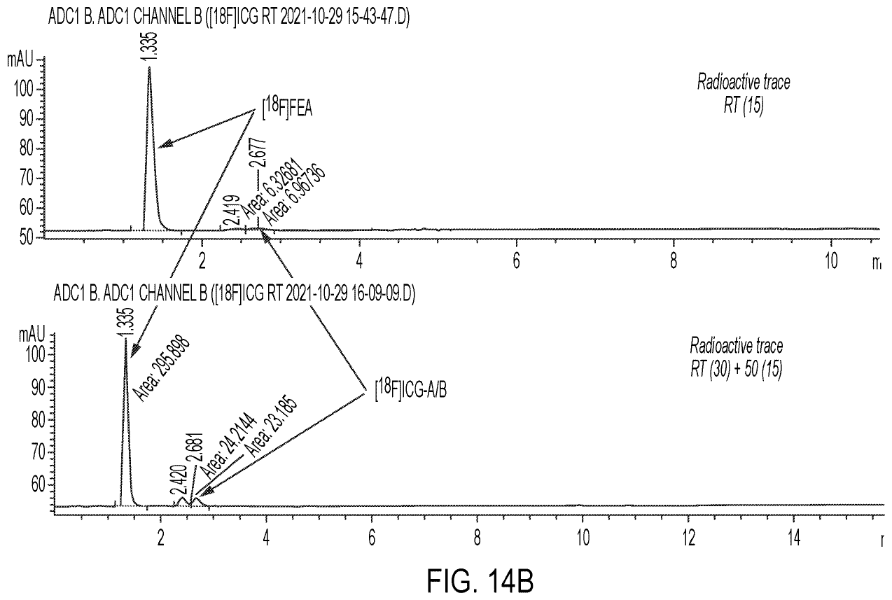

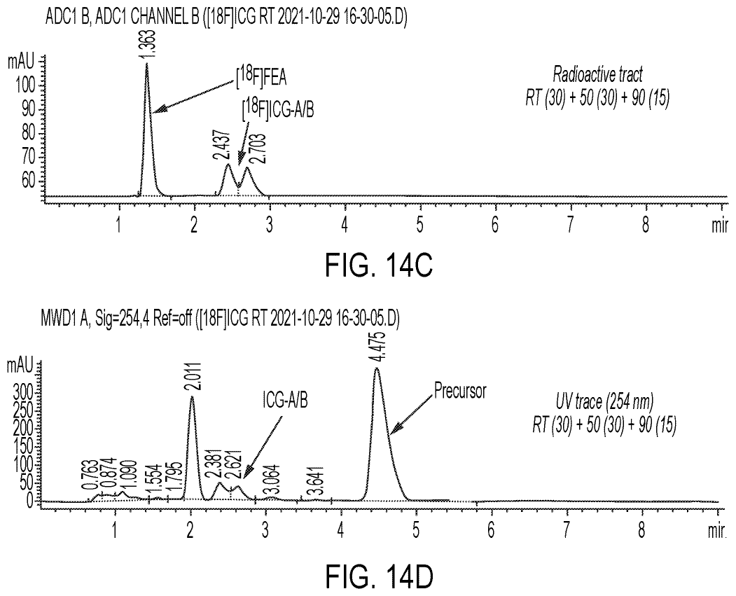

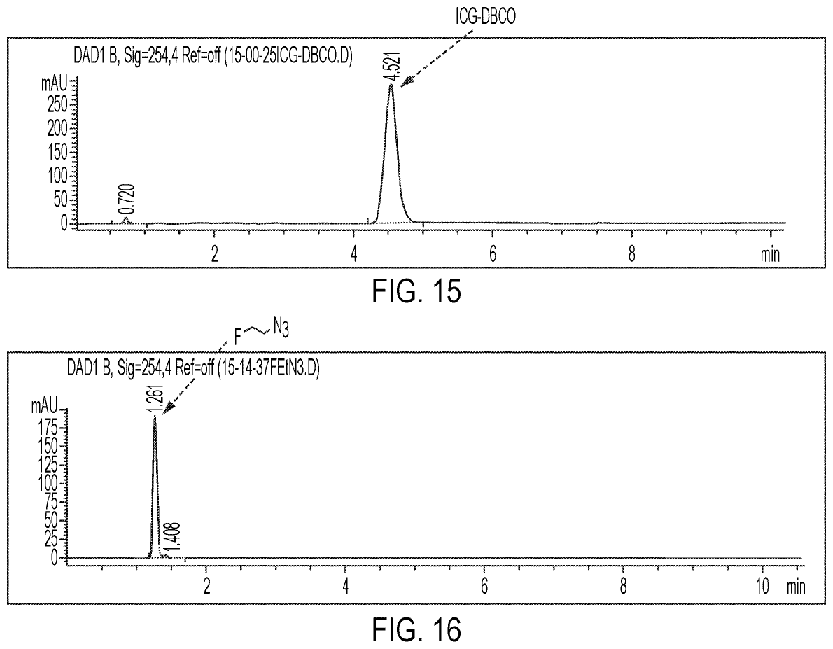

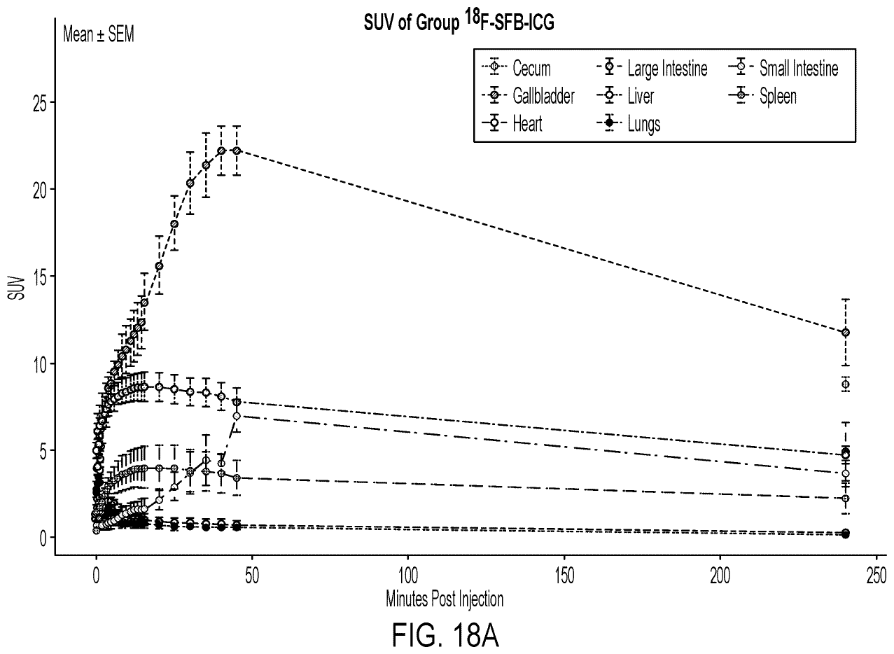

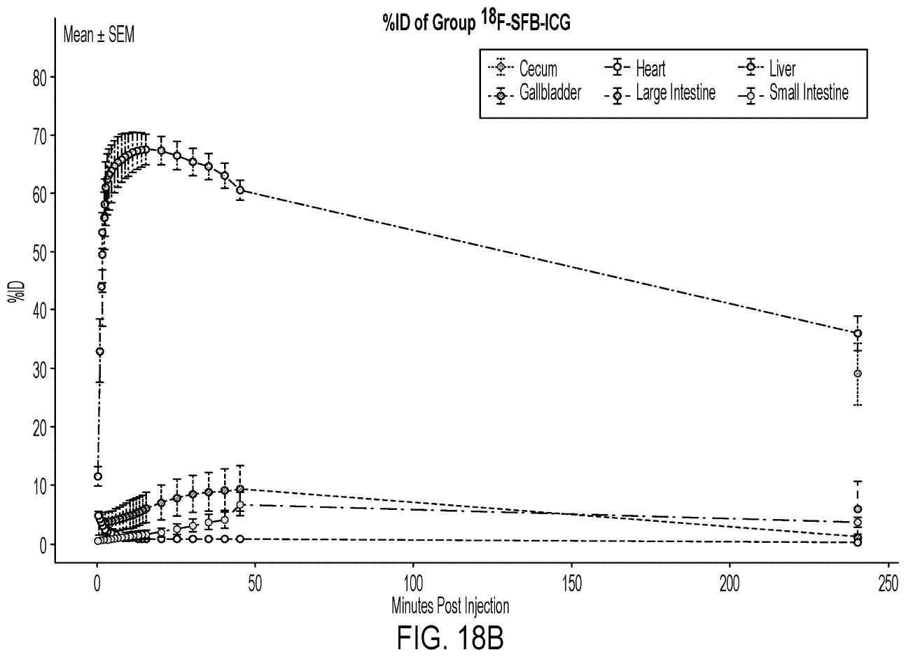

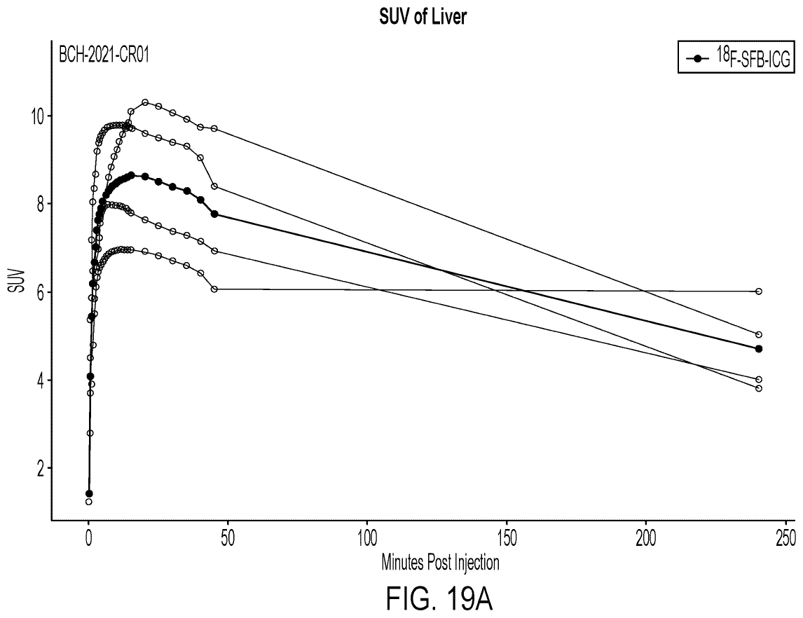

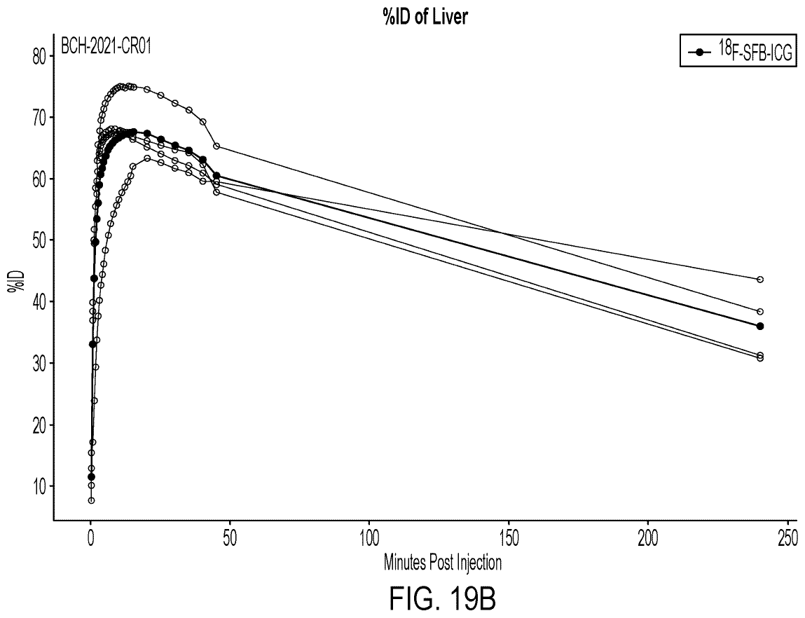

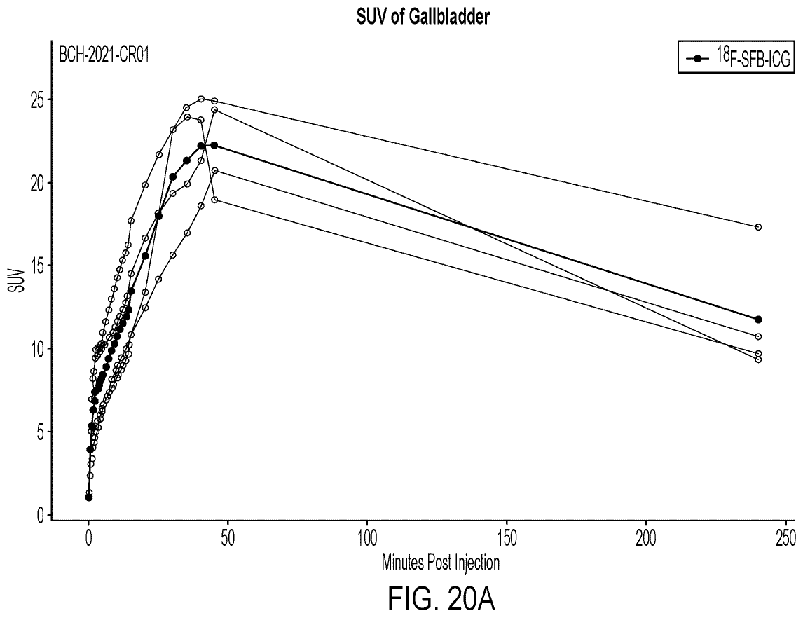

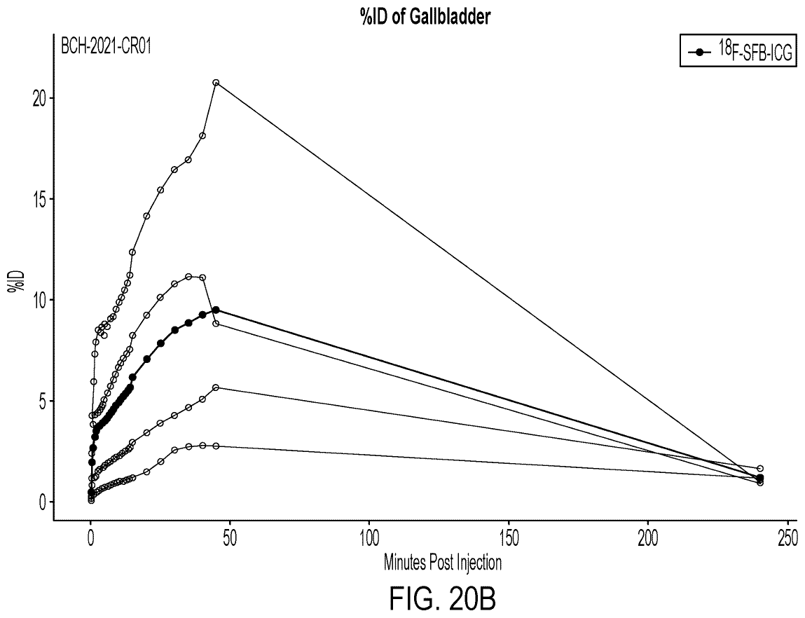

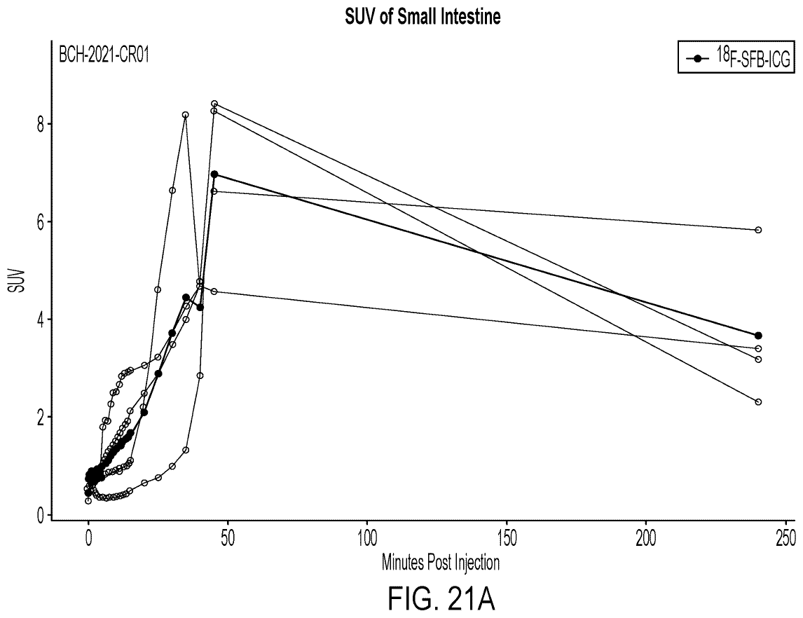

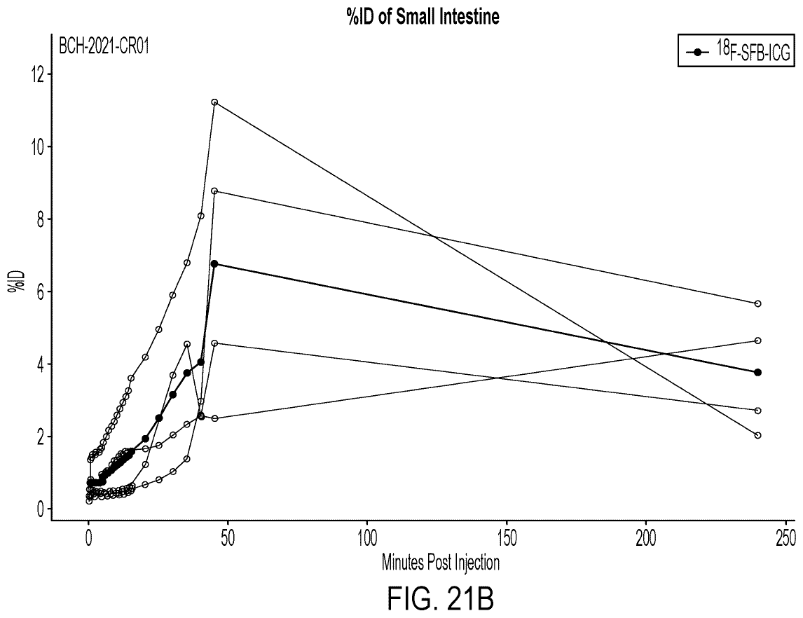

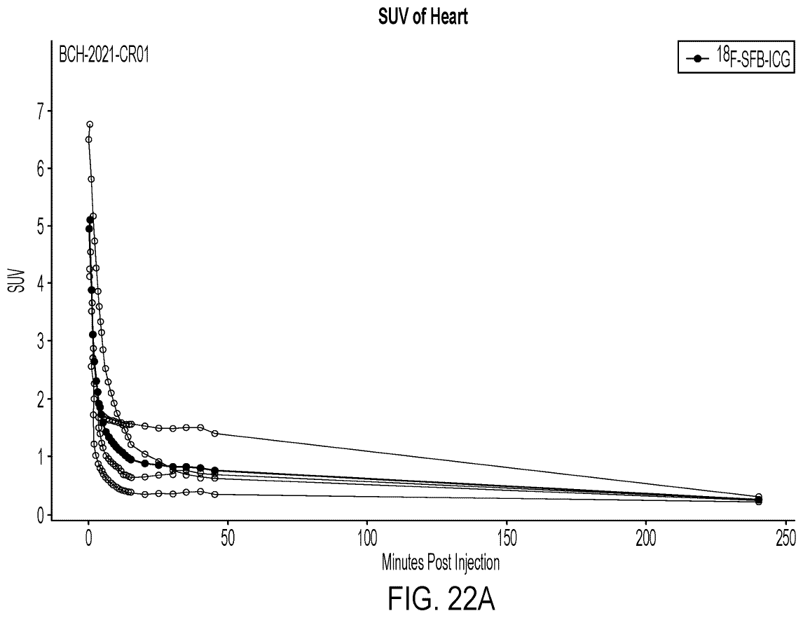

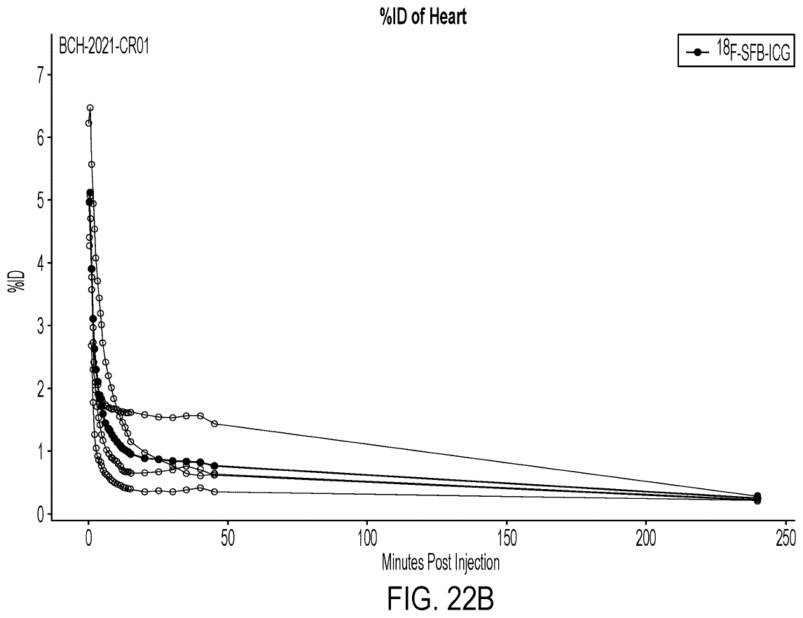

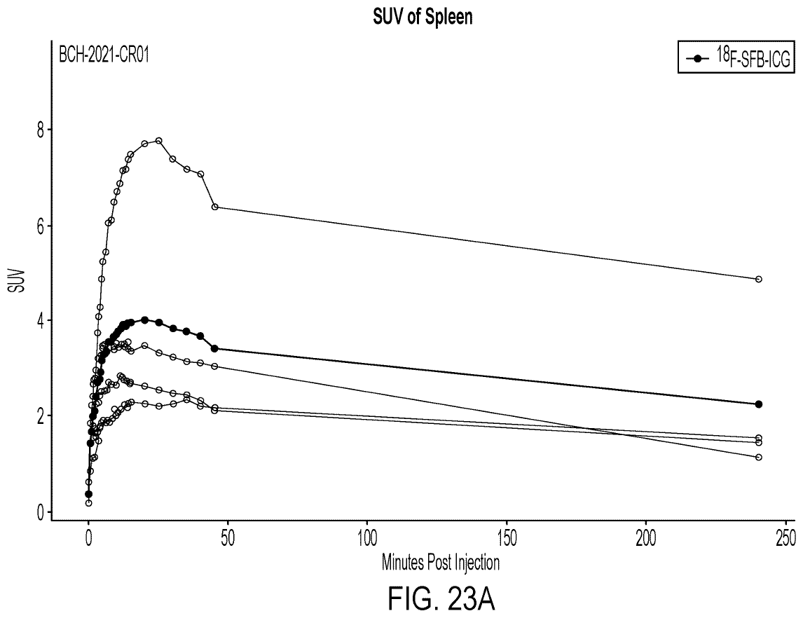

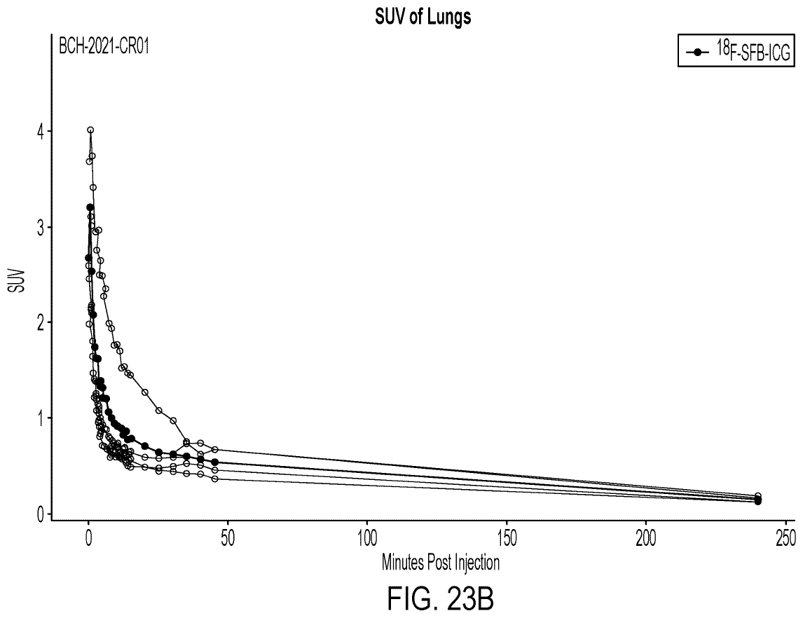

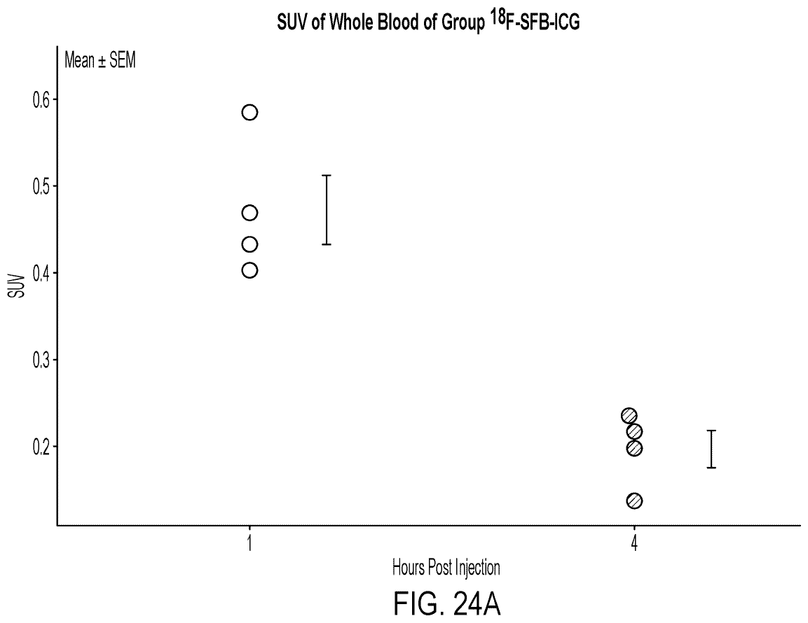

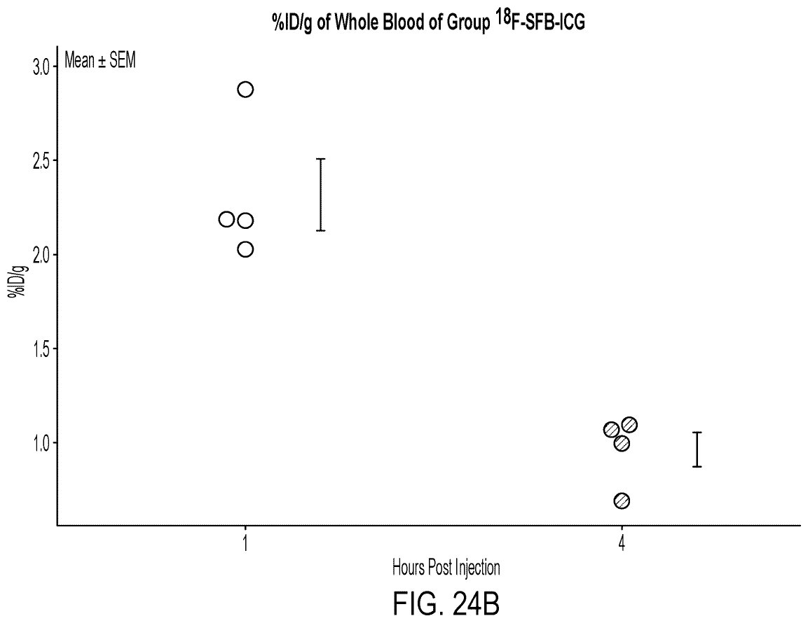

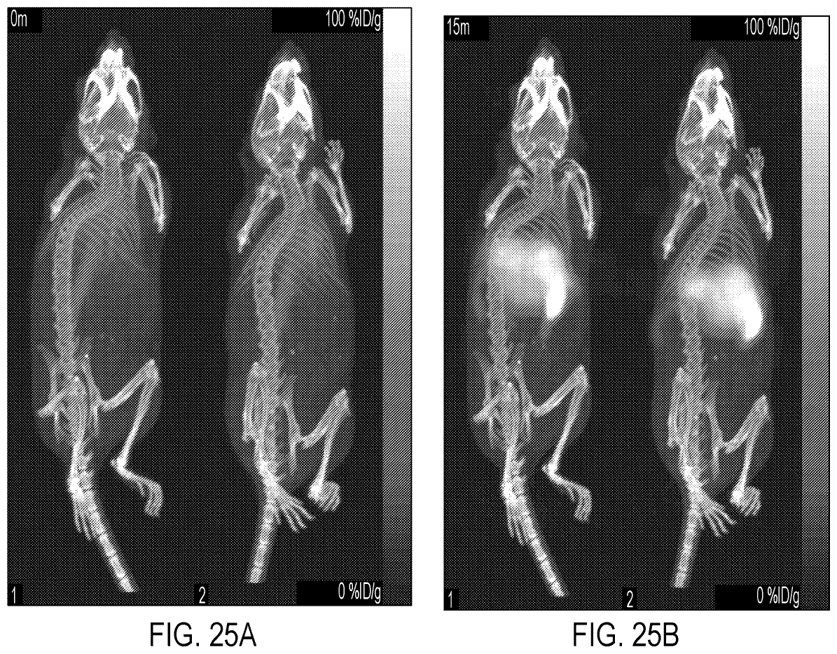

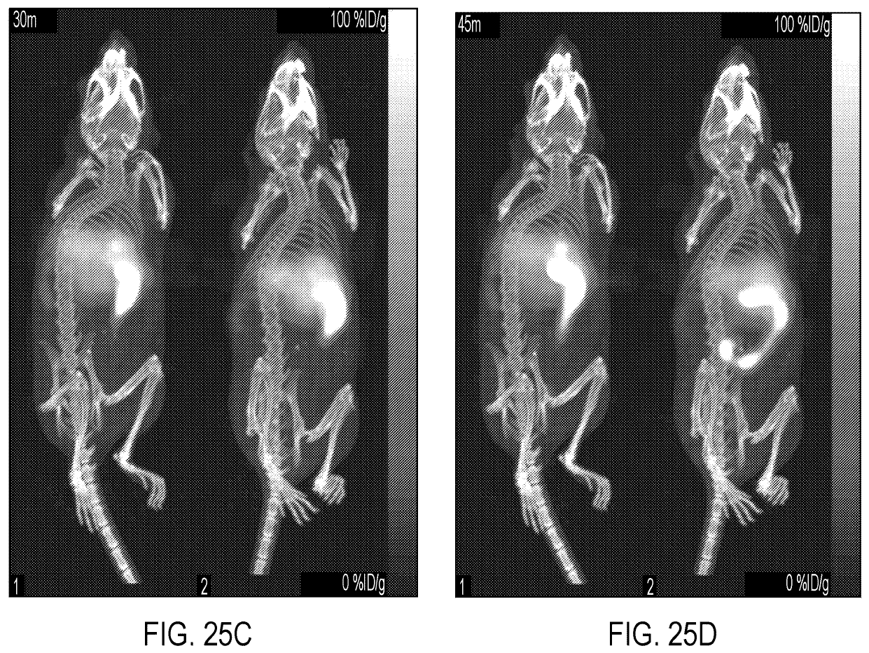

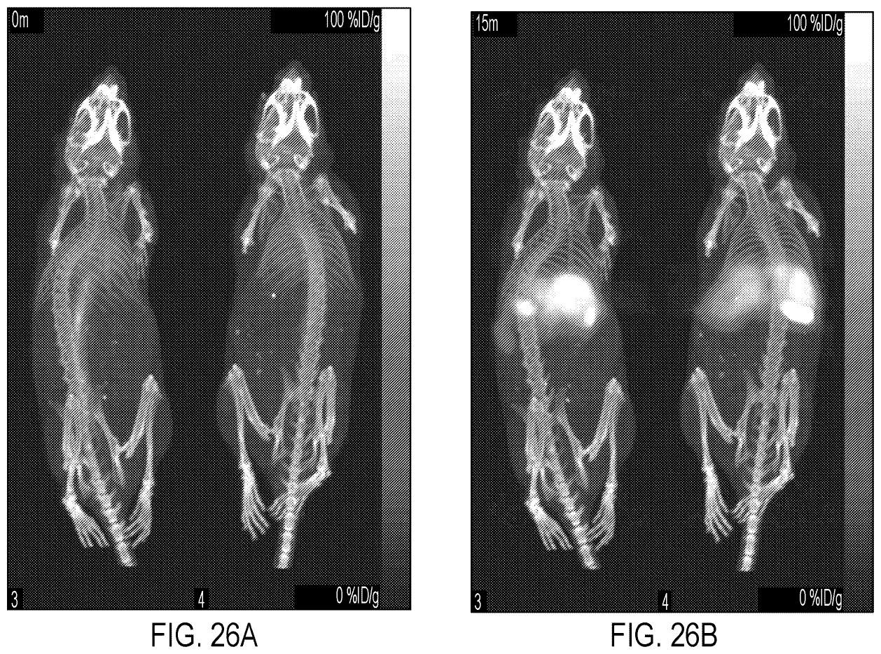

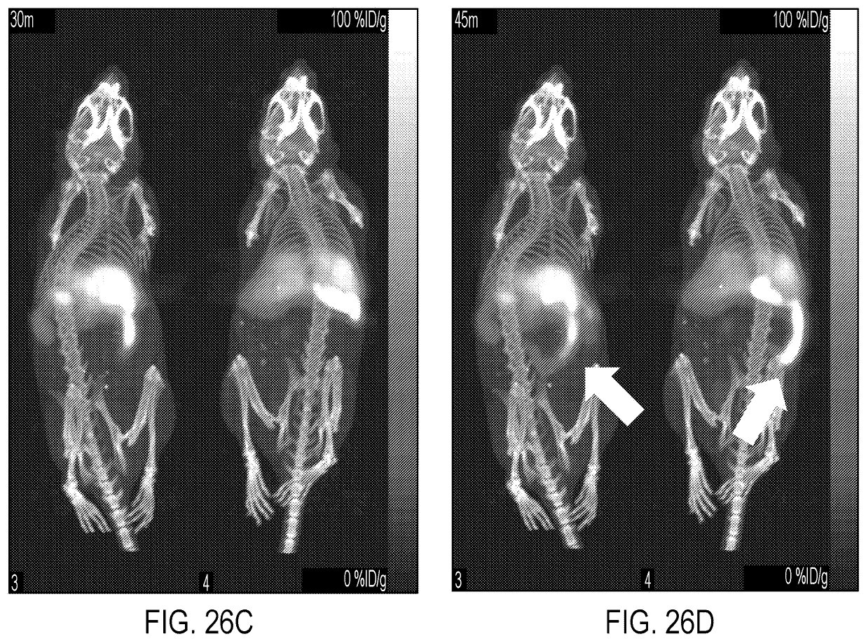

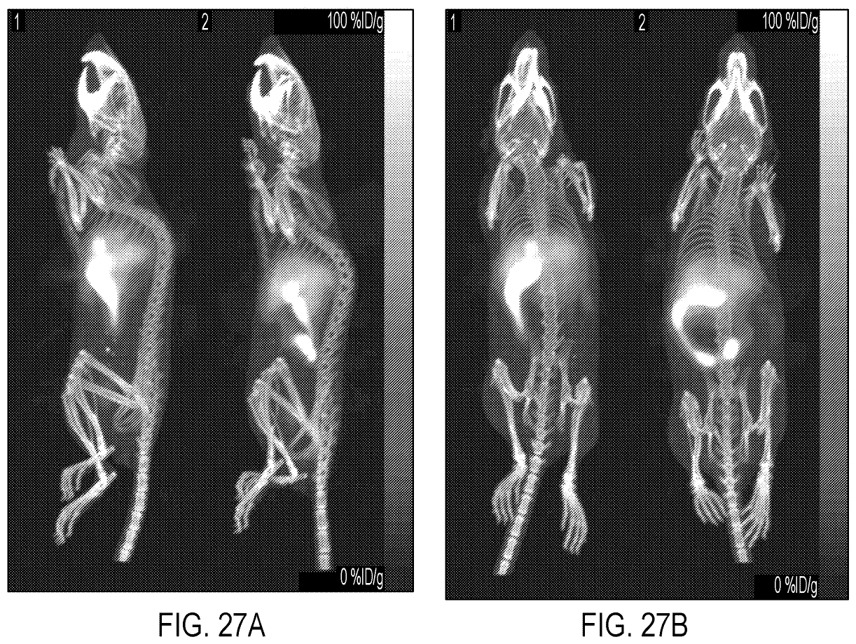

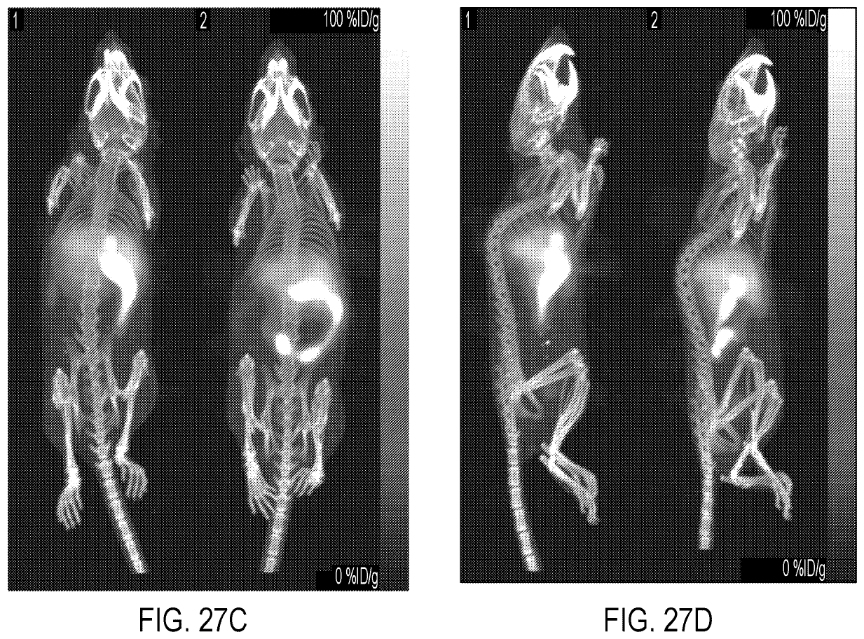

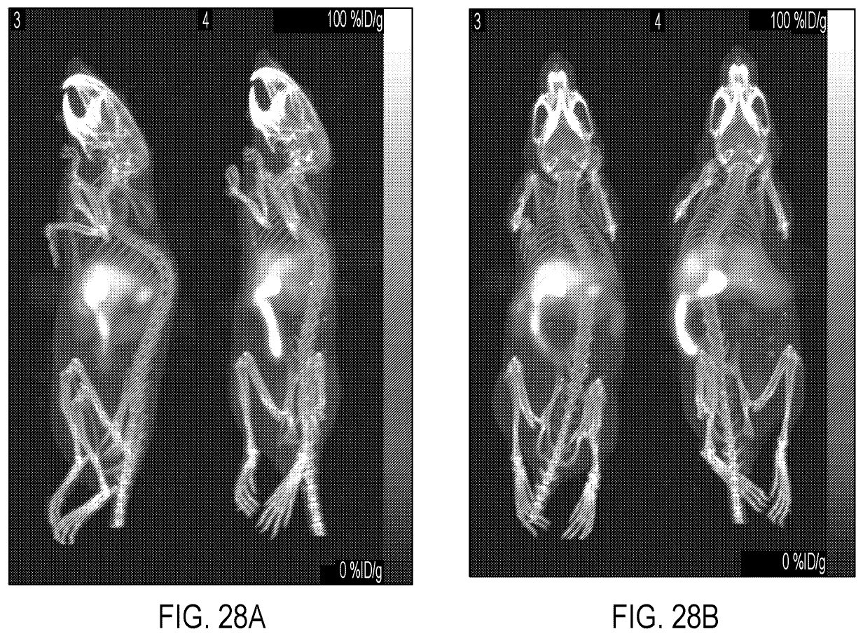

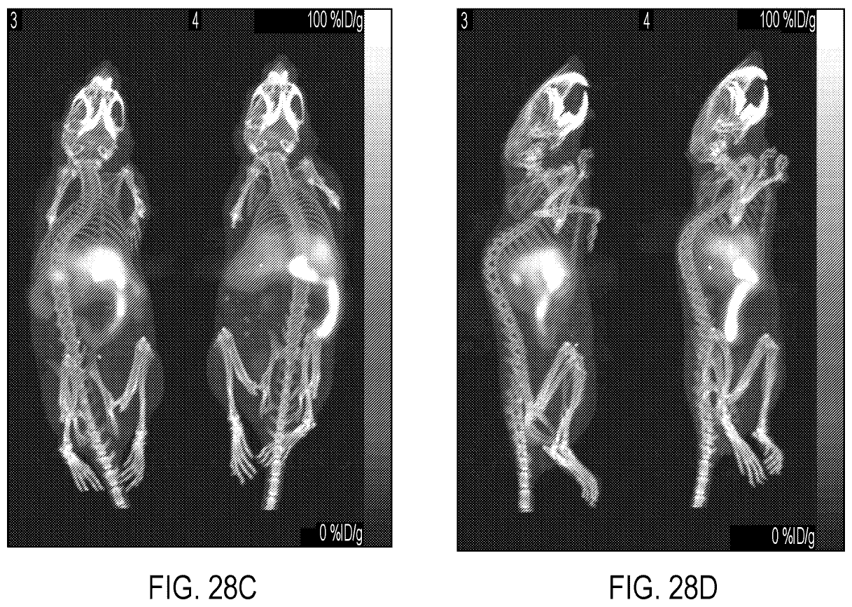

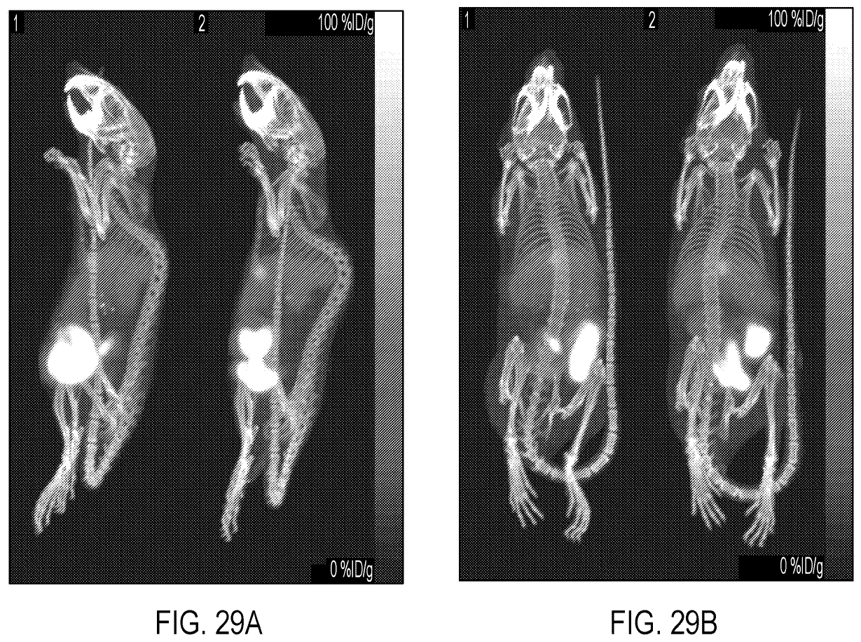

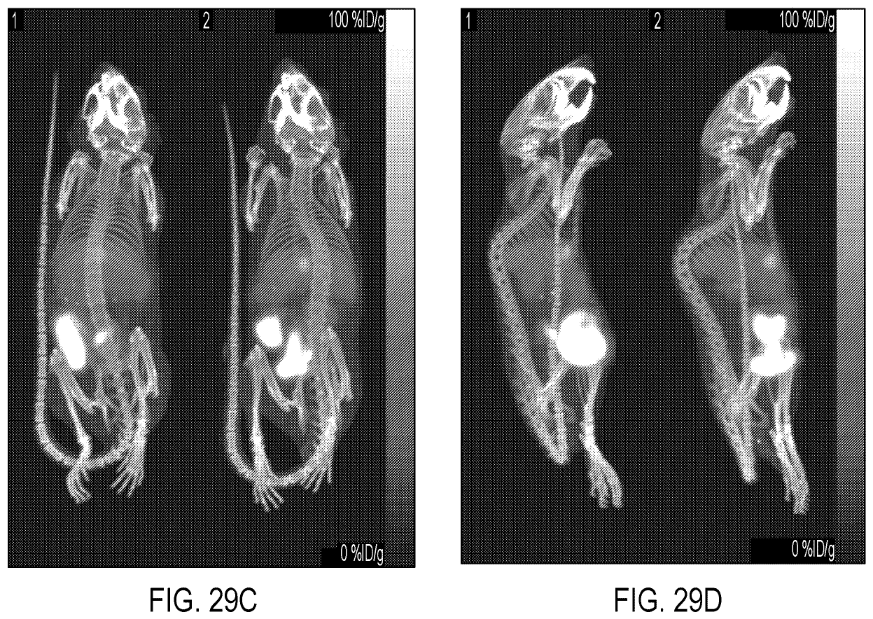

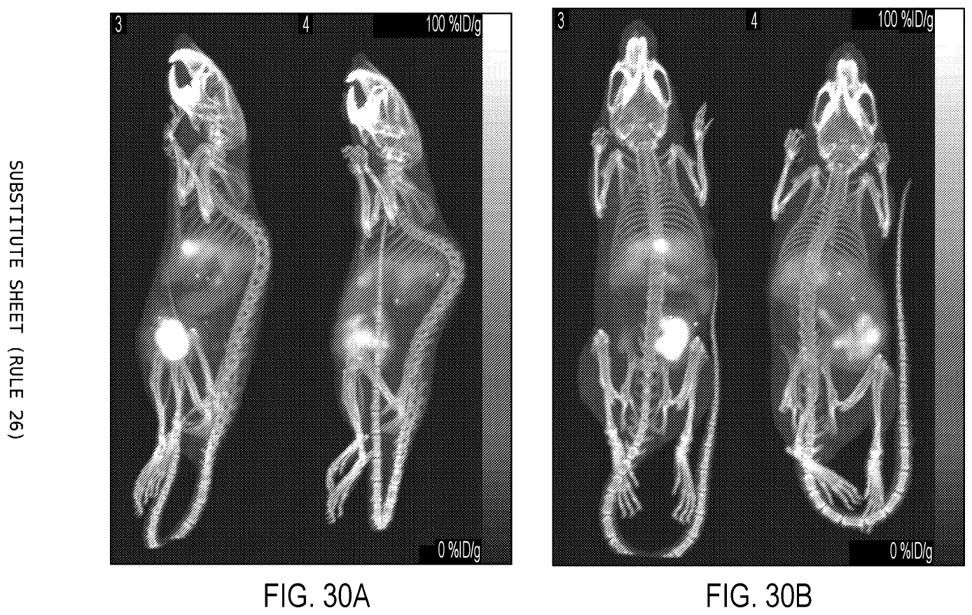

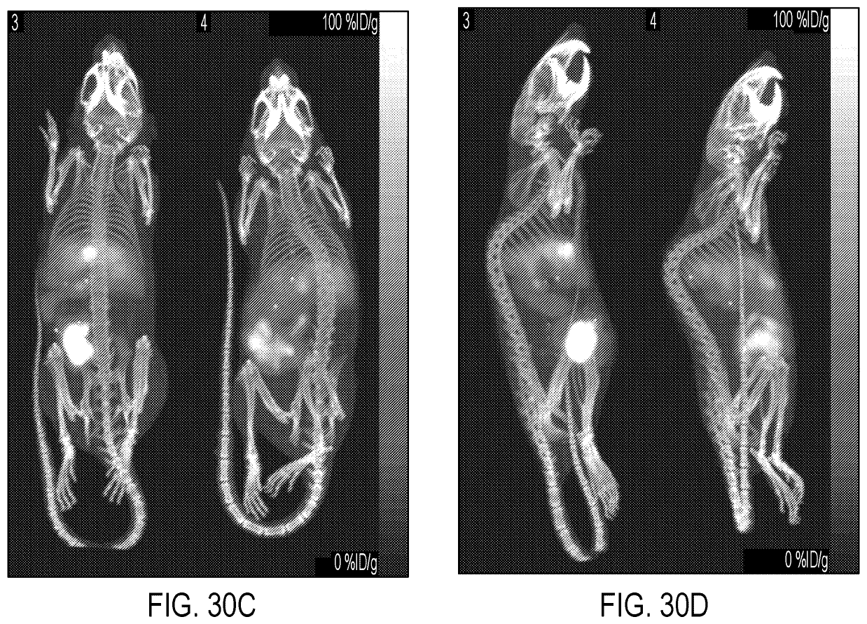

The use of ICG for localization of HCC and hepatoblastoma has been reported^{16-18,21,22,30} however, there are no published reports regarding its use in FLC. Surgical experience in 2 patients with FLC demonstrated ICG uptake by tumor tissue in the liver as well as metastatic deposits (FIGS. 31A-31D). Analysis of RNA-seq data demonstrates that two of the known ICG uptake transporters are also expressed in FLC. Even though the expression is lower in tumor tissue compared to normal liver tissue, it is likely sufficient for uptake as demonstrated through clinical observation (FIGS. 31A-31D). The MDR3 transporter, which is responsible for export of ICG into the bile canaliculi is also expressed in tumor cells, albeit at lower levels. Despite the known differences in the oncogenic drivers and the transcriptome between FLC and HCC, these two cancers share similar expression levels of ICG transporters SLCO1B1 and SLCO2B1 which suggests that they may have comparable properties for ICG retention. ¹⁸F was selected as the initial radioisotope given that 1) it is the radioisotope used clinically in ¹⁸F-fluorodeoxyglucose (FDG) PET and PET/CT imaging, 2) it is safe and tolerable and 3) its properties are amenable for conjugation to ICG. FDG PET is a widely used modality and is based on cellular uptake of glucose and therefore cancers with a high rate of glucose uptake demonstrate a more intense signal on PET imaging.³¹⁻³³ As a detectable radionuclide on PET scan, ⁶⁴Cu has gained tremendous attention in recent years partly due to: 1) its longer half-life compared to ¹⁸F and 2) the pairing with a therapeutic ⁶⁷Cu-based compound as a theranostic modality.

[¹⁸F]ICG was administrated to wild-type mice and dynamic PET/CT images were obtained during the first hour (FIGS. 26A-26D) followed by a static image at 4 hours (FIGS. 30A-3D). [¹⁸F]ICG is cleared from the circulation and localized to the liver under similar kinetics as ICG itself. As illustrated in FIGS. 26A-26D, at time 0, the tracer is localized to the heart and the major vessels and by 5 minutes, the tracer is predominantly localized to the liver. This rapid clearance of [¹⁸F]ICG from the serum and concentration in the liver is in line with the known 4-5 minute serum half-life of ICG¹⁴. There is no significant uptake by any other organs via the circulation. By 20 minutes, there is evidence of excretion of the tracer into the gallbladder via the biliary system and by 45 minutes, there is evidence of the tracer in the lumen of the small intestine. At 4 hours (FIGS. 30A-3D), following [¹⁸F]ICG injection, the signal intensity in the liver has decreased significantly and the majority of the tracer is localized to the lumen of the intestines. FIG. 18B illustrates the percentage of injected dose (% ID) of [¹⁸F]ICG in several organs of interest. Nearly 70% of [¹⁸F]ICG is localized to the liver by 10 minutes and by 4 hours about 40% ID is localized to the liver. At 4 hours, approximately 30% ID is localized to the cecum demonstrating the transit of [¹⁸F]ICG through the small intestine and into the right colon. [¹⁸F]ICG is rapidly cleared from plasma with 2-2.5% ID and 1% ID present in whole blood at 1 and 4 hours, respectively (FIG. 24B). The pattern and timing of [¹⁸F]ICG uptake by liver and excretion into the biliary system and the small intestine is similar to the known kinetics of ICG.^{13,14} These studies demonstrate that conjugation of ¹⁸F to ICG maintains hepatocyte uptake and clearance. Based on these data, [¹⁸F]ICG may also accumulate in liver cancer cells which express the transporters responsible for the uptake of ICG. In addition, modification of ICG with cytotoxic radionuclides may result in tumor localization and subsequent tumor cell destruction.

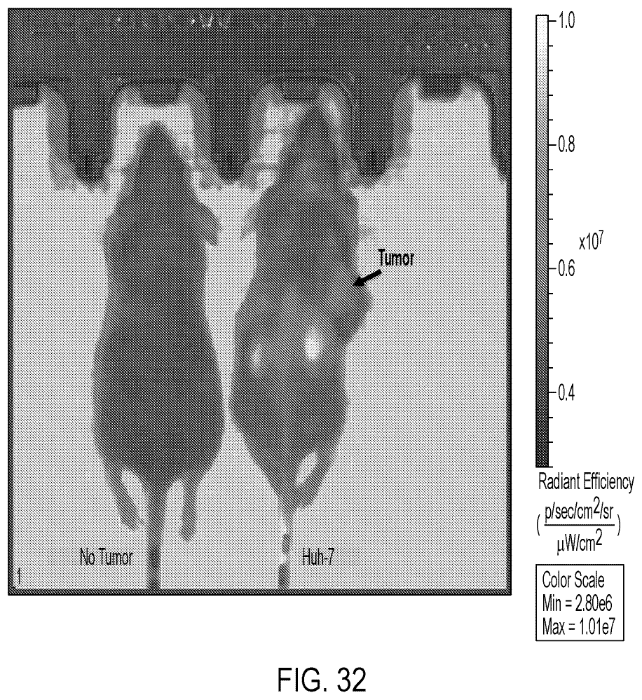

Xenotransplantation of the following cell lines is performed: FLX1 (FLC), Huh-7 (HCC) and HepG2 (well-differentiated HCC) in immunodeficient mice. Since the ability of Huh-7 and HepG2 cells lines to uptake ICG has been previously demonstrated in mouse models³⁴, these cell line xenograft models provide an efficient approach to assess uptake of [¹⁸F]ICG and [⁶⁴Cu]ICG. Given the general challenges with the establishment of in vivo and in vitro FLC models, using Huh-7 and HepG2 cell lines is important in demonstrating the ability of [¹⁸F]ICG and [⁶⁴Cu]ICG to localize to metastatic liver tumors. Following expansion of these cells, they are transferred to LFIC for subcutaneous injection (5×10⁶ cells/mouse in 1:1 v: v with 50 μL Matrigel) and establishment of xenograft models.³⁴ In conjunction with LFIC, the development process for these models is established, demonstrating ICG uptake by Huh7 tumor cells at 0.5 mg/kg ICG dose which is 10-20 fold lower than previously reported animal dosing (FIG. 32). Therefore, the dose is escalated to an appropriate level. Even though the majority of the models use HCC cells, the findings are applicable to FLC since FLC also has the ability to retain ICG (FIGS. 31A-31D). Currently, there is only one readily available FLC cell line, which also has the property to be engrafted in an immunodeficient mouse model.

In order to evaluate the ability of ICG-radionuclide conjugates to detect intrahepatic tumors, the NRAS^{G12V}/shP53 induced orthotopic HCC model is used.³⁵ This model is created via hyperdynamic tail vein injection of NRAS^{G12V}/shP53 plasmids in 8-week old mice resulting in intrahepatic tumor development after 2 months.

### Determine Tumor Tissue Localization and Retention Time of ‘Cold’ ICG-Conjugates.

It can be determined 1) whether ICG conjugates localize to tumor cells and 2) the duration of ICG retention within the tumor. Developing a precise understanding of ICG retention in the tumor bed facilitates the determination of dosing frequency for therapeutic agents. Since synthesis of ICG-radionuclide conjugates is more labor intensive and costly, tumor localization of ICG conjugates is initially assessed using ‘cold’ reference compounds. The reference compounds are synthesized by conjugating ICG to non-emitting isotopes (e.g. fluorine-19 instead of fluorine-18). The cold ICG-conjugate has the same chemical structure as the radionuclide conjugate and therefore it is expected to have similar pharmacokinetics. The minimal dose of the reference ICG compounds that localizes to the tumor is determined by injecting the tumor-bearing mice with ICG-conjugates at 2, 5, and 10 mg/kg followed by in vivo fluorescence imaging at the following intervals post-injection: 1 hour, 4 hours, 1 day, 3 days, 5 days, and 7 days. The timeline and dosing are based on previously published results. 34,36 If there is significant tumor signal at 7 days, the imaging is extended until the time at which there is complete signal dissipation from the tumor. The dose range for each cold ICG-conjugate is determined, which guides the dosing of ‘hot’ ICG-radionuclide in subsequent experiments.

[¹⁸F]ICG has been synthesized with purity >95%. For assessment of [¹⁸F]ICG uptake by tumors, the tumor-bearing mice undergo intravenous injection with [¹⁸F]ICG and imaging under the following schedule: 1) dynamic PET/CT imaging for 1 hour; and 2) static imaging at 4 hours following injection. This schedule is designed based on the 110-minute half-life of ¹⁸F. Dosing is determined by data obtained using the cold reference compound, [¹⁹F]ICG.

The compound [⁶⁴Cu]ICG has also been synthesized. Given the 12.7 hour half-life of ⁶⁴Cu, imaging can be performed at a later time point following injection compared to [¹⁸F]ICG. For [⁶⁴Cu]ICG imaging in tumor-bearing mice, the following imaging schedule is used: 1) dynamic PET/CT imaging for 1 hour after injection; and 2) static imaging at 24 and 48 hours. The ability to perform the imaging beyond 24-48 hours provides the necessary time for [⁶⁴Cu]ICG to be completely cleared from the liver, thereby increasing the signal to background ratio of [⁶⁴Cu]ICG retaining nodules within the liver. Dosing is determined by data obtained using cold reference compound.

NRAS^{G12V}/shP53 induced HCC mice undergo imaging following [¹⁸F]ICG and [⁶⁴Cu]ICG administration using the above-mentioned respective imaging protocols.

Measure the anti-tumor activity of [⁶⁷Cu]ICG. There are a number of cytotoxic radionuclides, which are used for cancer therapeutics as conjugates to antibodies and small molecules. ⁶⁷Cu is selected since it is a short-range beta-emitting nuclide and has gained significant attention in recent years due to its favorable properties for nuclear medicine applications as well as improvements in its production.^{37,38 67}Cu has a mean energy of 141 keV, similar to the energy of ¹³¹I (180 keV), a widely used radionuclide. Advantages of ⁶⁷Cu over 131I is the lower gamma radiation associated with ⁶⁷Cu, thereby delivering a lower whole-body dose to the patient as well as the clinical personnel.³⁸ Thus the development of [⁶⁴Cu]ICG and [⁶⁷Cu]ICG as a theranostic pair of agents has advantages in clinical practice as they provide a pair of agents, which complement each other as diagnostic and radiotherapeutic agents with similar mechanisms of action.

Validate the radiolabeling of ICG with ⁶⁷Cu. [⁶⁷Cu]ICG can be synthesized through a similar methodology to [⁶⁴Cu]ICG synthesis. Formulation stability testing is performed and radiochemical purity data at end-of-synthesis are determined.

Assess tumor cytotoxic effects of ICG-radionuclide conjugates in mouse liver cancer models. Tumor-bearing mice are established with representative model shown in FIG. 32. Mice are monitored closely and tumor size is assessed every 3 days by physical and ultrasound examinations. Once the tumor volume reaches 1000 mm³ based on US or MRI, the mice undergo PET/CT imaging with [⁶⁴Cu]ICG tracer at a dose previously determined to establish a baseline standardized uptake value (SUV) at the site of the tumor. On the following day, mice (n=4 per experimental group) undergo a tail vein injection with [⁶⁷Cu]ICG at the same dose as [⁶⁴Cu]ICG. The control group (n=4) undergoes injection with ICG alone. Another experimental group undergoes injection with only [⁶⁷Cu]ICG, without initial imaging with [⁶⁴Cu]ICG. In vivo fluorescence imaging is used to confirm localization of ICG-radionuclide conjugates to tumors. Tumor volume (mm³) is measured and recorded every 3 days for 15 days to generate a tumor growth curve for each animal. The mice are monitored for any adverse effects during this time. The serum levels of AST, ALT, GGT, and bilirubin are also measured as biochemical markers for liver injury. On day 15, the mice undergo MRI to assess tumor size. PET/CT with [⁶⁴Cu]ICG is performed to assess SUV at tumor site and compared to baseline SUV at the start of the treatment. The mice subsequently undergo euthanasia on day 15 and the tumor and liver tissue are removed for histologic analysis. The tumor and liver sections are examined to assess tumor cell viability as well as evidence of acute liver injury. The tumor sections are also examined for the localization of ICG or [⁶⁷Cu]ICG (cytoplasmic vs. extracellular) using near-infrared confocal microscopy. This data demonstrate whether there are any differences between ICG and [⁶⁷Cu]ICG efflux from the tumor cells as well as a quantitative measure of residual ICG in the tumor bed.

The data obtained from the first series of experiments are used to guide the effect size of [67Cu]ICG on tumor tissue. If the tumor effect size is not large, the following experimental conditions can be adjusted in order to achieve a larger effect size: 1) Thirty-day duration of treatment +/−additional [⁶⁷Cu]ICG dose on day 15; and 2) administer [⁶⁷Cu]ICG dose at higher doses for 15-day treatment. The results of these experiments are then used to guide further adjustments to obtain optimal treatment effect.

### Compound Synthesis

Compounds of the disclosure are prepared following means known in the art of organic synthesis. For example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof are representative and instructive. Methods for optimizing reaction conditions, and if necessary minimizing competing by-products, are known in the art.

### Cold Reaction

For cold reaction conditions, initial development quality control conditions were established to distinguish parent compound from precursor. Stock solutions of the fluoro ethyl azide (FEA) in MeCN and precursor in MeCN were combined and left at room temperature for min. QC analysis of the reaction showed high conversion of precursors to form two chemically similar peaks more hydrophilic than the parent.

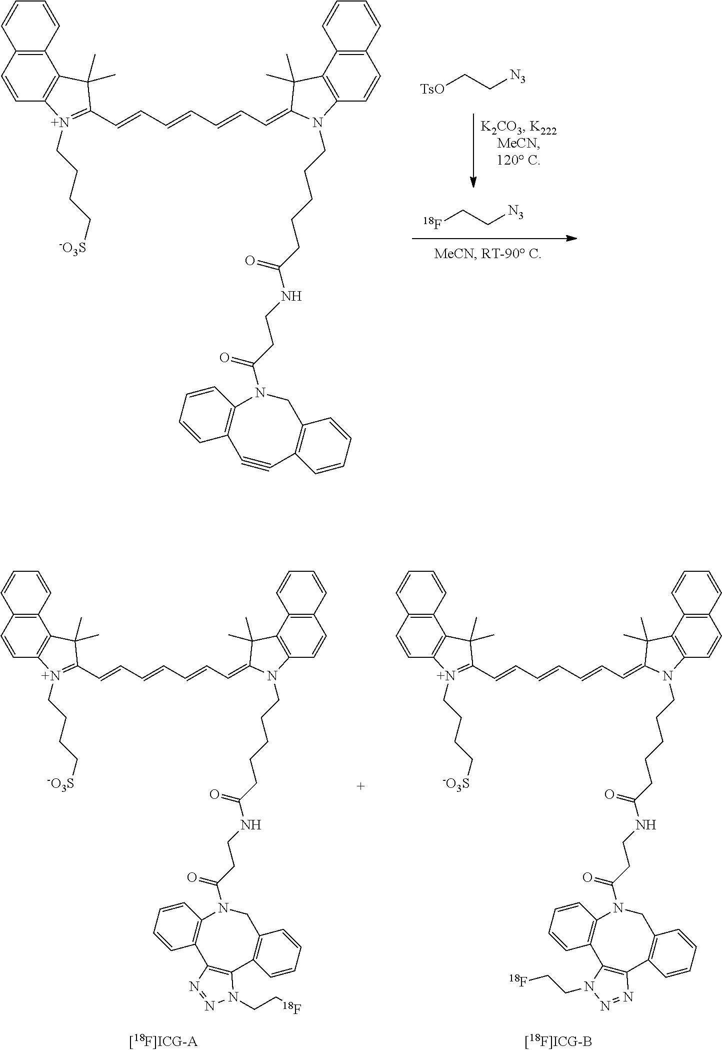

### [¹⁸F]ICG-A/B Reaction

Initial radiolabelling conditions were attempted. [¹⁸F]FEA was prepared by fluorination of the tosyl precursor and distillation into the reaction vessel containing MeCN (300 μL). Precursor (0.32 mg) in MeCN (800 μL) was added. Aliquots of the crude mixture were analysed over time with increasing temperature. Two radiopeaks believed to correspond to [¹⁸F]ICG-A and [¹⁸F]ICG-B were obtained. At 90° C. 40% conversion from [¹⁸F]FEA was obtained. The initial results for the formation of [¹⁸F]ICG-A/B were promising, with stability and reactivity of the precursor up to at least 90° C. tested. Conversions obtained are suitable for in vivo imaging, and a radiolabelling process for the ICG derivative allows for imaging in vivo.

### Synthesis of ICG-NOTA

Materials: ICG Amine precursor-AAT BIO-221208-02-Ref #188-5 MG-Lot #127139; p-SCN-Bn-NOTA-Macrocyclics-230206-02-Ref #B605-Lot #B60510015-2203032; DMSO, anhydrous, >99.9%-Sigma Aldrich-Ref #276855-Lot #BJ3695; N,N-Diisopropylethylamine, ReagentPlus®, >99% (DIPEA)-Sigma Aldrich-Ref #D125806-Lot #SHBQ0433; Mobile phase: 45% Ethanol in water; HPLC Column: 221123-01-XBridge BEH C18 OBD Prep Column, 130 Å, 5 μm, 10 mm×250 mm-SN #368132313111206; HPLC Flow 1.5 mL/min.

A solution of ICG-amine (5.0 mg dissolved in 110 μL DMSO) was transferred to a glass vial containing p-SCN-Bn-NOTA (8.3 mg), followed by addition of 10 μL of DIPEA. The vial was mixed overnight in the thermomixer (550 RPM) at room temperature. The following day, 400 μL of 45% ethanol in water was added to the reaction vial and the product was purified by HPLC through a series of 100 μL injections onto a XBridge BEH C18 OBD Prep Column (130 Å, 5 μm, 10 mm×250 mm) using 45% ethanol and water as the mobile phase and a flow rate of 1.5 mL/min. Fractions containing the product were collected from 10.0-13.0 minutes, pooled, and lyophilized to give the purified ICG-NOTA (0.6 mg, 7.6%) as a blue-green dry powder.

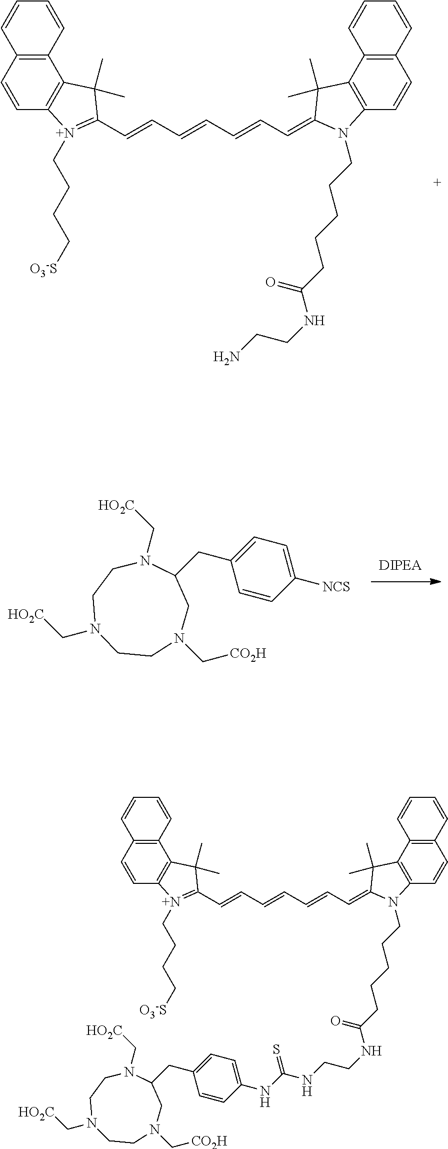

⁶⁴Cu or ⁶⁷Cu are complexed to ICG-NOTA. A solution of [⁶⁴Cu] or [⁶⁷Cu] CuCl₂ in 0.1M HCl is mixed with ammonium acetate buffer and ICG-NOTA. The resulting mixture (pH 4) is placed on an agitating mixer and shaken at 300 rpm for 30 min at room temperature. Then, the solution is diluted with water and passed through a Sep-Pak C18 Plus Light cartridge (rinsed with EtOH first, then with H₂O before use). The cartridge is rinsed with water, then with 10% MeOH in water, then eluted with MeOH. Flow direction used for the elution is opposite to the flow direction used for trapping crude material and rinsing. The eluate is evaporated to dryness at 60° C. under nitrogen flow and the residue is redissolved in 0.9% saline. The molar activity of the chelation complex solution used for the biodistribution studies and imaging experiments is 3 MBq/nmol.

### Study Design for In Vivo Biodistribution of ¹⁸F-SFB-ICG in Wild Type Mice

The study was comprised of female C₅₇BL/6 mice (n=4, 20.4+0.8 g, age 12.9 weeks). The modalities were whole body PET/CT and whole blood gamma counting which were performed on the mice using the imaging agent ¹⁸F-SFB-ICG as outlined in Table 1. Briefly, the mice were imaged in a 2-bed imaging hotel with imaging agent administered intravenously while animals were on camera (volume dosed was maintained constant to keep mass dose constant). 1 hour dynamic PET data was acquired, followed by CT. A static 30 minute PET dataset was acquired at 4 hours, also followed by CT. A blood sample was taken immediately following each imaging time point for gamma counting.

TABLE 1

| | | | Injected | Volume. | PET/CT Imaging | Gamma Counting |
|---|---|---|---|---|---|---|
| Group | N | Test Article | Activity | Rate | Time Points | Time Point |
| 1a | 2 | ¹⁸F-SFB-ICG | 321.1 ± 6.7 | 200 μL, IV | 0-1 h (frames: 10 × | Blood sample |
| | | | | | 30 sec, 10 × 1 min, 6 × | immediately |
| 1b | 2 | | 175.8 ± 4.1 | 200 μL, IV | 5 min, 1 × 15 min) | following each |
| | | | | | 4 h (1 × 30 min) | imaging time point |

### Methods of Analyzing In Vivo Biodistribution of ¹⁸F-SFB-ICG in Wild Type Mice

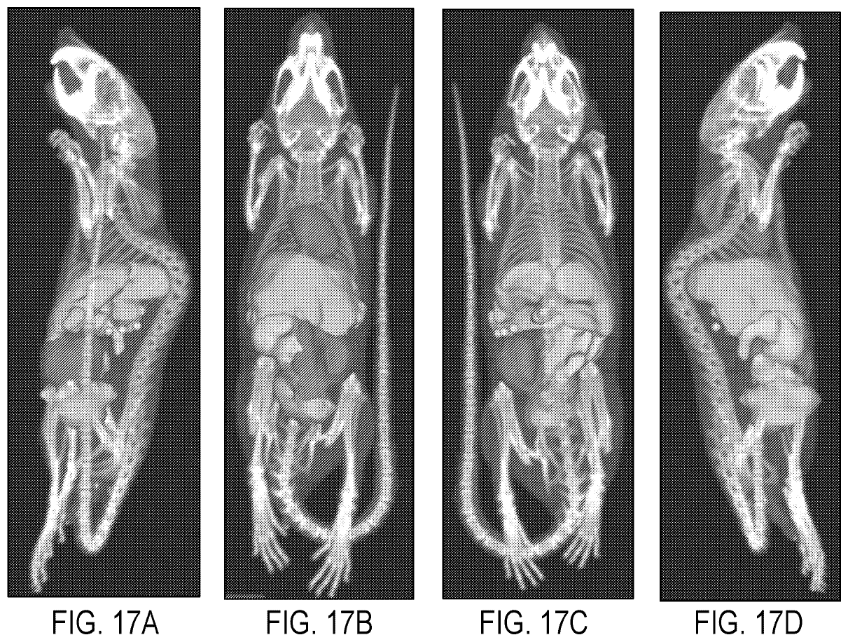

PET/CT: PET images were co-registered to CT images and resampled to uniform voxel size (0.2 mm³). The injected activity doses were normalized to the averaged activity in the field of view (FOV) in the final 6 frames of the dynamic scan (FOV-based normalization). The Regions of Interest (ROIs) were defined using various methods in VivoQuant software: A fixed volume ellipsoidal ROI was placed in the center of the heart, to encompass a volume of representative concentration for that region. The fixed volume sphere ROIs were placed in the center of the lobes of the lungs (2), and throughout the spleen (3) to encompass a volume of representative concentration for those regions. Based on the CT and PET, the following organs were segmented as completely as possible: liver, gallbladder, small intestines, cecum, and large intestines. The activity concentration was reported as Standardized Uptake Value (SUV), equating to the percentage injected dose per gram of tissue (% ID/g) normalized to subject body weight. For fully segmented organs, uptake was also reported in percentage injected dose (% ID). FIGS. 17A-17D show resulting PET/CT images of a representative wild type mouse.

Gamma Counting: Whole blood samples were collected after each imaging session for ex vivo radioactivity analysis by gamma counting. Triplicates of ¹⁸F-SFB-ICG were also counted to calculate an efficiency factor from which counts could be converted to units of activity (μCi). The sample activities were corrected for background radiation and decay-corrected to the injection time. The tissue volumes were estimated through measurement of sample weights, and blood activity was reported as % ID/g and SUV.

### REFERENCES

1. Craig J R, Peters R L, Edmondson H A, Omata M. Fibrolamellar carcinoma of the liver: a tumor of adolescents and young adults with distinctive clinico-pathologic features. Cancer. 1980; 46 (2): 372-379.
2. Farhi D C, Shikes R H, Murari P J, Silverberg S G. Hepatocellular carcinoma in young people. Cancer. 1983; 52 (8): 1516-1525.
3. Katzenstein H M, Krailo M D, Malogolowkin M H, et al. Fibrolamellar hepatocellular carcinoma in children and adolescents. Cancer. 2003; 97 (8): 2006-2012.
4. Shibata T, Arai Y, Totoki Y. Molecular genomic landscapes of hepatobiliary cancer. Cancer science. 2018; 109 (5): 1282-1291.
5. Honeyman J N, Simon E P, Robine N, et al. Detection of a recurrent DNAJB1-PRKACA chimeric transcript in fibrolamellar hepatocellular carcinoma. Science. 2014; 343 (6174): 1010-1014.
6. Xu L, Hazard F K, Zmoos A F, et al. Genomic analysis of fibrolamellar hepatocellular carcinoma. Human molecular genetics. 2015; 24 (1): 50-63.
7. Nunes R F, Zuppani R M F, Coutinho A M, et al. General Concepts in Theranostics. PET Clin. 2021; 16 (3): 313-326.
8. Gaze M N, Chang Y C, Flux G D, Mairs R J, Saran F H, Meller S T. Feasibility of dosimetry-based high-dose 131I-meta-iodobenzylguanidine with topotecan as a radiosensitizer in children with metastatic neuroblastoma. Cancer Biother Radiopharm. 2005; 20 (2): 195-199.
9. Lashford L S, Hancock J P, Kemshead J T. Meta-iodobenzylguanidine (mIBG) uptake and storage in the human neuroblastoma cell line SK-N-B E (2C). Int J Cancer. 1991; 47 (1): 105-109.
10. Matthay K K, Shulkin B, Ladenstein R, et al. Criteria for evaluation of disease extent by (123) I-metaiodobenzylguanidine scans in neuroblastoma: a report for the International Neuroblastoma Risk Group (INRG) Task Force. Br J Cancer. 2010; 102 (9): 1319-1326.
11. Murthy R, Nunez R, Szklaruk J, et al. Yttrium-90 microsphere therapy for hepatic malignancy: devices, indications, technical considerations, and potential complications. Radiographics. 2005; 25 Suppl 1: S41-55.
12. Alander J T, Kaartinen I, Laakso A, et al. A review of indocyanine green fluorescent imaging in surgery. Int J Biomed Imaging. 2012; 2012:940585.
13. Desmettre T, Devoisselle J M, Mordon S. Fluorescence properties and metabolic features of indocyanine green (ICG) as related to angiography. Surv Ophthalmol. 2000; 45 (1): 15-27.
14. Cherrick G R, Stein S W, Leevy C M, Davidson C S. Indocyanine green: observations on its physical properties, plasma decay, and hepatic extraction. J Clin Invest. 1960; 39 (4): 592-600.
15. Leevy C M, Mendenhall C L, Lesko W, Howard M M. Estimation of hepatic blood flow with indocyanine green. J Clin Invest. 1962; 41 (5): 1169-1179.
16. Lim C, Vibert E, Azoulay D, et al. Indocyanine green fluorescence imaging in the surgical management of liver cancers: current facts and future implications. J Visc Surg. 2014; 151 (2): 117-124.
17. Satou S, Ishizawa T, Masuda K, et al. Indocyanine green fluorescent imaging for detecting extrahepatic metastasis of hepatocellular carcinoma. J Gastroenterol. 2013; 48 (10): 1136-1143.
18. Kudo H, Ishizawa T, Tani K, et al. Visualization of subcapsular hepatic malignancy by indocyanine-green fluorescence imaging during laparoscopic hepatectomy. Surg Endosc. 2014; 28 (8): 2504-2508.
19. Terasawa M, Ishizawa T, Mise Y, et al. Applications of fusion-fluorescence imaging using indocyanine green in laparoscopic hepatectomy. Surg Endosc. 2017; 31 (12): 5111-5118.
20. Ishizawa T, Saiura A, Kokudo N. Clinical application of indocyanine green-fluorescence imaging during hepatectomy. Hepatobiliary Surg Nutr. 2016; 5 (4): 322-328.
21. Kaibori M, Matsui K, Ishizaki M, et al. Intraoperative Detection of Superficial Liver Tumors by Fluorescence Imaging Using Indocyanine Green and 5-aminolevulinic Acid. Anticancer Res. 2016; 36 (4): 1841-1849.
22. Gotoh K, Yamada T, Ishikawa O, et al. A novel image-guided surgery of hepatocellular carcinoma by indocyanine green fluorescence imaging navigation. J Surg Oncol. 2009; 100 (1): 75-79.
23. Ishizawa T, Fukushima N, Shibahara J, et al. Real-time identification of liver cancers by using indocyanine green fluorescent imaging. Cancer. 2009; 115 (11): 2491-2504.
24. Cui Y, Konig J, Leier I, Buchholz U, Keppler D. Hepatic uptake of bilirubin and its conjugates by the human organic anion transporter SLC21A6. J Biol Chem. 2001; 276 (13): 9626-9630.
25. de Graaf W, Hausler S, Heger M, et al. Transporters involved in the hepatic uptake of (99m) Tc-mebrofenin and indocyanine green. J Hepatol. 2011; 54 (4): 738-745.
26. Slijepcevic D, Roscam Abbing R L P, Katafuchi T, et al. Hepatic uptake of conjugated bile acids is mediated by both sodium taurocholate cotransporting polypeptide and organic anion transporting polypeptides and modulated by intestinal sensing of plasma bile acid levels in mice. Hepatology. 2017; 66 (5): 1631-1643.
27. Huang L, Vore M. Multidrug resistance p-glycoprotein 2 is essential for the biliary excretion of indocyanine green. Drug Metab Dispos. 2001; 29 (5): 634-637.
28. Shibasaki Y, Sakaguchi T, Hiraide T, et al. Expression of indocyanine green-related transporters in hepatocellular carcinoma. J Surg Res. 2015; 193 (2): 567-576.
29. Lake C M, Bondoc A J, Dasgupta R, et al. Indocyanine green is a sensitive adjunct in the identification and surgical management of local and metastatic hepatoblastoma. Cancer Med. 2021; 10 (13): 4322-4343.
30. Cho Y J, Namgoong J M, Kwon H H, Kwon Y J, Kim D Y, Kim S C. The Advantages of Indocyanine Green Fluorescence Imaging in Detecting and Treating Pediatric Hepatoblastoma: A Preliminary Experience. Front Pediatr. 2021; 9:635394.
31. Munkley J, Elliott D J. Hallmarks of glycosylation in cancer. Oncotarget. 2016; 7 (23): 35478-35489.
32. Bahri H, Laurence L, Edeline J, et al. High prognostic value of ¹⁸F-FDG PET for metastatic gastroenteropancreatic neuroendocrine tumors: a long-term evaluation. J Nucl Med. 2014; 55 (11): 1786-1790.
33. Warburg O. On the origin of cancer cells. Science. 1956; 123 (3191): 309-314.
34. Kaneko J, Inagaki Y, Ishizawa T, et al. Photodynamic therapy for human hepatoma-cell-line tumors utilizing biliary excretion properties of indocyanine green. J Gastroenterol. 2014; 49 (1): 110-116.
35. Feng J, Lu H, Ma W, et al. Genome-wide CRISPR screen identifies synthetic lethality between DOCK1 inhibition and metformin in liver cancer. Protein Cell. 2022; 13 (11): 825-841.
36. Shirata C, Kaneko J, Inagaki Y, et al. Near-infrared photothermal/photodynamic therapy with indocyanine green induces apoptosis of hepatocellular carcinoma cells through oxidative stress. Scientific reports. 2017; 7 (1): 13958.
37. Chhabra A, Thakur M L. Theragnostic Radionuclide Pairs for Prostate Cancer Management: (64) Cu/(67) Cu, Can Be a Budding Hot Duo. Biomedicines. 2022; 10 (11).
38. Novak-Hofer I, Schubiger P A. Copper-67 as a therapeutic nuclide for radioimmunotherapy. Eur J Nucl Med Mol Imaging. 2002; 29 (6): 821-830.

### INCORPORATION BY REFERENCE

The present application refers to various issued patent, published patent applications, scientific journal articles, and other publications, all of which are incorporated herein by reference. The details of one or more embodiments of the disclosure are set forth herein. Other features, objects, and advantages of the disclosure will be apparent from the Detailed Description, the Figures, the Examples, and the Claims.

### Equivalents and Scope

In the claims and throughout, articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the disclosure can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present disclosure, as defined in the following claims.

What is claimed is:

1. A compound of Formula I: 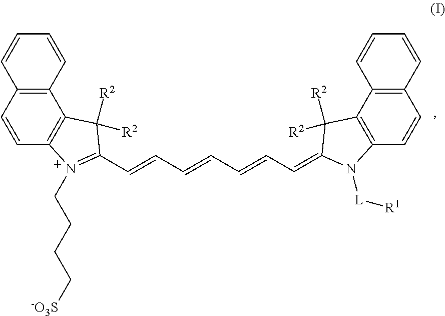
or a pharmaceutically acceptable salt, solvate, hydrate, polymorph, co-crystal, tautomer, stereoisomer, or prodrug thereof, wherein:
L is a linker;
R¹ is halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂, and contains at least one radioisotope;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring; and
each occurrence of R² is independently hydrogen, halogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl, or two occurrences of R² are joined together with their intervening atom to form an optionally substituted carbocyclic ring or optionally substituted heterocyclic ring.

2. A compound of Formula I′: 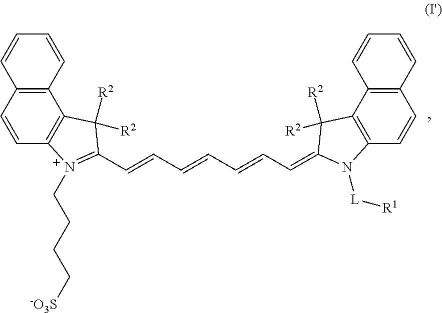
or a pharmaceutically acceptable salt, solvate, hydrate, polymorph, co-crystal, tautomer, stereoisomer, or prodrug thereof, wherein:
L is a linker;
R¹ is halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring; and
each occurrence of R² is independently hydrogen, halogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl, or two occurrences of R² are joined together with their intervening atom to form an optionally substituted carbocyclic ring or optionally substituted heterocyclic ring.

3. The compound of any one of the preceding claims, wherein the compound is of Formula I-a-i, Formula I-a-ii, or Formula I-a-iii: 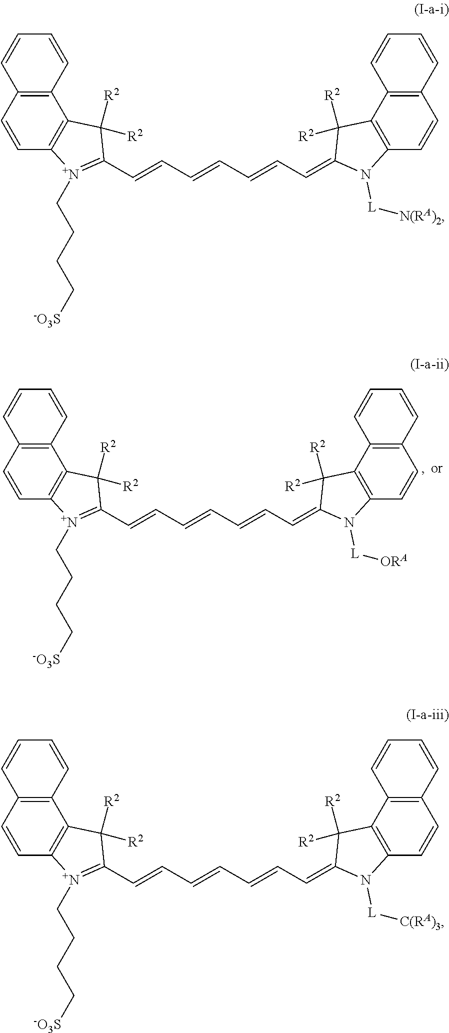
or a pharmaceutically acceptable salt, solvate, hydrate, polymorph, co-crystal, tautomer, stereoisomer, or prodrug thereof.

4. The compound of any one of the preceding claims, wherein the compound is of Formula I-b: 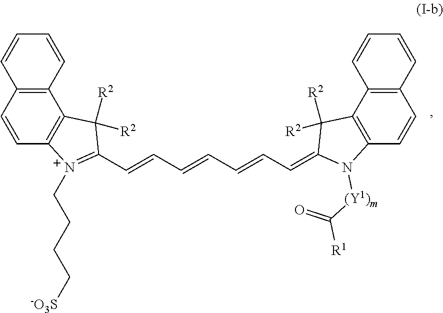
or a pharmaceutically acceptable salt, solvate, hydrate, polymorph, co-crystal, tautomer, stereoisomer, or prodrug thereof, wherein:
each occurrence of Y¹ is independently C(R^{E1})₂, NR^{E2}, or —O—;
each occurrence of R^{E1} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂, or two occurrences of R^{E1} are optionally joined together with the intervening atoms to form an optionally substituted cyclic moiety;
each occurrence of R^{E2} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, or a nitrogen protecting group, or two occurrences of R^{E2} are optionally joined together with their intervening atoms to form an optionally substituted cyclic moiety; and
m is 0, 1, 2, 3, 4, 5, 6, 7, 8, 9, or 10.

5. The compound of any one of the preceding claims, wherein the compound is of Formula I-b-i: 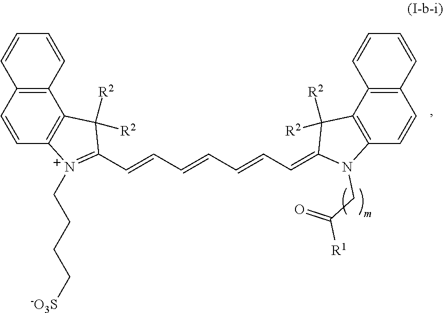
or a pharmaceutically acceptable salt, solvate, hydrate, polymorph, co-crystal, tautomer, stereoisomer, or prodrug thereof.

6. The compound of any one of the preceding claims, wherein the compound is of Formula I-c: 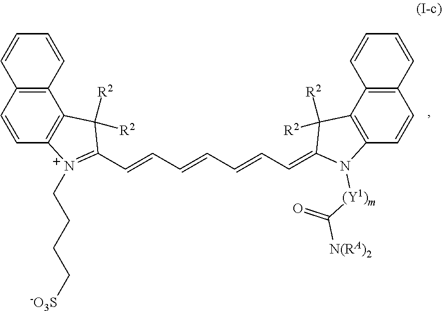
or a pharmaceutically acceptable salt, solvate, hydrate, polymorph, co-crystal, tautomer, stereoisomer, or prodrug thereof.

7. The compound of any one of the preceding claims, wherein the compound is of Formula I-c-i: 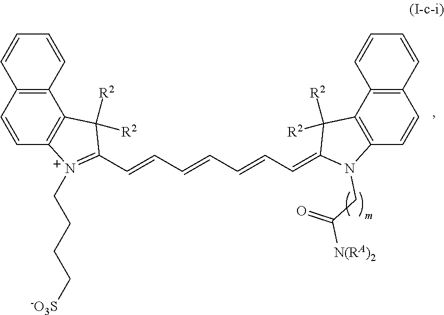
or a pharmaceutically acceptable salt, solvate, hydrate, polymorph, co-crystal, tautomer, stereoisomer, or prodrug thereof.

8. The compound of any one of the preceding claims, wherein the compound is of Formula I-d: 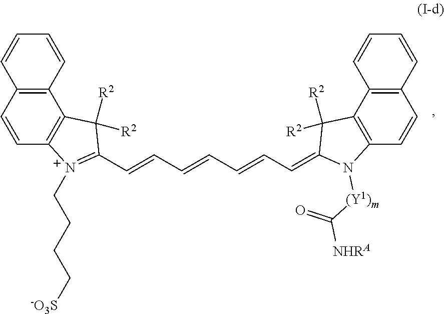
or a pharmaceutically acceptable salt, solvate, hydrate, polymorph, co-crystal, tautomer, stereoisomer, or prodrug thereof.

9. The compound of any one of the preceding claims, wherein the compound is of Formula I-d-i: 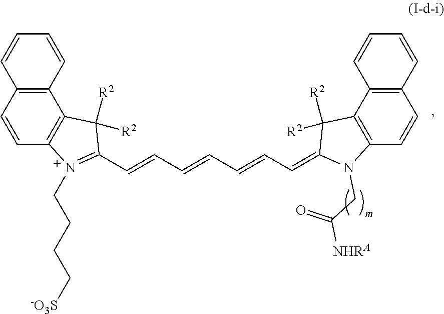
or a pharmaceutically acceptable salt, solvate, hydrate, polymorph, co-crystal, tautomer, stereoisomer, or prodrug thereof.

10. The compound of any one of the preceding claims, wherein each occurrence of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl.

11. The compound of any one of the preceding claims, wherein at least one occurrence of R^{A} is optionally substituted alkyl.

12. The compound of any one of the preceding claims, wherein at least one occurrence of R^{A} is alkyl substituted with one or more optionally substituted amino groups.

13. The compound of any one of the preceding claims, wherein at least one occurrence of R^{A} is alkyl substituted with one or more acyl groups.

14. The compound of any one of the preceding claims, wherein at least one occurrence of R^{A} is alkyl substituted with halogen.

15. The compound of any one of the preceding claims, wherein at least one occurrence of R^{A} is alkyl substituted with fluorine.

16. The compound of any one of the preceding claims, wherein at least one occurrence of R^{A} is alkyl substituted with ¹⁸F.

17. The compound of any one of the preceding claims, wherein the compound is of Formula I-e: 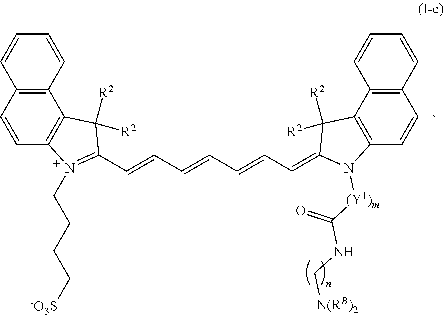
or a pharmaceutically acceptable salt, solvate, hydrate, polymorph, co-crystal, tautomer, stereoisomer, or prodrug thereof, wherein:
each occurrence of R^{B} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or two occurrences of R^{B} are joined together with their intervening atom to form an optionally substituted heterocyclic ring or optionally substituted heteroaryl ring; and
n is 1, 2, 3, 4, 5, 6, 7, 8, 9, or 10.

18. The compound of any one of the preceding claims, wherein the compound is of Formula I-e-i: 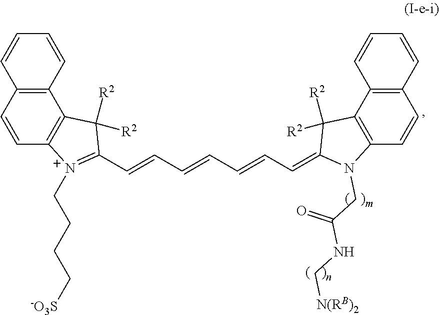
or a pharmaceutically acceptable salt, solvate, hydrate, polymorph, co-crystal, tautomer, stereoisomer, or prodrug thereof.

19. The compound of any one of the preceding claims, wherein the compound is of Formula I-f: 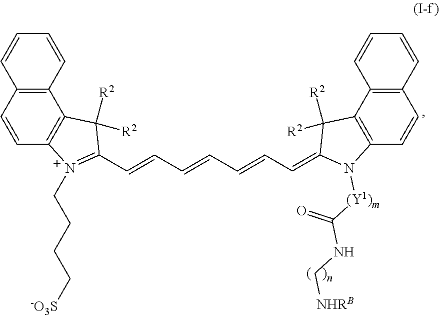
or a pharmaceutically acceptable salt, solvate, hydrate, polymorph, co-crystal, tautomer, stereoisomer, or prodrug thereof.

20. The compound of any one of the preceding claims, wherein the compound is of Formula I-f-i: 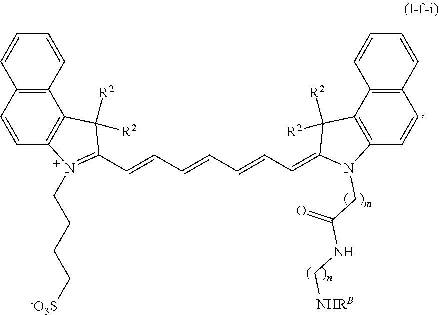
or a pharmaceutically acceptable salt, solvate, hydrate, polymorph, co-crystal, tautomer, stereoisomer, or prodrug thereof.

21. The compound of any one of the preceding claims, wherein the compound is of Formula I-g: 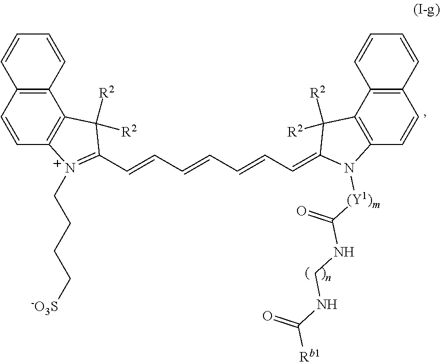
or a pharmaceutically acceptable salt, solvate, hydrate, polymorph, co-crystal, tautomer, stereoisomer, or prodrug thereof, wherein:
R^{b1} is optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl.

22. The compound of any one of the preceding claims, wherein the compound is of Formula I-g-i: 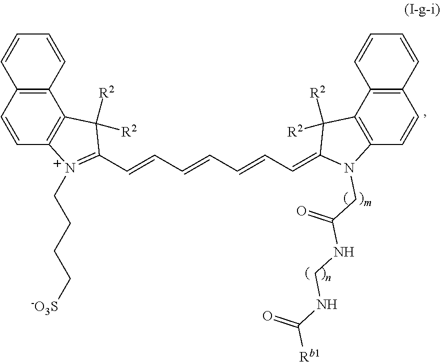
or a pharmaceutically acceptable salt, solvate, hydrate, polymorph, co-crystal, tautomer, stereoisomer, or prodrug thereof.

23. The compound of any one of the preceding claims, wherein R^{b1} is optionally substituted aryl or optionally substituted heteroaryl.

24. The compound of any one of the preceding claims, wherein R^{b1} is aryl substituted with halogen.

25. The compound of any one of the preceding claims, wherein R^{b1} is aryl substituted with fluorine.

26. The compound of any one of the preceding claims, wherein R^{b1} is aryl substituted with ¹⁸F.

27. The compound of any one of the preceding claims, wherein R^{b1} is heteroaryl substituted with halogen.

28. The compound of any one of the preceding claims, wherein R^{b1} is heteroaryl substituted with fluorine.

29. The compound of any one of the preceding claims, wherein R^{b1} is heteroaryl substituted with ¹⁸F.

30. The compound of any one of the preceding claims, wherein the compound is of Formula I-h: 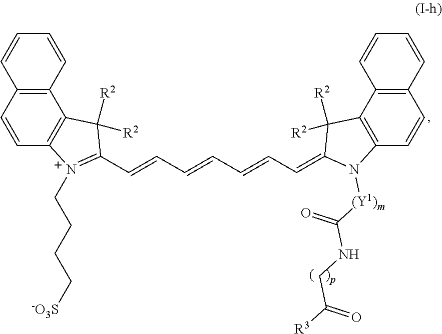
or a pharmaceutically acceptable salt, solvate, hydrate, polymorph, co-crystal, tautomer, stereoisomer, or prodrug thereof, wherein:
R³ is halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —ORC, —SCN, —SR^{C}, —SSR^{C}, —N₃, —NO, —N(R^{C})₂, —NO₂, —C(═O)R^{C}, —C(═O)OR^{C}, —C(═O)SR^{C}, —C(═O)N(R^{C})₂, —C(═NR^{C})R^{C}, —C(═NR^{C})OR^{C}, —C(═NR^{C})SR^{C}, —C(═NR^{C})N(R^{C})₂, —S(═O)R^{C}, —S(═O)OR^{C}, —S(═O)SR^{C}, —S(═O)N(R^{C})₂, —S(═O)₂R^{C}, —S(═O)₂OR^{C}, —S(═O)₂SR^{C}, —S(═O)₂N(R^{C})₂, —OC(═O)R^{C}, —OC(═O)OR^{C}, —OC(═O)SR^{C}, —OC(═O)N(R^{C})₂, —OC(═NR^{C})R^{C}, —OC(═NR^{C})OR^{C}, —OC(═NR^{C})SR^{C}, —OC(═NR^{C})N(R^{C})₂, —OS(═O)R^{C}, —OS(═O)OR^{C}, —OS(═O)SR^{C}, —OS(═O)N(R^{C})₂, —OS(═O)₂R^{C}, —OS(═O)₂OR^{C}, —OS(═O)₂SR^{C}, —OS(═O)₂N(R^{C})₂, —ON(R^{C})₂, —SC(═O)R^{C}, —SC(═O)OR^{C}, —SC(═O)SR^{C}, —SC(═O)N(R^{C})₂, —SC(═NR^{C})R^{C}, —SC(═NR^{C})OR^{C}, —SC(═NR^{C})SR^{C}, —SC(═NR^{C})N(R^{C})₂, —NR^{C}C(═O)R^{C}, —NR^{C}C(═O)OR^{C}, —NR^{C}C(═O)SR^{C}, —NR^{C}C(═O)N(R^{C})₂, —NR^{C}C(═NR^{C})R^{C}, —NR^{C}C(═NR^{C})OR^{C}, —NR^{C}C(═NR^{C})SR^{C}, —NR^{C}C(═NR^{C})N(R^{C})₂, —NRCS(═O)R^{C}, —NRCS(═O)OR^{C}, —NRCS(═O)SR^{C}, —NRCS(═O)N(R^{C})₂, —NRCS(═O)₂R^{C}, —NRCS(═O)₂OR^{C}, —NRCS(═O)₂SR^{C}, —NRCS(═O)₂N(R^{C})₂, —Si(R^{C})₃, —Si(R^{C})₂OR^{C}, —Si(R^{C}) (OR^{C})₂, —Si(OR^{C})₃, —OSi(R^{C})₃, —OSi(R^{C})₂OR^{C}, —OSi(R^{C}) (OR^{C})₂, —OSi(OR^{C})₃, or —B(OR^{C})₂;
each occurrence of R^{C} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{C} are joined together with their intervening atom to form an optionally substituted heterocyclic ring or optionally substituted heteroaryl ring; and
p is 0, 1, 2, 3, 4, 5, 6, 7, 8, 9, or 10.

31. The compound of any one of the preceding claims, wherein the compound is of Formula I-h-i: 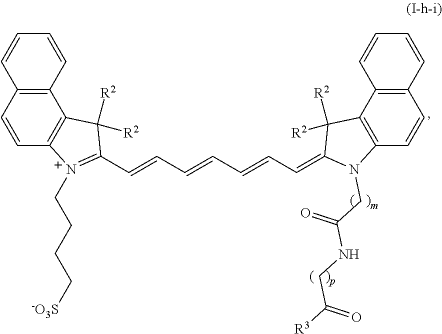
or a pharmaceutically acceptable salt, solvate, hydrate, polymorph, co-crystal, tautomer, stereoisomer, or prodrug thereof.

32. The compound of any one of the preceding claims, wherein R³ is optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl.

33. The compound of any one of the preceding claims, wherein R³ is optionally substituted heterocyclyl.

34. The compound of any one of the preceding claims, wherein R³ is an optionally substituted azacyclooctyne.

35. The compound of any one of the preceding claims, wherein R³ is an optionally substituted aza-dibenzocyclooctyne.

36. The compound of any one of the preceding claims, wherein the compound is of Formula I-h-ii or I-h-iii: 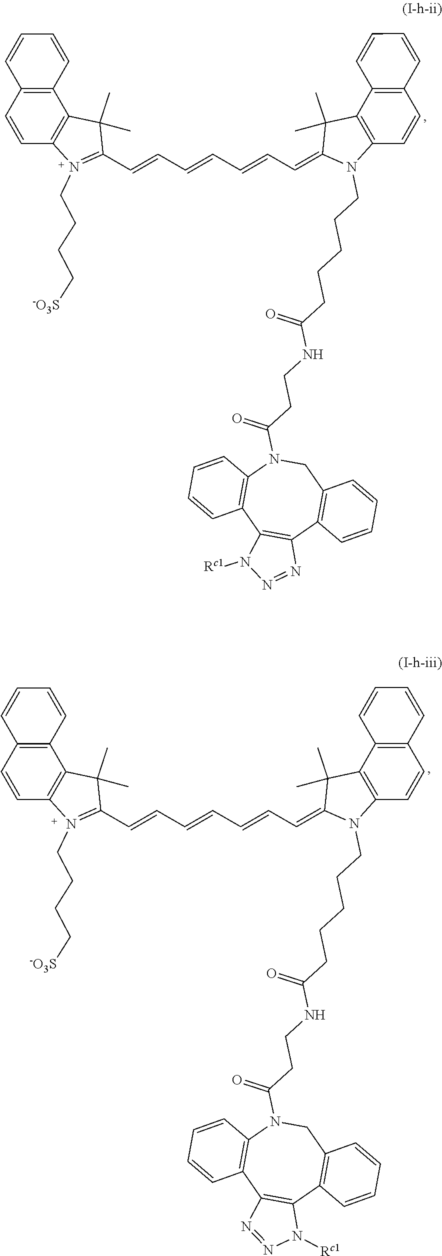
or a pharmaceutically acceptable salt, solvate, hydrate, polymorph, co-crystal, tautomer, stereoisomer, or prodrug thereof, wherein:
R^{c1} is optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl.

37. The compound of any one of the preceding claims, wherein R^{c1} is optionally substituted alkyl.

38. The compound of any one of the preceding claims, wherein R^{c1} is alkyl substituted with halogen.

39. The compound of any one of the preceding claims, wherein R^{c1} is alkyl substituted with fluorine.

40. The compound of any one of the preceding claims, wherein R^{c1} is alkyl substituted with ¹⁸F.

41. The compound of any one of the preceding claims, wherein the compound is of Formula 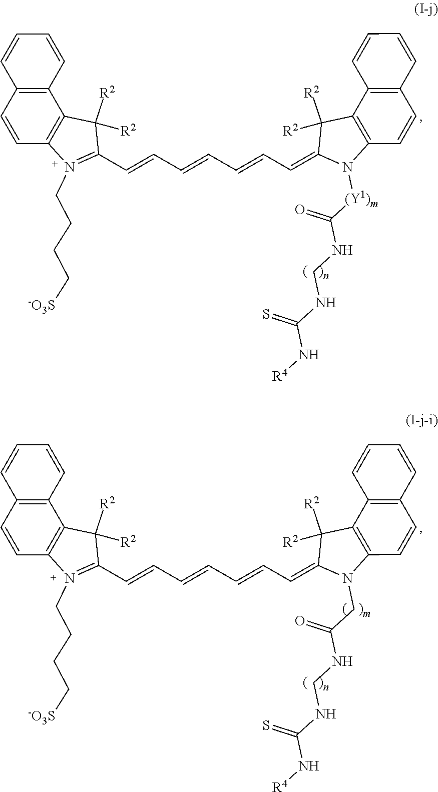
or a pharmaceutically acceptable salt, solvate, hydrate, polymorph, co-crystal, tautomer, stereoisomer, or prodrug thereof, wherein:
R⁴ is optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl.

42. The compound of claim 41, wherein R⁴ is optionally substituted aryl.

43. The compound of claim 41 or 42, wherein R⁴ is phenyl substituted with optionally substituted alkyl substituted with optionally substituted heterocyclyl.

44. The compound of any of claims 41-43, wherein R⁴ comprises a chelating moiety.

45. The compound of any of claims 41-44, wherein R⁴ comprises

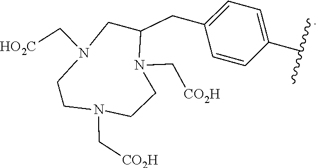 [SMILES: *c1ccc(CC2CN(CC(=O)O)CCN(CC(=O)O)CCN2CC(=O)O)cc1]

46. The compound of any of claims 41-45, wherein R⁴ is

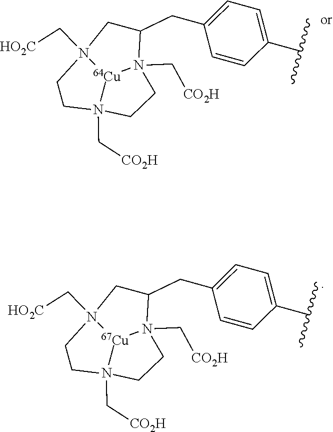

47. The compound of any one of the preceding claims, wherein m is 5.

48. The compound of any one of the preceding claims, wherein n is 2.

49. The compound of any one of the preceding claims, wherein p is 2.

50. The compound of any one of the preceding claims, or pharmaceutically acceptable salt, solvate, hydrate, polymorph, co-crystal, tautomer, stereoisomer, or prodrug thereof, wherein the at least one radioisotope is ¹¹C, ¹⁸F, ¹³N, ¹⁵O, ⁶⁴Cu, ⁶⁷Cu, ⁶⁷Ga, ⁶⁸Ga, ⁷⁵Se, ^{81m}Kr, ⁸²Rb, ^{99m}Tc, ¹¹¹In, ¹²³I, ¹²⁵I, ¹³¹I, or ¹³³Xe.

51. The compound of any one of the preceding claims, or pharmaceutically acceptable salt, solvate, hydrate, polymorph, co-crystal, tautomer, stereoisomer, or prodrug thereof, wherein the at least one radioisotope is ¹¹C, ¹⁸F, ¹³N, ¹⁵O, ⁷⁵Se, ¹²³I, ¹²⁵I, or ¹³¹I.

52. The compound of any one of the preceding claims, or pharmaceutically acceptable salt, solvate, hydrate, polymorph, co-crystal, tautomer, stereoisomer, or prodrug thereof, wherein the radioisotope is ¹⁸F.

53. The compound of any one of the preceding claims, wherein the compound is of formula: 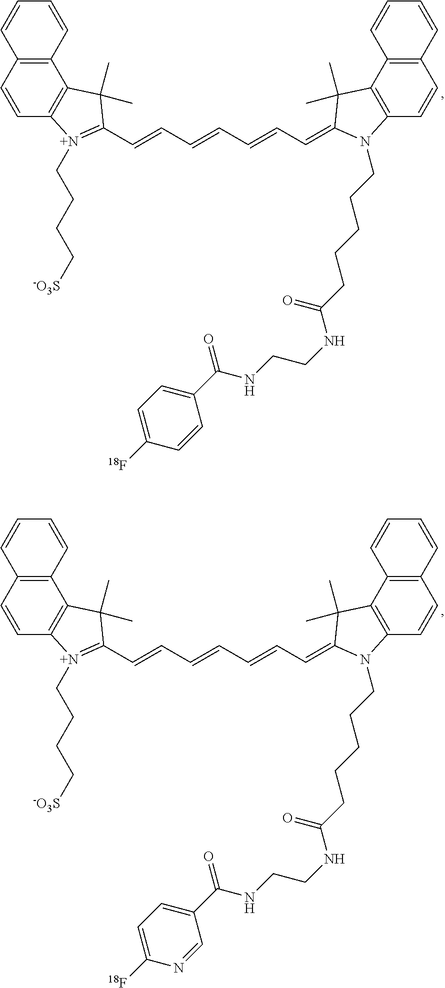 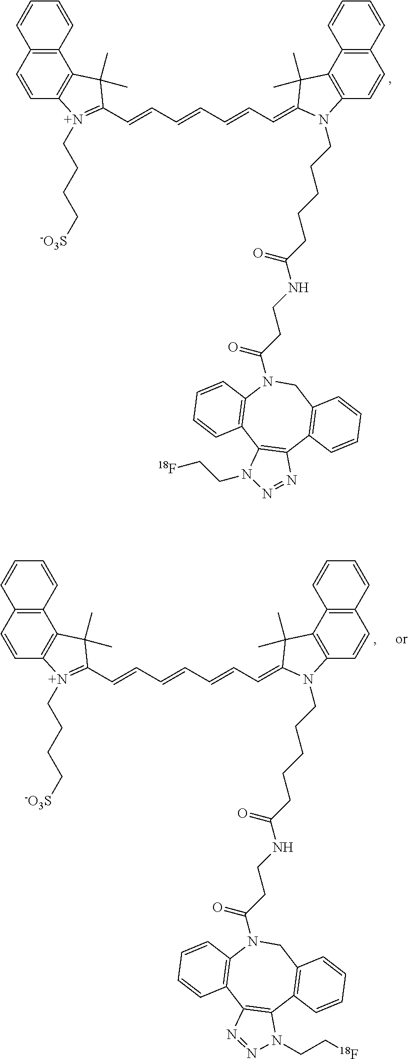 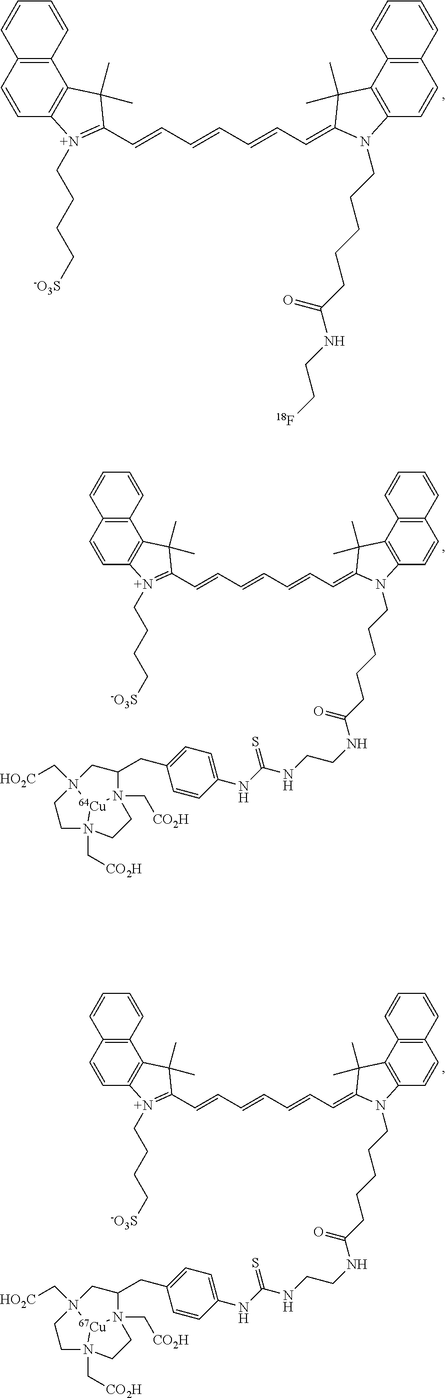 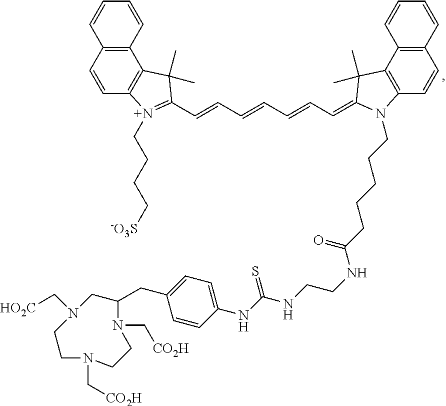
or a pharmaceutically acceptable salt, solvate, hydrate, polymorph, co-crystal, tautomer, stereoisomer, or prodrug thereof.

54. The compound of any one of the preceding claims, wherein the compound degrades less than 10% one hour after preparing the compound.

55. The compound of any one of the preceding claims, wherein the compound degrades less than 10% two hours after preparing the compound.

56. A pharmaceutical composition comprising:
the compound of any one of the preceding claims, or pharmaceutically acceptable salt, solvate, hydrate, polymorph, co-crystal, tautomer, stereoisomer, or prodrug thereof; and
a pharmaceutically acceptable excipient.

57. The pharmaceutical composition of claim 56 further comprising one or more additional pharmaceutical agents.

58. A method of diagnosing a disease in a subject in need thereof comprising administering to the subject an effective amount of:
the compound of any one of claims 1-55, or pharmaceutically acceptable salt, solvate, hydrate, polymorph, co-crystal, tautomer, stereoisomer, or prodrug thereof; or
the pharmaceutical composition of any one of claim 56 or 57.

59. The compound of any one of claims 1-55, or pharmaceutically acceptable salt, solvate, hydrate, polymorph, co-crystal, tautomer, stereoisomer, or prodrug thereof, or the pharmaceutical composition of any one of claim 56 or 57, for use in diagnosing a disease in a subject in need thereof.

60. The compound of any one of claims 1-55, or pharmaceutically acceptable salt, solvate, hydrate, polymorph, co-crystal, tautomer, stereoisomer, or prodrug thereof, or the pharmaceutical composition of any one of claim 56 or 57, for use in the manufacture of a medicament for diagnosis of a disease in a subject in need thereof.

61. The method or compound for use, or pharmaceutically acceptable salt, solvate, hydrate, polymorph, co-crystal, tautomer, stereoisomer, or prodrug thereof for use, or pharmaceutical composition for use of any one of claims 58-60, comprising imaging a tissue.

62. The method or compound for use, or pharmaceutically acceptable salt, solvate, hydrate, polymorph, co-crystal, tautomer, stereoisomer, or prodrug thereof for use, or pharmaceutical composition for use of any one of claims 58-61, comprising imaging liver tissue.

63. The method or compound for use, or pharmaceutically acceptable salt, solvate, hydrate, polymorph, co-crystal, tautomer, stereoisomer, or prodrug thereof for use, or pharmaceutical composition for use of any one of claims 58-62, comprising imaging through positron emission tomography (PET).

64. The method or compound for use, or pharmaceutically acceptable salt, solvate, hydrate, polymorph, co-crystal, tautomer, stereoisomer, or prodrug thereof for use, or pharmaceutical composition for use of any one of claims 58-63, comprising imaging through computed tomography (CT).

65. A method of treating a disease in a subject in need thereof comprising administering to the subject an effective amount of:
the compound of any one of claims 1-55, or pharmaceutically acceptable salt, solvate, hydrate, polymorph, co-crystal, tautomer, stereoisomer, or prodrug thereof; or
the pharmaceutical composition of any one of claim 56 or 57.

66. The compound of any one of claims 1-55, or pharmaceutically acceptable salt, solvate, hydrate, polymorph, co-crystal, tautomer, stereoisomer, or prodrug thereof, or the pharmaceutical composition of any one of claim 56 or 57, for use in treating a disease in a subject in need thereof.

67. The compound of any one of claims 1-55, or pharmaceutically acceptable salt, solvate, hydrate, polymorph, co-crystal, tautomer, stereoisomer, or prodrug thereof, or the pharmaceutical composition of any one of claim 56 or 57, for use in the manufacture of a medicament for treatment of a disease in a subject in need thereof.

68. A method of preventing a disease in a subject in need thereof comprising administering to the subject an effective amount of:
the compound of any one of claims 1-55, or pharmaceutically acceptable salt, solvate, hydrate, polymorph, co-crystal, tautomer, stereoisomer, or prodrug thereof; or
the pharmaceutical composition of any one of claim 56 or 57.

69. The compound of any one of claims 1-55, or pharmaceutically acceptable salt, solvate, hydrate, polymorph, co-crystal, tautomer, stereoisomer, or prodrug thereof, or the pharmaceutical composition of any one of claim 56 or 57, for use in preventing a disease in a subject in need thereof.

70. The compound of any one of claims 1-55, or pharmaceutically acceptable salt, solvate, hydrate, polymorph, co-crystal, tautomer, stereoisomer, or prodrug thereof, or the pharmaceutical composition of any one of claim 56 or 57, for use in the manufacture of a medicament for prevention of a disease in a subject in need thereof.

71. A method of inhibiting cell proliferation or promoting apoptosis in a subject in need thereof or in a cell, tissue, or biological sample, comprising administering to the subject or contacting the cell, tissue, or biological sample with an effective amount of:
the compound of any one of claims 1-55, or pharmaceutically acceptable salt, solvate, hydrate, polymorph, co-crystal, tautomer, stereoisomer, or prodrug thereof; or
the pharmaceutical composition of any one of claim 56 or 57;
wherein the cell, tissue, or biological sample is in vivo.

72. A method of inhibiting cell proliferation or promoting apoptosis in a subject in need thereof or in a cell, tissue, or biological sample, comprising administering to the subject or contacting the cell, tissue, or biological sample with an effective amount of:
the compound of any one of claims 1-55, or pharmaceutically acceptable salt, solvate, hydrate, polymorph, co-crystal, tautomer, stereoisomer, or prodrug thereof; or
the pharmaceutical composition of any one of claim 56 or 57;
wherein the cell, tissue, or biological sample is in vitro.

73. A method of imaging a subject in need thereof comprising administering to the subject an effective amount of:
the compound of any one of claims 1-55, or pharmaceutically acceptable salt, solvate, hydrate, polymorph, co-crystal, tautomer, stereoisomer, or prodrug thereof; or
the pharmaceutical composition of any one of claim 56 or 57; and
detecting the at least one radioisotope.

74. The compound of any one of claims 1-55, or pharmaceutically acceptable salt, solvate, hydrate, polymorph, co-crystal, tautomer, stereoisomer, or prodrug thereof, or the pharmaceutical composition of any one of claim 56 or 57, for use in diagnosing a disease in a subject in need thereof.

75. The compound of any one of claims 1-55, or pharmaceutically acceptable salt, solvate, hydrate, polymorph, co-crystal, tautomer, stereoisomer, or prodrug thereof, or the pharmaceutical composition of any one of claim 56 or 57, for use in the manufacture of a medicament for diagnosis of a disease in a subject in need thereof.

76. The method or compound for use, or pharmaceutically acceptable salt, solvate, hydrate, polymorph, co-crystal, tautomer, stereoisomer, or prodrug thereof for use, or pharmaceutical composition for use of any one of claims 58-75, wherein the disease is a cancer.

77. The method or compound for use, or pharmaceutically acceptable salt, solvate, hydrate, polymorph, co-crystal, tautomer, stereoisomer, or prodrug thereof for use, or pharmaceutical composition for use of any one of claims 58-76, wherein the disease is liver cancer.

78. The method or compound for use, or pharmaceutically acceptable salt, solvate, hydrate, polymorph, co-crystal, tautomer, stereoisomer, or prodrug thereof for use, or pharmaceutical composition for use of any one of claims 58-77, wherein the liver cancer is hepatocellular carcinoma (HCC).

79. The method or compound for use, or pharmaceutically acceptable salt, solvate, hydrate, polymorph, co-crystal, tautomer, stereoisomer, or prodrug thereof for use, or pharmaceutical composition for use of any one of claims 58-78, wherein the subject is a human.

80. The method or compound for use, or pharmaceutically acceptable salt, solvate, hydrate, polymorph, co-crystal, tautomer, stereoisomer, or prodrug thereof for use, or pharmaceutical composition for use of any one of claims 58-79, wherein the subject has been administered one or more additional pharmaceutical agents and/or radiation.

81. The method or compound for use, or pharmaceutically acceptable salt, solvate, hydrate, polymorph, co-crystal, tautomer, stereoisomer, or prodrug thereof for use, or pharmaceutical composition for use of any one of claims 58-80, wherein the subject suffers from underlying liver disease, chronic hepatitis, and/or cirrhosis.

82. The method or compound for use, or pharmaceutically acceptable salt, solvate, hydrate, polymorph, co-crystal, tautomer, stereoisomer, or prodrug thereof for use, or pharmaceutical composition for use of any one of claims 58-81, wherein the compound is detected in the liver within 15 minutes of administration, within 30 minutes of administration, within 45 minutes of administration, within 1 hour of administration, within 2 hours of administration, within 3 hours of administration, or within 4 hours of administration.

83. The method or compound for use, or pharmaceutically acceptable salt, solvate, hydrate, polymorph, co-crystal, tautomer, stereoisomer, or prodrug thereof for use, or pharmaceutical composition for use of any one of claims 58-82, wherein the activity concentration, as measured by standardized uptake value (SUV), in the liver after administration is at least 5, at least 10, at least 15, at least 20, or at least 25.

84. The method or compound for use, or pharmaceutically acceptable salt, solvate, hydrate, polymorph, co-crystal, tautomer, stereoisomer, or prodrug thereof for use, or pharmaceutical composition for use of any one of claims 58-83, wherein the activity uptake, as measured by percentage injected dose (% ID), in the liver after administration is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99%.

85. A method of preparing a compound of Formula I-g-i: 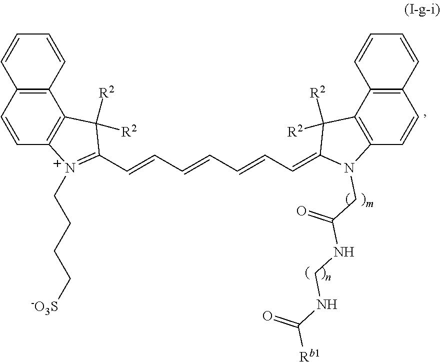
or pharmaceutically acceptable salt, solvate, hydrate, polymorph, co-crystal, tautomer, stereoisomer, or prodrug thereof, the method comprising reacting an amine of Formula I-f-ii: 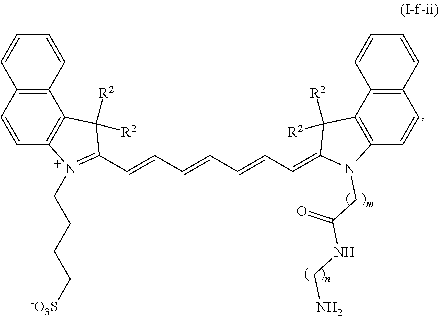
or pharmaceutically acceptable salt, solvate, hydrate, polymorph, co-crystal, tautomer, stereoisomer, or prodrug thereof, with a compound of Formula II: 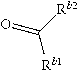
or salt thereof, wherein:
each occurrence of R² is independently hydrogen, halogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl, or two occurrences of R² are joined together with their intervening atom to form an optionally substituted carbocyclic ring or optionally substituted heterocyclic ring;
R^{b1} is optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl, and contains at least one radioisotope;
R^{b2} is halogen or —ORA;
R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl;
m is 0, 1, 2, 3, 4, 5, 6, 7, 8, 9, or 10; and
n is 1, 2, 3, 4, 5, 6, 7, 8, 9, or 10.

86. The method of claim 85, wherein the compound of Formula II is an activated ester of Formula II-a or Formula II-b: 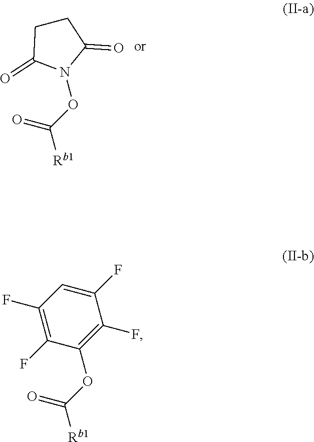
or salt thereof.

87. The method of any one of claim 85 or 86, further comprising converting an ester of Formula II-c: 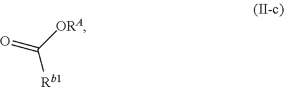
or salt thereof, to an activated ester of Formula II-a or Formula II-b: 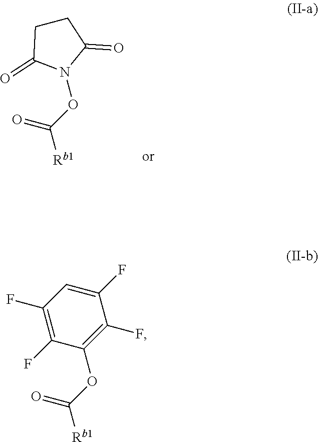
or salt thereof.

88. The method of any one of claims 85-87, comprising reacting the ester of formula II-c: 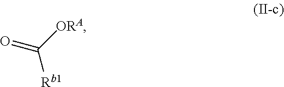
or salt thereof, with 2-(2,5-dioxopyrrolidin-1-yl)-1,1,3,3-tetramethylisouronium tetrafluoroborate (TSTU) to provide the activated ester of Formula II-a: 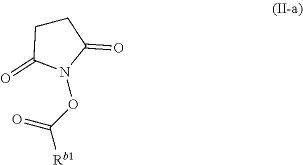
or salt thereof.

89. The method of any one of claims 85-88, further comprising reacting a compound of Formula II-d: 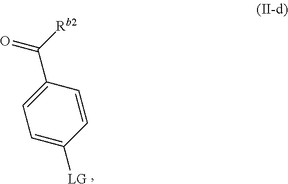
or salt thereof, with ¹⁸F⁻ to provide a compound of Formula II-e: 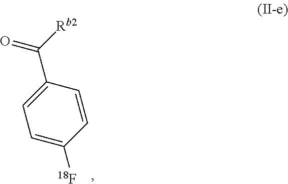
or salt thereof, wherein:
LG is a leaving group.

90. The method of claim 89, wherein the leaving group is NMe₃.

91. The method of any one of claim 89 or 90, wherein reacting the compound of Formula II-d with ¹⁸F⁻ comprises adding [₁₈F]KF/K222.

92. The method of any one of claim 89 or 90, wherein reacting the compound of Formula II-d with ¹⁸F⁻ comprises adding [¹⁸F]TBAF.

93. A kit comprising:
the compound of any one of claims 1-55, or pharmaceutically acceptable salt, solvate, hydrate, polymorph, co-crystal, tautomer, stereoisomer, or prodrug thereof; or the pharmaceutical composition of any one of claim 56 or 57; and
instructions for using the compound, or pharmaceutically acceptable salt, solvate, hydrate, polymorph, co-crystal, tautomer, stereoisomer, or prodrug thereof, or pharmaceutical composition.
